(19) United States
(12) Patent Application Publication
(10) Pub. No.: US 2024/0327320 A1
(43) Pub. Date: Oct. 3, 2024

(54) NOVEL ROUTES OF CHEMICAL SYNTHESIS OF 20S(OH)D3, 20S,25(OH)2D3, 20S,23S(OH)2D3, AND 20S,23R(OH)2D3 AND THEIR MODIFICATION OF THE IMMUNE ACTIVITY OF PBMCS

(71) Applicants: United State Government as represen ted by the Department of Veterans Affairs, Washington, DC (US); University of Warsaw, Warszawa (PL)

(72) Inventors: Andrzej Slominski, Birmingham, AL (US); Pawel Brzeminski, Mogielnica (PL); Adrian Fabisiak, Nowy Dwor Mazowiecki (PL)

(21) Appl. No.: 18/433,045

(22) Filed: Feb. 5, 2024

Related U.S. Application Data
(60) Provisional application No. 63/483,530, filed on Feb. 6, 2023.
(60) Provisional application No. 63/546,086, filed on Oct. 27, 2023.

(51) Int. Cl. C07C 35/21; C07C 29/48

(52) U.S. Cl. CPC C07C 35/21; C07C 29/48; C07C 2601/14; C07C 2602/24

(57) ABSTRACT

The present disclosure is concerned with methods of making hydroxy derivatives of vitamin D₃, compounds useful as intermediates in the preparation of the hydroxy derivatives, and methods of making the intermediates. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/483,530, filed on Feb. 6, 2023, and U.S. Application No. 63/546,086, filed on Oct. 27, 2023, the contents of which are hereby incorporated by reference in their entireties.

### STATEMENT REGARDING FEDERALLY SPONSORED RESEARCH

This invention was made with government support under grant numbers 1R01AR073004-01A1 and R01AR071189-01A1 awarded by the National Institutes of Health (NIH) and grant numbers 1I01BX004293-01A1 and A1152047-01A1 awarded by the U.S. Department of Veterans Affairs. The government has certain rights in the invention.

### BACKGROUND

Vitamin D₃ (1) (FIG. 1) is formed from 7-dehydrocholesterol (7DHC) by the action of ultraviolet (UVB) radiation (Holick M F, et. al. (1981), Science 211(4482) 590-3; Holick M F, et. al., (1995) Proc Natl Acad Sci USA 92(8) 3124-6; Bikle D D, (2011) Exp Dermatol 20(1) 7-13; Bikle D D, (2010) Trends Endocrinol Metab 21(6) 375-84). The electrocyclic B-ring opening generates previtamin D₃, which is converted to vitamin D₃ by a first-order thermal isomerization in the skin (Holick M F, et. al., (1981) Science 211(4482) (1981) 590-3; Holick M F, et. al., (1995) Proc Natl Acad Sci USA 92(8) (1995) 3124-6; Bikle D D, (2011) Exp Dermatol 20(1) 7-13). Vitamin D₃ is activated by hydroxylation at C-25 by CYP2R1 or CYP27A1 then at C1α by CYP27B1, to produce 1α,25(OH)₂D₃ (2) (Bikle D D, (2010) Trends Endocrinol Metab 21(6) 375-84, Holick M F, (2003) J Cell Biochem 88(2) 296-307; Jenkinson C, (2019) Cell Biochem Funct; Tuckey R C, et. al., (2019) J Steroid Biochem Mol Biol 186 (2019) 4-21). In addition to regulating calcium homeostasis, 1α,25(OH)₂D₃ has pleiotropic effects affecting almost all body functions through an interaction with vitamin D receptor (VDR) (Bikle D D, (2010) Trends Endocrinol Metab 21(6) 375-84; Holick M F, (2003) J Cell Biochem 88(2) 296-307; Bikle D D, (2008) J Invest Dermatol 128(10) 2357-61; Bikle D D, (2011) Mol Cell Endocrinol 347(1-2) 80-9, Holick M F, (2007) N Engl J Med 357(3) 266-81; Plum L A, et. al., (2010) Nat Rev Drug Discov 9(12) 941-55; Bikle D D, et. al., (2020) J Endocr Soc 4(2) bvz038; Carlberg C, (2018) Front Endocrinol (Lausanne) 9 250; Zmijewski M A, Carlberg C, (2020) Exp Dermatol, Bouillon R, et. al., (2018) Endocr Rev 40(4) 1109-1151; Slominski A T, et. al., (2017), Lab Invest 97(6) 706-724). 1α,25(OH)₂D₃ regulates cell proliferation and differentiation, exhibits anticancer properties, regulates skin functions, inhibits adaptive immunity, and upregulates innate immune responses among other physiological functions (Holick M F, (2007) N Engl J Med 357(3) 266-81; Hewison M, et. al., (2012) Clin Endocrinol 76(3) 315-325; Suaini N H A, et. al., (2015) Nutrients 7(8) 6088-6108; Bikle D D, et. al., (2012) Rev Endocr Metab Disord 13(1) 3-19; Reichrath J, et. al., (2017) Mol Cell Endocrinol 453 96-102; Grant W B, et. al., (2016) Anticancer Res 36(3) 1357-70). It is clinically used to treat autoimmune and inflammatory diseases, including various skin disorders (Chang S H, et. al., (2010) J Biol Chem 285(50) 38751-38755; Szodoray P, et. al., (2008) Scand J Immunol 68(3) 261-9; Daniel C, et. al., (2008) J Pharmacol Exp Ther 324(1) 23-33; Wobke T K, et. al., (2014) Front Physiol 5 244, Chun R F, et. al., (2014) Front Physiol 5 151; Rossini M, et. al., (2010) Arthritis Res Ther 12(6) R216; Palm N W, et. al., (2012) Nature 484(7395) 465-472; Ikeda U, et. al., (2010) Immunology Letters 134(1) 7-16; Mayne C G, et. al., (2011) Eur J Immunol 41(3) 822-832; Hartmann B, et. al., (2012) J Invest Dermatol 132(2) 330-336). However, toxic/calcemic effects of 1α,25(OH)₂D₃ at pharmacological doses constrains its use to treat autoimmune diseases and other pathological states (Holick M F, (2007) N Engl J Med 357(3) 266-81; Bikle D D, et. al., (2020) J Endocr Soc 4(2) bvz038; Slominski A T, et. al., (2010) PloS One 5(3) e9907; Wang J, et. al., (2012) Anticancer Res 32(3) 739-46; Biankin A V, et. al., (2012) Nature 491(7424) 399-405).

New pathways of vitamin D₃ activation by CYP11A1 producing many hydroxy derivatives have now been well characterized (Tuckey R C, et. al., (2019) J Steroid Biochem Mol Biol 186 (2019) 4-21; Slominski A T, et. al., (2015) J Steroid Biochem Mol Biol 151 25-37). The main sequence of the reactions starts with hydroxylation at C-20 to produce 20S(OH)D₃ (3) which is sequentially hydroxylated to 20S,23S(OH)₂D₃ (4a) and 17R,20S,23S(OH)₃D₃ (5) (Tuckey R C, et. al., (2019) J Steroid Biochem Mol Biol 186 (2019) 4-21; Tuckey R C, et. al., (2008) FEBS J 275(10) 2585-96; Slominski R M, et. al., (2021) Mol Cell Endocrinol Available online 12 Mar. 2021, 111238). 20S(OH)D₃ can also be hydroxylated by CYP27A1, CYP24A1 and CYP2R1 to produce 20S,25(OH)₂D₃ (6), which is an excellent substrate for CYP27B1 (Tuckey R C, et. al., (2019) J Steroid Biochem Mol Biol 186 (2019) 4-21). These metabolites are produced ex vivo in skin cells, placenta, and adrenal glands in a CYP11A1 dependent fashion (Slominski A T, et. al., (2012) FASEB J 26(9) 3901-15; Slominski A T, et. al., (2016) Exp Dermatol 25(3) 231-2). Some, including 20S(OH)D₃ and 1α,20S(OH)₂D₃, have been measured in the nM range in all human serum samples tested (Slominski A T, et. al., (2015) Sci Rep 5 14875, Jenkinson C, et. al., (2021) Clin Chem Lab Med) and 20S(OH)D₃ was recently detected in honey (Kim T K, et. al., (2020) Molecules 25(11)). Thus, in addition to functioning as hormones in humans (see below), some of these secosteroids can be defined as natural products (Slominski A T, et. al., (2020) Cell Biochem Biophys 78(2) 165-180).

In vitro studies have demonstrated that 20S(OH)D₃ and 20S,23S(OH)₂D₃ show anti-proliferative, pro-differentiation, anticancer, photoprotective, anti-inflammatory and anti-fibrinogenic properties (Janjetovic Z, et. al., (2021) Endocrinology 162(1); Chaiprasongsuk A, et. al., (2020) Free Radic Biol Med 155 87-98; Chaiprasongsuk A, et. al., (2019) Redox Biol 24 101206; Slominski A T, et. al., (2020) Adv Exp Med Biol in press; Slominski A T, et. al., (2014) J Steroid Biochem Mol Biol 144PA 28-39; Janjetovic Z, et. al., (2010) Journal of cellular physiology 223(1) 36-48; Slominski A, et. al., (2013) J Clin Endocrinol Metab 98(2) E298-303; Slominski A T, et. al., (2014) J Steroid Biochem Mol Biol 144 Pt A 28-39). 20S,25(OH)₂D₃ has also been reported to exhibit anti-melanoma activity in cell culture (Podgorska E, et. al., (2021) Cancers (Basel) 13(13)). Anti-fibrogenic, photoprotective and anti-melanoma properties for 20S(OH)D₃ were also demonstrated in in vivo models (Slominski A, et. al., (2013) J Clin Endocrinol Metab 98(2) E298-303; Tieu E W, et. al., (2012) Biochem Pharmacol 84(12) 1696-704; Tongkao-On W, et. al., (2015) J Steroid Biochem Mol Biol 148 72-8). Of high clinical relevance is that CYP11A-derived 20S(OH)D₃ and 20S,23S(OH)₂D₃ are non-calcemic at doses that are highly toxic for 1α,25(OH)₂D₃ and 25(OH)D₃ (Slominski A T, et. al., (2010) PloS One 5(3) e9907; Slominski A, et. al., (2013) J Clin Endocrinol Metab 98(2) E298-303; Skobowiat C, et. al., (2017) Oncotarget 8(6) 9823-9834). The CYP11A1-derived secosteroids have been shown to mediate their effects by acting as biased agonists on the VDR (Slominski A T, et. al., (2014) J Steroid Biochem Mol Biol 144PA 28-39; Chen J, et. al., (2014) Anticancer Res 34(5) 2153-63; Kim T K, et. al., (2012) Mol Cell Endocrinol 361(1-2) 143-52; Slominski A T, et. al., (2017) J Steroid Biochem Mol Biol 173 42-56) inverse agonists on retinoic acid orphan receptors (ROR) α and γ (Kim T K, et. al., (2012) Mol Cell Endocrinol 361(1-2) 143-52; Lin Z, et. al., (2018) Sci Rep 8(1) 1478), and agonists on liver X receptors (LXRs) (Slominski A T, et. al., (2014) FASEB J 28(7) 2775-89). 20S(OH)D₃ and 20S,23S(OH)₂D₃ are also agonists for the aryl hydrocarbon receptor (AhR) (Slominski A T, et. al., (2021) Sci Rep 11(1) 8002).

Many hydroxyl-derivatives of vitamin D₃ metabolites have been characterized, however, such metabolites require a more robust source to enable better characterization of these metabolites and to further investigate the advantages of their biological activity. Therefore, new chemical routes and intermediates useful in the large-scale synthesis of hydroxy derivatives of D₃ are needed.

### SUMMARY

In accordance with the purpose(s) of the invention, as embodied and broadly described herein, the invention, in one aspect, relates to methods of making hydroxy derivatives of vitamin D₃, compounds useful as intermediates in the preparation of the hydroxy derivatives, and methods of making the intermediates.

Thus, disclosed are compounds having a structure represented by a formula:

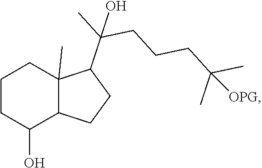

wherein PG is a silyl protecting group, or a pharmaceutically acceptable salt thereof.

Also disclosed are methods of making a compound having a structure represented by a formula:

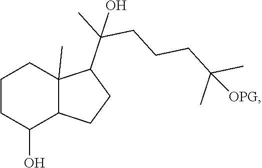

wherein PG is a silyl protecting group, or a pharmaceutically acceptable salt thereof, the method comprising reacting a methyl ketone having a structure represented by a formula:

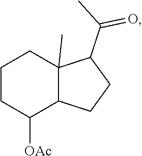 [SMILES: CC(=O)OC1CCCC2(C)C(C(C)=O)CCC12]

or a pharmaceutically acceptable salt thereof, and an alkyl halide having a structure represented by a formula:

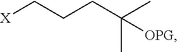

or a pharmaceutically acceptable salt thereof, wherein X is a halide.

Also disclosed are compounds having a structure represented by a formula:

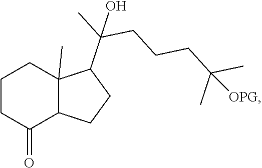

wherein PG is a protecting group selected from acetyl (Ac), pivaloyl (Piv), methoxymethyl (MOM), 2-methoxyethoxymethyl ether (MEM), benzyloxymethyl (BOM), tetrahydropyranyl (THP), trimethylsilyl (TMS), triethylsilyl (TES), tert-butyldimethylsilyl (TBS), tert-butyldiphenylsilyl (TBDPS), and triisopropylsilyl (TIPS), and trimethylsilylethoxymethyl (SEM), or a pharmaceutically acceptable salt thereof.

Also disclosed are methods of making a compound having a structure represented by a formula:

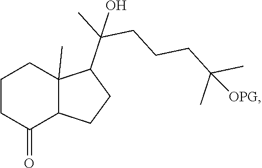

wherein PG is a protecting group selected from acetyl (Ac), pivaloyl (Piv), methoxymethyl (MOM), 2-methoxyethoxymethyl ether (MEM), benzyloxymethyl (BOM), tetrahydropyranyl (THP), trimethylsilyl (TMS), triethylsilyl (TES), tert-butyldimethylsilyl (TBS), tert-butyldiphenylsilyl (TBDPS), and triisopropylsilyl (TIPS), and trimethylsilylethoxymethyl (SEM), or a pharmaceutically acceptable salt thereof, the method comprising oxidizing an alcohol having a structure represented by a formula:

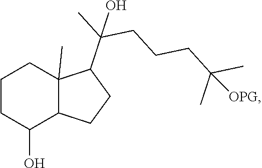

or a pharmaceutically acceptable salt thereof.

Also disclosed are compounds having a structure represented by a formula:

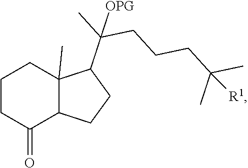

wherein R¹ is hydrogen or —OPG; and wherein each occurrence of PG is independently a protecting group selected from acetyl (Ac), pivaloyl (Piv), methoxymethyl (MOM), 2-methoxyethoxymethyl ether (MEM), benzyloxymethyl (BOM), tetrahydropyranyl (THP), trimethylsilyl (TMS), triethylsilyl (TES), tert-butyldimethylsilyl (TBS), tert-butyldiphenylsilyl (TBDPS), and triisopropylsilyl (TIPS), and trimethylsilylethoxymethyl (SEM), or a pharmaceutically acceptable salt thereof.

Also disclosed are methods of making a compound having a structure represented by a formula:

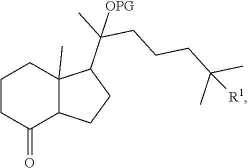

wherein R¹ is hydrogen or —OPG; and wherein each occurrence of PG is independently a protecting group selected from acetyl (Ac), pivaloyl (Piv), methoxymethyl (MOM), 2-methoxyethoxymethyl ether (MEM), benzyloxymethyl (BOM), tetrahydropyranyl (THP), trimethylsilyl (TMS), triethylsilyl (TES), tert-butyldimethylsilyl (TBS), tert-butyldiphenylsilyl (TBDPS), and triisopropylsilyl (TIPS), and trimethylsilylethoxymethyl (SEM), or a pharmaceutically acceptable salt thereof, the method comprising protecting an alcohol having a structure represented by a formula:

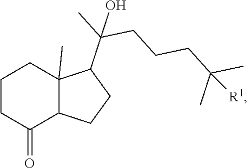

or a pharmaceutically acceptable salt thereof.

Also disclosed are compounds having a structure represented by a formula:

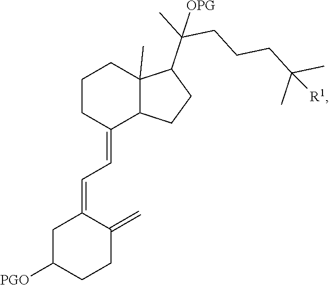

wherein R¹ is hydrogen or —OPG; and wherein each occurrence of PG is independently a silyl protecting group, or a pharmaceutically acceptable salt thereof.

Also disclosed are methods of making a compound having a structure represented by a formula:

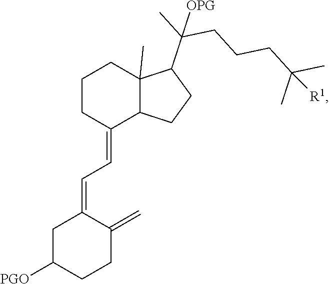

wherein R¹ is hydrogen or —OPG; and wherein each occurrence of PG is independently a silyl protecting group, or a pharmaceutically acceptable salt thereof, the method comprising coupling a ketone having a structure represented by a formula:

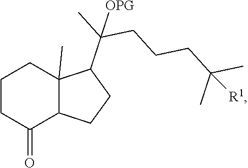

or a pharmaceutically acceptable salt thereof, and a phosphine oxide having a structure represented by a formula:

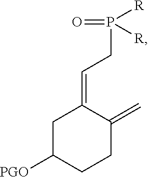

wherein each occurrence of R is independently C₆H₅ substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

Also disclosed are methods of making a compound having a structure represented by a formula:

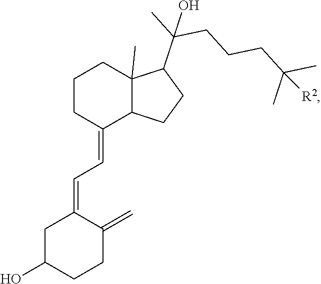

wherein R² is hydrogen or —OH, or a pharmaceutically acceptable salt thereof, the method comprising deprotecting a protected alcohol having a structure represented by a formula:

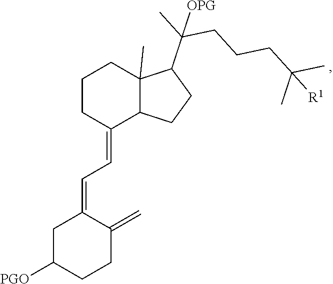

wherein R¹ is hydrogen or —OPG; and wherein each occurrence of PG is independently a silyl protecting group, or a pharmaceutically acceptable salt thereof.

Also disclosed are compounds having a structure represented by a formula:

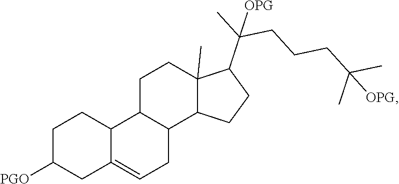

wherein each occurrence of PG is a different silyl protecting group, or a pharmaceutically acceptable salt thereof.

Also disclosed are compounds having a structure represented by a formula:

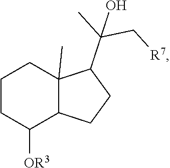

wherein R³ is selected from hydrogen and an alcohol protecting group, and wherein R⁷ is selected from —CN and —CO₂R¹⁰; and wherein R¹⁰ is C1-C4 alkyl, or a pharmaceutically acceptable salt thereof.

Also disclosed are methods of making a compound having a structure represented by a formula:

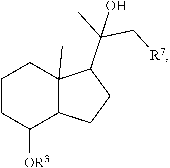

wherein R³ is selected from hydrogen and an alcohol protecting group; and wherein R⁷ is selected from —CN and —CO₂R¹⁰; and wherein R¹⁰ is C1-C4 alkyl, or a pharmaceutically acceptable salt thereof, the method comprising reacting a methyl ketone having a structure represented by a formula:

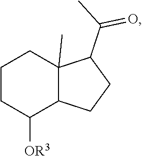

and a nitrile or CH₃CO₂R¹⁰, or a pharmaceutically acceptable salt thereof.

Also disclosed are methods of making a compound having a structure represented by a formula:

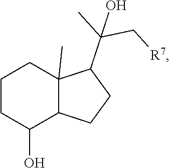

wherein R⁷ is selected from —CN and —CO₂R¹⁰; and wherein R¹⁰ is C1-C4 alkyl, or a pharmaceutically acceptable salt thereof, the method comprising deprotecting an alcohol having a structure represented by a formula:

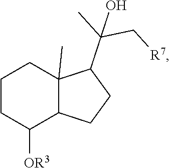

wherein R³ is selected from hydrogen and an alcohol protecting group, or a pharmaceutically acceptable salt thereof.

Also disclosed are compounds having a structure represented by a formula:

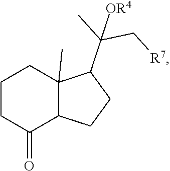

wherein R⁴ is selected from hydrogen and an alcohol protecting group; and wherein R⁷ is selected from —CN and —CO₂R¹⁰; and wherein R¹⁰ is C1-C4 alkyl, or a pharmaceutically acceptable salt thereof.

Also disclosed are methods of making a compound having a structure represented by a formula:

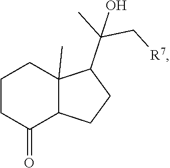

wherein R⁷ is selected from —CN and —CO₂R¹⁰; and wherein R¹⁰ is C1-C4 alkyl, or a pharmaceutically acceptable salt thereof, the method comprising oxidizing an alcohol having a structure represented by a formula:

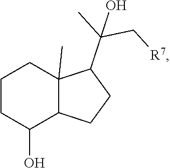

or a pharmaceutically acceptable salt thereof.

Also disclosed are methods of making a compound having a structure represented by a formula:

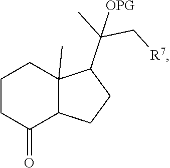

wherein PG is an alcohol protecting group; wherein R⁷ is selected from —CN and —CO₂R¹⁰; and wherein R¹⁰ is C1-C4 alkyl, or a pharmaceutically acceptable salt thereof, the method comprising protecting an alcohol having a structure represented by a formula:

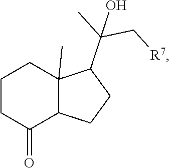

or a pharmaceutically acceptable salt thereof.

Also disclosed are compounds having a structure represented by a formula:

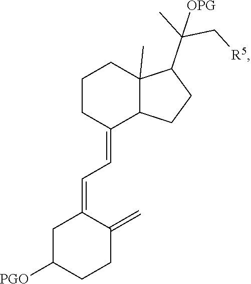

wherein R⁵ is selected from —CN, —CHO, —CO₂R¹⁰; wherein R¹⁰ is C1-C4 alkyl; and wherein each occurrence of PG is independently an alcohol protecting group, or a pharmaceutically acceptable salt thereof.

Also disclosed are methods of making a compound having a structure represented by a formula:

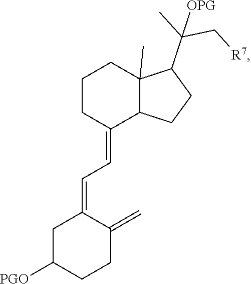

wherein R⁷ is selected from —CN and —CO₂R¹⁰; and wherein R¹⁰ is C1-C4 alkyl, wherein each occurrence of PG is independently a silyl protecting group, or a pharmaceutically acceptable salt thereof, the method comprising coupling a ketone having a structure represented by a formula:

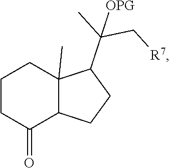

and a phosphine oxide having a structure represented by a formula:

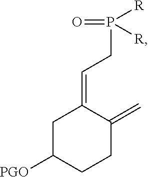

wherein each occurrence of R is independently C₆H₅ substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

Also disclosed are methods of making a compound having a structure represented by a formula:

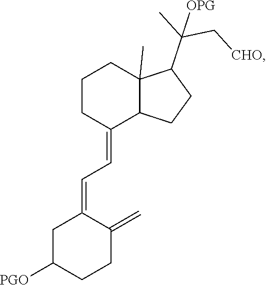

wherein each occurrence of PG is independently a hydroxyl protecting group, or a pharmaceutically acceptable salt thereof, the method comprising reducing a triene having a structure represented by a formula:

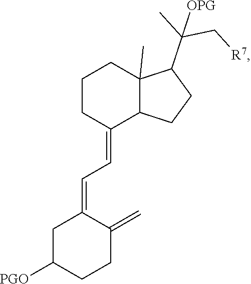

wherein R⁷ is selected from —CN and —CO₂R¹⁰; and wherein R¹⁰ is C1-C4 alkyl, or a pharmaceutically acceptable salt thereof.

Also disclosed are compounds having a structure represented by a formula:

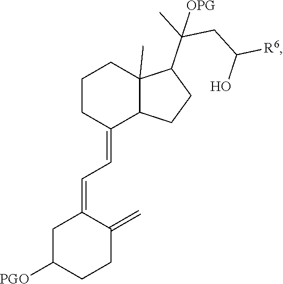

wherein R⁶ is a C1-C8 alkyl; and wherein each occurrence of PG is independently a silyl protecting group, or a pharmaceutically acceptable salt thereof.

Also disclosed are methods of making a compound having a structure represented by a formula:

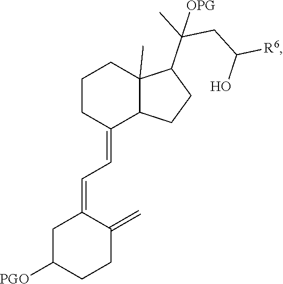

wherein R⁶ is a C1-C8 alkyl; and wherein each occurrence of PG is independently a silyl protecting group, or a pharmaceutically acceptable salt thereof, the method comprising reacting an aldehyde having a structure represented by a formula:

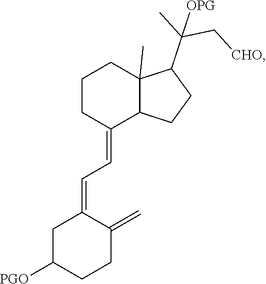

or a pharmaceutically acceptable salt thereof, and a Grignard reagent having a structure represented by a formula:

R⁶—Mg—X,

wherein X is a halide.

Also disclosed are methods of making a compound having a structure represented by a formula:

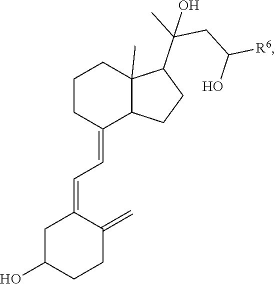

wherein R⁶ is a C1-C8 alkyl, or a pharmaceutically acceptable salt thereof, the method comprising deprotecting an alcohol having a structure represented by a formula:

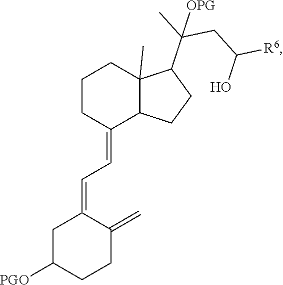

wherein each occurrence of PG is independently a silyl protecting group, or a pharmaceutically acceptable salt thereof.

Also disclosed are compounds having a structure represented by a formula:

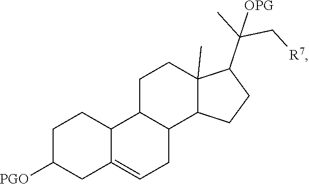

wherein R⁷ is selected from —CN and —CO₂R¹⁰; and wherein R¹⁰ is C1-C4 alkyl; and wherein each occurrence of PG is independently a hydroxyl protecting group, or a pharmaceutically acceptable salt thereof.

Also disclosed are compounds having a structure represented by a formula:

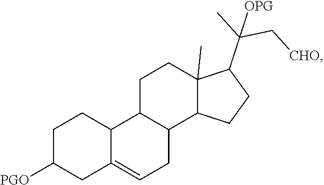

wherein each occurrence of PG is independently a hydroxyl protecting group, or a pharmaceutically acceptable salt thereof.

Also disclosed are methods of making an aldehyde having a structure represented by a formula:

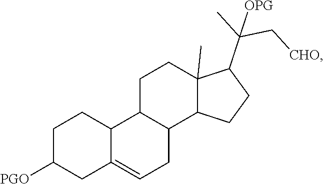

wherein each occurrence of PG is independently a hydroxyl protecting group, or a pharmaceutically acceptable salt thereof, the method comprising reducing a compound having a structure represented by a formula:

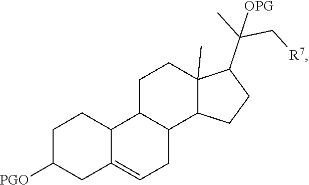

wherein R⁷ is selected from —CN and —CO₂R¹⁰; and wherein R¹⁰ is C1-C4 alkyl, or a pharmaceutically acceptable salt thereof.

Also disclosed are compounds having a structure represented by a formula:

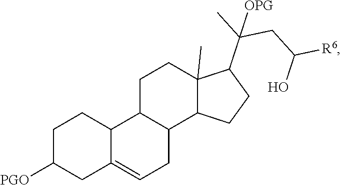

wherein R⁶ is a C1-C8 alkyl; and wherein each occurrence of PG is independently a silyl protecting group, or a pharmaceutically acceptable salt thereof.

Also disclosed are methods of making a compound having a structure represented by a formula:

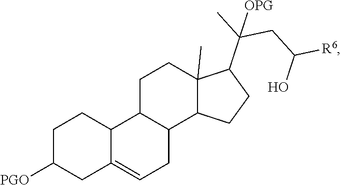

wherein R⁶ is a C1-C8 alkyl; and wherein each occurrence of PG is independently a silyl protecting group, or a pharmaceutically acceptable salt thereof, the method comprising reacting an aldehyde having a structure represented by a formula:

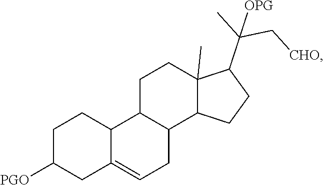

or a pharmaceutically acceptable salt thereof, and a Grignard reagent having a structure represented by a formula:

R⁶—Mg—X,

wherein X is a halide.

Also disclosed are methods of making a compound having a structure represented by a formula:

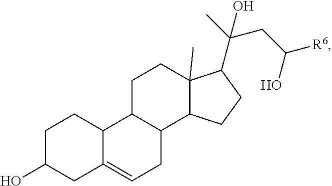

wherein R⁶ is a C1-C8 alkyl, or a pharmaceutically acceptable salt thereof, the method comprising deprotecting an alcohol having a structure represented by a formula:

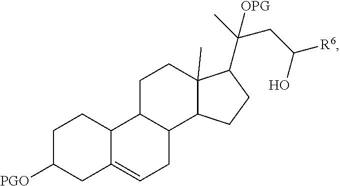

wherein each occurrence of PG is independently a silyl protecting group, or a pharmaceutically acceptable salt thereof.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows representative chemical structures of vitamin D₃ and some of its biologically important derivatives.

FIG. 2 shows representative chemical structures of vitamin D₂ (7) and the building blocks used as the key intermediates in the synthesis of target vitamin D compounds.

FIG. 3 shows a representative synthetic scheme for preparation of the A-ring (compounds 8 and 9) and CD-ring (compounds 11 and 12) building blocks used for the synthesis of target vitamins.

FIG. 4 shows a representative synthetic scheme for preparation of 20S(OH)D₃ and 20S,25(OH)₂D₃.

FIG. 5 shows a representative synthetic scheme for preparation of 20S,23S(OH)₂D₃ and 20S,23R(OH)₂D₃.

FIG. 6A-D show representative data illustrating the inhibition of proliferation by 20S(OH)D₃, 20S,25(OH)₂D₃, 20S,23S(OH)₂D₃, and 20S,23R(OH)₂D₃.

FIG. 7A-D show representative data illustrating the stimulation of keratinocyte differentiation by 20S(OH)D₃, 20S,25(OH)₂D₃, 20S,23S(OH)₂D₃, and 20S,23R(OH)₂D₃ in comparison to 1α,25(OH)₂D₃.

FIG. 8A-F show representative data illustrating the interaction of 20S(OH)D₃, 20S,25(OH)₂D₃, 20S,23S(OH)₂D₃, and 20S,23R(OH)₂D₃ with nuclear receptors.

FIG. 9 shows representative data illustrating the molecular docking results for the complexes of vitamin D₃ hydroxy derivatives with selected nuclear receptors.

FIG. 10A and FIG. 10B show representative data illustrating the binding pattern of selected vitamin D₃ derivatives with VDR.

FIG. 11A and FIG. 11B show representative data illustrating the binding pattern of selected vitamin D₃ derivatives with AhR.

FIG. 12A-D show representative data illustrating the binding pattern of selected vitamin D₃ derivatives with LXRs.

FIG. 13A-D show representative data of the binding pattern of selected vitamin D₃ derivatives with RORs.

FIG. 14A and FIG. 14B show representative data of the differential and overlapping effects of secosteroids on activation of CD4 and CD8 T cells from human peripheral blood.

FIG. 15 shows straight-phase (a) and reversed-phase (b) HPLC chromatograms of 20S(OH)D₃ (3).

FIG. 16 shows straight-phase (a) and reversed-phase (b) HPLC chromatograms of 20S,23S(OH)₂D₃ (4a).

FIG. 17 shows straight-phase (a) and reversed-phase (b) HPLC chromatograms of 20S,23R(OH)₂D₃ (4b).

FIG. 18 shows straight-phase (a) and reversed-phase (b) HPLC chromatograms of 20S,25(OH)₂D₃ (6).

FIG. 19A and FIG. 19B show representative ¹H and ¹³C NMR spectra of vitamin D₂ (7).

FIG. 20A and FIG. 20B show representative ¹H and ¹³C NMR spectra of (2Z,5′S)-2-[5′-[(tert-butyldimethylsilyl)oxy]-2′-methylenecyclohexylidene]ethanol (8).

FIG. 21A and FIG. 21B show representative ¹H and ¹³C NMR spectra of 7a-methyl-1-[(1′R,2′E,4′R)-1′,4′,5′-trimethyl-2′-hexen-1′-yl]octahydro-1H-inden-4-ol (10).

FIG. 22A and FIG. 22B show representative ¹H and ¹³C NMR spectra of (2Z,5′S)-2-{[5′-[(tert-butyldimethylsilyl)oxy]-2′-methylenecyclohexylidene]ethyl}diphenylphosphine oxide (9).

FIG. 23A and FIG. 23B show representative ¹H and ¹³C NMR spectra of (1R,3aR,4S,7aR)-7a-methyl-4-[(triethylsilyl)oxy]-1-[(1′R,2′E,4′R)-1′,4′,5′-trimethyl-2′-hexen-1′-yl]octahydro-1H-indene (ii).

FIG. 24A and FIG. 24B show representative ¹H and ¹³C NMR spectra 2-[(1′S,3a′R,4′S,7a′S)-7a′-methyl-4′-[(triethylsilyl)oxy]octahydro-1H-inden-1′-yl)]propanal (iii).

FIG. 25A and FIG. 25B show representative ¹H and ¹³C NMR spectra 3aR,4S,7aS)-1-acetyl-7a-methyl-4-[(triethylsilyl)oxy]octahydro-1H-indene (11).

FIG. 26A and FIG. 26B show representative ¹H and ¹³C NMR spectra 3aR,4S,7aS)-1-Acetyl-7a-methyloctahydro-1H-inden-4-ol (iv).

FIG. 27A and FIG. 27B show representative ¹H and ¹³C NMR spectra S,7aS)-1-acetyl-7a-methyloctahydro-1H-inden-4-yl acetate (12).

FIG. 28A and FIG. 28B show representative ¹H and ¹³C NMR of (1S,3aR,4S,7aS)-1-[(S)-2′-hydroxy-6′-methylheptan-2′-yl]-7a-methyloctahydro-1H-inden-4-ol (13).

FIG. 29A and FIG. 29B show representative ¹H and ¹³C NMR of (1S,3aR,4S,7aS)-1-[(S)-2′-hydroxy-6′-methyl-6′-[(triethylsilyl)oxy]heptan-2′-yl]-7a-methyloctahydro-1H-inden-4-ol (14).

FIG. 30A and FIG. 30B show representative ¹H and ¹³C NMR of (1S,3aR,7aR)-1-[(S)-2′-hydroxy-6′-methylheptan-2′-yl]-7a-methyloctahydro-4H-inden-4-one (15).

FIG. 31A and FIG. 31B show representative ¹H and ¹³C NMR of (1S,3aR,7aR)-1-[(S)-2′-hydroxy-6′-methyl-6′-[(triethylsilyl)oxy]heptan-2′-yl]-7a-methyloctahydro-4H-inden-4-one (16).

FIG. 32A and FIG. 32B show representative ¹H and ¹³C NMR of (1S,3aR,7aR)-7a-methyl-1-[(S)-6′-methyl-2′-[(trimethylsilyl)oxy]heptan-2′-yl]octahydro-4H-inden-4-one (17).

FIG. 33A and FIG. 33B show representative ¹H and ¹³C NMR of (1S,3aR,7aR)-7a-methyl-1-[(S)-6′-methyl-6′-[(triethylsilyl)oxy]-2′-[(trimethylsilyl)oxy]heptan-2′-yl]octahydro-4H-inden-4-one (18).

FIG. 34A and FIG. 34B show representative ¹H and ¹³C NMR of (20S)-3-[(tert-butyldimethylsilyl)oxy]-20-[(trimethylsilyl)oxy]vitamin D₃ (19).

FIG. 35A and FIG. 35B show representative ¹H and ¹³C NMR of (20S)-3-[(tert-butyldimethylsilyl)oxy]-25-[(triethylsilyl)oxy]-20-[(trimethylsilyl)oxy]-vitamin D₃ (20).

FIG. 36A and FIG. 36B show representative ¹H and ¹³C NMR of (20S)-20-hydroxyvitamin D₃ (3).

FIG. 37A and FIG. 37B show representative ¹H and ¹³C NMR of (20S)-20,25-dihydroxyvitamin D₃ (6).

FIG. 38A and FIG. 38B show representative ¹H and ¹³C NMR of (3S)-3-[(1′S,3a′R,4′S,7a′S)-7a′-methyl-4′-[(triethylsilyl)oxy]-octahydro-1H-inden-1′-yl)]-3-hydroxybutanenitrile (21).

FIG. 39A and FIG. 39B show representative ¹H and ¹³C NMR of (3S)-3-[(1′S,3a′R,4′S,7a′S)-4′-hydroxy-7a′-methyl-octahydro-1H-inden-1′-yl)]-3-hydroxybutanenitrile (22).

FIG. 40A and FIG. 40B show representative ¹H and ¹³C NMR of (3S)-3-hydroxy-3-[(1′S,3a′R,7a′R)-7a′-methyl-4′-oxo-octahydro-1H-inden-1′-yl)butanenitrile (23).

FIG. 41A and FIG. 41B show representative ¹H and ¹³C NMR of (3S)-3-[(1′S,3a′R,7a′R)-7a′-methyl-4′-oxo-octahydro-1H-inden-1′-yl)]-3-[(trimethylsilyl)oxy]butanenitrile (24).

FIG. 42A and FIG. 42B show representative ¹H and ¹³C NMR of (20S)-3-[(tert-butyldimethylsilyl)oxy]-22-cyano-20-[(trimethylsilyl)oxy]-23,24,25,26,27-pentanorvitamin D₃ (25).

FIG. 43A and FIG. 43B show representative ¹H and ¹³C NMR of (20S)-3-[(tert-butyldimethylsilyl)oxy]-22-formyl-20-[(trimethylsilyl)oxy]-23,24,25,26,27-pentanorvitamin D₃ (26).

FIG. 44A and FIG. 44B show representative ¹H and ¹³C NMR of (20S,23R)-dihydroxyvitamin D₃ (4a).

FIG. 45A and FIG. 45B show representative ¹H and ¹³C NMR of (20S,23S)-dihydroxyvitamin D₃ (4b).

FIG. 46 shows a representative synthetic scheme for the preparation of hydroxy derivatives (closed form) of vitamin D₃.

FIG. 47 shows a representative synthetic scheme for an alternative route to prepare hydroxy derivatives (closed form) of vitamin D₃.

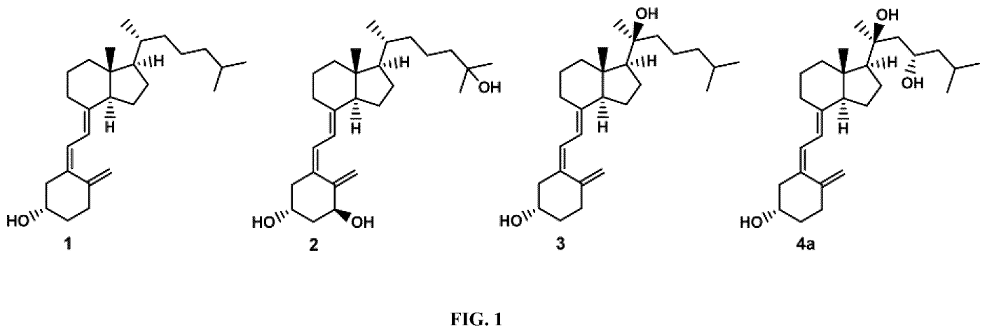

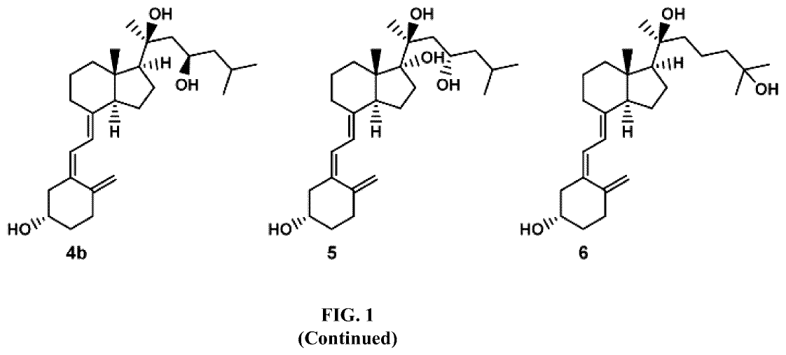

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2)π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula (A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide” as used herein can be used interchangeably and refer to F, Cl, Br, or I.

The terms “pseudohalide,” “pseudohalogen,” or “pseudohalo” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula N₃.

The term “nitro” as used herein is represented by the formula NO₂.

The term “nitrile” or “cyano” as used herein is represented by the formula CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C1-4 straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)O₂C(O)R^{●}, —(CH₂)O₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR*, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

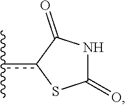

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di-, or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

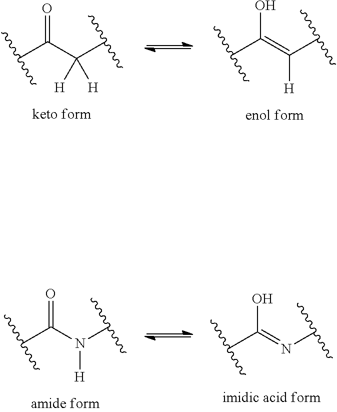 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

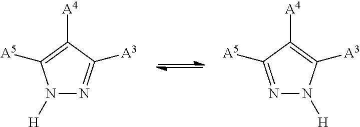

Unless stated to the contrary, the invention includes all such possible tautomers.

It is known that chemical substances form solids, which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

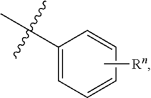

which is understood to be equivalent to a formula:

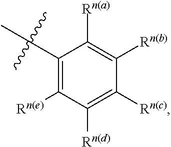

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds Useful as Intermediates Towards the Preparation of Hydroxy Derivatives of Vitamin D₃(Open Chain-Route I)

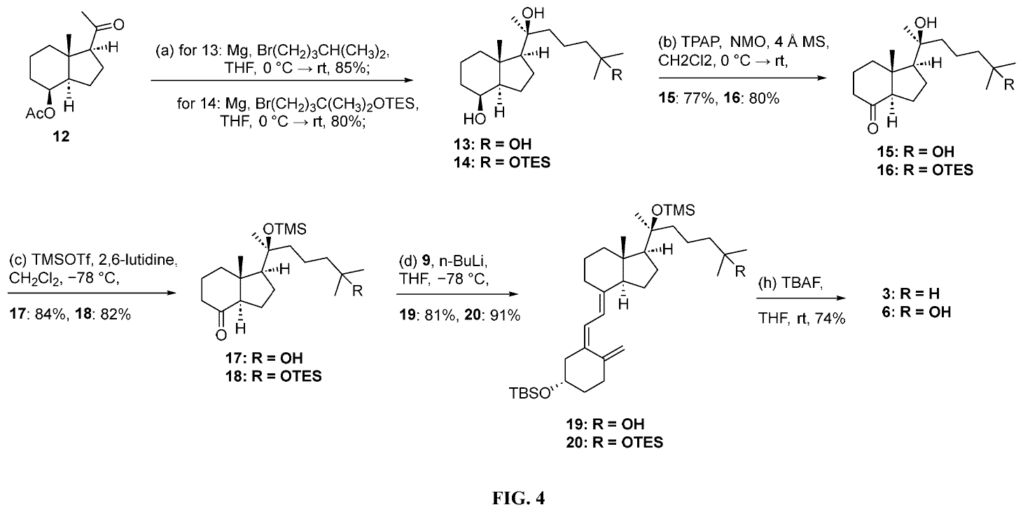

In one aspect, disclosed are compounds useful as intermediates towards the preparation of open chain hydroxy derivatives of vitamin D₃. See, e.g., FIG. 4. Hydroxy derivatives of vitamin D₃ have demonstrated utility as therapeutic agents for a variety of autoimmune disorders including, but not limited to, lupus erythematosus, rheumatoid arthritis, inflammatory bowel disease, psoriasis, and multiple sclerosis. Thus, in various aspects, disclosed are secondary alcohols, cyclohexanones, protected tertiary alcohols, and trienes useful as intermediates in the synthesis of open chain hydroxy derivatives of vitamin D₃.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Secondary Alcohols

In one aspect, disclosed are secondary alcohols having a structure represented by a formula:

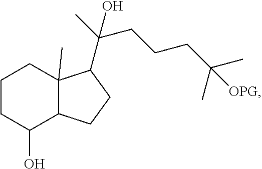

wherein PG is a silyl protecting group, or a pharmaceutically acceptable salt thereof. Exemplary silyl protecting groups include, but are not limited to, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS).

Thus, in various aspects, PG is a silyl protecting group. In a further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, the silyl protecting group is selected from trimethylsilyl (TMS), triethylsilyl (TES), and tert-butyldimethylsilyl (TBS). In a yet further aspect, the silyl protecting group is selected from trimethylsilyl (TMS) and triethylsilyl (TES). In an even further aspect, the silyl protecting group is triethylsilyl (TES).

In various aspects, the compound has a structure:

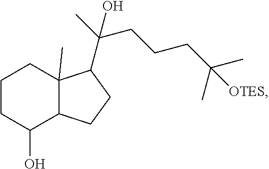

or a pharmaceutically acceptable salt thereof.

In various aspects, the compound has a structure represented by a formula:

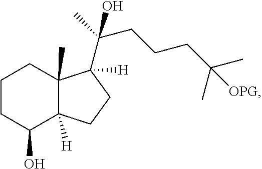

or a pharmaceutically acceptable salt thereof.

In various aspects, the compound has a structure:

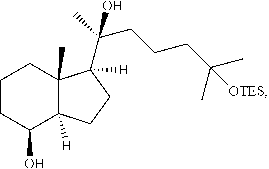

or a pharmaceutically acceptable salt thereof.

### A. Exemplary Secondary Alcohols

In one aspect, a secondary alcohol can be present as:

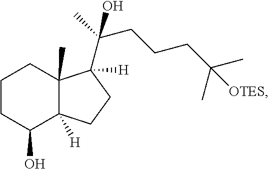

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 2. Cyclohexanones

In one aspect, disclosed are cyclohexanones having a structure represented by a formula:

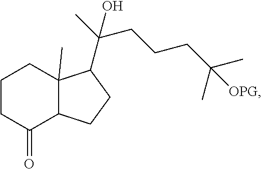

wherein PG is a protecting group selected from acetyl (Ac), pivaloyl (Piv), methoxymethyl (MOM), 2-methoxyethoxymethyl ether (MEM), benzyloxymethyl (BOM), tetrahydropyranyl (THP), trimethylsilyl (TMS), triethylsilyl (TES), tert-butyldimethylsilyl (TBS), tert-butyldiphenylsilyl (TBDPS), and triisopropylsilyl (TIPS), and trimethylsilylethoxymethyl (SEM), or a pharmaceutically acceptable salt thereof.

In various aspects, PG is a silyl protecting group. In a further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, the silyl protecting group is selected from trimethylsilyl (TMS), triethylsilyl (TES), and tert-butyldimethylsilyl (TBS). In a yet further aspect, the silyl protecting group is selected from trimethylsilyl (TMS) and triethylsilyl (TES). In an even further aspect, the silyl protecting group is triethylsilyl (TES).

In various aspects, the compound has a structure:

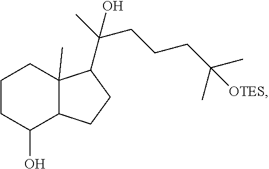

or a pharmaceutically acceptable salt thereof.

In various aspects, the compound has a structure represented by a formula:

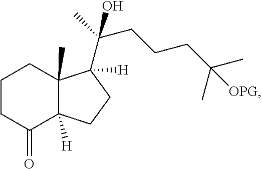

or a pharmaceutically acceptable salt thereof.

In various aspects, the compound has a structure:

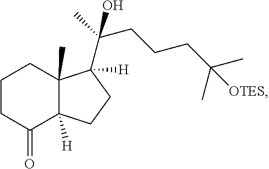

or a pharmaceutically acceptable salt thereof.

### A. Exemplary Cyclohexanones

In one aspect, a cyclohexanone can be present as:

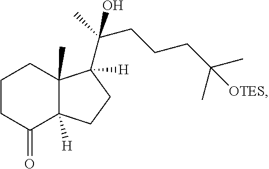

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 3. Protected Tertiary Alcohols

In one aspect, disclosed are protected tertiary alcohols having a structure represented by a formula:

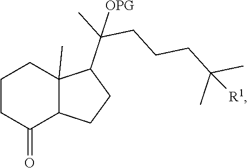

wherein R¹ is hydrogen or —OPG; and wherein each occurrence of PG is independently a protecting group selected from acetyl (Ac), pivaloyl (Piv), methoxymethyl (MOM), 2-methoxyethoxymethyl ether (MEM), benzyloxymethyl (BOM), tetrahydropyranyl (THP), trimethylsilyl (TMS), triethylsilyl (TES), tert-butyldimethylsilyl (TBS), tert-butyldiphenylsilyl (TBDPS), and triisopropylsilyl (TIPS), and trimethylsilylethoxymethyl (SEM), or a pharmaceutically acceptable salt thereof.

In various aspects, each occurrence of PG is independently a silyl protecting group. In a further aspect, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, each occurrence of PG is independently a silyl protecting group selected from trimethylsilyl (TMS) and triethylsilyl (TES).

In various aspects, the compound has a structure represented by a formula:

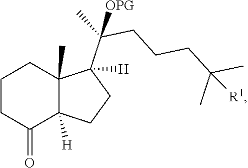

or a pharmaceutically acceptable salt thereof.

In various aspects, the compound has a structure represented by a formula:

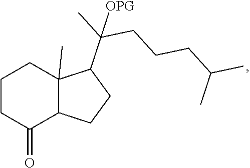

or a pharmaceutically acceptable salt thereof.

In various aspects, the compound has a structure represented by a formula:

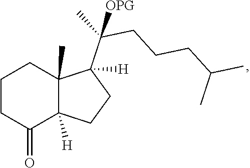

or a pharmaceutically acceptable salt thereof.

In various aspects, the compound has a structure:

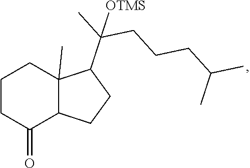

or a pharmaceutically acceptable salt thereof.

In various aspects, the compound has a structure:

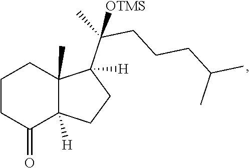

or a pharmaceutically acceptable salt thereof.

In various aspects, the compound has a structure represented by a formula:

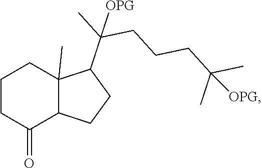

or a pharmaceutically acceptable salt thereof.

In various aspects, the compound has a structure represented by a formula:

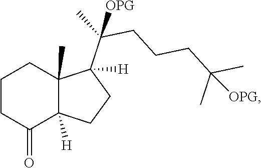

or a pharmaceutically acceptable salt thereof.

In various aspects, the compound has a structure:

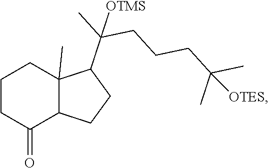

or a pharmaceutically acceptable salt thereof.

In various aspects, the compound has a structure:

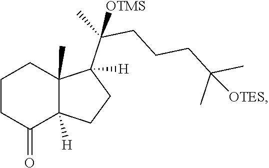

or a pharmaceutically acceptable salt thereof.

### A. R¹ Groups

In one aspect, R¹ is hydrogen or —OPG. In a further aspect, R¹ is hydrogen. In a still further aspect, R¹ is —OPG. In yet a further aspect, R¹ is —OTES.

b. Exemplary Protected Tertiary Alcohols

In one aspect, a protected tertiary alcohol can be present as:

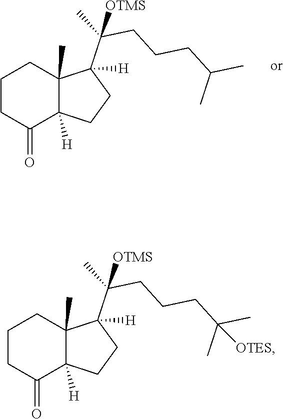

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 4. Trienes

In one aspect, disclosed are trienes having a structure represented by a formula:

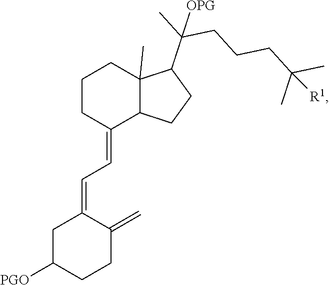

wherein R¹ is hydrogen or —OPG; and wherein each occurrence of PG is independently a silyl protecting group, or a pharmaceutically acceptable salt thereof.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In further aspect, each occurrence of PG is independently a silyl protecting group selected from trimethylsilyl (TMS), triethylsilyl (TES), and tert-butyldimethylsilyl (TBS). In a still further aspect, each occurrence of PG is a different silyl protecting group.

In various aspects, the compound has a structure represented by a formula:

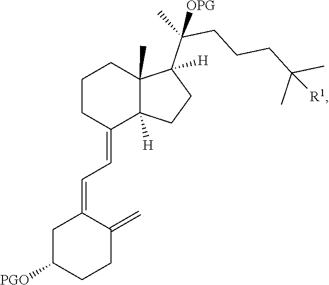

or a pharmaceutically acceptable salt thereof.

In various aspects, the compound has a structure represented by a formula:

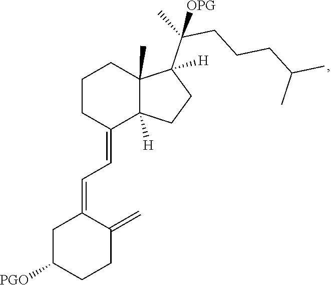

or a pharmaceutically acceptable salt thereof.

In various aspects, the compound has a structure represented by a formula:

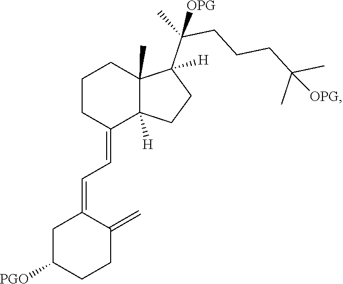

or a pharmaceutically acceptable salt thereof.

In various aspects, the compound has a structure represented by a formula:

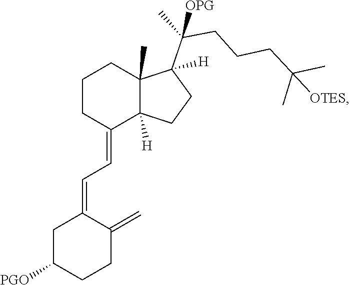

or a pharmaceutically acceptable salt thereof.

In various aspects, the compound has a structure represented by a formula:

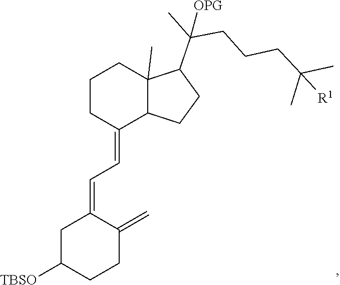

or a pharmaceutically acceptable salt thereof.

In various aspects, the compound has a structure represented by a formula:

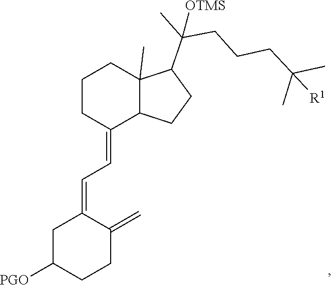

or a pharmaceutically acceptable salt thereof.

In various aspects, the compound has a structure represented by a formula:

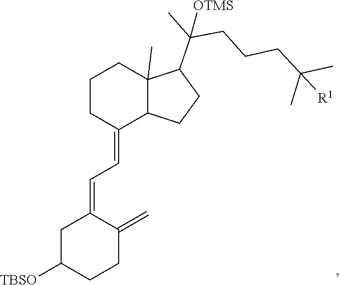

or a pharmaceutically acceptable salt thereof.

In various aspects, the compound has a structure:

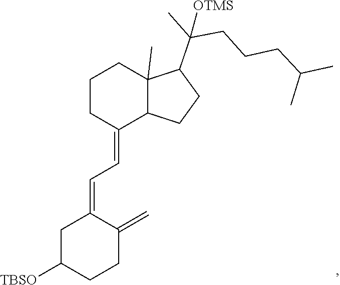

or a pharmaceutically acceptable salt thereof.

In various aspects, the compound has a structure:

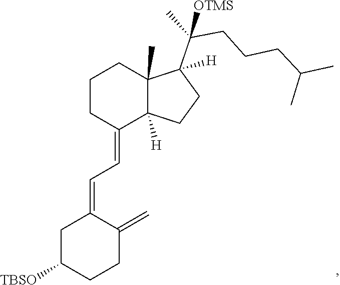

or a pharmaceutically acceptable salt thereof.

In various aspects, the compound has a structure represented by a formula:

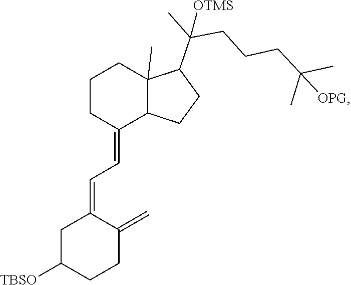

or a pharmaceutically acceptable salt thereof.

In various aspects, the compound has a structure represented by a formula:

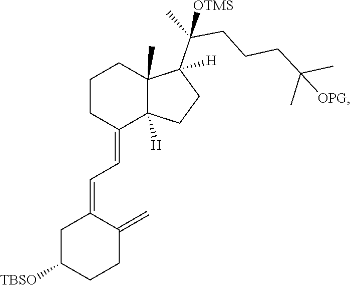

or a pharmaceutically acceptable salt thereof.

In various aspects, the compound has a structure:

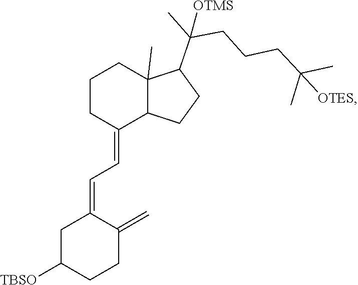

or a pharmaceutically acceptable salt thereof.

In various aspects, the compound has a structure:

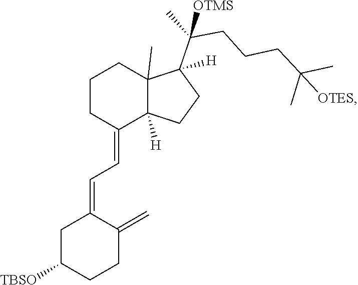

or a pharmaceutically acceptable salt thereof.

### A. R¹ Groups

In one aspect, R¹ is hydrogen or —OPG. In a further aspect, R¹ is hydrogen. In a still further aspect, R¹ is —OPG. In a yet further aspect, R¹ is —OTES.

b. Example Compounds

In one aspect, a triene can be present as:

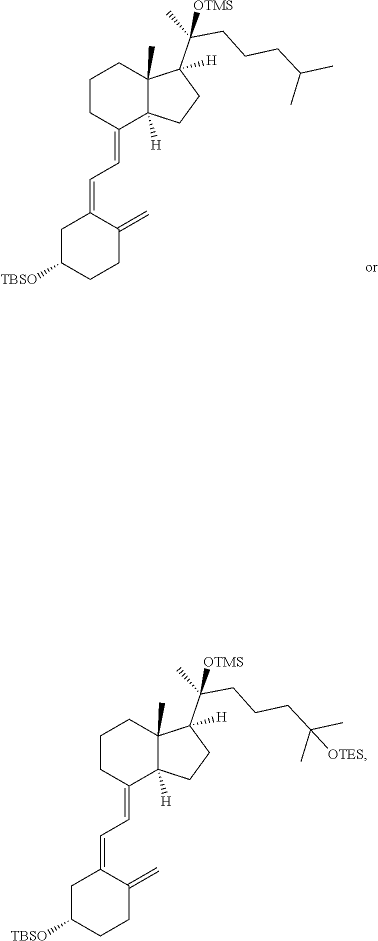

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 5. Open Chain Hydroxy Derivatives of Vitamin D₃

In one aspect, disclosed are open chain hydroxy derivatives of vitamin D₃ having a structure represented by a formula:

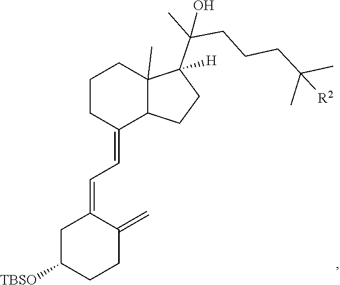

wherein R² is hydrogen or —OH, or a pharmaceutically acceptable salt thereof.

In various aspects, the compound has a structure represented by a formula:

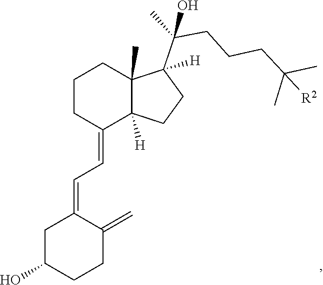

or a pharmaceutically acceptable salt thereof.

In various aspects, the compound has a structure:

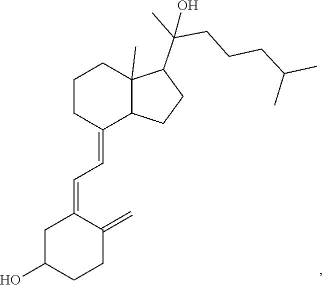 [SMILES: C=C1CCC(O)C/C1=C/C=C1\CCCC2(C)C1CCC2C(C)(O)CCCC(C)C]

or a pharmaceutically acceptable salt thereof.

In various aspects, the compound has a structure:

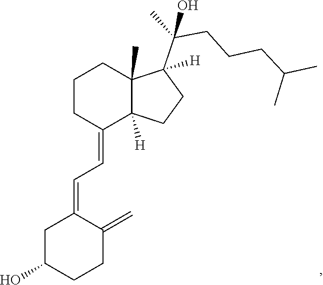 [SMILES: C=C1CC[C@H](O)C/C1=C/C=C1\CCC[C@@]2(C)[C@H]1CC[C@@H]2[C@@](C)(O)CCCC(C)C]

or a pharmaceutically acceptable salt thereof.

In various aspects, the compound has a structure:

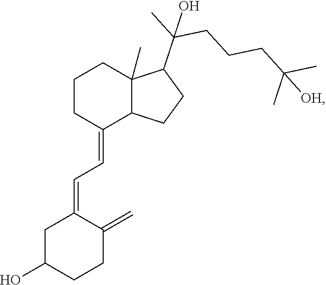 [SMILES: C=C1CCC(O)C/C1=C/C=C1\CCCC2(C)C1CCC2C(C)(O)CCCC(C)(C)O]

or a pharmaceutically acceptable salt thereof.

In various aspects, the compound has a structure:

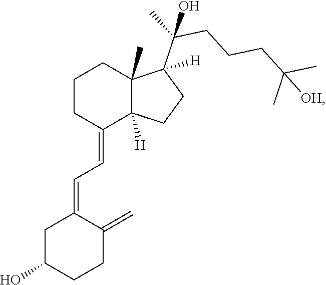 [SMILES: C=C1CC[C@H](O)C/C1=C/C=C1\CCC[C@@]2(C)[C@H]1CC[C@@H]2[C@@](C)(O)CCCC(C)(C)O]

or a pharmaceutically acceptable salt thereof.

### A. R² Groups

In one aspect, R² is hydrogen or —OH. In a further aspect, R² is hydrogen. In a still further aspect, R² is —OH.

b. Exemplary Vitamin D₃ Derivatives

In one aspect, a compound can be present as:

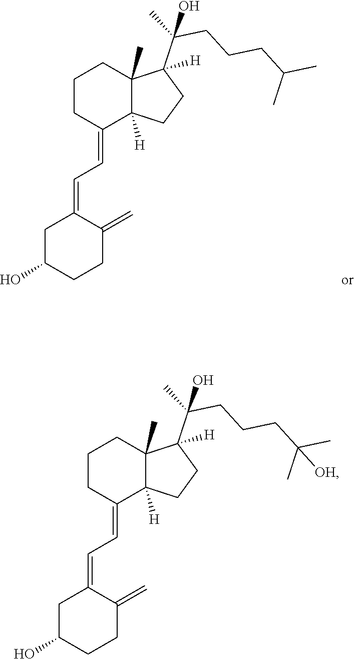 [SMILES: C=C1CC[C@H](O)C/C1=C/C=C1\CCC[C@@]2(C)[C@H]1CC[C@@H]2[C@@](C)(O)CCCC(C)C ; C=C1CC[C@H](O)C/C1=C/C=C1\CCC[C@@]2(C)[C@H]1CC[C@@H]2[C@@](C)(O)CCCC(C)(C)O]

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### C. Methods of Making Hydroxy Derivatives of Vitamin D₃ (Open Chain-Route I)

In one aspect, disclosed are methods of making open chain hydroxy derivatives of vitamin D₃. See, e.g., FIG. 4. As detailed herein, hydroxy derivatives of vitamin D₃ have demonstrated utility as therapeutic agents for a variety of autoimmune disorders including, but not limited to, lupus erythematosus, rheumatoid arthritis, inflammatory bowel disease, psoriasis, and multiple sclerosis. Thus, in various aspects, hydroxy derivatives of vitamin D₃ can be prepared by treating an appropriate ketone with two Grignard reagents to provide the desired diol (i.e., a secondary alcohol as detailed herein). The secondary hydroxyl groups can then be oxidized to the corresponding ketone (i.e., a cyclohexanone as detailed herein) before protection of the tertiary alcohol (i.e., to provide a protected tertiary alcohol as detailed herein) and coupling with an appropriate phosphine oxide (i.e., to provide a triene as detailed herein). At that time, the protecting groups can be removed, thereby providing an open chain hydroxy derivative of vitamin D₃.

### 1. Methods of Making Secondary Alcohols

In one aspect, disclosed are methods of making a compound having a structure represented by a formula:

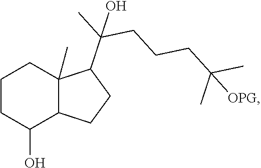

wherein PG is a silyl protecting group, or a pharmaceutically acceptable salt thereof, the method comprising reacting a methyl ketone having a structure represented by a formula:

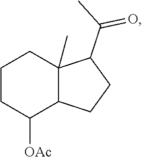 [SMILES: CC(=O)OC1CCCC2(C)C(C(C)=O)CCC12]

or a pharmaceutically acceptable salt thereof, and an alkyl halide having a structure represented by a formula:

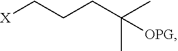

or a pharmaceutically acceptable salt thereof, wherein X is a halide.

In various aspects, reacting is in the presence of a magnesium metal.

In various aspects, reacting is in an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, reacting is a temperature from about 0° C. to about 22° C.

In various aspects, X is a halide such as, for example, a fluoride, chloride, bromide, or iodide. In a further aspect, X is a fluoride, a chloride, or a bromide. In a still further aspect, X is a chloride or a bromide. In yet a further aspect, X is a bromide.

In various aspects, PG is a silyl protecting group. In a further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, the silyl protecting group is selected from trimethylsilyl (TMS), triethylsilyl (TES), and tert-butyldimethylsilyl (TBS). In yet all further aspect, the silyl protecting group is selected from trimethylsilyl (TMS) and triethylsilyl (TES). In an even further aspect, the silyl protecting group is triethylsilyl (TES).

In various aspects, the methyl ketone has a structure:

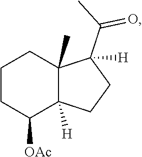 [SMILES: CC(=O)O[C@H]1CCC[C@]2(C)[C@@H](C(C)=O)CC[C@@H]12]

or a pharmaceutically acceptable salt thereof.

In various aspects, the alkyl halide has a structure:

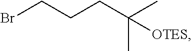

or a pharmaceutically acceptable salt thereof.

In various aspects, the compound has a structure:

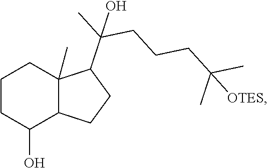

or a pharmaceutically acceptable salt thereof.

In various aspects, the compound has a structure:

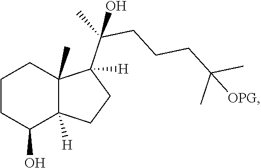

or a pharmaceutically acceptable salt thereof.

In various aspects, the compound has a structure:

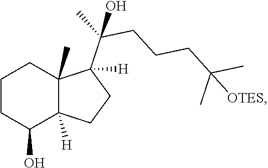

or a pharmaceutically acceptable salt thereof.

### A. Route I

In one aspect, substituted secondary alcohols can be prepared as shown below.

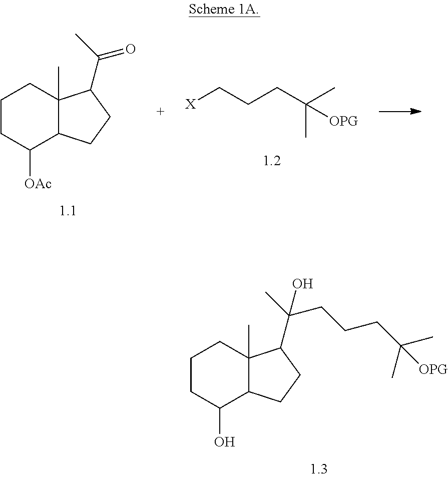

Compounds are represented in generic form, with substituents as noted in compound descriptions elsewhere herein. A more specific example is set forth below.

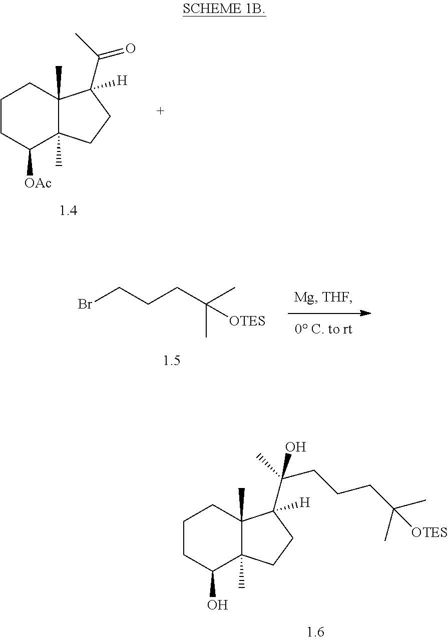

In one aspect, compounds of type 1.6, and similar compounds, can be prepared according to reaction Scheme 1B above. Thus, compounds of type 1.6 can be prepared by reacting an appropriate methyl ketone, e.g., 1.4 as shown above and an appropriate halide, e.g., 1.5 as shown above. Appropriate halides and appropriate methyl ketones are commercially available or prepared by methods known to one skilled in the art. The reaction is carried out in the presence of an appropriate aprotic solvent, e.g., tetrahydrofuran (THF), at an appropriate temperature, e.g., 0° C., wherein the alkyl halide is initially reacted magnesium metal to form a Grignard reagent. As can be appreciated by one skilled in the art, the above reaction provides an example of a generalized approach wherein compounds similar in structure to the specific reactants above (compounds similar to compounds of type 1.1 and 1.2), can be substituted in the reaction to provide substituted secondary alcohols similar to Formula 1.3.

### 2. Methods of Making Cyclohexanones

In one aspect, disclosed are methods of making a compound having a structure represented by a formula:

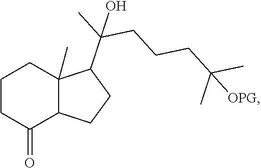

wherein PG is a protecting group selected from acetyl (Ac), pivaloyl (Piv), methoxymethyl (MOM), 2-methoxyethoxymethyl ether (MEM), benzyloxymethyl (BOM), tetrahydropyranyl (THP), trimethylsilyl (TMS), triethylsilyl (TES), tert-butyldimethylsilyl (TBS), tert-butyldiphenylsilyl (TBDPS), and triisopropylsilyl (TIPS), and trimethylsilylethoxymethyl (SEM), or a pharmaceutically acceptable salt thereof, the method comprising oxidizing an alcohol having a structure represented by a formula:

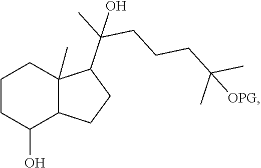

or a pharmaceutically acceptable salt thereof.

In various aspects, oxidizing is in the presence of an oxidizing agent selected from tetrapropylammonium perruthenate (TPAP), hydrogen peroxide, di-tert-butyl peroxide, oxone, chromic acid, pyridinium dichromate, and pyridinium chlorochromate. In a further aspect, oxidizing is in the presence of tetrapropylammonium perruthenate (TPAP).

In various aspects, oxidizing is in the presence of an organic, nonpolar solvent. In a further aspect, the organic, nonpolar solvent is selected from acetic acid, dichloromethane (DCM), and ethyl acetate (EtOAc). In a still further aspect, the organic, nonpolar solvent is dichloromethane (DCM).

In various aspects, oxidizing is at a temperature of from about 0° C. to about 22° C.

In various aspects, the protecting group is a silyl protecting group. In a further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, the silyl protecting group is selected from trimethylsilyl (TMS), triethylsilyl (TES), and tert-butyldimethylsilyl (TBS). In yet a further aspect, the silyl protecting group is selected from trimethylsilyl (TMS) and triethylsilyl (TES). In an even further aspect, the silyl protecting group is triethylsilyl (TES).

In various aspects, the alcohol has a structure:

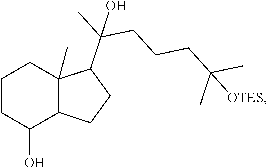

or a pharmaceutically acceptable salt thereof.

In various aspects, the alcohol has a structure represented by a formula:

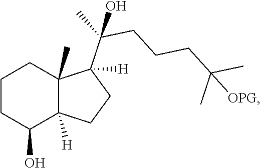

or a pharmaceutically acceptable salt thereof.

In various aspects, the alcohol has a structure:

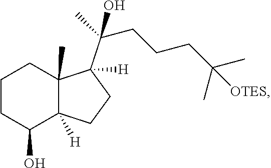

or a pharmaceutically acceptable salt thereof.

In various aspects, the compound has a structure:

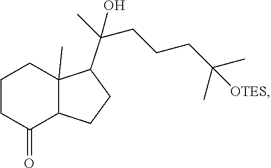

or a pharmaceutically acceptable salt thereof.

In various aspects, the compound has a structure represented by a formula:

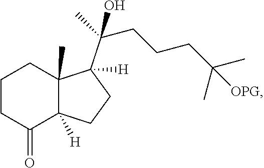

or a pharmaceutically acceptable salt thereof.

In various aspects, the compound has a structure:

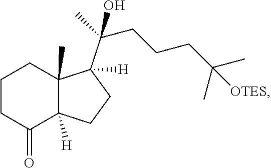

or a pharmaceutically acceptable salt thereof.

### A. Route I

In one aspect, substituted cyclohexanones can be prepared as shown below.

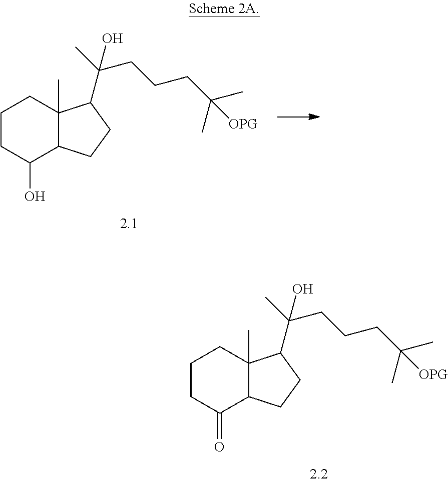

Compounds are represented in generic form, with substituents as noted in compound descriptions elsewhere herein. A more specific example is set forth below.

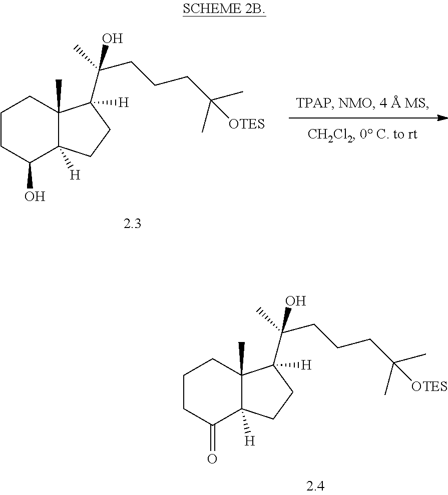

In one aspect, compounds of type 2.4, and similar compounds, can be prepared according to reaction Scheme 2B above. Thus, compounds of type 2.4 can be prepared by oxidation of an appropriate secondary alcohol, e.g., 2.3 as shown above. The oxidation is carried out in the presence of appropriate oxidizing agents, e.g., tetrapropylammonium perruthenate (TPAP) and 4-methylmorpholine N-oxide (NMO), in an appropriate solvent, e.g., dichloromethane (DCM), at an appropriate temperature, e.g., 0° C. As can be appreciated by one skilled in the art, the above reaction provides an example of a generalized approach wherein compounds similar in structure to the specific reactants above (compounds similar to compounds of type 2.1), can be substituted in the reaction to provide substituted cyclohexanones similar to Formula 2.2.

### 3. Methods of Making Protected Tertiary Alcohols

In one aspect, disclosed are methods of making a compound having a structure represented by a formula:

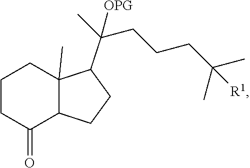

wherein R¹ is hydrogen or —OPG; and wherein each occurrence of PG is independently a protecting group selected from acetyl (Ac), pivaloyl (Piv), methoxymethyl (MOM), 2-methoxyethoxymethyl ether (MEM), benzyloxymethyl (BOM), tetrahydropyranyl (THP), trimethylsilyl (TMS), triethylsilyl (TES), tert-butyldimethylsilyl (TBS), tert-butyldiphenylsilyl (TBDPS), and triisopropylsilyl (TIPS), and trimethylsilylethoxymethyl (SEM), or a pharmaceutically acceptable salt thereof, the method comprising protecting an alcohol having a structure represented by a formula:

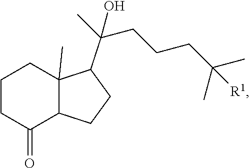

or a pharmaceutically acceptable salt thereof.

In various aspects, protecting is via addition of a triflate. In a further aspect, the triflate is a silyl triflate. In a still further aspect, the silyl triflate is trimethylsilyl triflate.

In various aspects, protecting is in the presence of a base. In a further aspect, the base is selected from 2,6-lutidine, quinuclidine, 2,2,6,6-tetramethylpiperidine (TMP), pempidine (PMP), tributylamine, 1,5,7-triazabicyclo(4.4.0)dec-5-ene (TBD), 7-methyl-1,5,7-triazabicyclo(4.4.0)dec-5-ene (MTBD), 1,8-diazabicyclo[5.4.0]undec-7-ene (DBU), 1,5-diazabicyclo[4.3.0]non-5-ene (DBN), 1,1,3,3-tetramethylguanidine (TMG), 1,4-diazabicyclo[2.2.2]octan (TED), and collidine. In a still further aspect, the base is 2,6-lutidine.

In various aspects, protecting is in an organic, nonpolar solvent. In a further aspect, the organic, nonpolar solvent is selected from acetic acid, dichloromethane (DCM), ethyl acetate (EtOAc), diethyl ether, and pyridine. In a still further aspect, the organic, nonpolar solvent is dichloromethane (DCM).

In various aspects, R¹ is hydrogen or —OPG. In a further aspect, R¹ is hydrogen. In a still further aspect, R¹ is —OPG. In a yet further aspect, R¹ is —OTES.

In various aspects, each occurrence of PG is independently a silyl protecting group. In a further aspect, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, each occurrence of PG is independently a silyl protecting group selected from trimethylsilyl (TMS) and triethylsilyl (TES). In yet a further aspect, each occurrence of PG is independently a silyl protecting group selected from trimethylsilyl (TMS) and triethylsilyl (TES).

In various aspects, the alcohol has a structure represented by a formula:

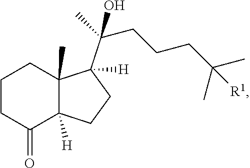

or a pharmaceutically acceptable salt thereof.

In various aspects, the alcohol has a structure:

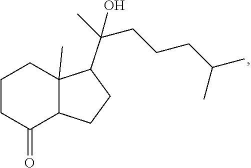 [SMILES: CC(C)CCCC(C)(O)C1CCC2C(=O)CCCC21C]

or a pharmaceutically acceptable salt thereof.

In various aspects, the alcohol has a structure:

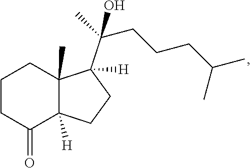 [SMILES: CC(C)CCC[C@](C)(O)[C@H]1CC[C@H]2C(=O)CCC[C@@]21C]

or a pharmaceutically acceptable salt thereof.

In various aspects, the alcohol has a structure represented by a formula:

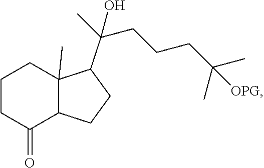

or a pharmaceutically acceptable salt thereof.

In various aspects, the alcohol has a structure represented by a formula:

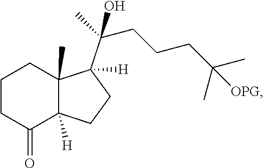

or a pharmaceutically acceptable salt thereof.

In various aspects, the alcohol has a structure:

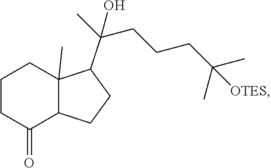

or a pharmaceutically acceptable salt thereof.

In various aspects, the alcohol has a structure:

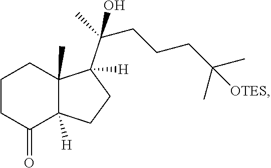

or a pharmaceutically acceptable salt thereof.

In various aspects, the alcohol has a structure represented by a formula:

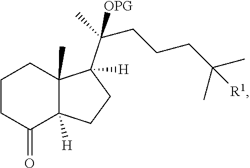

or a pharmaceutically acceptable salt thereof.

In various aspects, the alcohol has a structure represented by a formula:

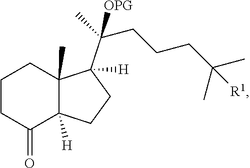

or a pharmaceutically acceptable salt thereof.

In various aspects, the alcohol has a structure represented by a formula:

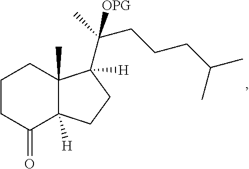

or a pharmaceutically acceptable salt thereof.

In various aspects, the alcohol has a structure:

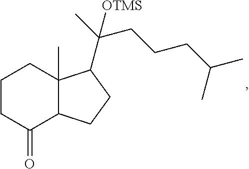

or a pharmaceutically acceptable salt thereof.

In various aspects, the alcohol has a structure:

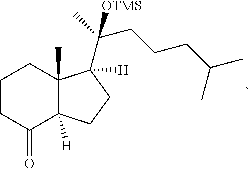

or a pharmaceutically acceptable salt thereof.

In various aspects, the compound has a structure represented by a formula:

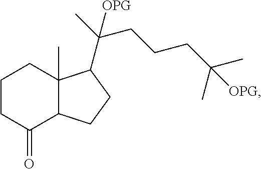

or a pharmaceutically acceptable salt thereof.

In various aspects, the compound has a structure represented by a formula:

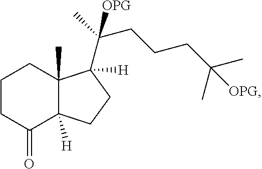

or a pharmaceutically acceptable salt thereof.

In various aspects, the compound has a structure:

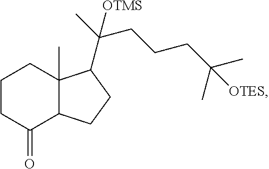

or a pharmaceutically acceptable salt thereof.

In various aspects, the compound has a structure:

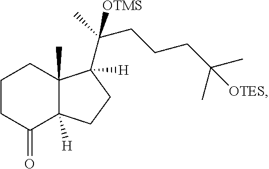

or a pharmaceutically acceptable salt thereof.

### A. Route I

In one aspect, substituted protected tertiary alcohols can be prepared as shown below.

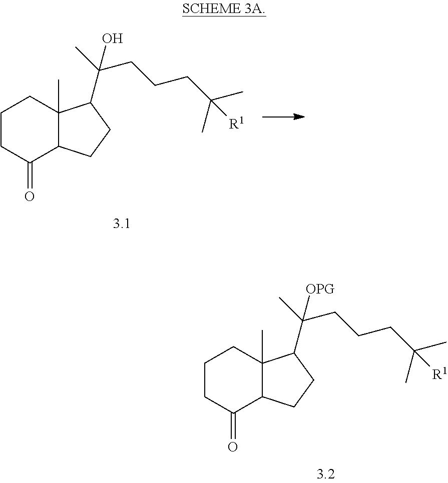

Compounds are represented in generic form, with substituents as noted in compound descriptions elsewhere herein. A more specific example is set forth below.

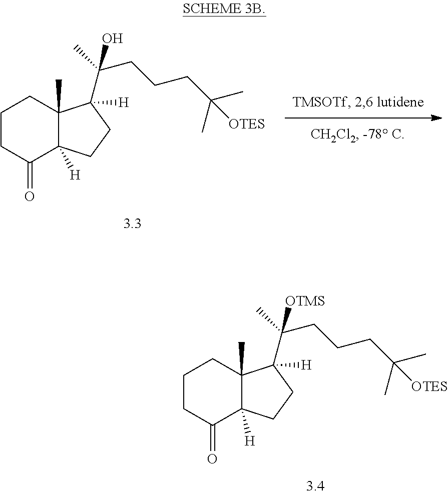

In one aspect, compounds of type 3.4, and similar compounds, can be prepared according to reaction Scheme 3B above. Thus, compounds of type 3.4 can be prepared by protection of an appropriate tertiary alcohol, e.g., 3.3 as shown above. The protection is carried out in the presence of an appropriate activated protecting group, e.g., trimethylsilyl triflate, in an appropriate solvent, e.g., dichloromethane (DCM), at an appropriate temperature, e.g., −78° C., in the presence of an appropriate base, e.g., 2,6-lutidine, for an appropriate amount of time, e.g., 1 h. As can be appreciated by one skilled in the art, the above reaction provides an example of a generalized approach wherein compounds similar in structure to the specific reactants above (compounds similar to compounds of type 3.1), can be substituted in the reaction to provide substituted protected tertiary alcohol analogs similar to Formula 3.2.

### 4. Methods of Making Trienes

In one aspect, disclosed are methods of making a compound having a structure represented by a formula:

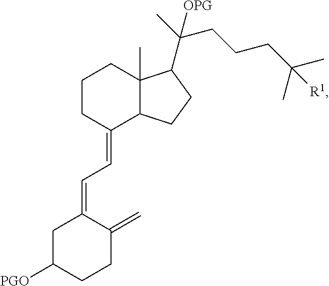

wherein R¹ is hydrogen or —OPG; and wherein each occurrence of PG is independently a silyl protecting group, or a pharmaceutically acceptable salt thereof, the method comprising coupling a ketone having a structure represented by a formula:

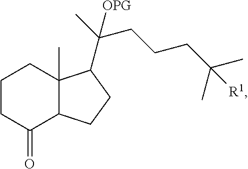

or a pharmaceutically acceptable salt thereof, and a phosphine oxide having a structure represented by a formula:

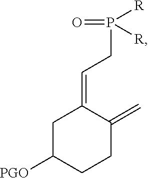

wherein each occurrence of R is independently C₆H₅ substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In various aspects, coupling is in the presence of a base. In a further aspect, the base is selected from n-butyl lithium, 1,8-diazabicyclo[5.4.0]undec-7-ene (DBU), sodium hydride, and potassium tert-butoxide. In a still further aspect, the base is n-butyl lithium.

In various aspects, coupling is in an aprotic solvent. In a further aspect, the aprotic solvent is selected from diethyl ether (Et₂O), cyclopentyl methyl ether (CPME), and tetrahydrofuran (THF). In a still further apect, the aprotic solvent is tetrahydrofuran (THF).

In various aspects, coupling is at a temperature of about −78° C.

In various aspects, R¹ is hydrogen or —OPG. In a further aspect, R¹ is hydrogen. In a still further aspect, R¹ is —OPG. In a yet further aspect, R¹ is —OTES.

In various aspects, each occurrence of PG is independently a silyl protecting group. In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a further aspect, each occurrence of PG is independently a silyl protecting group selected from trimethylsilyl (TMS) and triethylsilyl (TES). In a further aspect, each occurrence of PG is independently a silyl protecting group selected from trimethylsilyl (TMS) and triethylsilyl (TES).

In various aspects, each occurrence of R is independently C₆H₅ substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of R is independently C₆H₅ substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of R is independently C₆H₅ substituted with 0 or 1 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of R is independently C₆H₅ substituted with 1 group independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of R is unsubstituted C₆H.

In various aspects, the ketone has a structure represented by a formula:

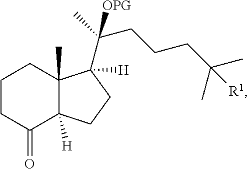

or a pharmaceutically acceptable salt thereof.

In various aspects, the ketone has a structure represented by a formula:

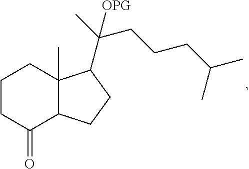

or a pharmaceutically acceptable salt thereof.

In various aspects, the ketone has a structure represented by a formula:

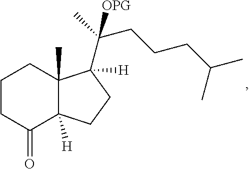

or a pharmaceutically acceptable salt thereof.

In various aspects, the ketone has a structure represented by a formula:

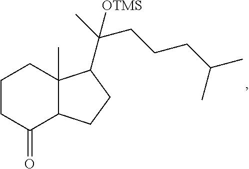

or a pharmaceutically acceptable salt thereof.

In various aspects, the ketone has a structure represented by a formula:

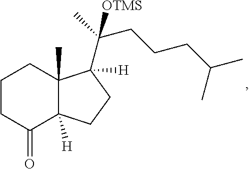

or a pharmaceutically acceptable salt thereof.

In various aspects, the ketone has a structure represented by a formula:

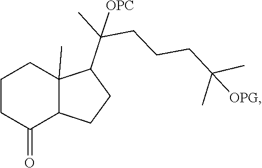

or a pharmaceutically acceptable salt thereof.

In various aspects, the ketone has a structure represented by a formula:

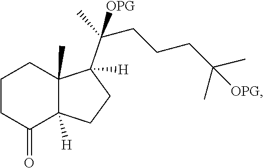

or a pharmaceutically acceptable salt thereof.

In various aspects, the ketone has a structure:

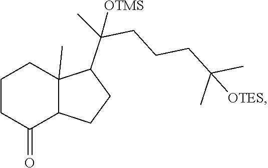

or a pharmaceutically acceptable salt thereof.

In various aspects, the ketone has a structure:

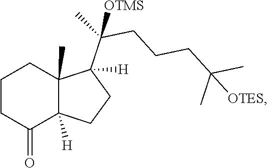

or a pharmaceutically acceptable salt thereof.

In various aspects, the phosphine oxide has a structure represented by a formula:

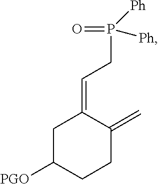

or a pharmaceutically acceptable salt thereof.

In various aspects, the phosphine oxide has a structure represented by a formula:

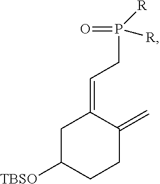

or a pharmaceutically acceptable salt thereof.

In various aspects, the phosphine oxide has a structure:

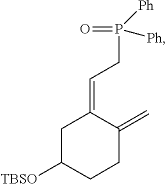 [SMILES: C=C1CCC(O[Si](C)(C)C(C)(C)C)C/C1=C/CP(=O)(c1ccccc1)c1ccccc1]

or a pharmaceutically acceptable salt thereof.

In various aspects, the phosphine oxide has a structure represented by a formula:

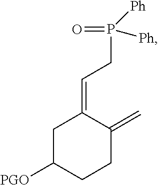

or a pharmaceutically acceptable salt thereof.

In various aspects, wherein the phosphine oxide has a structure:

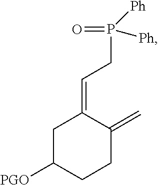

or a pharmaceutically acceptable salt thereof.

In various aspects, the compound has a structure represented by a formula:

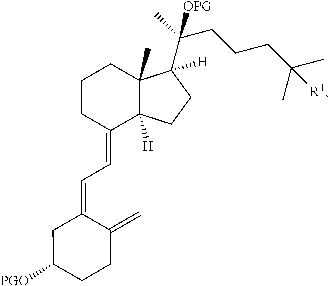

or a pharmaceutically acceptable salt thereof.

In various aspects, the compound has a structure represented by a formula:

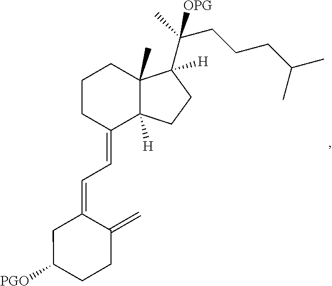

or a pharmaceutically acceptable salt thereof.

In various aspects, the compound has a structure represented by a formula:

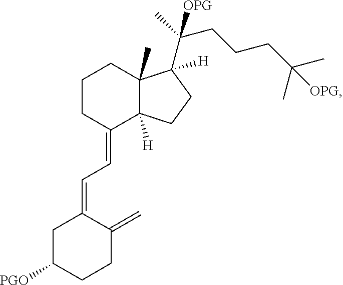

or a pharmaceutically acceptable salt thereof.

In various aspects, the compound has a structure represented by a formula:

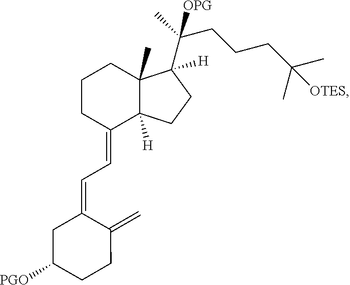

or a pharmaceutically acceptable salt thereof.

In various aspects, the compound has a structure represented by a formula:

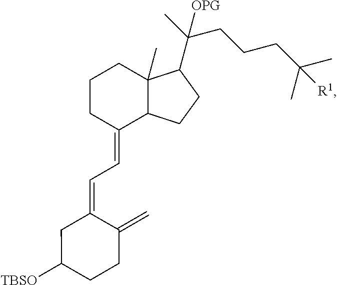

or a pharmaceutically acceptable salt thereof.

In various aspects, the compound has a structure represented by a formula:

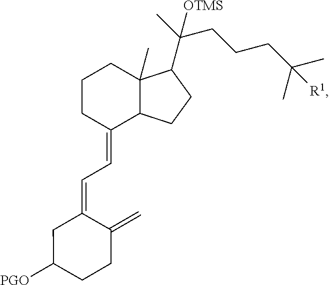

or a pharmaceutically acceptable salt thereof.

In various aspects, the compound has a structure represented by a formula:

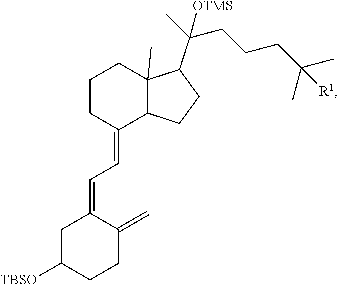

or a pharmaceutically acceptable salt thereof.

In various aspects, the compound has a structure:

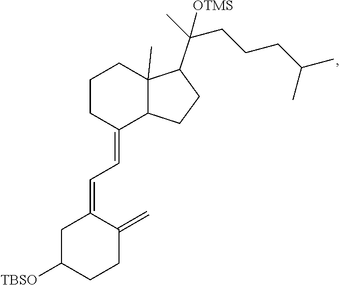

or a pharmaceutically acceptable salt thereof.

In various aspects, the compound has a structure:

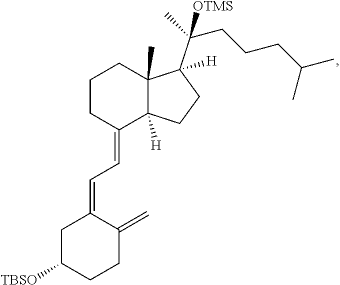

or a pharmaceutically acceptable salt thereof.

In various aspects, the compound has a structure:

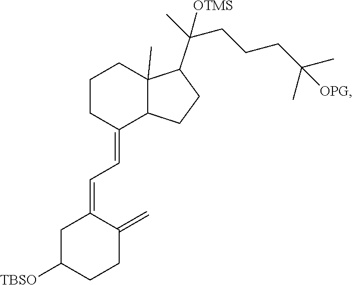

or a pharmaceutically acceptable salt thereof.

In various aspects, the compound has a structure:

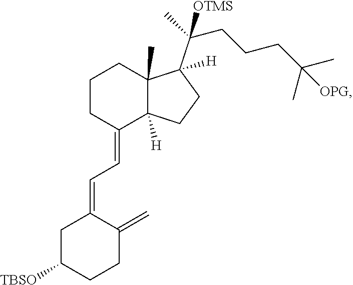

or a pharmaceutically acceptable salt thereof.

In various aspects, the compound has a structure:

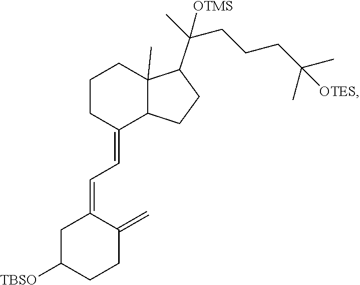

or a pharmaceutically acceptable salt thereof.

In various aspects, the compound has a structure:

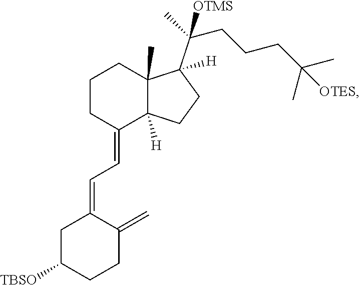

or a pharmaceutically acceptable salt thereof.

### A. Route I

In one aspect, substituted triene analogs can be prepared as shown below.

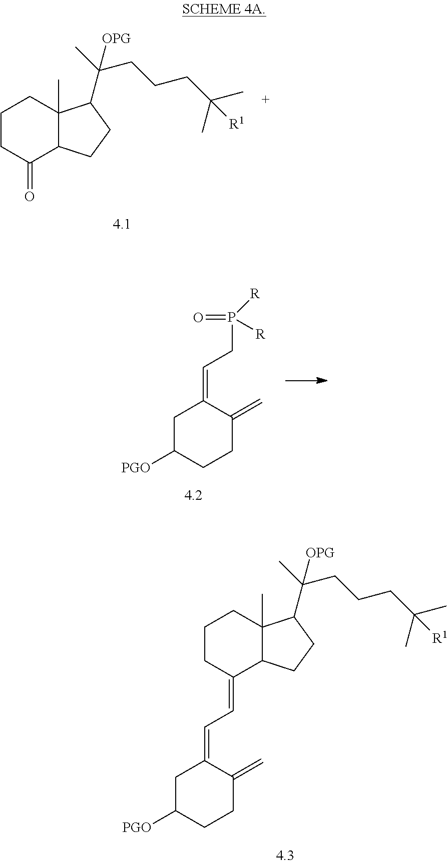

Compounds are represented in generic form, with substituents as noted in compound descriptions elsewhere herein. A more specific example is set forth below.

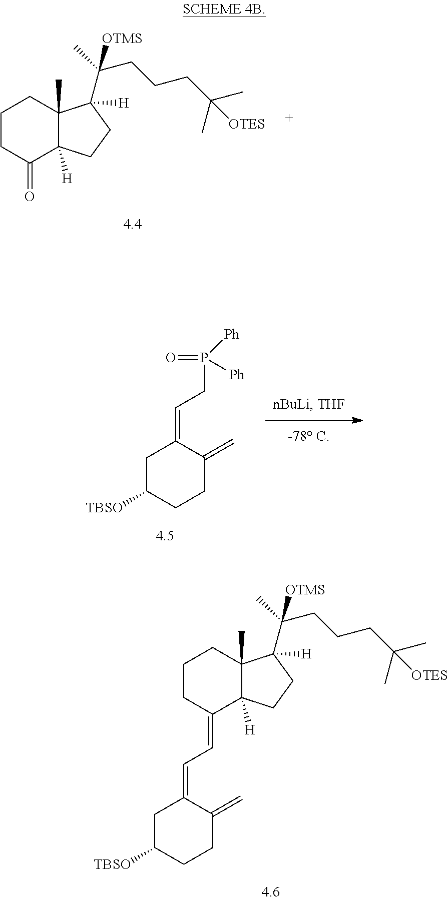

In one aspect, compounds of type 4.6, and similar compounds, can be prepared according to reaction Scheme 4B above. Thus, compounds of type 4.6 can be prepared by a Wittig reaction between an appropriate ketone, e.g., 4.4, and an appropriate phosphine oxide, e.g., 4.5, as shown above. Appropriate phosphine oxides are commercially available or prepared by methods known to one skilled in the art. The Wittig reaction is carried out in an appropriate solvent, e.g., anhydrous THF, at an appropriate temperature, e.g., −78° C., in the presence of an appropriate base, e.g., n-BuLi, for an appropriate amount of time, e.g., 4 h. As can be appreciated by one skilled in the art, the above reaction provides an example of a generalized approach wherein compounds similar in structure to the specific reactants above (compounds similar to compounds of type 4.1 and 4.2), can be substituted in the reaction to provide substituted triene analogs similar to Formula 4.3.

### 5. Methods of Making Open Chain Hydroxy Derivatives of Vitamin D₃

In one aspect, disclosed are methods of making a compound having a structure represented by a formula:

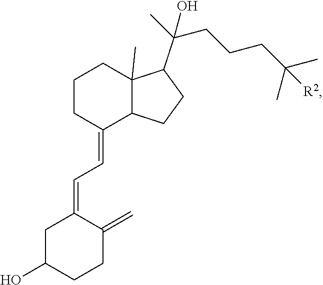

wherein R² is hydrogen or —OH, or a pharmaceutically acceptable salt thereof, the method comprising deprotecting a protected alcohol having a structure represented by a formula:

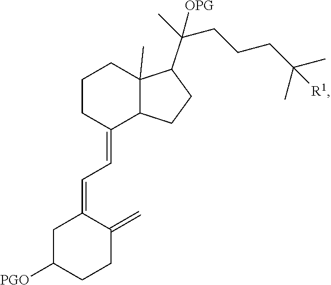

wherein R¹ is hydrogen or —OPG; and, wherein each occurrence of PG is independently a silyl protecting group, or a pharmaceutically acceptable salt thereof.

In various aspects, deprotecting is in the presence of a deprotecting agent selected from potassium fluoride, cesium fluoride, tetrabutylammonium fluoride (TBAF), tris(dimethylamino)sulfur (trimethylsilyl)difluoride (TASF), tetrabutylammonium triphenyldifluorosilicate (TBAT), tetraphenylbismuth fluoride, cadmium fluoride, acidic aqueous tetrahydrofuran (THF), acidic methanol, and acetic acid. In a further aspect, deprotecting is in the presence of tetrabutylammonium fluoride (TBAF).

In various aspects, deprotecting is in an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, R¹ is hydrogen or —PG. In a further aspect, R¹ is hydrogen. In a still further aspect, R¹ is —PG. In a still further aspect, R¹ is —OTES.

In various aspects, R² is hydrogen or —OH. In a further aspect, R² is hydrogen. In a still further aspect, R² is —OH.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In further aspect, each occurrence of PG is independently a silyl protecting group selected from trimethylsilyl (TMS), triethylsilyl (TES), and tert-butyldimethylsilyl (TBS). In a still further aspect, each occurrence of PG is a different silyl protecting group.

In various aspects, the protected alcohol has a structure represented by a formula:

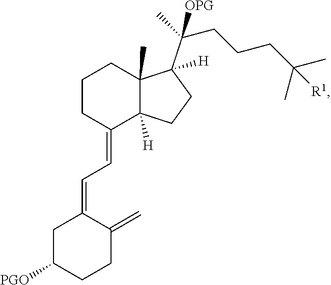

or a pharmaceutically acceptable salt thereof.

In various aspects, the protected alcohol has a structure represented by a formula:

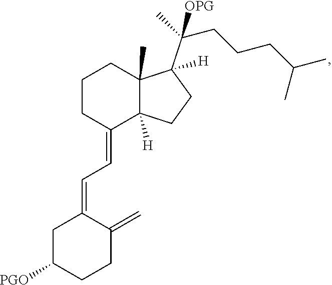

or a pharmaceutically acceptable salt thereof.

In various aspects, the protected alcohol has a structure represented by a formula:

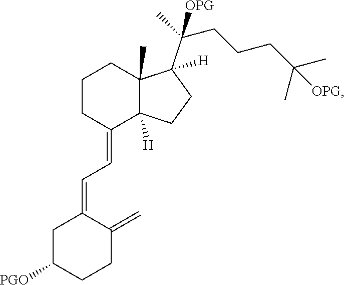

or a pharmaceutically acceptable salt thereof.

In various aspects, the protected alcohol has a structure represented by a formula:

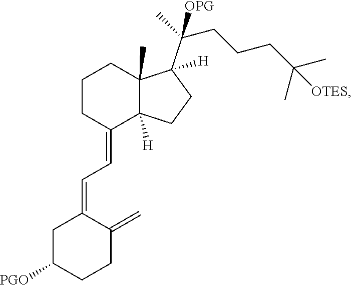

or a pharmaceutically acceptable salt thereof.

In various aspects, the protected alcohol has a structure represented by a formula:

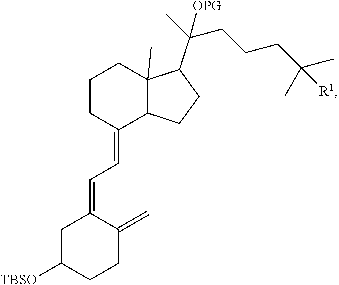

or a pharmaceutically acceptable salt thereof.

In various aspects, the protected alcohol has a structure represented by a formula:

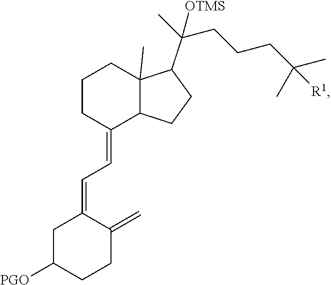

or a pharmaceutically acceptable salt thereof.

In various aspects, the protected alcohol has a structure represented by a formula:

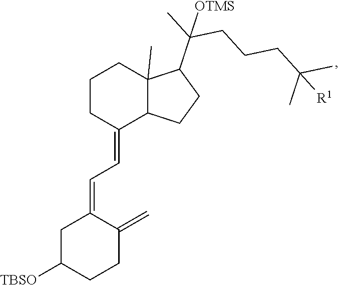

or a pharmaceutically acceptable salt thereof.

In various aspects, the protected alcohol has a structure:

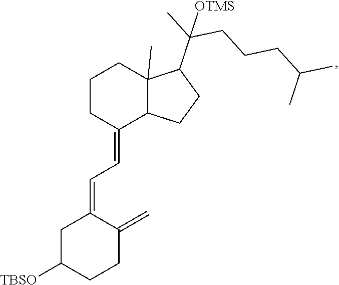

or a pharmaceutically acceptable salt thereof.

In various aspects, the protected alcohol has a structure:

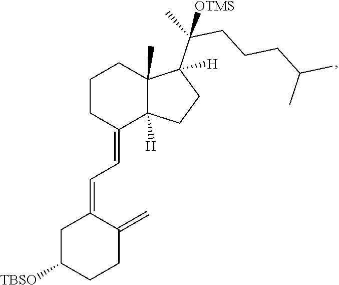

or a pharmaceutically acceptable salt thereof.

In various aspects, the protected alcohol has a structure:

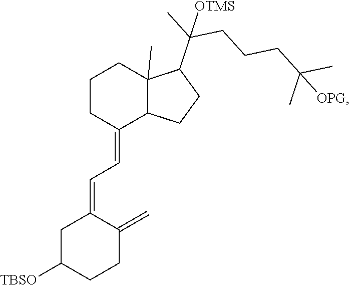

or a pharmaceutically acceptable salt thereof.

In various aspects, the protected alcohol has a structure:

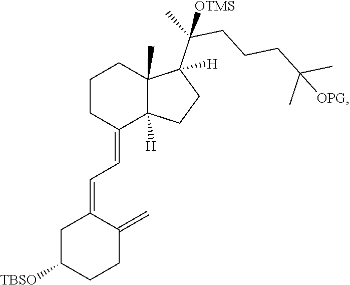

or a pharmaceutically acceptable salt thereof.

In various aspects, the protected alcohol has a structure:

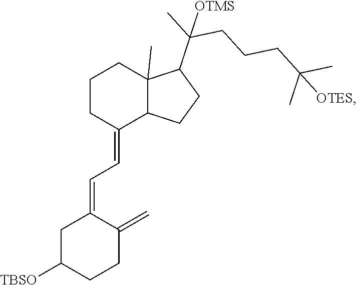

or a pharmaceutically acceptable salt thereof.

In various aspects, the protected alcohol has a structure:

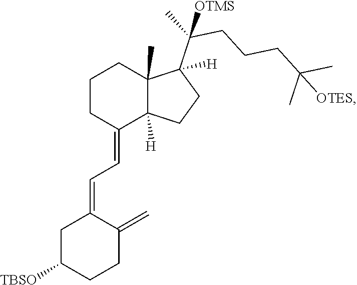

or a pharmaceutically acceptable salt thereof.

In various aspects, the compound has a structure represented by a formula:

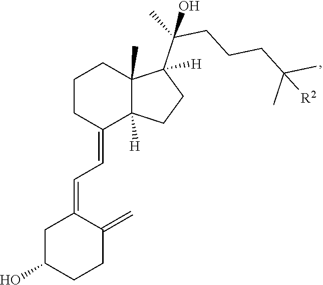

or a pharmaceutically acceptable salt thereof.

In various aspects, the compound has a structure:

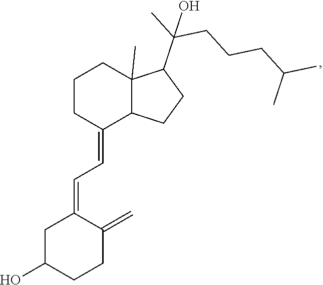 [SMILES: C=C1CCC(O)C/C1=C/C=C1\CCCC2(C)C1CCC2C(C)(O)CCCC(C)C]

or a pharmaceutically acceptable salt thereof.

In various aspects, the compound has a structure:

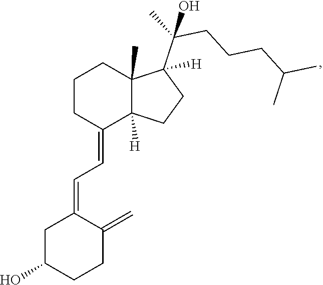 [SMILES: C=C1CC[C@H](O)C/C1=C/C=C1\CCC[C@@]2(C)[C@H]1CC[C@@H]2[C@@](C)(O)CCCC(C)C]

or a pharmaceutically acceptable salt thereof.

In various aspects, the compound has a structure:

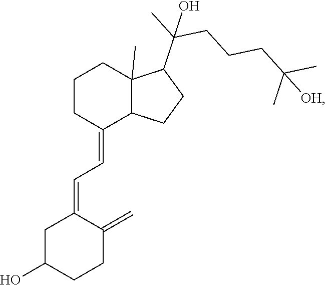 [SMILES: C=C1CCC(O)C/C1=C/C=C1\CCCC2(C)C1CCC2C(C)(O)CCCC(C)(C)O]

or a pharmaceutically acceptable salt thereof.

In various aspects, the compound has a structure:

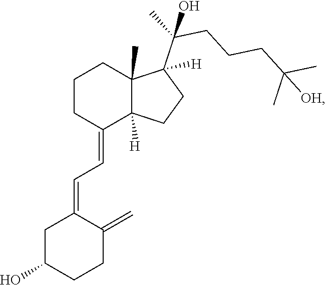 [SMILES: C=C1CC[C@H](O)C/C1=C/C=C1\CCC[C@@]2(C)[C@H]1CC[C@@H]2[C@@](C)(O)CCCC(C)(C)O]

or a pharmaceutically acceptable salt thereof.

### A. Route I

In one aspect, substituted open chain hydroxy derivatives of vitamin D₃ can be prepared as shown below.

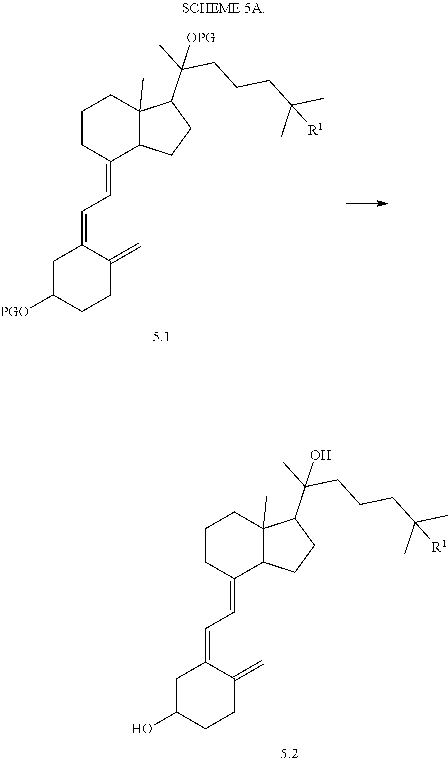

Compounds are represented in generic form, with substituents as noted in compound descriptions elsewhere herein. A more specific example is set forth below.

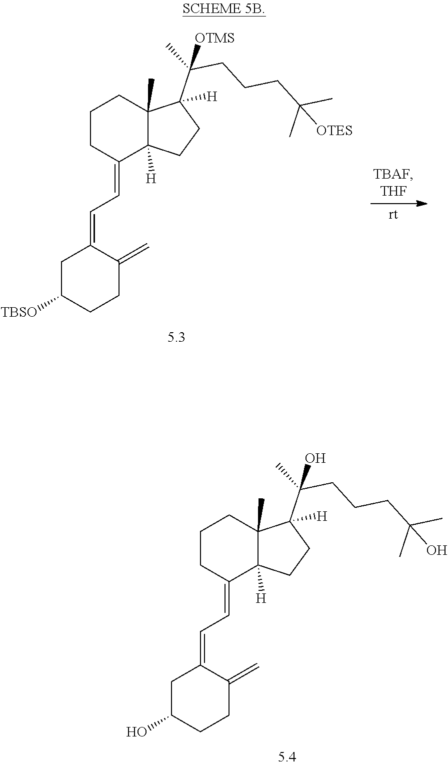

In one aspect, compounds of type 5.4, and similar compounds, can be prepared according to reaction Scheme 5B above. Thus, compounds of type 5.4 can be prepared by deprotection of an appropriate triene, e.g., 5.3, as shown above. The deprotection is carried out in an appropriate solvent, e.g., anhydrous tetrahydroduran (THF), at an appropriate temperature, e.g., room temperature, in the presence of a deprotecting agent, e.g., tetra-n-butylammonium fluoride, for an appropriate amount of time, e.g., 18 h. As can be appreciated by one skilled in the art, the above reaction provides an example of a generalized approach wherein compounds similar in structure to the specific reactants above (compounds similar to compounds of type 5.1), can be substituted in the reaction to provide substituted open chain hydroxy derivatives of vitamin D₃ similar to Formula 5.2.

### D. Compounds Useful as Intermediates Towards the Preparation of Hydroxy Derivatives of Vitamin D₃(Open Chain-Route II)

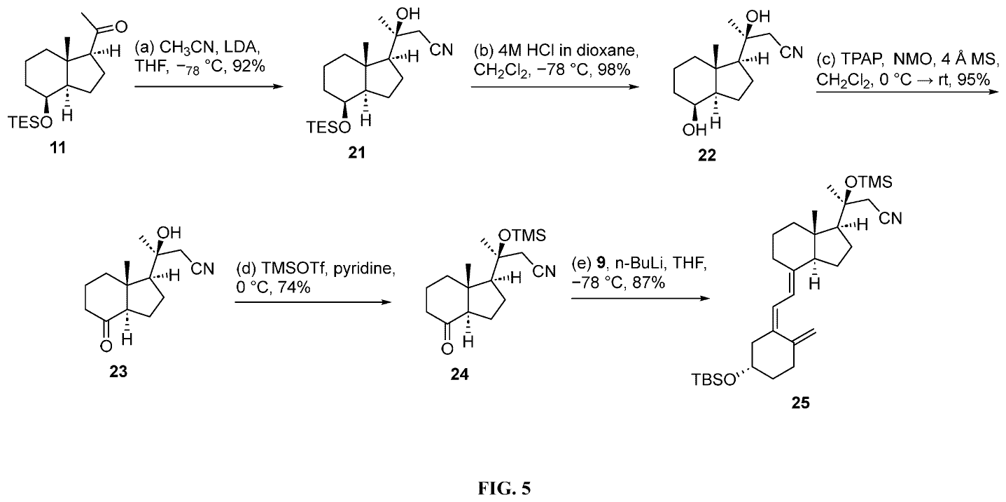

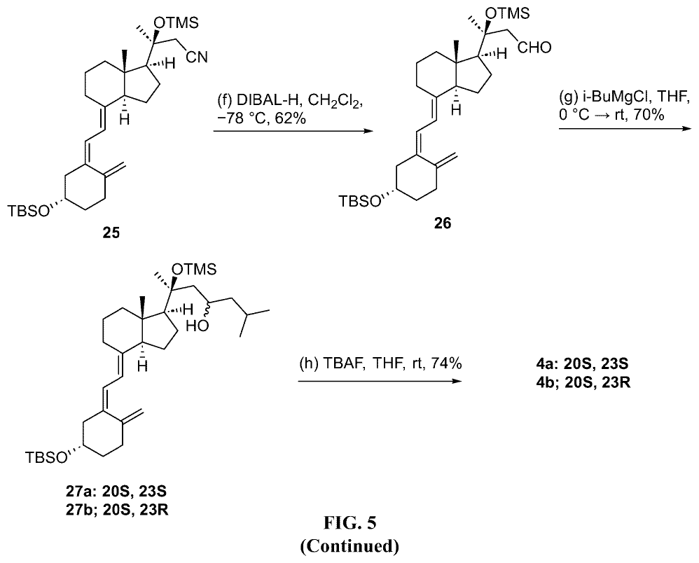

In one aspect, disclosed are compounds useful as intermediates towards the preparation of open chain hydroxy derivatives of vitamin D₃. See, e.g., FIG. 5. Hydroxy derivatives of vitamin D₃ have demonstrated utility as therapeutic agents for a variety of autoimmune disorders including, but not limited to, lupus erythematosus, rheumatoid arthritis, inflammatory bowel disease, psoriasis, and multiple sclerosis. Thus, in various aspects, disclosed are cyano derivatives, secondary alcohols, cyclohexanones, protected tertiary alcohols, trienes, aldehydes, and alkyl alcohols useful as intermediates in the synthesis of open chain hydroxy derivatives of vitamin D₃.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Nitrile and Ester Derivatives

In one aspect, disclosed are nitrile and ester derivatives having a structure represented by a formula:

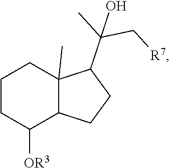

wherein R³ is selected from hydrogen and an alcohol protecting group; wherein R⁷ is selected from —CN and —CO₂R¹⁰; and wherein R¹⁰ is C1-C4 alkyl, or a pharmaceutically acceptable salt thereof.

In various aspect, the compound has a structure represented by a formula:

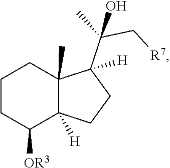

or a pharmaceutically acceptable salt thereof.

In various aspect, the compound has a structure represented by a formula:

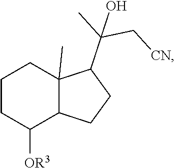

or a pharmaceutically acceptable salt thereof.

In various aspects, the compound has a structure represented by a formula:

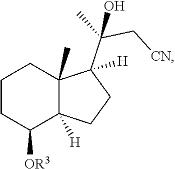

or a pharmaceutically acceptable salt thereof.

In various aspects, the compound has a structure represented by a formula:

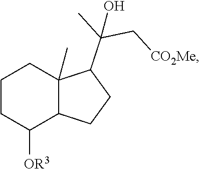

or a pharmaceutically acceptable salt thereof.

In various aspects, the compound has a structure represented by a formula:

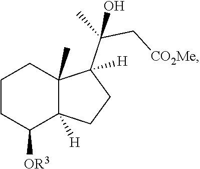

or a pharmaceutically acceptable salt thereof.

In various aspects, the compound has a structure represented by a formula:

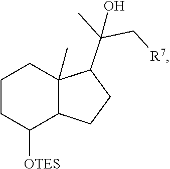

or a pharmaceutically acceptable salt thereof.

In various aspects, the compound has a structure represented by a formula:

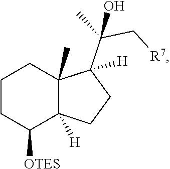

or a pharmaceutically acceptable salt thereof.

In various aspects, the compound has a structure:

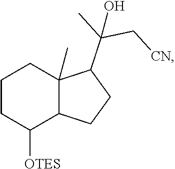

or a pharmaceutically acceptable salt thereof.

In various aspects, the compound has a structure:

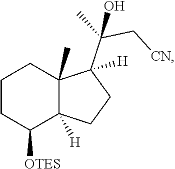

or a pharmaceutically acceptable salt thereof.

In various aspects, the compound has a structure:

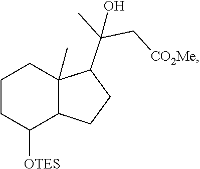

or a pharmaceutically acceptable salt thereof.

In various aspects, the compound has a structure:

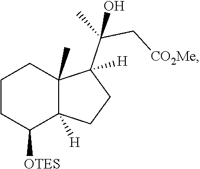

or a pharmaceutically acceptable salt thereof.

### A. R³ Groups

In one aspect, R³ is selected from hydrogen and an alcohol protecting group. In a further aspect, R³ is selected from hydrogen, acetyl, benzoyl, benzyl, methoxyethoxymethyl ether (MEM), methoxymethyl ether (MOM), para-methoxybenzyl ether (PMB), para-methoxyphenyl ether (PMP), pivaloyl, tert-butyl ether, tetrahydropyranyl (THP), trityl, trimethylsilyl (TMS), triethylsilyl (TES), tert-butyldimethylsilyl (TBS), tert-butyldiphenylsilyl (TBDPS), and triisopropylsilyl (TIPS). In a still further aspect, R³ is selected from hydrogen and a silyl protecting group. In yet a further aspect, R³ is selected from hydrogen, trimethylsilyl (TMS), triethylsilyl (TES), tert-butyldimethylsilyl (TBS), tert-butyldiphenylsilyl (TBDPS), and triisopropylsilyl (TIPS). In an even further aspect, R³ is selected from hydrogen and triethylsilyl (TES).

In various aspects, R³ is an alcohol protecting group. In a further aspect, the alcohol protecting group is selected from acetyl, benzoyl, benzyl, methoxyethoxymethyl ether (MEM), methoxymethyl ether (MOM), para-methoxybenzyl ether (PMB), para-methoxyphenyl ether (PMP), pivaloyl, tert-butyl ether, tetrahydropyranyl (THP), trityl, trimethylsilyl (TMS), triethylsilyl (TES), tert-butyldimethylsilyl (TBS), tert-butyldiphenylsilyl (TBDPS), and triisopropylsilyl (TIPS). In a yet further aspect, the alcohol protecting group is a silyl protecting group. In a still further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In an even further aspect, the silyl protecting group is triethylsilyl (TES).

In various aspects, R³ is hydrogen.

b. R⁷ Groups

In one aspect, R⁷ is selected from —CN and —CO₂R¹⁰. In a further aspect, R⁷ is —CN. In a still further aspect, R⁷ is —CO₂R¹⁰. In an even still further aspect, R⁷ is —CO₂Me.

c. R¹⁰ Groups

In one aspect, R¹⁰ is a C1-C4 alkyl. In a further aspect, R¹⁰ is selected from methyl, ethyl, n-propyl, and i-propyl. In a still further aspect, R¹⁰ is selected from methyl and ethyl. In a yet further aspect, R¹⁰ is methyl. In an even still further aspect, R¹⁰ is ethyl.

d. Exemplary Cyano Derivatives

In one aspect, a cyano derivative can be present as:

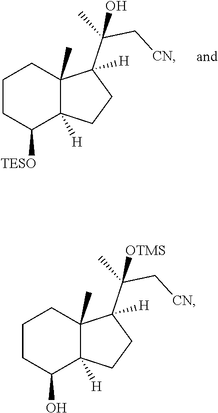

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 2. Secondary Alcohols

In one aspect, disclosed secondary alcohols having a structure represented by a formula:

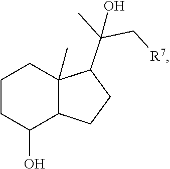

wherein R⁷ is selected from —CN and —CO₂R¹⁰; and wherein R¹⁰ is C1-C4 alkyl, or a pharmaceutically acceptable salt thereof, the method comprising deprotecting an alcohol having a structure represented by a formula:

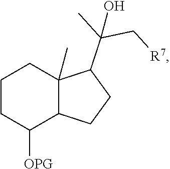

wherein PG is selected from an alcohol protecting group, or a pharmaceutically acceptable salt thereof.

In various aspects, the alcohol protecting group is selected from acetyl, benzoyl, benzyl, methoxyethoxymethyl ether (MEM), methoxymethyl ether (MOM), para-methoxybenzyl ether (PMB), para-methoxyphenyl ether (PMP), pivaloyl, tert-butyl ether, tetrahydropyranyl (THP), trityl, trimethylsilyl (TMS), triethylsilyl (TES), tert-butyldimethylsilyl (TBS), tert-butyldiphenylsilyl (TBDPS), and triisopropylsilyl (TIPS). In a still further aspect, the alcohol protecting group is a silyl protecting group. In a yet further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In an even still further aspect, the silyl protecting group is triethylsilyl (TES).

In various aspects, R⁷ is —CN.

In various aspects, R⁷ is —CO₂R¹⁰. In a further aspect, R⁷ is —CO₂Me.

In various aspects, the alcohol has a structure represented by a formula:

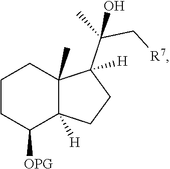

or a pharmaceutically acceptable salt thereof.

In various aspects, the alcohol has a structure represented by a formula:

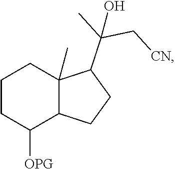

or a pharmaceutically acceptable salt thereof.

In various aspects, the alcohol has a structure represented by a formula:

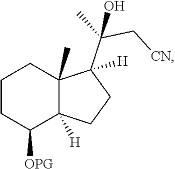

or a pharmaceutically acceptable salt thereof.

In various aspects, the alcohol has a structure represented by a formula:

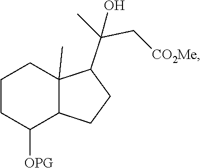

or a pharmaceutically acceptable salt thereof.

In various aspects, the alcohol has a structure represented by a formula:

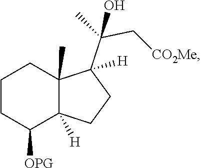

or a pharmaceutically acceptable salt thereof.

In various aspects, the alcohol has a structure represented by a formula:

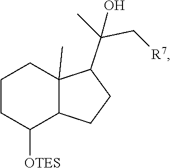

or a pharmaceutically acceptable salt thereof.

In various aspects, the alcohol has a structure represented by a formula:

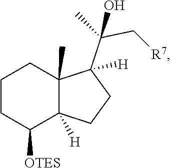

or a pharmaceutically acceptable salt thereof.

In various aspects, the alcohol has a structure:

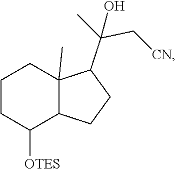

or a pharmaceutically acceptable salt thereof.

In various aspects, the alcohol has a structure:

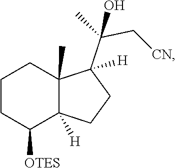

or a pharmaceutically acceptable salt thereof.

In various aspects, the alcohol has a structure:

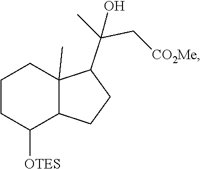

or a pharmaceutically acceptable salt thereof.

In various aspects, the alcohol has a structure:

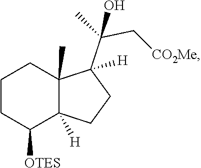

or a pharmaceutically acceptable salt thereof.

In various aspects, the compound has a structure represented by a formula:

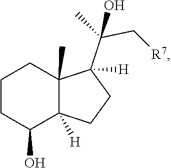

or a pharmaceutically acceptable salt thereof.

In various aspects, the compound has a structure represented by a formula:

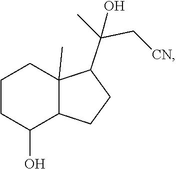 [SMILES: CC(O)(CC#N)C1CCC2C(O)CCCC21C]

or a pharmaceutically acceptable salt thereof.

In various aspects, the compound has a structure:

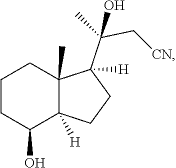 [SMILES: C[C@]12CCC[C@H](O)[C@@H]1CC[C@@H]2[C@@](C)(O)CC#N]

or a pharmaceutically acceptable salt thereof.

In various aspects, the compound has a structure:

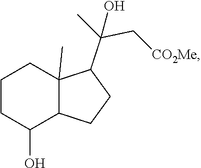 [SMILES: COC(=O)CC(C)(O)C1CCC2C(O)CCCC21C]

or a pharmaceutically acceptable salt thereof.

In various aspects, the compound has a structure:

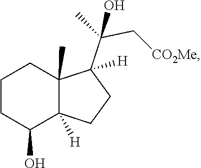 [SMILES: COC(=O)C[C@](C)(O)[C@H]1CC[C@H]2[C@@H](O)CCC[C@@]21C]

or a pharmaceutically acceptable salt thereof.

### A. Exemplary Secondary Alcohols

In one aspect, a secondary alcohol can be present as:

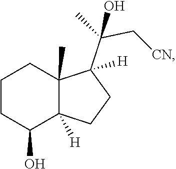 [SMILES: C[C@]12CCC[C@H](O)[C@@H]1CC[C@@H]2[C@@](C)(O)CC#N]

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 3. Cyclohexanones

In one aspect, disclosed are cyclohexanones having a structure represented by a formula:

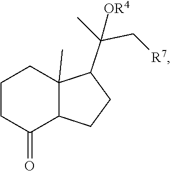

wherein R⁴ is selected from hydrogen and an alcohol protecting group; wherein R⁷ is selected from —CN and —CO₂R¹⁰; and wherein R¹⁰ is C1-C4 alkyl, or a pharmaceutically acceptable salt thereof.

In various aspects, the compound has a structure represented by a formula:

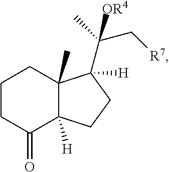

or a pharmaceutically acceptable salt thereof.

In various aspects, the compound has a structure represented by a formula:

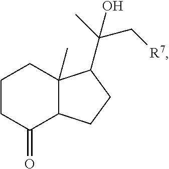

or a pharmaceutically acceptable salt thereof.

In various aspects, the compound has a structure represented by a formula:

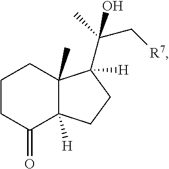

or a pharmaceutically acceptable salt thereof.

In various aspects, the compound has a structure represented by a formula:

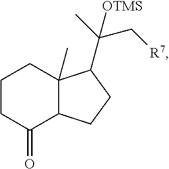

or a pharmaceutically acceptable salt thereof.

In various aspects, the compound has a structure represented by a formula:

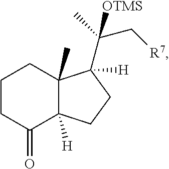

or a pharmaceutically acceptable salt thereof.

In various aspects, the compound has a structure represented by a formula:

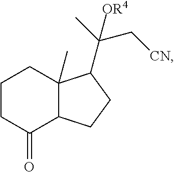

or a pharmaceutically acceptable salt thereof.

In various aspects, the compound has a structure represented by a formula:

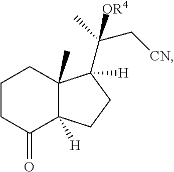

or a pharmaceutically acceptable salt thereof.

In various aspects, the compound has a structure represented by a formula:

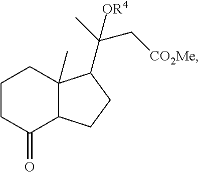

or a pharmaceutically acceptable salt thereof.

In various aspects, the compound has a structure represented by a formula:

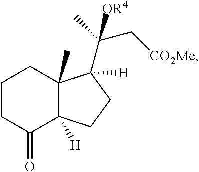

or a pharmaceutically acceptable salt thereof.

In various aspects, the compound has a structure:

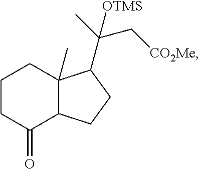

or a pharmaceutically acceptable salt thereof.

In various aspects, the compound has a structure:

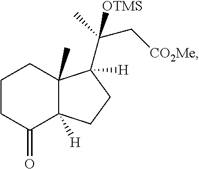

or a pharmaceutically acceptable salt thereof.

### A. R⁴ Groups

In one aspect, R⁴ is selected from hydrogen and an alcohol protecting group. In a further aspect, R⁴ is hydrogen. In a still further aspect, R⁴ is an alcohol protecting group. In a further aspect, the alcohol protecting group is selected from acetyl, benzoyl, benzyl, methoxyethoxymethyl ether (MEM), methoxymethyl ether (MOM), para-methoxybenzyl ether (PMB), para-methoxyphenyl ether (PMP), pivaloyl, tert-butyl ether, tetrahydropyranyl (THP), trityl, trimethylsilyl (TMS), triethylsilyl (TES), tert-butyldimethylsilyl (TBS), tert-butyldiphenylsilyl (TBDPS), and triisopropylsilyl (TIPS). In a yet further aspect, the alcohol protecting group is a silyl protecting group. In a still further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In an even further aspect, the silyl protecting group is triethylsilyl (TES). In a still further aspect, the silyl protecting group is trimethylsilyl (TMS).

b. R⁷ Groups

In one aspect, R⁷ is selected from —CN and —CO₂R¹⁰. In a further aspect, R⁷ is —CN. In a still further aspect, R⁷ is —CO₂R¹⁰. In an even still further aspect, R⁷ is —CO₂Me.

c. Exemplary Cyclohexanones

In one aspect, a cyclohexanone can be present as:

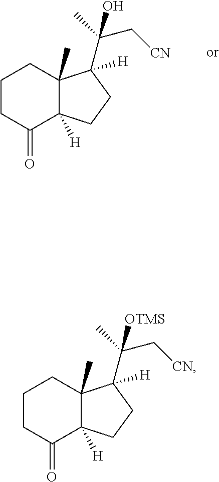

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 4. Protected Tertiary Alcohols

In one aspect, disclosed are protected tertiary alcohols having a structure represented by a formula:

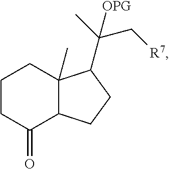

wherein PG is a silyl protecting group; and wherein R⁷ is selected from —CN and —CO₂R¹¹; and wherein R¹⁰ is C1-C4 alkyl, or a pharmaceutically acceptable salt thereof, the method comprising protecting an alcohol having a structure represented by a formula:

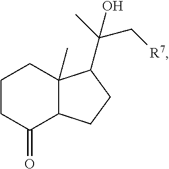

or a pharmaceutically acceptable salt thereof.

In various aspects, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a further aspect, the silyl protecting group is triethylsilyl (TES). In a still further aspect, the silyl protecting group is trimethylsilyl (TMS)

In various aspects, R⁷ is —CN.

In various aspects, R⁷ is —CO₂R¹⁰. In a further aspect, R⁷ is —CO₂Me.

In various aspects, the alcohol has a structure represented by a formula:

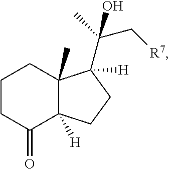

or a pharmaceutically acceptable salt thereof.

In various aspects, the alcohol has a structure:

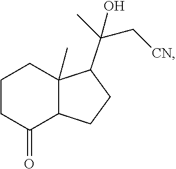 [SMILES: CC(O)(CC#N)C1CCC2C(=O)CCCC21C]

or a pharmaceutically acceptable salt thereof.

In various aspects, the alcohol has a structure:

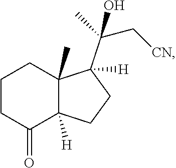 [SMILES: C[C@]12CCCC(=O)[C@@H]1CC[C@@H]2[C@@](C)(O)CC#N]

or a pharmaceutically acceptable salt thereof.

In various aspects, the alcohol has a structure:

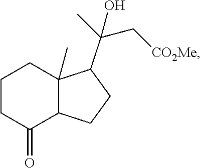 [SMILES: COC(=O)CC(C)(O)C1CCC2C(=O)CCCC21C]

or a pharmaceutically acceptable salt thereof.

In various aspects, the alcohol has a structure:

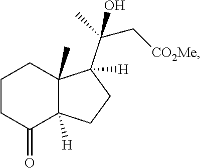 [SMILES: COC(=O)C[C@](C)(O)[C@H]1CC[C@H]2C(=O)CCC[C@@]21C]

or a pharmaceutically acceptable salt thereof.

In various aspects, the compound has a structure represented by a formula:

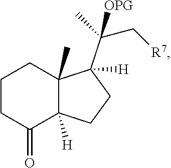

or a pharmaceutically acceptable salt thereof.

In various aspects, the compound has a structure represented by a formula:

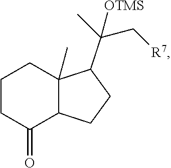

or a pharmaceutically acceptable salt thereof.

In various aspects, the compound has a structure represented by a formula:

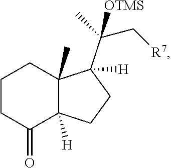

or a pharmaceutically acceptable salt thereof.

In various aspects, the compound has a structure represented by a formula:

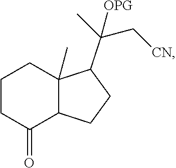

or a pharmaceutically acceptable salt thereof.

In various aspects, the compound has a structure represented by a formula:

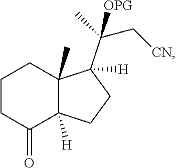

or a pharmaceutically acceptable salt thereof.

In various aspects, the compound has a structure represented by a formula:

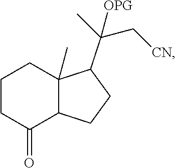

or a pharmaceutically acceptable salt thereof.

In various aspects, the compound has a structure represented by a formula:

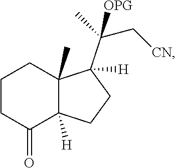

or a pharmaceutically acceptable salt thereof.

In various aspects, the compound has a structure represented by a formula:

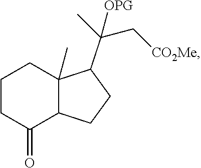

or a pharmaceutically acceptable salt thereof.

In various aspects, the compound has a structure represented by a formula:

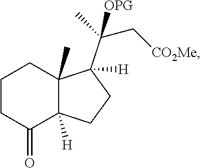

or a pharmaceutically acceptable salt thereof.

In various aspects, the compound has a structure:

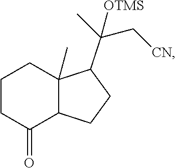

or a pharmaceutically acceptable salt thereof.

In various aspects, the compound has a structure:

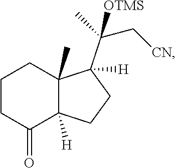

or a pharmaceutically acceptable salt thereof.

In various aspects, the compound has a structure:

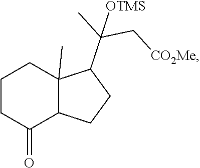

or a pharmaceutically acceptable salt thereof.

In various aspects, the compound has a structure:

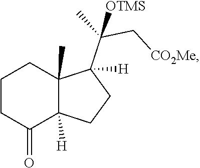

or a pharmaceutically acceptable salt thereof.

### A. Exemplary Protected Tertiary Alcohols

In one aspect, a protected tertiary alcohol can be present as:

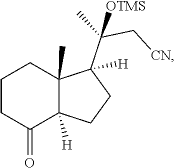

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 5. Trienes

In one aspect, disclosed are compounds having a structure represented by a formula:

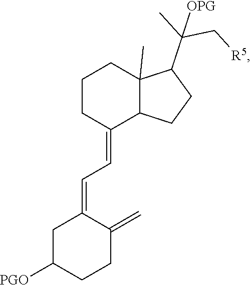

wherein R⁵ is selected from —CN, —CHO, —CO₂R¹⁰; wherein R¹⁰ is C1-C4 alkyl; and wherein each occurrence of PG is independently a silyl protecting group, or a pharmaceutically acceptable salt thereof.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In further aspect, each occurrence of PG is independently a silyl protecting group selected from trimethylsilyl (TMS), triethylsilyl (TES), and tert-butyldimethylsilyl (TBS). In a still further aspect, each occurrence of PG is a different silyl protecting group.

In various aspects, the compound has a structure represented by a formula:

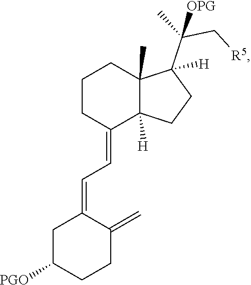

or a pharmaceutically acceptable salt thereof.

In various aspects, the compound has a structure represented by a formula:

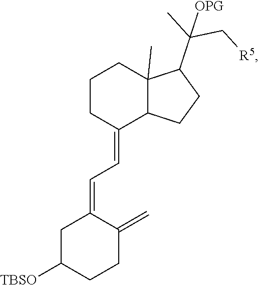

or a pharmaceutically acceptable salt thereof.

In various aspects, the compound has a structure represented by a formula:

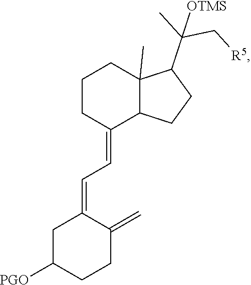

or a pharmaceutically acceptable salt thereof.

In various aspects, the compound has a structure represented by a formula:

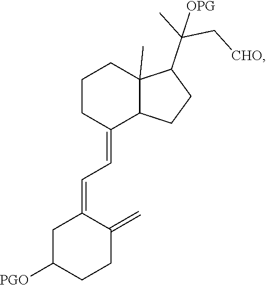

or a pharmaceutically acceptable salt thereof.

In various aspects, the compound has a structure represented by a formula:

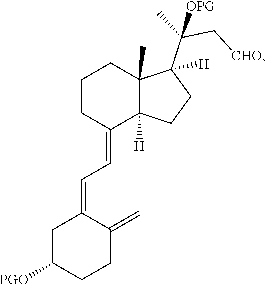

or a pharmaceutically acceptable salt thereof.

In various aspects, the compound has a structure represented by a formula:

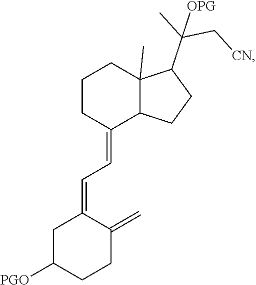

or a pharmaceutically acceptable salt thereof.

In various aspects, the compound has a structure represented by a formula:

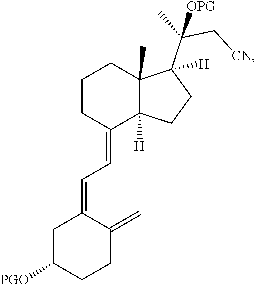

or a pharmaceutically acceptable salt thereof.

In various aspects, the compound has a structure represented by a formula:

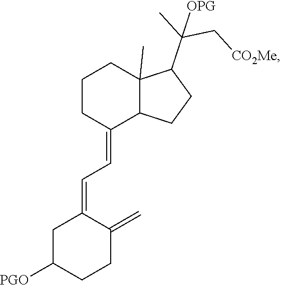

or a pharmaceutically acceptable salt thereof.

In various aspects, the compound has a structure represented by a formula:

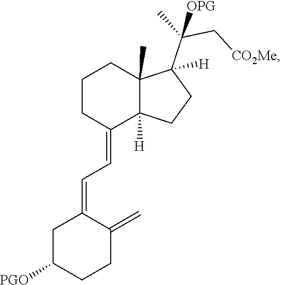

or a pharmaceutically acceptable salt thereof.

In various aspects, the compound has a structure:

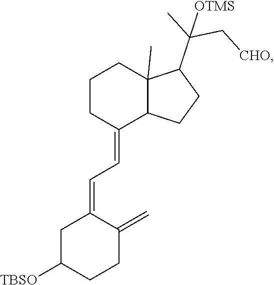

or a pharmaceutically acceptable salt thereof.

In various aspects, the compound has a structure:

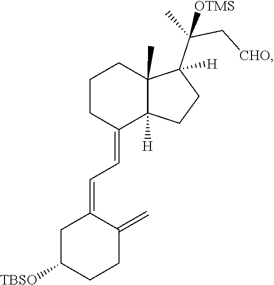

or a pharmaceutically acceptable salt thereof.

In various aspects, the compound has a structure:

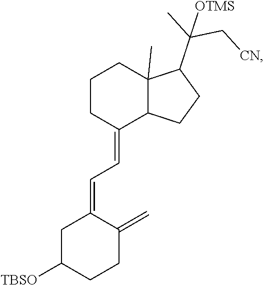

or a pharmaceutically acceptable salt thereof.

In various aspects, the compound has a structure:

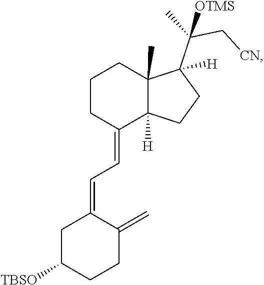

or a pharmaceutically acceptable salt thereof.

In various aspects, the compound has a structure:

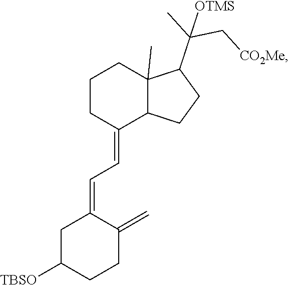

or a pharmaceutically acceptable salt thereof.

In various aspects, the compound has a structure:

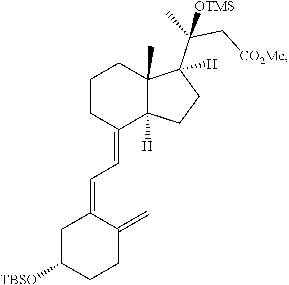

or a pharmaceutically acceptable salt thereof.

### A. R⁵ Groups

In one aspect, R⁵ is selected from —CN, —CHO, and —CO₂R¹⁰. In a further aspect, R⁵ is —CN. In a still further aspect, R⁵ is —CHO. In a yet further aspect, R⁵ is —CO₂R¹⁰. In an even still further aspect, R⁵ is —CO₂Me.

b. Exemplary Trienes

In one aspect, a triene can be present as:

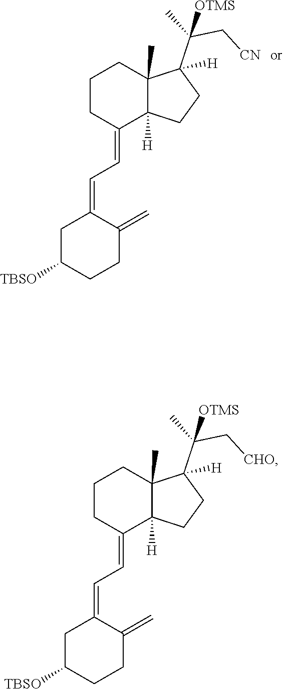

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 6. Aldehydes

In one aspect, disclosed are aldehydes having a structure represented by a formula:

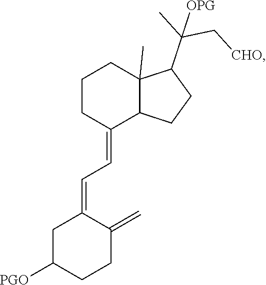

wherein each occurrence of PG is independently a silyl protecting group, or a pharmaceutically acceptable salt thereof, the method comprising reducing a triene having a structure represented by a formula:

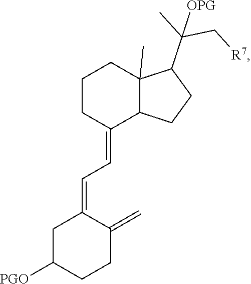

wherein R⁷ is selected from —CN and —CO₂R¹⁰; wherein R¹⁰ is C1-C4 alkyl, or a pharmaceutically acceptable salt thereof.

In various aspects, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In various aspects, the silyl protecting group is triethylsilyl (TES).

In various aspects, R⁷ is —CN. In a further aspect, R⁷ is —CO₂R¹⁰. In a yet further aspect, R⁷ is —CO₂Me.

In various aspects, the triene has a structure represented by a formula:

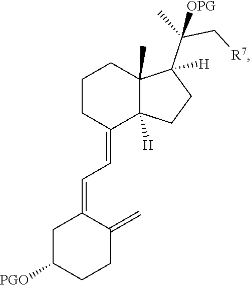

or a pharmaceutically acceptable salt thereof.

In various aspects, the triene has a structure represented by a formula:

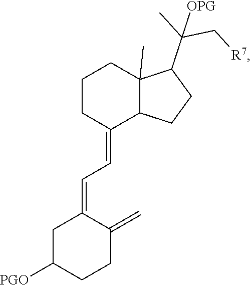

or a pharmaceutically acceptable salt thereof.

In various aspects, the triene has a structure represented by a formula:

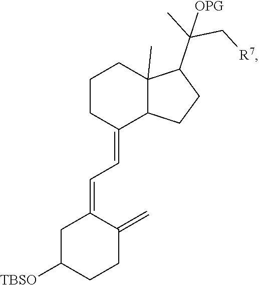

or a pharmaceutically acceptable salt thereof.

In various aspects, the triene has a structure represented by a formula:

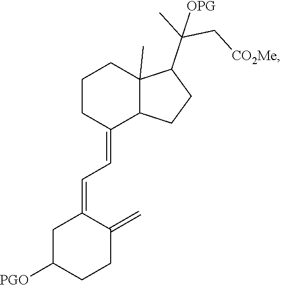

or a pharmaceutically acceptable salt thereof.

In various aspects, the triene has a structure represented by a formula:

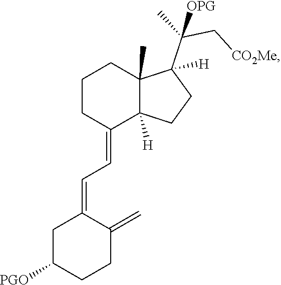

or a pharmaceutically acceptable salt thereof.

In various aspects, the triene has a structure represented by a formula:

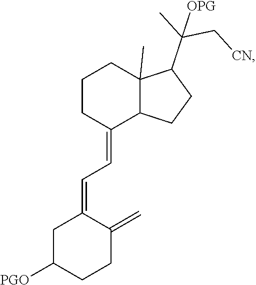

or a pharmaceutically acceptable salt thereof.

In various aspects, the triene has a structure represented by a formula:

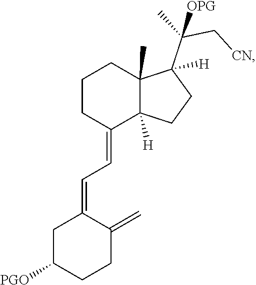

or a pharmaceutically acceptable salt thereof.

In various aspects, the triene has a structure:

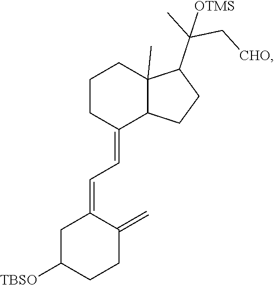

or a pharmaceutically acceptable salt thereof.

In various aspects, the triene has a structure:

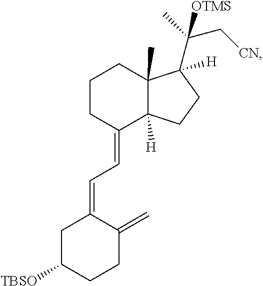

or a pharmaceutically acceptable salt thereof.

In various aspects, the compound has a structure represented by a formula:

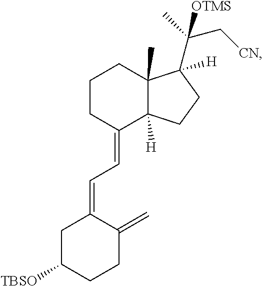

or a pharmaceutically acceptable salt thereof.

In various aspects, the compound has a structure represented by a formula:

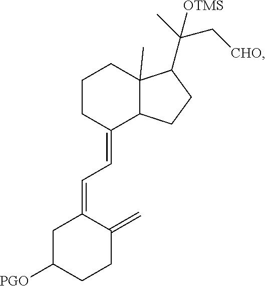

or a pharmaceutically acceptable salt thereof.

In various aspects, the compound has a structure represented by a formula:

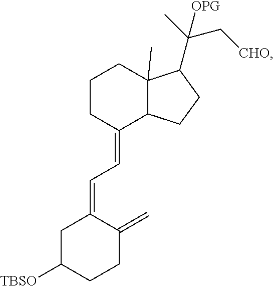

or a pharmaceutically acceptable salt thereof.

In various aspects, the compound has a structure:

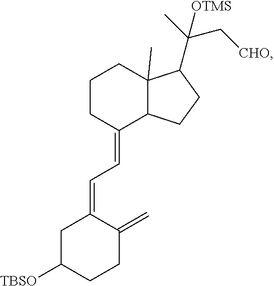

or a pharmaceutically acceptable salt thereof.

In various aspects, the compound has a structure:

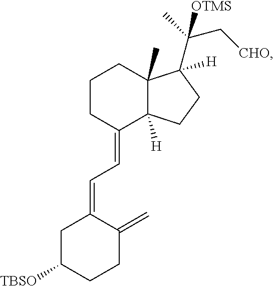

or a pharmaceutically acceptable salt thereof.

### A. Exemplary Aldehydes

In one aspect, an aldehyde can be present as:

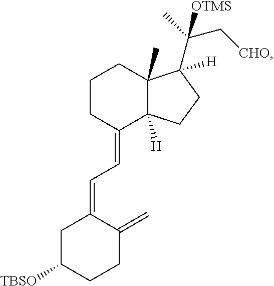

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 7. Alkyl Alcohols

In one aspect, disclosed are alkyl alcohols having a structure represented by a formula:

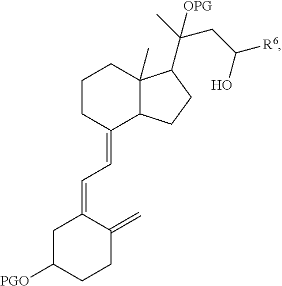

wherein R⁶ is a C1-C8 alkyl; and wherein each occurrence of PG is independently a silyl protecting group, or a pharmaceutically acceptable salt thereof.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In further aspect, each occurrence of PG is independently a silyl protecting group selected from trimethylsilyl (TMS) and tert-butyldimethylsilyl (TBS).

In various aspects, the compound has a structure represented by a formula:

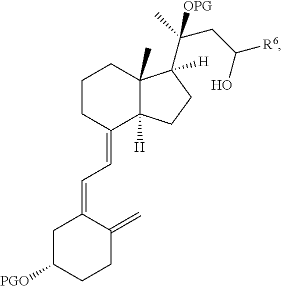

or a pharmaceutically acceptable salt thereof.

In various aspects, the compound has a structure represented by a formula:

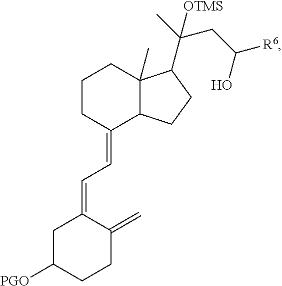

or a pharmaceutically acceptable salt thereof.

In various aspects, the compound has a structure represented by a formula:

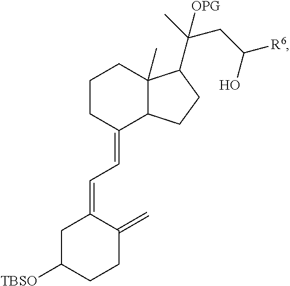

or a pharmaceutically acceptable salt thereof.

In various aspects, the compound has a structure represented by a formula:

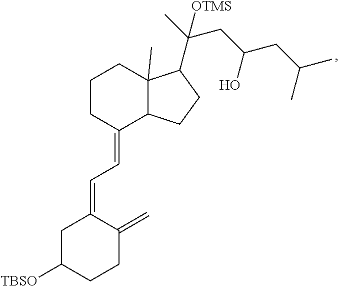

or a pharmaceutically acceptable salt thereof.

In various aspects, the compound has a structure represented by a formula:

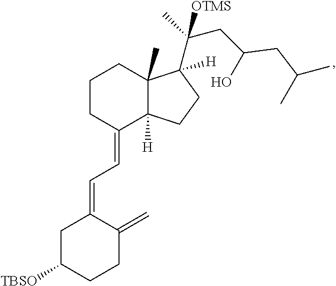

or a pharmaceutically acceptable salt thereof.

### A. R⁶ Groups

In one aspect, R⁶ is a C1-C8 alkyl. In a further aspect, R⁶ is a C1-C4 alkyl. In a still further aspect, R⁶ is selected from methyl, ethyl, n-propyl, i-propyl, n-butyl, isobutyl, secbutyl, and tert-butyl. In a yet further aspect, R⁶ is selected from methyl, ethyl, n-propyl, and i-propyl. In an even further aspect, R⁶ is selected from methyl and ethyl. In a still further aspect, R⁶ is ethyl. In yet a further aspect, R⁶ is methyl.

In various aspects, R⁶ is isobutyl.

b. Exemplary Alkyl Alcohols

In one aspect, an alkyl alcohol can be present as:

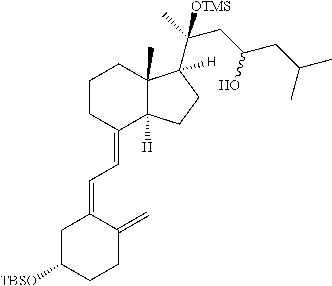

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### 8. Open Chain Hydroxy Derivatives of Vitamin D₃

In one aspect, disclosed are open chain hydroxy derivatives of vitamin D₃ having a structure represented by a formula:

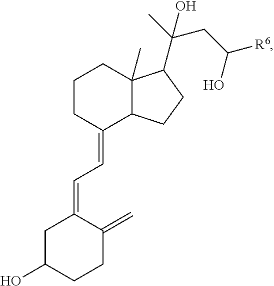

wherein R⁶ is a C1-C8 alkyl, or a pharmaceutically acceptable salt thereof.

In various aspects, the compound has a structure represented by a formula:

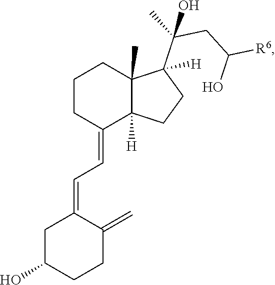

or a pharmaceutically acceptable salt thereof.

In various aspects, the compound has a structure:

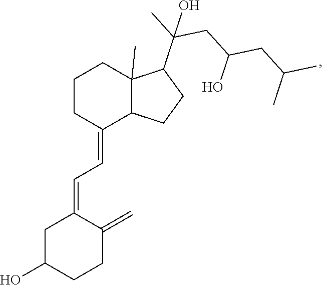 [SMILES: C=C1CCC(O)C/C1=C/C=C1\CCCC2(C)C1CCC2C(C)(O)CC(O)CC(C)C]

or a pharmaceutically acceptable salt thereof.

In various aspects, the compound has a structure:

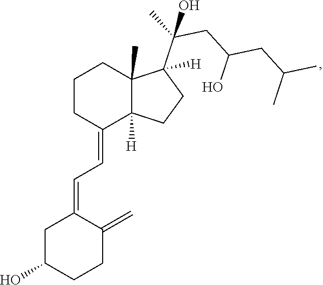 [SMILES: C=C1CC[C@H](O)C/C1=C/C=C1\CCC[C@@]2(C)[C@H]1CC[C@@H]2[C@@](C)(O)CC(O)CC(C)C]

or a pharmaceutically acceptable salt thereof.

In various aspects, the compound has a structure:

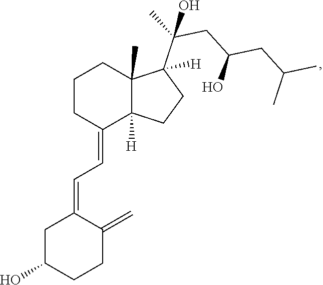 [SMILES: C=C1CC[C@H](O)C/C1=C/C=C1\CCC[C@@]2(C)[C@H]1CC[C@@H]2[C@@](C)(O)C[C@H](O)CC(C)C]

or a pharmaceutically acceptable salt thereof.

In various aspects, the compound has a structure:

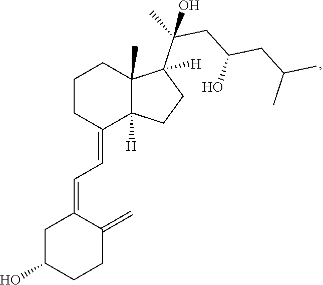 [SMILES: C=C1CC[C@H](O)C/C1=C/C=C1\CCC[C@@]2(C)[C@H]1CC[C@@H]2[C@@](C)(O)C[C@@H](O)CC(C)C]

or a pharmaceutically acceptable salt thereof.

In various aspects, the compound ahs a structure selected from:

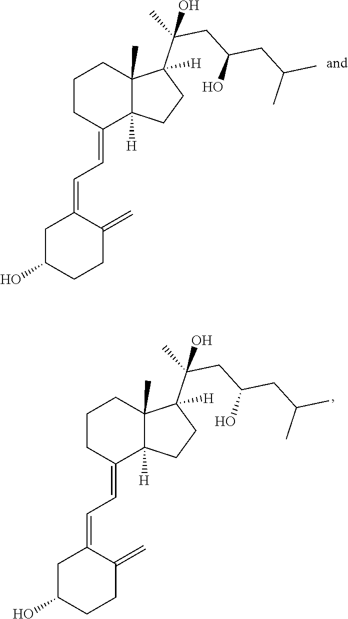 [SMILES: C=C1CC[C@H](O)C/C1=C/C=C1\CCC[C@@]2(C)[C@H]1CC[C@@H]2[C@@](C)(O)C[C@H](O)CC(C)C ; C=C1CC[C@H](O)C/C1=C/C=C1\CCC[C@@]2(C)[C@H]1CC[C@@H]2[C@@](C)(O)C[C@@H](O)CC(C)C]

or a pharmaceutically acceptable salt thereof.

### A. R⁶ Groups

In one aspect, R⁶ is a C1-C8 alkyl. In a further aspect, R⁶ is a C1-C4 alkyl. In a still further aspect, R⁶ is selected from methyl, ethyl, n-propyl, i-propyl, n-butyl, isobutyl, secbutyl, and tert-butyl. In a yet further aspect, R⁶ is selected from methyl, ethyl, n-propyl, and i-propyl. In an even further aspect, R⁶ is selected from methyl and ethyl. In a still further aspect, R⁶ is ethyl. In yet a further aspect, R⁶ is methyl.

In various aspects, R⁶ is isobutyl.

b. Exemplary Open Chain Hydroxy Derivatives of Vitamin D₃

In one aspect, an open chain hydroxy derivative of vitamin D₃ can be present as:

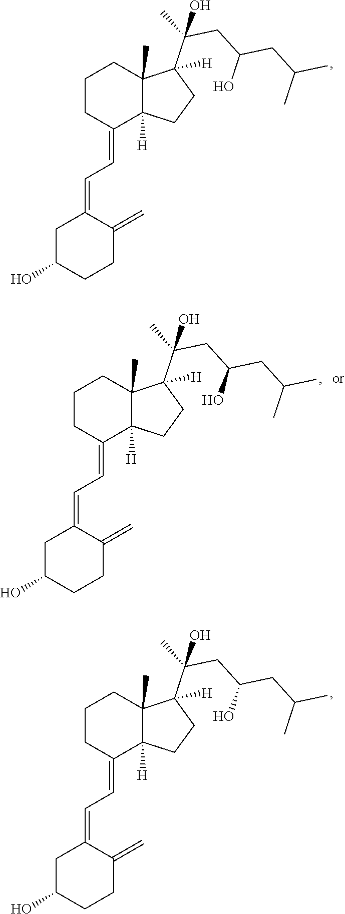 [SMILES: C=C1CC[C@H](O)C/C1=C/C=C1\CCC[C@@]2(C)[C@H]1CC[C@@H]2[C@@](C)(O)CC(O)CC(C)C ; C=C1CC[C@H](O)C/C1=C/C=C1\CCC[C@@]2(C)[C@H]1CC[C@@H]2[C@@](C)(O)C[C@H](O)CC(C)C ; C=C1CC[C@H](O)C/C1=C/C=C1\CCC[C@@]2(C)[C@H]1CC[C@@H]2[C@@](C)(O)C[C@@H](O)CC(C)C]

or a pharmaceutically acceptable salt thereof.

It is contemplated that one or more compounds can optionally be omitted from the disclosed invention.

It is understood that the disclosed compounds can be used in connection with the disclosed methods.

It is understood that pharmaceutically acceptable derivatives of the disclosed compounds can be used also in connection with the disclosed methods. The pharmaceutically acceptable derivatives of the compounds can include any suitable derivative, such as pharmaceutically acceptable salts as discussed below, isomers, radiolabeled analogs, tautomers, and the like.

### E. Methods of Making Hydroxy Derivatives of Vitamin D₃(Open Chain-Route II)

In one aspect, disclosed are methods of making open chain hydroxy derivatives of vitamin D₃. See, e.g., FIG. 5. As detailed herein, hydroxy derivatives of vitamin D₃ have demonstrated utility as therapeutic agents for a variety of autoimmune disorders including, but not limited to, lupus erythematosus, rheumatoid arthritis, inflammatory bowel disease, psoriasis, and multiple sclerosis. Thus, in various aspects, hydroxy derivatives of vitamin D₃ can be prepared by adding an appropriate nucleophile to an appropriate ketone to provide the desired functionality (i.e., a nitrile or an ester derivative as detailed herein). Deprotection of the secondary alcohol (i.e., to provide a secondary alcohol as detailed herein) followed by oxidation affords the corresponding ketone (i.e., a cyclohexanone as detailed herein). The tertiary alcohol is then protected (i.e., to provide a protected tertiary alcohol as detailed herein) before coupling with an appropriate phosphine oxide (i.e., to provide a triene as detailed herein). Next, the triene is reduced (i.e., to provide an aldehyde as detailed herein), and coupled to an appropriate Grignard reagent (i.e., to provide an alkyl alcohol as detailed herein). At that time, the protecting groups can be removed, thereby providing an open chain hydroxy derivative of vitamin D₃.

### 1. Methods of Making Nitrile and Ester Derivatives

In one aspect, disclosed are methods of making a compound having a structure represented by a formula:

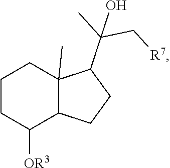

wherein R³ is selected from hydrogen and an alcohol protecting group; wherein R⁷ is selected from —CN and —CO₂R¹⁰; and wherein R¹⁰ is C1-C4 alkyl, or a pharmaceutically acceptable salt thereof, the method comprising reacting a methyl ketone having a structure represented by a formula:

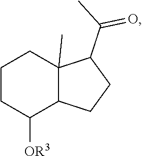

and a nitrile or CH₃CO₂R¹⁰, or a pharmaceutically acceptable salt thereof, and a nucleophile having a structure represented by a formula:

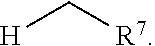

In various aspects, reacting is in the presence of a base. In a further aspect, the base is selected from n-butyl lithium, 1,8-diazabicyclo[5.4.0]undec-7-ene (DBU), lithium diisopropylamide (LDA), sodium hydride, and potassium tert-butoxide. In a yet further aspect, the base is lithium diisopropylamide (LDA).

In various aspects, reacting is in an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, reacting is at a temperature of about −78° C.

In various aspects, the nitrile is selected from acetonitrile, propionitrile, and benzonitrile. In a yet further aspect, the nitrile is acetonitrile.

In various aspects, R³ is hydrogen.

In various aspects, R³ is the alcohol protecting group. In a further aspect, the alcohol protecting group is selected from acetyl, benzoyl, benzyl, methoxyethoxymethyl ether (MEM), methoxymethyl ether (MOM), para-methoxybenzyl ether (PMB), para-methoxyphenyl ether (PMP), pivaloyl, tert-butyl ether, tetrahydropyranyl (THP), trityl, trimethylsilyl (TMS), triethylsilyl (TES), tert-butyldimethylsilyl (TBS), tert-butyldiphenylsilyl (TBDPS), and triisopropylsilyl (TIPS). In a still further aspect, the alcohol protecting group is a silyl protecting group. In a yet further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In an even further aspect, the silyl protecting group is triethylsilyl (TES).

In various aspects, R⁷ is —CN.

In various aspects, R⁷ is —CO₂R¹¹. In a further aspect, R⁷ is —CO₂Me.

In various aspects, the methyl ketone has a structure represented by a formula:

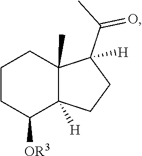

or a pharmaceutically acceptable salt thereof.

In various aspects, the methyl ketone has a structure:

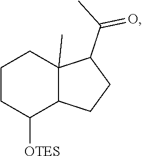

or a pharmaceutically acceptable salt thereof.

In various aspects, the methyl ketone has a structure:

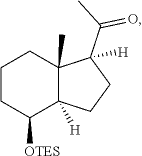

or a pharmaceutically acceptable salt thereof.

In various aspects, the compound has a structure represented by a formula:

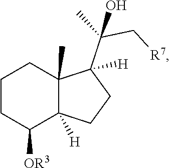

or a pharmaceutically acceptable salt thereof.

In various aspects, the compound has a structure represented by a formula:

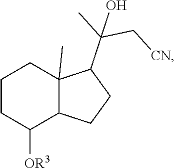

or a pharmaceutically acceptable salt thereof.

In various aspects, the compound has a structure represented by a formula:

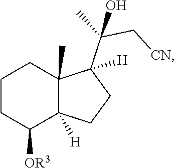

or a pharmaceutically acceptable salt thereof.

In various aspects, the compound has a structure represented by a formula:

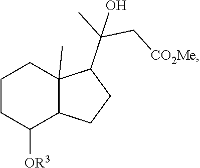

or a pharmaceutically acceptable salt thereof.

In various aspects, the compound has a structure represented by a formula:

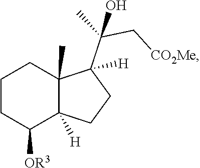

or a pharmaceutically acceptable salt thereof.

In various aspects, the compound has a structure represented by a formula:

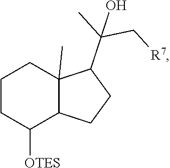

or a pharmaceutically acceptable salt thereof.

In various aspects, the compound has a structure represented by a formula:

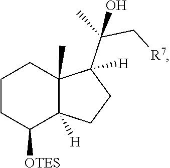

or a pharmaceutically acceptable salt thereof.

In various aspects, the compound has a structure:

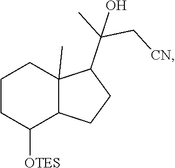

or a pharmaceutically acceptable salt thereof.

In various aspects, the compound has a structure:

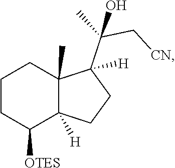

or a pharmaceutically acceptable salt thereof.

In various aspects, the compound has a structure:

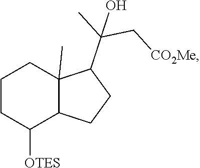

or a pharmaceutically acceptable salt thereof.

In various aspects, the compound has a structure:

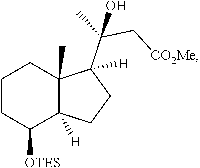

or a pharmaceutically acceptable salt thereof.

### A. Route I

In one aspect, substituted cyano derivatives can be prepared as shown below.

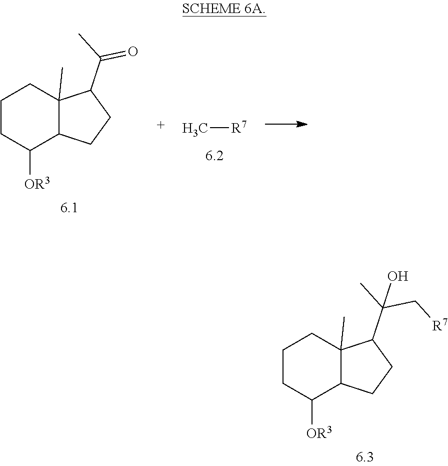

Compounds are represented in generic form with substituents as noted in compound descriptions elsewhere herein. A more specific example is set forth below.

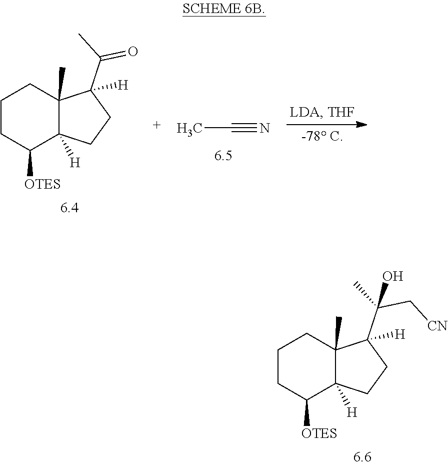

In one aspect, compounds of type 6.6, and similar compounds, can be prepared according to reaction Scheme 1B above. Thus, compounds of type 6.6 can be prepared by addition of an appropriate nucleophile, e.g., 6.5, as shown above, and an appropriate ketone, e.g., 6.4 as shown above. Appropriate nucleophiles and appropriate ketones are commercially available or prepared by methods known to one skilled in the art. The reaction is carried out in the presence of an appropriate base, e.g., Lithium diisopropylamide, in an appropriate solvent, e.g., anhydrous THF, at an appropriate temperature, e.g., −78° C., for an appropriate amount of time, e.g., 2 h. As can be appreciated by one skilled in the art, the above reaction provides an example of a generalized approach wherein compounds similar in structure to the specific reactants above (compounds similar to compounds of type 6.1 and 6.2), can be substituted in the reaction to provide nitrile or ester derivatives similar to Formula 6.3.

### 2. Methods of Making Secondary Alcohols

In one aspect, disclosed are methods of making a compound having a structure represented by a formula:

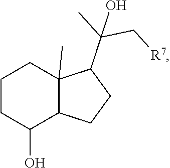

wherein R⁷ is selected from —CN and —CO₂R¹⁰; and wherein R¹⁰ is C1-C4 alkyl, or a pharmaceutically acceptable salt thereof, the method comprising deprotecting an alcohol having a structure represented by a formula:

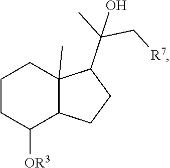

wherein R³ is selected from hydrogen and an alcohol protecting group, or a pharmaceutically acceptable salt thereof.

In various aspects, deprotecting is in the presence of an acid. In a further aspect, the acid is selected from hydrochloric acid, hydrobromic acid, and acetic acid. In a still further aspect, the acid is hydrochloric acid.

In various aspects, deprotecting is in the presence of an organic, nonpolar solvent. In a further aspect, the organic, nonpolar solvent is selected from acetic acid, dichloromethane (DCM), ethyl acetate (EtOAc), diethyl ether, and pyridine. In a still further aspect, the organic, nonpolar solvent is dichloromethane (DCM).

In various aspects, deprotecting is at a temperature of about −78° C.

In various aspects, the alcohol protecting group is selected from acetyl, benzoyl, benzyl, methoxyethoxymethyl ether (MEM), methoxymethyl ether (MOM), para-methoxybenzyl ether (PMB), para-methoxyphenyl ether (PMP), pivaloyl, tert-butyl ether, tetrahydropyranyl (THP), trityl, trimethylsilyl (TMS), triethylsilyl (TES), tert-butyldimethylsilyl (TBS), tert-butyldiphenylsilyl (TBDPS), and triisopropylsilyl (TIPS). In a still further aspect, the alcohol protecting group is a silyl protecting group. In a yet further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In an even further aspect, the silyl protecting group is triethylsilyl (TES).

In various aspects, R⁷ is —CN.

In various aspects, R⁷ is —CO₂R¹⁰. In a further aspect, R⁷ is —CO₂Me.

In various aspects, the alcohol has a structure represented by a formula:

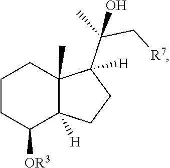

or a pharmaceutically acceptable salt thereof.

In various aspects, the alcohol has a structure represented by a formula:

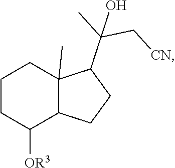

or a pharmaceutically acceptable salt thereof.

In various aspects, the alcohol has a structure represented by a formula:

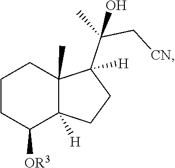

or a pharmaceutically acceptable salt thereof.

In various aspects, the alcohol has a structure represented by a formula:

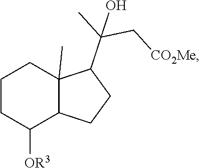

or a pharmaceutically acceptable salt thereof.

In various aspects, the alcohol has a structure represented by a formula:

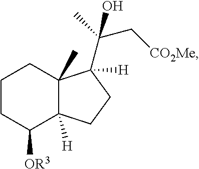

or a pharmaceutically acceptable salt thereof.

In various aspects, the alcohol has a structure represented by a formula:

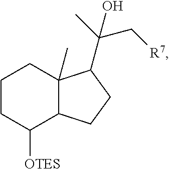

or a pharmaceutically acceptable salt thereof.

In various aspects, the alcohol has a structure represented by a formula:

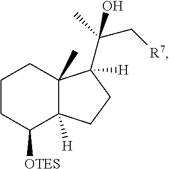

or a pharmaceutically acceptable salt thereof.

In various aspects, the alcohol has a structure:

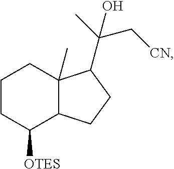

or a pharmaceutically acceptable salt thereof.

In various aspects, the alcohol has a structure:

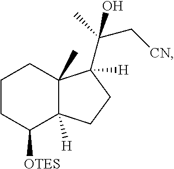

or a pharmaceutically acceptable salt thereof.

In various aspects, the alcohol has a structure:

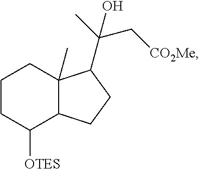

or a pharmaceutically acceptable salt thereof.

In various aspects, the alcohol has a structure:

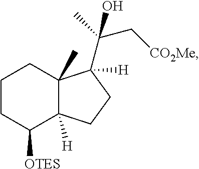

or a pharmaceutically acceptable salt thereof.

In various aspects, the compound has a structure represented by a formula:

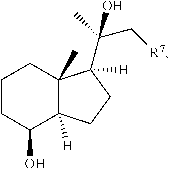

or a pharmaceutically acceptable salt thereof.

In various aspects, the compound has a structure represented by a formula:

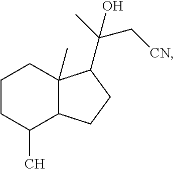 [SMILES: CC1CCCC2(C)C1CCC2C(C)(O)CC#N]

or a pharmaceutically acceptable salt thereof.

In various aspects, the compound has a structure:

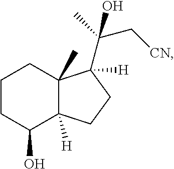 [SMILES: C[C@]12CCC[C@H](O)[C@@H]1CC[C@@H]2[C@@](C)(O)CC#N]

or a pharmaceutically acceptable salt thereof.

In various aspects, the compound has a structure:

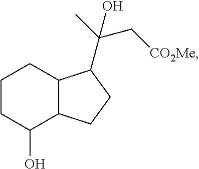 [SMILES: COC(=O)CC(C)(O)C1CCC2C(O)CCCC21]

or a pharmaceutically acceptable salt thereof.

In various aspects, the compound has a structure:

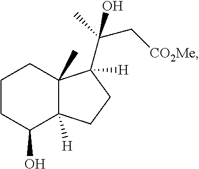 [SMILES: COC(=O)C[C@](C)(O)[C@H]1CC[C@H]2[C@@H](O)CCC[C@@]21C]

or a pharmaceutically acceptable salt thereof.

### A. Route I

In one aspect, substituted secondary alcohols can be prepared as shown below.

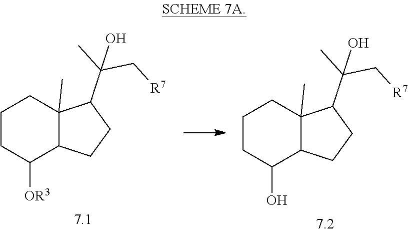

Compounds are represented in generic form with substituents as noted in compound descriptions elsewhere herein. A more specific example is set forth below.

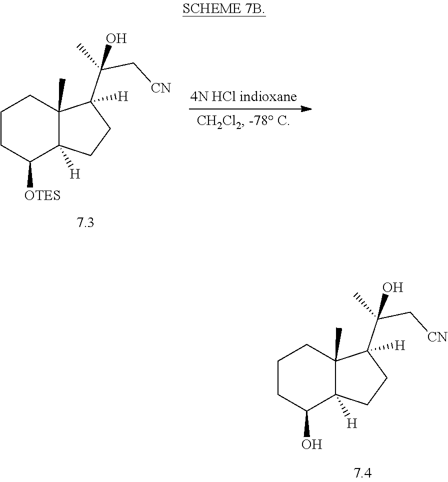

In one aspect, compounds of type 7.4, and similar compounds, can be prepared according to reaction Scheme 7B above. Thus, compounds of type 7.4 can be prepared by deprotection of an appropriate protected alcohol, e.g., 7.3, as shown above. The reaction is carried out under appropriate deprotecting conditions, e.g., 4M HCl, in an appropriate solvent, e.g., dichloromethane (CH₂Cl₂), at an appropriate temperature, e.g., −78° C. As can be appreciated by one skilled in the art, the above reaction provides an example of a generalized approach wherein compounds similar in structure to the specific reactants above (compounds similar to compounds of type 7.1), can be substituted in the reaction to provide secondary alcohol analogs similar to Formula 7.2.

### 3. Methods of Making Cyclohexanones

In one aspect, disclosed are methods of making a compound having a structure represented by a formula:

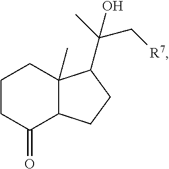

wherein R⁷ is selected from —CN and —CO₂R¹⁰; and wherein R¹⁰ is C1-C4 alkyl, or a pharmaceutically acceptable salt thereof, the method comprising oxidizing an alcohol having a structure represented by a formula:

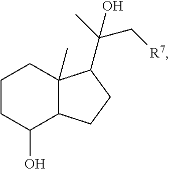

or a pharmaceutically acceptable salt thereof.

In various aspects, oxidizing is in the presence of an oxidizing agent selected from tetrapropylammonium perruthenate (TPAP), hydrogen peroxide, di-tert-butyl peroxide, oxone, chromic acid, pyridinium dichromate, and pyridinium chlorochromate. In a further aspect, oxidizing is in the presence of tetrapropylammonium perruthenate (TPAP).

In various aspects, oxidizing is in the presence of an organic, nonpolar solvent.

In a further aspect, the organic, nonpolar solvent is selected from acetic acid, dichloromethane (DCM), and ethyl acetate (EtOAc). In a still further aspect, the organic, nonpolar solvent is dichloromethane (DCM).

In various aspects, oxidizing is at a temperature of from about 0° C. to about 22° C.

In various aspects, R⁷ is —CN.

In various aspects, R⁷ is —CO₂R¹⁰. In a further aspect, R⁷ is —CO₂Me.

In various aspects, the alcohol has a structure represented by a formula:

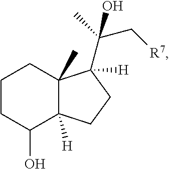

or a pharmaceutically acceptable salt thereof.

In various aspects, the alcohol has a structure:

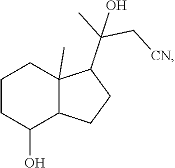 [SMILES: CC(O)(CC#N)C1CCC2C(O)CCCC21C]

or a pharmaceutically acceptable salt thereof.

In various aspects, the alcohol has a structure:

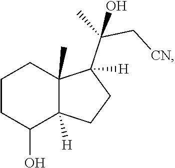 [SMILES: C[C@]12CCCC(O)[C@@H]1CC[C@@H]2[C@@](C)(O)CC#N]

or a pharmaceutically acceptable salt thereof.

In various aspects, the alcohol has a structure:

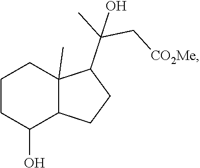 [SMILES: COC(=O)CC(C)(O)C1CCC2C(O)CCCC21C]

or a pharmaceutically acceptable salt thereof.

In various aspects, the alcohol has a structure:

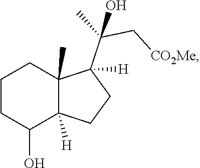 [SMILES: COC(=O)C[C@](C)(O)[C@H]1CC[C@H]2C(O)CCC[C@@]21C]

or a pharmaceutically acceptable salt thereof.

In various aspects, the compound has a structure represented by a formula:

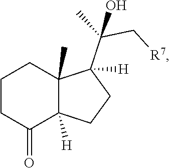

or a pharmaceutically acceptable salt thereof.

In various aspects, the compound has a structure:

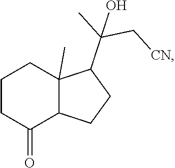 [SMILES: CC(O)(CC#N)C1CCC2C(=O)CCCC21C]

or a pharmaceutically acceptable salt thereof.

In various aspects, the compound has a structure:

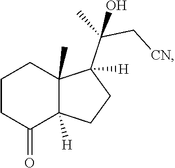 [SMILES: C[C@]12CCCC(=O)[C@@H]1CC[C@@H]2[C@@](C)(O)CC#N]

or a pharmaceutically acceptable salt thereof.

In various aspects, the compound has a structure:

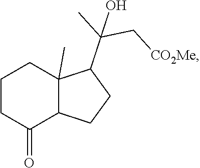 [SMILES: COC(=O)CC(C)(O)C1CCC2C(=O)CCCC21C]

or a pharmaceutically acceptable salt thereof.

In various aspects, the compound has a structure:

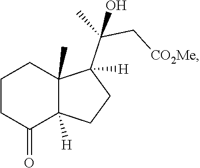 [SMILES: COC(=O)C[C@](C)(O)[C@H]1CC[C@H]2C(=O)CCC[C@@]21C]

or a pharmaceutically acceptable salt thereof.

### A. Route I

In one aspect, substituted cyclohexanone analogs can be prepared as shown below.

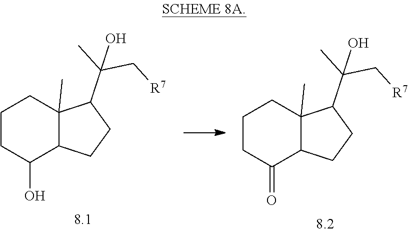

Compounds are represented in generic form, with substituents as noted in compound descriptions elsewhere herein. A more specific example is set forth below.

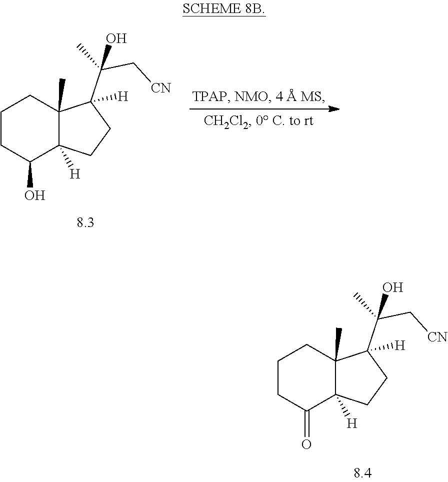 [SMILES: C[C@]12CCC[C@H](O)[C@@H]1CC[C@@H]2[C@@](C)(O)CC#N ; C[C@]12CCCC(=O)[C@@H]1CC[C@@H]2[C@@](C)(O)CC#N]

In one aspect, compounds of type 8.4, and similar compounds, can be prepared according to reaction Scheme 8B above. Thus, compounds of type 8.4 can be prepared by oxidation of an appropriate secondary alcohol, e.g., 8.3 as shown above. The oxidation is carried out in the presence of an appropriate oxidizing agent, e.g., tetrapropylammonium perruthenate (TPAP) and 4-methylmorpholine N-oxide (NMO), in an appropriate solvent, e.g., dichloromethane (DCM), at an appropriate temperature, e.g., 0° C. As can be appreciated by one skilled in the art, the above reaction provides an example of a generalized approach wherein compounds similar in structure to the specific reactants above (compounds similar to compounds of type 8.1), can be substituted in the reaction to provide substituted cyclohexanone analogs similar to Formula 8.2.

### 4. Methods of Making Protected Tertiary Alcohols

In one aspect, disclosed are methods of making a compound having a structure represented by a formula:

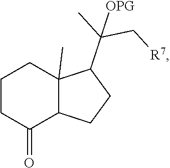

wherein PG is a silyl protecting group; wherein R⁷ is selected from —CN and —CO₂R¹⁰; and wherein R¹⁰ is C1-C4 alkyl, or a pharmaceutically acceptable salt thereof, the method comprising protecting an alcohol having a structure represented by a formula:

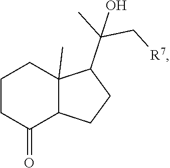

or a pharmaceutically acceptable salt thereof.

In various aspects, protecting is via addition of a silyl triflate. In a further aspect, the silyl triflate is trimethylsilyl triflate.

In various aspects, protecting is in the presence of a base. In a further aspect, the base is selected from pyridine, pyridinium chlorochromate, pyridinium dichromate, triethylamine, and N,N-diethylaniline. In a still further aspect, the base is pyridine.

In various aspects, protecting is at a temperature of about 0° C.

In various aspects, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a further aspect, the silyl protecting group is triethylsilyl (TES). In a still further aspect, the silyl protecting group is trimethylsilyl (TMS).

In various aspects, R⁷ is —CN.

In various aspects, R⁷ is —CO₂R¹⁰. In a further aspect, R⁷ is —CO₂Me.

In various aspects, the alcohol has a structure represented by a formula:

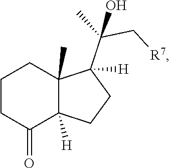

or a pharmaceutically acceptable salt thereof.

In various aspects, the alcohol has a structure:

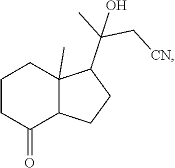 [SMILES: CC(O)(CC#N)C1CCC2C(=O)CCCC21C]

or a pharmaceutically acceptable salt thereof.

In various aspects, the alcohol has a structure:

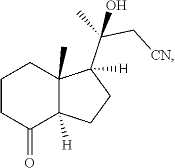 [SMILES: C[C@]12CCCC(=O)[C@@H]1CC[C@@H]2[C@@](C)(O)CC#N]

or a pharmaceutically acceptable salt thereof.

In various aspects, the alcohol has a structure:

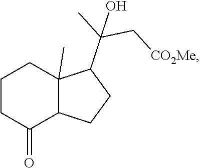 [SMILES: COC(=O)CC(C)(O)C1CCC2C(=O)CCCC21C]

or a pharmaceutically acceptable salt thereof.

In various aspects, the alcohol has a structure:

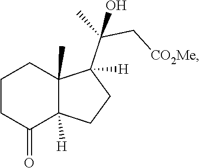 [SMILES: COC(=O)C[C@](C)(O)[C@H]1CC[C@H]2C(=O)CCC[C@@]21C]

or a pharmaceutically acceptable salt thereof.

In various aspects, the compound has a structure represented by a formula:

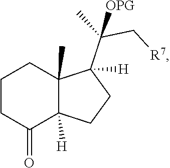

or a pharmaceutically acceptable salt thereof.

In various aspects, the compound has a structure represented by a formula:

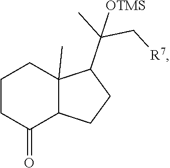

or a pharmaceutically acceptable salt thereof.

In various aspects, the compound has a structure represented by a formula:

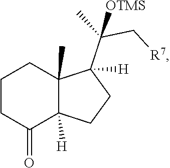

or a pharmaceutically acceptable salt thereof.

In various aspects, the compound has a structure represented by a formula:

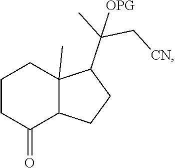

or a pharmaceutically acceptable salt thereof.

In various aspects, the compound has a structure represented by a formula:

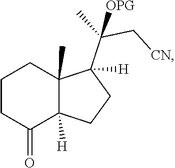

or a pharmaceutically acceptable salt thereof.

In various aspects, the compound has a structure represented by a formula:

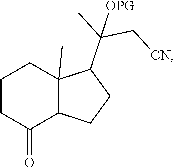

or a pharmaceutically acceptable salt thereof.

In various aspects, the compound has a structure represented by a formula:

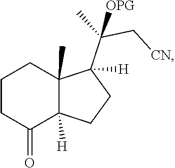

or a pharmaceutically acceptable salt thereof.

In various aspects, the compound has a structure represented by a formula:

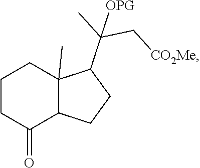

or a pharmaceutically acceptable salt thereof.

In various aspects, the compound has a structure represented by a formula:

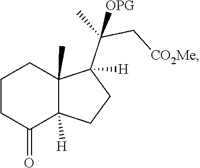

or a pharmaceutically acceptable salt thereof.

In various aspects, the compound has a structure:

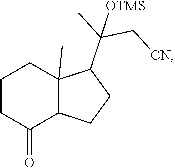

or a pharmaceutically acceptable salt thereof.

In various aspects, the compound has a structure:

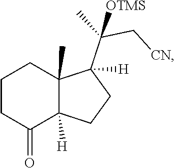

or a pharmaceutically acceptable salt thereof.

In various aspects, the compound has a structure:

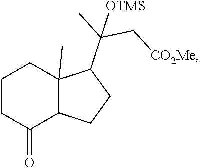

or a pharmaceutically acceptable salt thereof.

In various aspects, the compound has a structure:

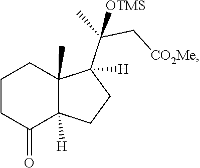

or a pharmaceutically acceptable salt thereof.

### A. Route I

In one aspect, substituted protected tertiary alcohols can be prepared as shown below.

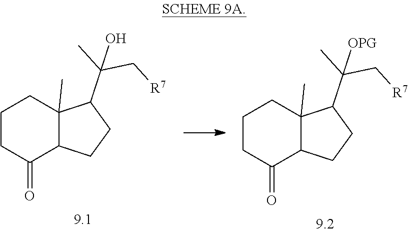

Compounds are represented in generic form, with substituents as noted in compound descriptions elsewhere herein. A more specific example is set forth below.

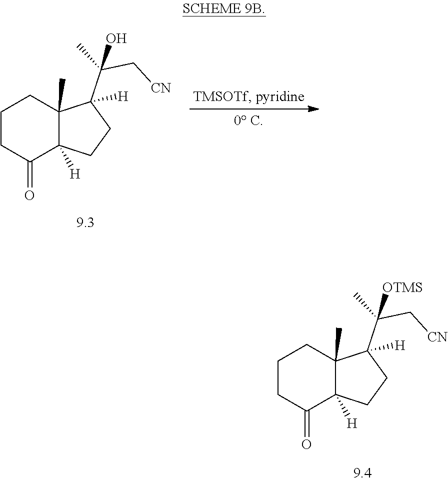

In one aspect, compounds of type 9.4, and similar compounds, can be prepared according to reaction Scheme 9B above. Thus, compounds of type 9.4 can be prepared by protection of an appropriate tertiary alcohol, e.g., 9.3 as shown above. The protection is carried out in the presence of an appropriate activated protecting group, e.g., trimethylsilyl triflate, in an appropriate solvent, e.g., pyridine, at an appropriate temperature, e.g., 0° C., for an appropriate amount of time, e.g., lh. As can be appreciated by one skilled in the art, the above reaction provides an example of a generalized approach wherein compounds similar in structure to the specific reactants above (compounds similar to compounds of type 9.1), can be substituted in the reaction to provide substituted protected tertiary alcohols similar to Formula 9.2.

### 5. Methods of Making Trienes

In one aspect, disclosed are methods of making a compound having a structure represented by a formula:

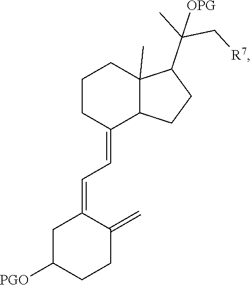

wherein R⁷ is selected from —CN and —CO₂R¹⁰; wherein R¹⁰ is C1-C4 alkyl; wherein each occurrence of PG is independently a silyl protecting group, or a pharmaceutically acceptable salt thereof, the method comprising coupling a ketone having a structure represented by a formula:

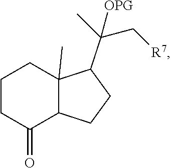

and a phosphine oxide having a structure represented by a formula:

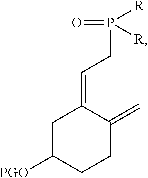

wherein each occurrence of R is independently C₆H₅ substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In various aspects, coupling is in the presence of a base. In a further aspect, the base is selected from n-butyl lithium, 1,8-diazabicyclo[5.4.0]undec-7-ene (DBU), lithium diisopropylamide (LDA), sodium hydride, and potassium tert-butoxide. In a still further aspect, the base is n-butyl lithium.

In various aspects, coupling is in an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, coupling is at a temperature of about −78° C.

In various aspects, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a further aspect, the silyl protecting group is triethylsilyl (TES).

In various aspects, R⁷ is —CN. In a further aspect, R⁷ is —CO₂R¹⁰. In a still further aspect, R⁷ is —CO₂Me.

In various aspects, each occurrence of R is an unsubstituted C₆H.

In various aspects, the ketone has a structure represented by a formula:

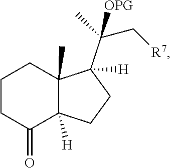

or a pharmaceutically acceptable salt thereof.

In various aspects, the ketone has a structure represented by a formula:

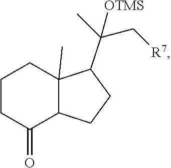

or a pharmaceutically acceptable salt thereof.

In various aspects, the ketone has a structure represented by a formula:

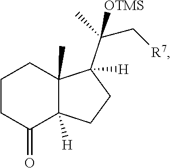

or a pharmaceutically acceptable salt thereof.

In various aspects, the ketone has a structure:

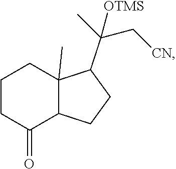

or a pharmaceutically acceptable salt thereof.

In various aspects, the ketone has a structure:

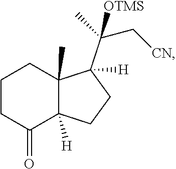

or a pharmaceutically acceptable salt thereof.

In various aspects, the ketone has a structure:

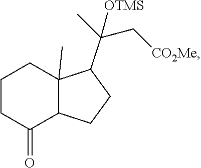

or a pharmaceutically acceptable salt thereof.

In various aspects, the ketone has a structure:

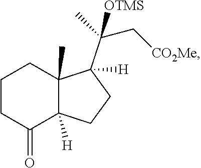

or a pharmaceutically acceptable salt thereof.

In various aspects, the phosphine oxide has a structure represented by a formula:

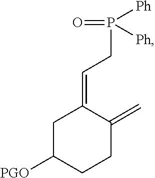

or a pharmaceutically acceptable salt thereof.

In various aspects, the phosphine oxide has a structure represented by a formula:

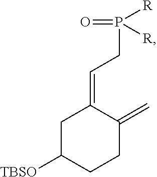

or a pharmaceutically acceptable salt thereof.

In various aspects, the phosphine oxide has a structure:

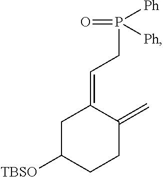 [SMILES: C=C1CCC(O[Si](C)(C)C(C)(C)C)C/C1=C/CP(=O)(c1ccccc1)c1ccccc1]

or a pharmaceutically acceptable salt thereof.

In various aspects, the compound has a structure represented by a formula:

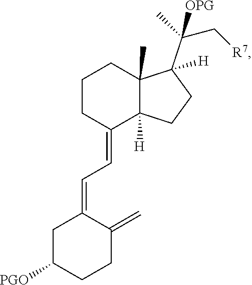

or a pharmaceutically acceptable salt thereof.

In various aspects, the compound has a structure represented by a formula:

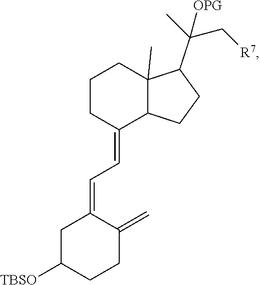

or a pharmaceutically acceptable salt thereof.

In various aspects, the compound has a structure represented by a formula:

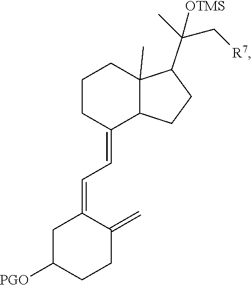

or a pharmaceutically acceptable salt thereof.

In various aspects, the compound has a structure represented by a formula:

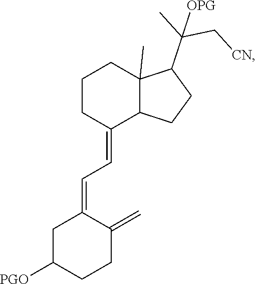

or a pharmaceutically acceptable salt thereof.

In various aspects, the compound has a structure represented by a formula:

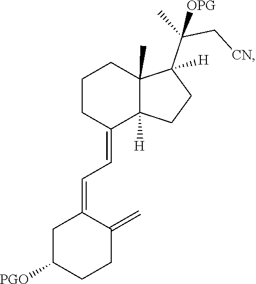

or a pharmaceutically acceptable salt thereof.

In various aspects, the compound has a structure represented by a formula:

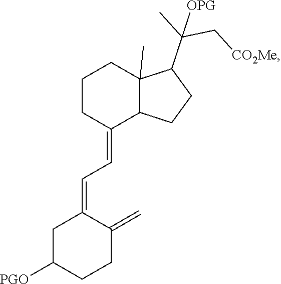

or a pharmaceutically acceptable salt thereof.

In various aspects, the compound has a structure represented by a formula:

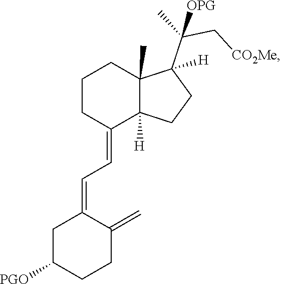

or a pharmaceutically acceptable salt thereof.

In various aspects, the compound has a structure:

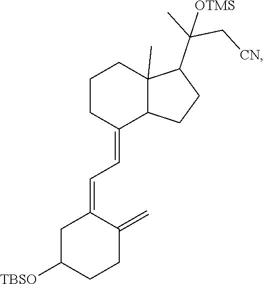

or a pharmaceutically acceptable salt thereof.

In various aspects, the compound has a structure:

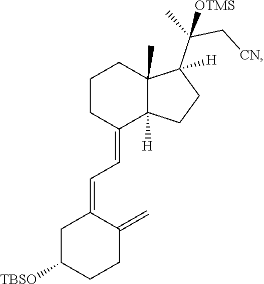

or a pharmaceutically acceptable salt thereof.

In various aspects, the compound has a structure:

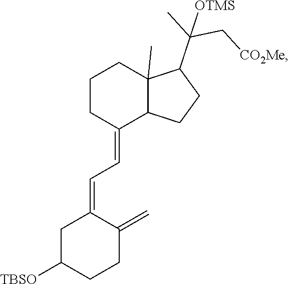

or a pharmaceutically acceptable salt thereof.

In various aspects, the compound has a structure:

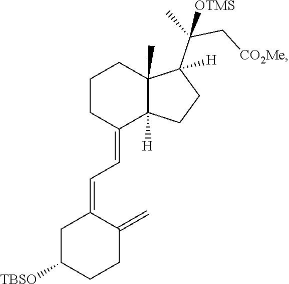

or a pharmaceutically acceptable salt thereof.

### A. Route I

In one aspect, substituted trienes can be prepared as shown below.

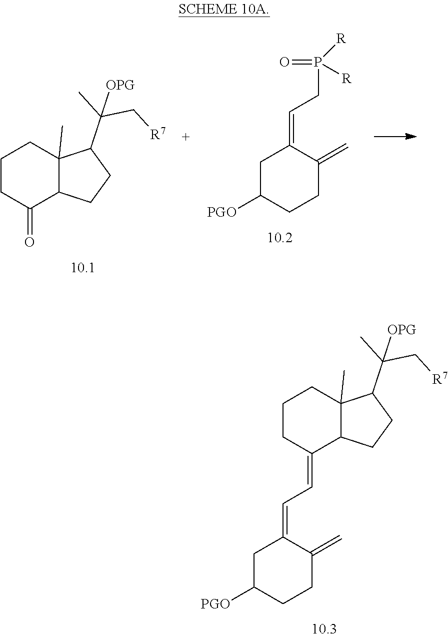

Compounds are represented in generic form, with substituents as noted in compound descriptions elsewhere herein. A more specific example is set forth below.

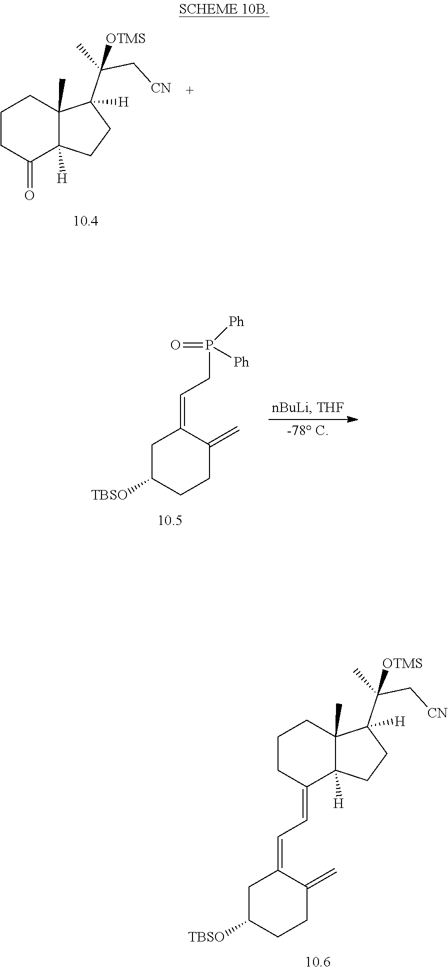

In one aspect, compounds of type 10.6, and similar compounds, can be prepared according to reaction Scheme 10B above. Thus, compounds of type 10.6 can be prepared by a Wittig reaction between an appropriate ketone, e.g., 10.4, andh an appropriate phosphine oxide, e.g., 10.5, as shown above. Appropriate phosphine oxides are commercially available or prepared by methods known to one skilled in the art. The Wittig reaction is carried out in an appropriate solvent, e.g., anhydrous THF, at an appropriate temperature, e.g., −78° C., in the presence of an appropriate base, e.g., n-BuLi, for an appropriate amount of time, e.g., 4 h. As can be appreciated by one skilled in the art, the above reaction provides an example of a generalized approach wherein compounds similar in structure to the specific reactants above (compounds similar to compounds of type 10.1 and 10.2), can be substituted in the reaction to provide substituted trienes similar to Formula 10.3.

### 6. Methods of Making Aldehydes

In one aspect, disclosed are methods of making a compound having a structure represented by a formula:

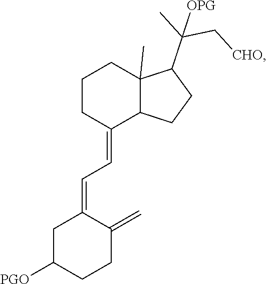

wherein each occurrence of PG is independently a silyl protecting group, or a pharmaceutically acceptable salt thereof, the method comprising reducing a triene having a structure represented by a formula:

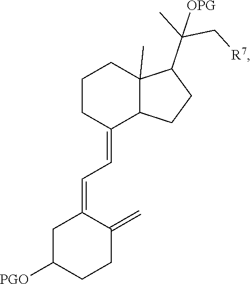

wherein R⁷ is selected from —CN and —CO₂R¹⁰; wherein R¹⁰ is C1-C4 alkyl, or a pharmaceutically acceptable salt thereof.

In various aspects, reducing is in the presence of a reducing agent selected from sodium borohydride, lithium aluminum hydride (LAH), and diisobutylaluminum hydride (DIBAL-H). In a further aspect, reducing is in the presence of diisobutylaluminum hydride (DIBAL-H).

In various aspects, reducing is in the presence of an organic, nonpolar solvent.

In a further aspect, the organic, nonpolar solvent is selected from dichloromethane (DCM), diethyl ether, and pyridine. In a still further aspect, the organic, nonpolar solvent is dichloromethane (DCM).

In various aspects, reducing is at a temperature of about −78° C.

In various aspects, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In various aspects, the silyl protecting group is triethylsilyl (TES).

In various aspects, R⁷ is —CN. In a further aspect, R⁷ is —CO₂R¹⁰. In a yet further aspect, R⁷ is —CO₂Me.

In various aspects, the triene has a structure represented by a formula:

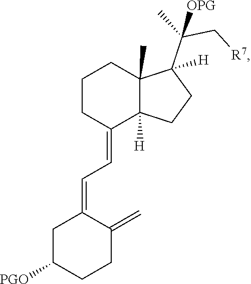

or a pharmaceutically acceptable salt thereof.

In various aspects, the triene has a structure represented by a formula:

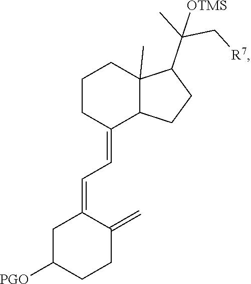

or a pharmaceutically acceptable salt thereof.

In various aspects, the triene has a structure represented by a formula:

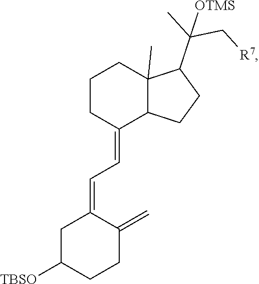

or a pharmaceutically acceptable salt thereof.

In various aspects, the triene has a structure represented by a formula:

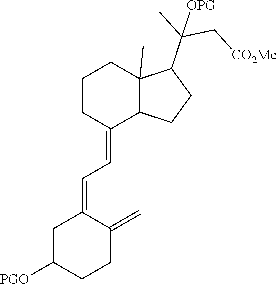

or a pharmaceutically acceptable salt thereof.

In various aspects, the triene has a structure represented by a formula:

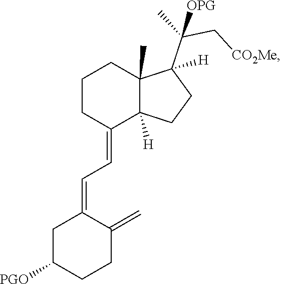

or a pharmaceutically acceptable salt thereof.

In various aspects, the triene has a structure represented by a formula:

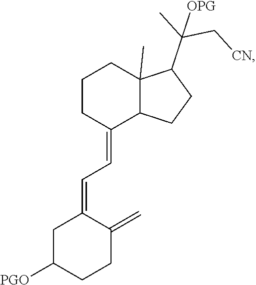

or a pharmaceutically acceptable salt thereof.

In various aspects, the triene has a structure represented by a formula:

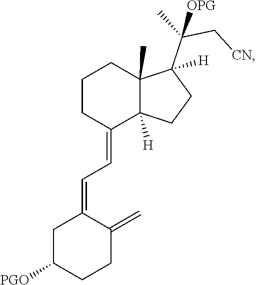

or a pharmaceutically acceptable salt thereof.

In various aspects, the triene has a structure:

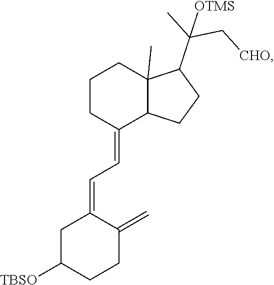

or a pharmaceutically acceptable salt thereof.

In various aspects, the triene has a structure:

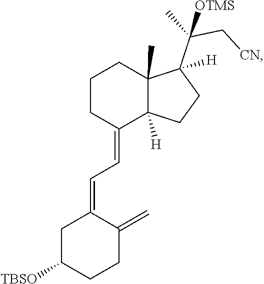

or a pharmaceutically acceptable salt thereof.

In various aspects, the compound has a structure represented by a formula:

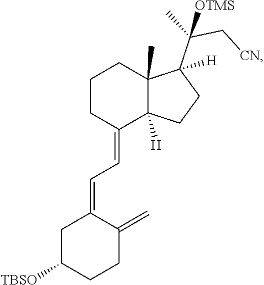

or a pharmaceutically acceptable salt thereof.

In various aspects, the compound has a structure represented by a formula:

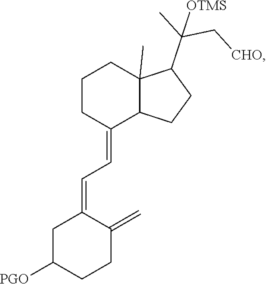

or a pharmaceutically acceptable salt thereof.

In various aspects, the compound has a structure represented by a formula:

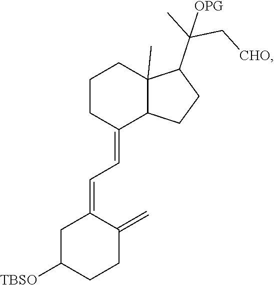

or a pharmaceutically acceptable salt thereof.

In various aspects, the compound has a structure:

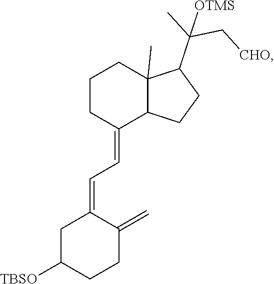

or a pharmaceutically acceptable salt thereof.

In various aspects, the compound has a structure:

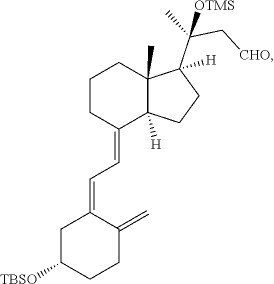

or a pharmaceutically acceptable salt thereof.

### A. Route I

In one aspect, substituted aldehyde analogs can be prepared as shown below.

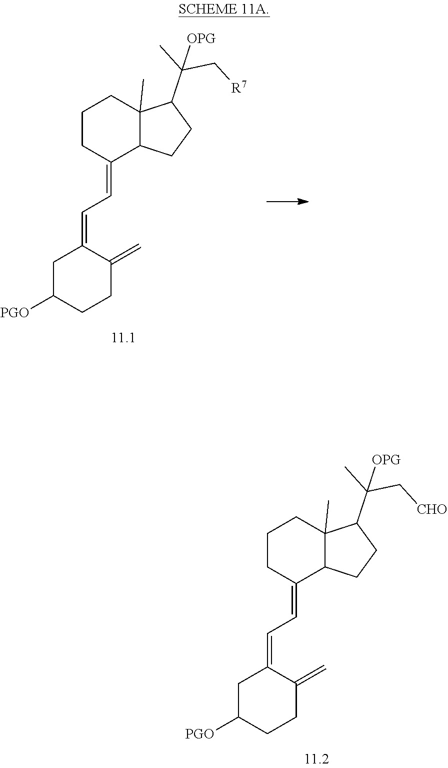

Compounds are represented in generic form, with substituents as noted in compound descriptions elsewhere herein. A more specific example is set forth below.

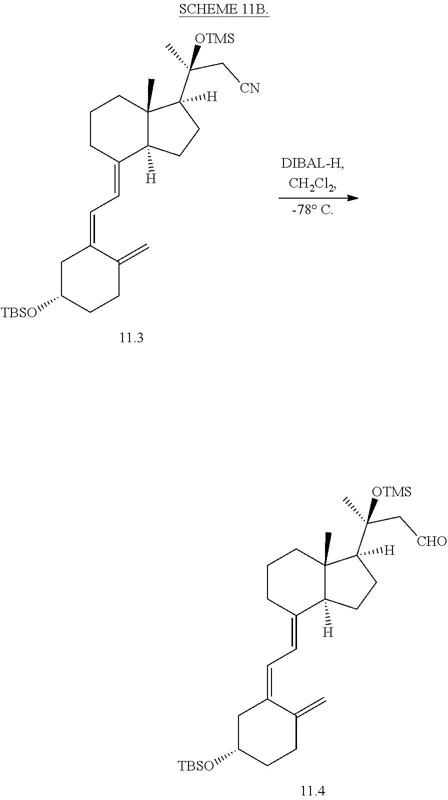

In one aspect, compounds of type 11.4, and similar compounds, can be prepared according to reaction Scheme 11B above. Thus, compounds of type 11.4 can be prepared by reduction of an appropriate nitrile or ester, e.g., 11.3 as shown above.

Appropriate nitriles and appropriate esters are commercially available or prepared by methods known to one skilled in the art. The reduction is carried out with an appropriate reducing agent, e.g., diisobutylaluminum hydride, in an appropriate solvent, e.g., dichloromethane (DCM), at an appropriate temperature, e.g., −10° C., in the presence of an appropriate base, e.g., n-BuLi, for an appropriate amount of time, e.g., 1 h. As can be appreciated by one skilled in the art, the above reaction provides an example of a generalized approach wherein compounds similar in structure to the specific reactants above (compounds similar to compounds of type 11.1), can be substituted in the reaction to provide substituted aldehyde analogs similar to Formula 11.2.

### 7. Methods of Making Alkyl Alcohols

In one aspect, disclosed are methods of making a compound having a structure represented by a formula:

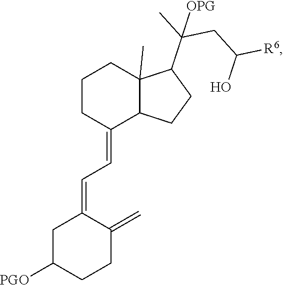

wherein R⁶ is a C1-C8 alkyl; and wherein each occurrence of PG is independently a silyl protecting group, or a pharmaceutically acceptable salt thereof, the method comprising reacting an aldehyde having a structure represented by a formula:

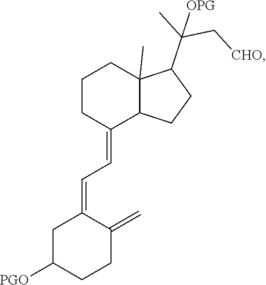

or a pharmaceutically acceptable salt thereof, and a Grignard reagent having a structure represented by a formula:

R⁶—Mg—X,

wherein X is a halide.

In various aspects, reacting is in the presence of an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, reacting is at a temperature of from about 0° C. to about 22° C.

In various aspects, R⁶ is a C1-C4 alkyl. In a further aspect, R⁶ is isobutyl.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a further aspect, each occurrence of PG is independently a silyl protecting group selected from trimethylsilyl (TMS) and tert-butyldimethylsilyl (TBS).

In various aspects, X is —Cl.

In various aspects, the aldehyde has a structure represented by a formula:

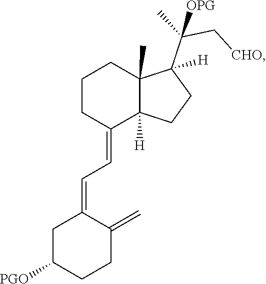

or a pharmaceutically acceptable salt thereof.

In various aspects, the aldehyde has a structure represented by a formula:

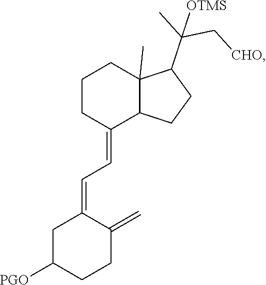

or a pharmaceutically acceptable salt thereof.

In various aspects, the aldehyde has a structure represented by a formula:

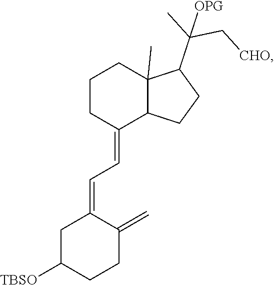

or a pharmaceutically acceptable salt thereof.

In various aspects, the aldehyde has a structure:

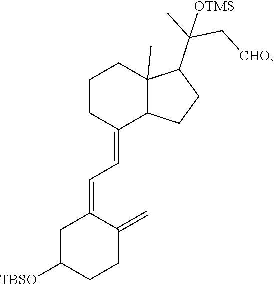

or a pharmaceutically acceptable salt thereof.

In various aspects, the aldehyde has a structure:

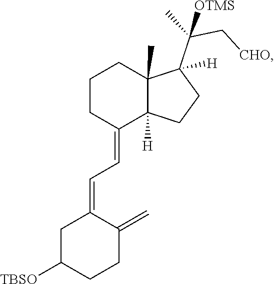

or a pharmaceutically acceptable salt thereof.

In various aspects, the Grignard reagent has a structure represented by a formula:

R⁶—MgCl.

In various aspects, the Grignard reagent has a structure:

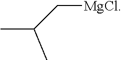

In various aspects, the compound has a structure represented by a formula:

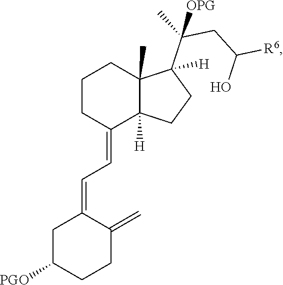

or a pharmaceutically acceptable salt thereof.

In various aspects, the compound has a structure represented by a formula:

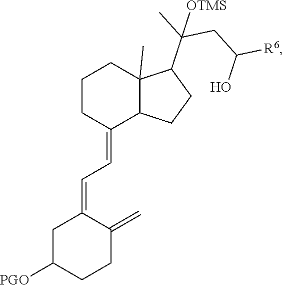

or a pharmaceutically acceptable salt thereof.

In various aspects, the compound has a structure represented by a formula:

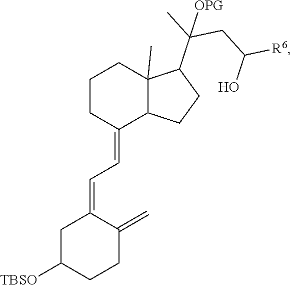

or a pharmaceutically acceptable salt thereof.

In various aspects, the compound has a structure:

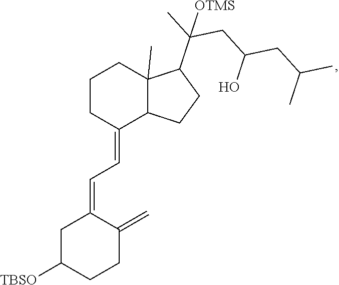

or a pharmaceutically acceptable salt thereof.

In various aspects, the compound has a structure:

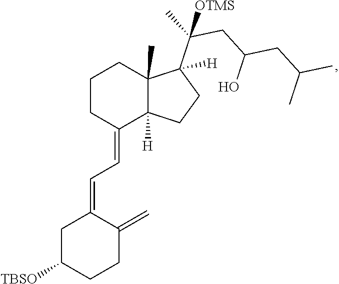

or a pharmaceutically acceptable salt thereof.

In various aspects, the compound has a structure:

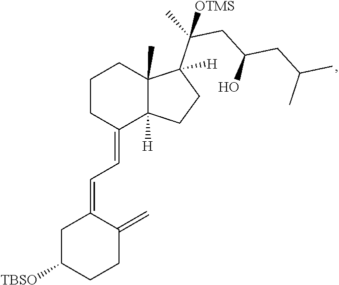

or a pharmaceutically acceptable salt thereof.

In various aspects, the compound has a structure:

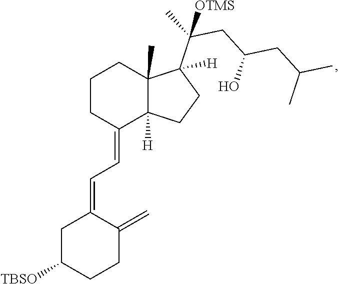

or a pharmaceutically acceptable salt thereof.

### A. Route XI

In one aspect, substituted alkyl alcohols can be prepared as shown below.

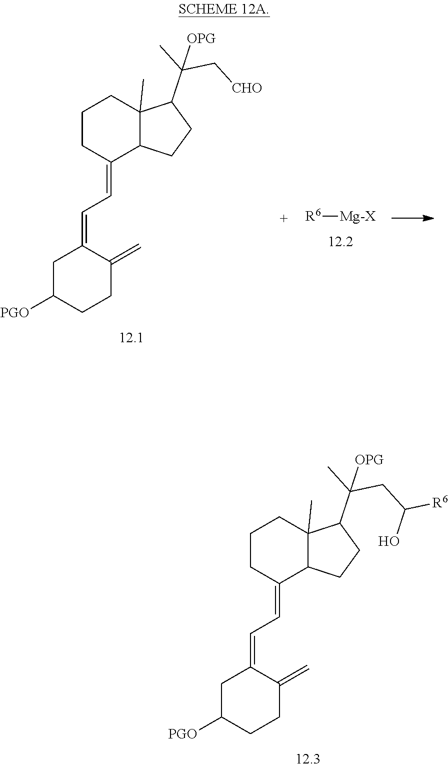

Compounds are represented in generic form, X is a halide and with substituents as noted in compound descriptions elsewhere herein. A more specific example is set forth below.

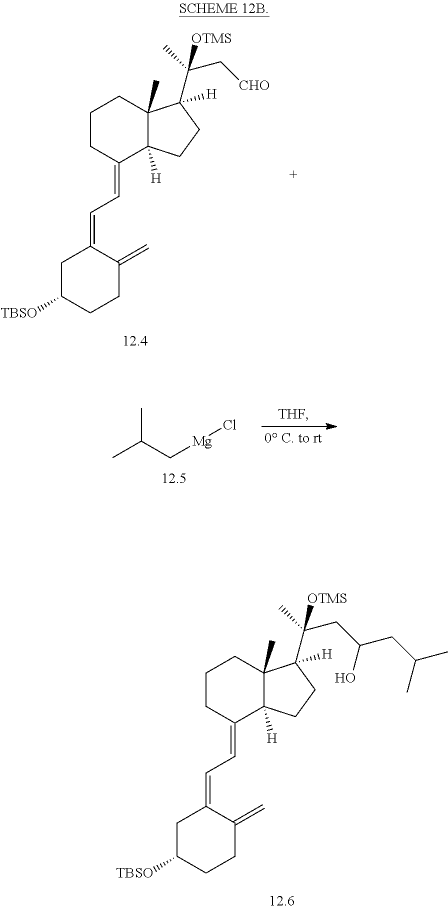

In one aspect, compounds of type 12.6, and similar compounds, can be prepared according to reaction Scheme 12B above. Thus, compounds of type 12.6 can be prepared using an appropriate Grignard reagent, where the Grignard reagent is commercially available or is prepared from appropriate halide, e.g., 12.5 as shown above, and an appropriate ketone, e.g., 12.4 as shown above. Appropriate halides are commercially available or prepared by methods known to one skilled in the art. The Grignard reaction is carried out in the presence of an appropriate aprotic solvent, e.g., tetrahydrofuran (THF), at an appropriate temperature, e.g., 0° C. As can be appreciated by one skilled in the art, the above reaction provides an example of a generalized approach wherein compounds similar in structure to the specific reactants above (compounds similar to compounds of type 12.1 and 12.2), can be substituted in the reaction to provide substituted alkyl alcohols similar to Formula 12.3.

### 8. Methods of Making Open Chain Hydroxy Derivatives of Vitamin D₃

In one aspect, disclosed are methods of making a compound having a structure represented by a formula:

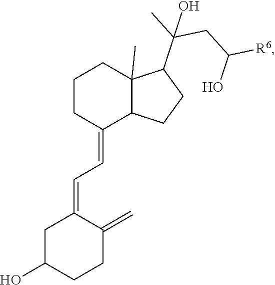

wherein R⁶ is a C1-C8 alkyl, or a pharmaceutically acceptable salt thereof, the method comprising deprotecting an alcohol having a structure represented by a formula:

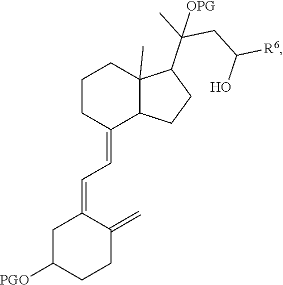

wherein each occurrence of PG is independently a silyl protecting group, or a pharmaceutically acceptable salt thereof.

In various aspects, deprotecting is in the presence of a deprotecting agent selected from potassium fluoride, cesium fluoride, tetrabutylammonium fluoride (TBAF), tris(dimethylamino)sulfur (trimethylsilyl)difluoride (TASF), tetrabutylammonium triphenyldifluorosilicate (TBAT), tetraphenylbismuth fluoride, cadmium fluoride, acidic aqueous tetrahydrofuran (THF), acidic methanol, and acetic acid. In a further aspect, deprotecting is in the presence of tetrabutylammonium fluoride (TBAF).

In various aspects, deprotecting is in an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, R⁶ is a C1-C4 alkyl. In a further aspect, R⁶ is isobutyl.

In various aspects, each occurrence of PG is independently is selected from trimethylsilyl (TMS), triethylsilyl (TES), tert-butyldimethylsilyl (TBS), tert-butyldiphenylsilyl (TBDPS), and triisopropylsilyl (TIPS). In a further aspect, each occurrence of PG is independently a silyl protecting group selected from trimethylsilyl (TMS) and tert-butyldimethylsilyl (TBS).

In various aspects, the alcohol has a structure represented by a formula:

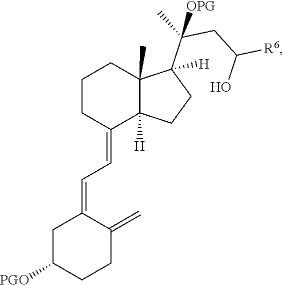

or a pharmaceutically acceptable salt thereof.

In various aspects, the alcohol has a structure represented by a formula:

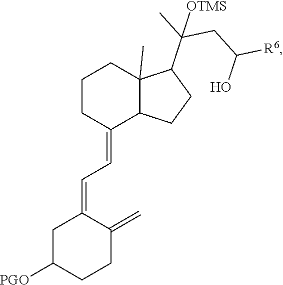

or a pharmaceutically acceptable salt thereof.

In various aspects, the alcohol has a structure represented by a formula:

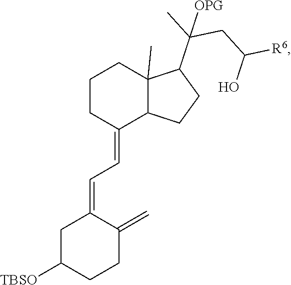

or a pharmaceutically acceptable salt thereof.

In various aspects, the alcohol has a structure:

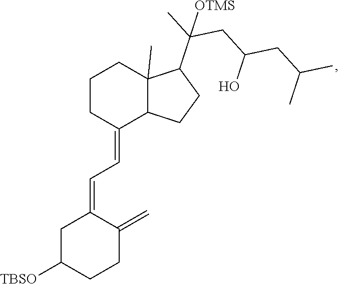

or a pharmaceutically acceptable salt thereof.

In various aspects, the alcohol has a structure:

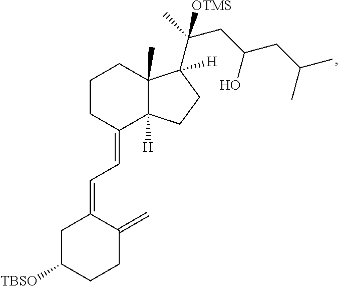

or a pharmaceutically acceptable salt thereof.

In various aspects, the alcohol has a structure:

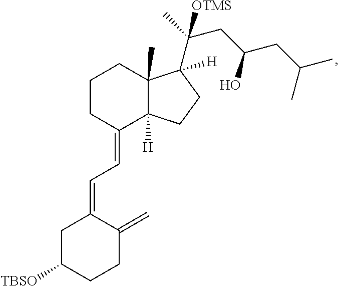

or a pharmaceutically acceptable salt thereof.

In various aspects, the alcohol has a structure:

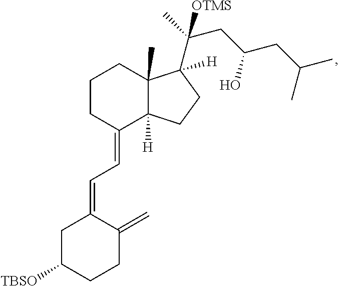

or a pharmaceutically acceptable salt thereof.

In various aspects, the compound has a structure represented by a formula:

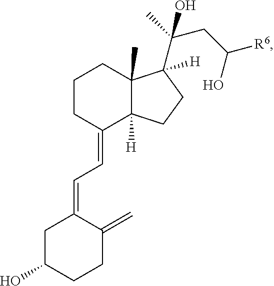

or a pharmaceutically acceptable salt thereof.

In various aspects, the compound has a structure:

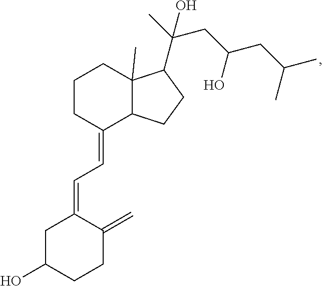 [SMILES: C=C1CCC(O)C/C1=C/C=C1\CCCC2(C)C1CCC2C(C)(O)CC(O)CC(C)C]

or a pharmaceutically acceptable salt thereof.

In various aspects, the compound has a structure:

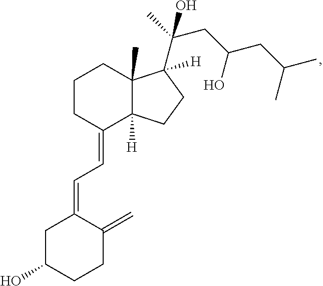 [SMILES: C=C1CC[C@H](O)C/C1=C/C=C1\CCC[C@@]2(C)[C@H]1CC[C@@H]2[C@@](C)(O)CC(O)CC(C)C]

or a pharmaceutically acceptable salt thereof.

In various aspects, the compound has a structure:

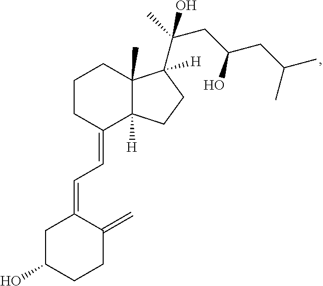 [SMILES: C=C1CC[C@H](O)C/C1=C/C=C1\CCC[C@@]2(C)[C@H]1CC[C@@H]2[C@@](C)(O)C[C@H](O)CC(C)C]

or a pharmaceutically acceptable salt thereof.

In various aspects, the compound has a structure:

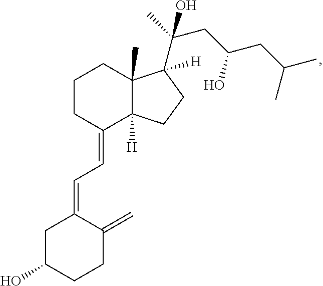 [SMILES: C=C1CC[C@H](O)C/C1=C/C=C1\CCC[C@@]2(C)[C@H]1CC[C@@H]2[C@@](C)(O)C[C@@H](O)CC(C)C]

or a pharmaceutically acceptable salt thereof.

### A. Route I

In one aspect, substituted open chain hydroxy derivatives of vitamin D₃ can be prepared as shown below.

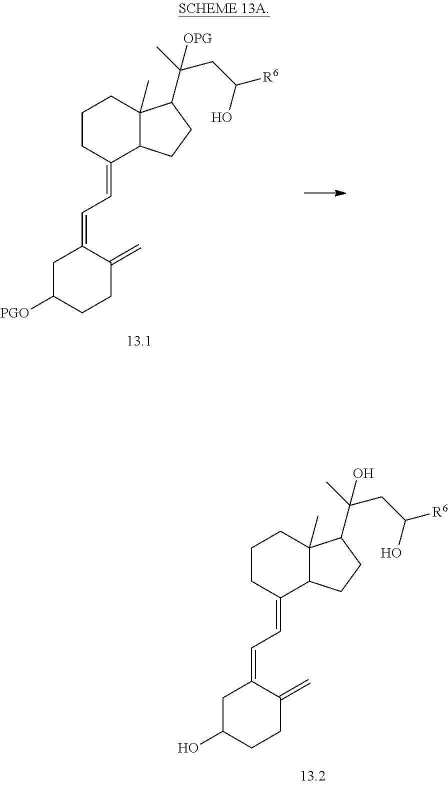

Compounds are represented in generic form, with substituents as noted in compound descriptions elsewhere herein. A more specific example is set forth below.

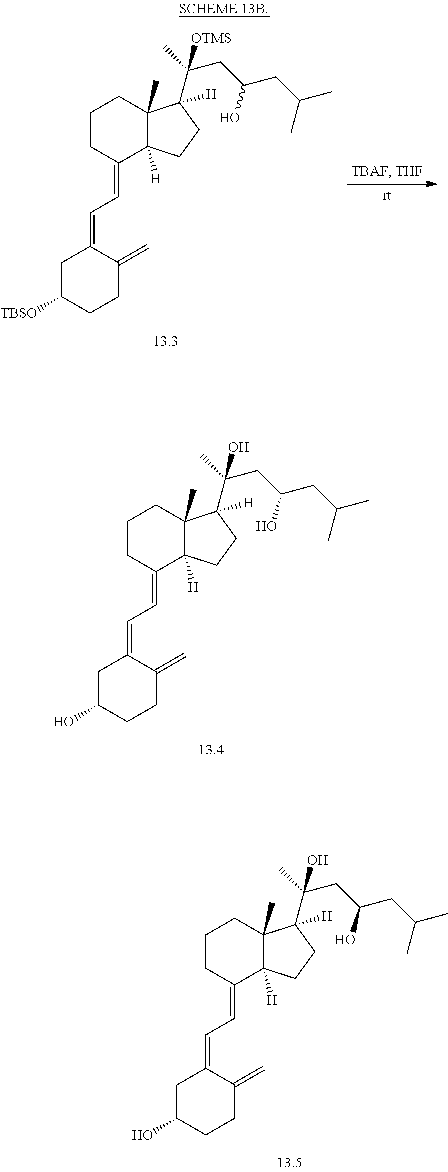

In one aspect, compounds of type 13.4 and 13.5, and similar compounds, can be prepared according to reaction Scheme 13B above. Thus, compounds of type 13.4 and 13.5 can be prepared by deprotection of an appropriate protected alcohol, e.g., 13.3, as shown above. The deprotection is carried out in an appropriate solvent, e.g., anhydrous THF, at an appropriate temperature, e.g., room temperature, in the presence of a deprotecting agent, e.g., tetra-n-butylammonium fluoride, for an appropriate amount of time, e.g., 18 h. As can be appreciated by one skilled in the art, the above reaction provides an example of a generalized approach wherein compounds similar in structure to the specific reactants above (compounds similar to compounds of type 13.1), can be substituted in the reaction to provide substituted open chain hydroxy derivatives of vitamin D₃ similar to Formula 13.2.

### F. Compounds Useful as Intermediates Towards the Preparation of Hydroxy Derivatives of Vitamin D₃ (Closed Form-Route I)

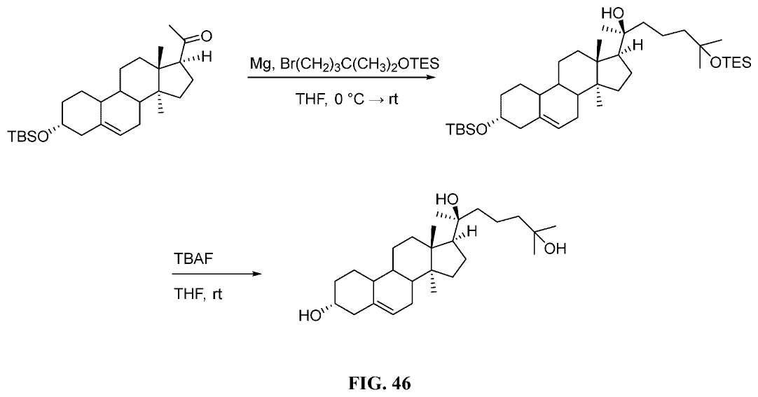

In one aspect, disclosed are compounds useful as intermediates towards the preparation of closed forms of chain hydroxy derivatives of vitamin D₃. See, e.g., FIG. 46. As detailed herein, hydroxy derivatives of vitamin D₃ have demonstrated utility as therapeutic agents for a variety of autoimmune disorders including, but not limited to, lupus erythematosus, rheumatoid arthritis, inflammatory bowel disease, psoriasis, and multiple sclerosis. Thus, in various aspects, disclosed are protected tertiary alcohols useful as intermediates in the synthesis of closed form hydroxy derivatives of vitamin D₃.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Protected Tertiary Alcohols

In one aspect, disclosed are protected tertiary alcohols having a structure represented by a formula:

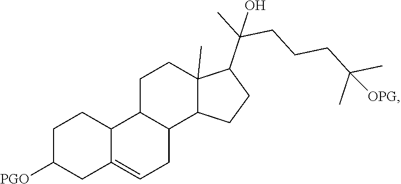

wherein each occurrence of PG is a silyl protecting group, or a pharmaceutically acceptable salt thereof.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a further aspect, each occurrence of PG is independently a silyl protecting group selected from trimethylsilyl (TMS), triethylsilyl (TES), and tert-butyldimethylsilyl (TBS).

In various aspects, each occurrence of PG is independently a different silyl protecting group. In a further aspect, each occurrence of PG is the same silyl protecting group.

In various aspects, the compound has a structure represented by a formula:

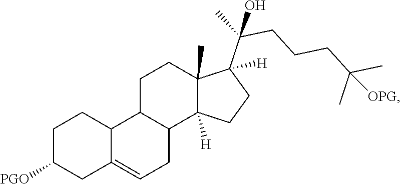

or a pharmaceutically acceptable salt thereof.

In various aspects, the compound has a structure represented by a formula:

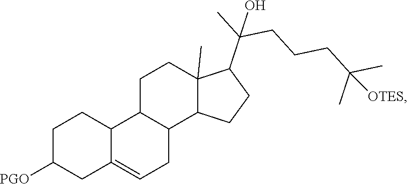

or a pharmaceutically acceptable salt thereof.

In various aspects, the compound has a structure represented by a formula:

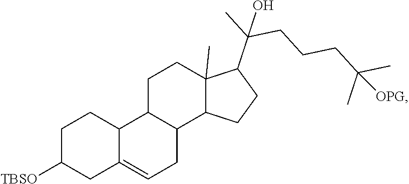

or a pharmaceutically acceptable salt thereof.

In various aspects, the compound has a structure:

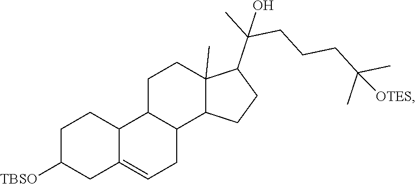

or a pharmaceutically acceptable salt thereof.

In various aspects, the compound has a structure:

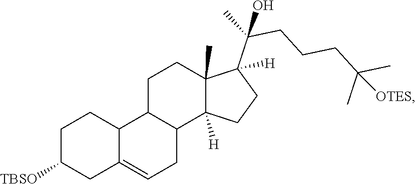

or a pharmaceutically acceptable salt thereof.

### A. Prophetic Exemplary Protected Tertiary Alcohols

The following compound examples are prophetic, and can be prepared using the synthesis methods described herein and other general methods as needed as would be known to one skilled in the art. It is anticipated that the prophetic compounds would be useful in the synthesis of closed form substituted vitamin D₃ derivatives.

In one aspect, a protected tertiary alcohol can be present as:

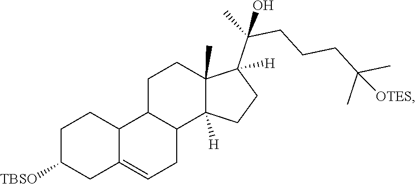

or a pharmaceutically acceptable salt thereof.

### 2. Closed Form Hydroxy Derivatives of Vitamin D₃

In one aspect, disclosed are closed form hydroxy derivatives of vitamin D₃ having a structure represented by a formula:

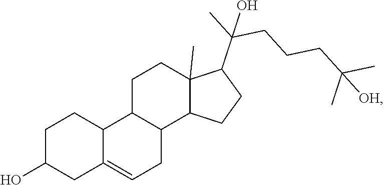 [SMILES: CC(C)(O)CCCC(C)(O)C1CCC2C3CC=C4CC(O)CCC4C3CCC21C]

or a pharmaceutically acceptable salt thereof.

In various aspects, the compound has a structure represented by a formula:

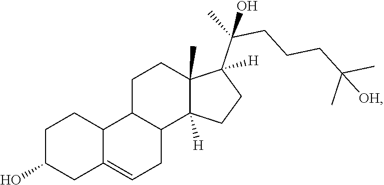 [SMILES: CC(C)(O)CCC[C@](C)(O)[C@H]1CC[C@H]2C3CC=C4C[C@H](O)CCC4C3CC[C@@]21C]

or a pharmaceutically acceptable salt thereof.

### A. Prophetic Exemplary Closed Form Hydroxy Derivatives of Vitamin D₃

The following compound examples are prophetic, and can be prepared using the synthesis methods described herein and other general methods as needed as would be known to one skilled in the art.

In one aspect, a closed form hydroxy derivative of vitamin D₃ can be present as:

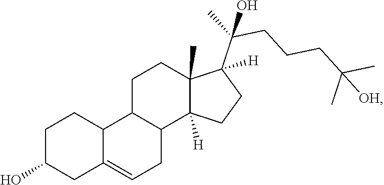 [SMILES: CC(C)(O)CCC[C@](C)(O)[C@H]1CC[C@H]2C3CC=C4C[C@H](O)CCC4C3CC[C@@]21C]

or a pharmaceutically acceptable salt thereof.

### G. Methods of Making Hydroxy Derivatives of Vitamin D₃ (Closed Form-Route I)

In one aspect, disclosed are methods of making closed form hydroxy derivatives of vitamin D₃. See, e.g., FIG. 46. As detailed herein, hydroxy derivatives of vitamin D₃ have demonstrated utility as therapeutic agents for a variety of autoimmune disorders including, but not limited to, lupus erythematosus, rheumatoid arthritis, inflammatory bowel disease, psoriasis, and multiple sclerosis. Thus, in various aspects, closed form hydroxy derivatives of vitamin D₃ can be prepared by adding an appropriate nucleophile to an appropriate ketone to provide the desired functionality, followed by removal of the protecting groups.

### 1. Methods of Making Protected Tertiary Alcohols

In one aspect, disclosed are methods of making a compound having a structure represented by a formula:

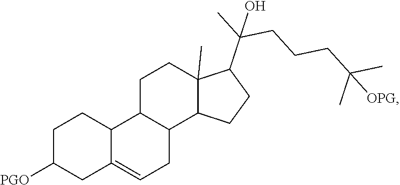

wherein each occurrence of PG is independently a silyl protecting group, or a pharmaceutically acceptable salt thereof, the method comprising reacting a ketone having a structure represented by a formula:

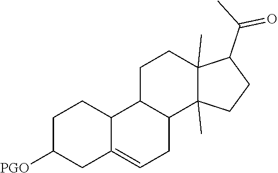

or a pharmaceutically acceptable salt thereof, and a Grignard reagent having a structure represented by a formula:

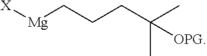

In various aspects, reacting is in the presence of an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, reacting is at a temperature of from about 0° C. to about 22° C.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a further aspect, each occurrence of PG is independently a silyl protecting group selected from trimethylsilyl (TMS) and tert-butyldimethylsilyl (TBS).

In various aspects, X is —Cl. In a further aspect, X is —Br. In a yet further aspect, X is —I.

In various aspects, the ketone has a structure represented by a formula:

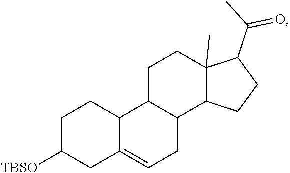 [SMILES: CC(=O)C1CCC2C3CC=C4CC(O[Si](C)(C)C(C)(C)C)CCC4C3CCC12C]

or a pharmaceutically acceptable salt thereof.

In various aspects, the ketone has a structure represented by a formula:

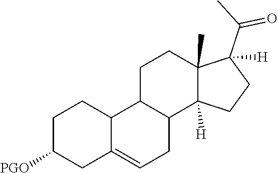

or a pharmaceutically acceptable salt thereof.

In various aspects, the ketone has a structure represented by a formula:

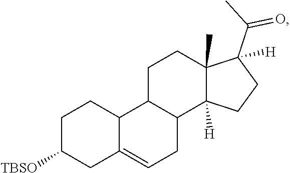 [SMILES: CC(=O)[C@H]1CC[C@H]2C3CC=C4C[C@H](O[Si](C)(C)C(C)(C)C)CCC4C3CC[C@]12C]

or a pharmaceutically acceptable salt thereof.

In various aspects, the Grignard reagent has a structure:

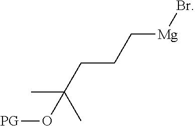

In various aspects, the Grignard reagent has a structure:

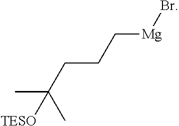

In various aspects, the compound has a structure represented by a formula:

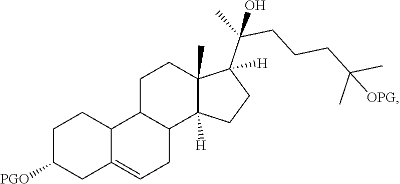

or a pharmaceutically acceptable salt thereof.

In various aspects, the compound has a structure represented by a formula:

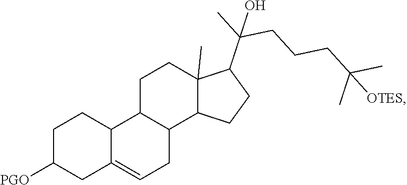

or a pharmaceutically acceptable salt thereof.

In various aspects, the compound has a structure represented by a formula:

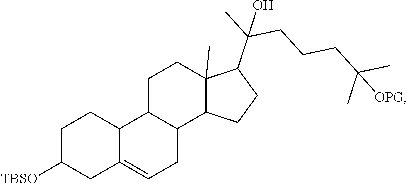

or a pharmaceutically acceptable salt thereof.

In various aspects, the compound has a structure:

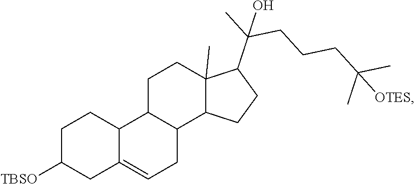

or a pharmaceutically acceptable salt thereof.

In various aspects, the compound has a structure:

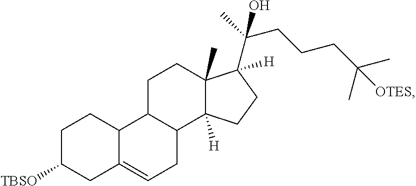

or a pharmaceutically acceptable salt thereof.

### A. Route I

In one aspect, protected tertiary alcohols can be prepared as shown below.

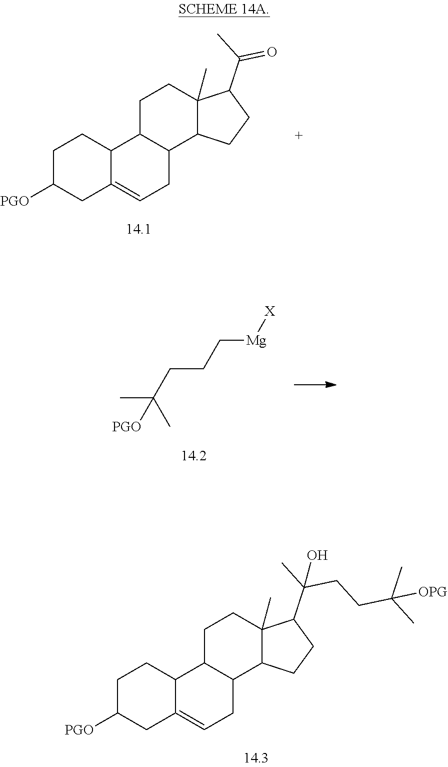

Compounds are represented in generic form, wherein X is a halide and with other substituents as noted in compound descriptions elsewhere herein. A more specific example is set forth below.

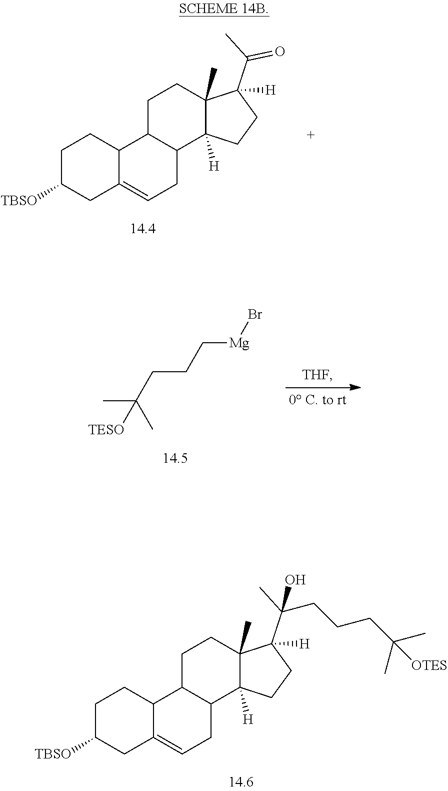

In one aspect, compounds of type 14.6, and similar compounds, can be prepared according to reaction Scheme 14B above. Thus, compounds of type 14.6 can be prepared from an appropriate ketone, e.g., 14.4 as shown above, and an appropriate Grignard reagent, e.g., 14.5 as shown above, where the Grignard reagent is commercially available or is prepared from an appropriate alkyl halide and magnesium metal. Appropriate halides and appropriate ketones are commercially available or prepared by methods known to one skilled in the art. The Grignard reaction is carried out in the presence of an appropriate aprotic solvent, e.g., tetrahydrofuran (THF), at an appropriate temperature, e.g., 0° C. to room temperature. As can be appreciated by one skilled in the art, the above reaction provides an example of a generalized approach wherein compounds similar in structure to the specific reactants above (compounds similar to compounds of type 14.1 and 14.2), can be substituted in the reaction to provide substituted protected tertiary alcohols similar to Formula 14.3.

### 2. Methods of Making Closed Form Hydroxy Derivatives of Vitamin D₃

In one aspect, disclosed are methods of making a compound having a structure represented by a formula:

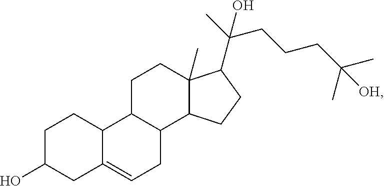 [SMILES: CC(C)(O)CCCC(C)(O)C1CCC2C3CC=C4CC(O)CCC4C3CCC21C]

the method comprising deprotecting a protected tertiary alcohol having a structure represented by a formula:

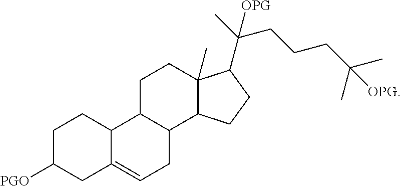

In various aspects, deprotecting is in the presence of a deprotecting agent selected from potassium fluoride, cesium fluoride, tetrabutylammonium fluoride (TBAF), tris(dimethylamino)sulfur (trimethylsilyl)difluoride (TASF), tetrabutylammonium triphenyldifluorosilicate (TBAT), tetraphenylbismuth fluoride, cadmium fluoride, acidic aqueous tetrahydrofuran (THF), acidic methanol, and acetic acid. In a further aspect, deprotecting is in the presence of tetrabutylammonium fluoride (TBAF).

In various aspects, deprotecting is in an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a further aspect, each occurrence of PG is independently a silyl protecting group selected from trimethylsilyl (TMS) and tert-butyldimethylsilyl (TBS).

In various aspects, the protected tertiary alcohol has a structure represented by a formula:

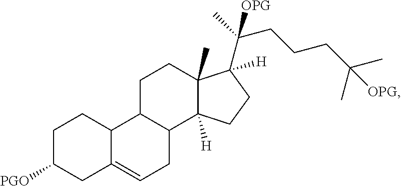

or a pharmaceutically acceptable salt thereof.

In various aspects, the protected tertiary alcohol has a structure represented by a formula:

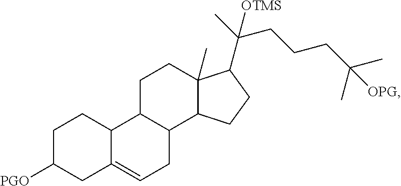

or a pharmaceutically acceptable salt thereof.

In various aspects, the protected tertiary alcohol has a structure represented by a formula:

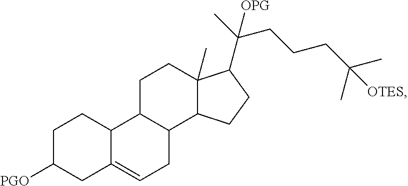

or a pharmaceutically acceptable salt thereof.

In various aspects, the protected tertiary alcohol has a structure represented by a formula:

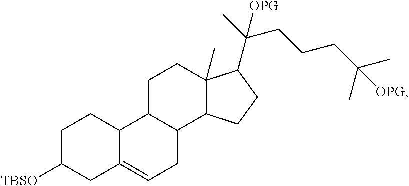

or a pharmaceutically acceptable salt thereof.

In various aspects, the protected tertiary alcohol has a structure:

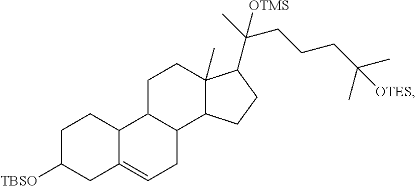

or a pharmaceutically acceptable salt thereof.

In various aspects, the protected tertiary alcohol has a structure:

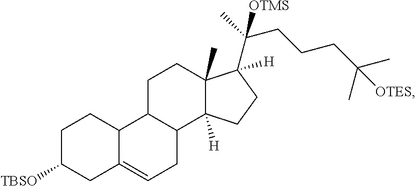

or a pharmaceutically acceptable salt thereof.

In various aspects, the closed form hydroxy derivative of D₃ has a structure:

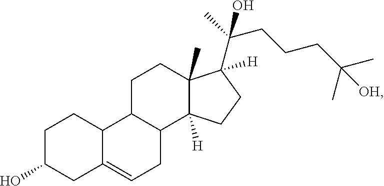 [SMILES: CC(C)(O)CCC[C@](C)(O)[C@H]1CC[C@H]2C3CC=C4C[C@H](O)CCC4C3CC[C@@]21C]

or a pharmaceutically acceptable salt thereof.

### A. Route I

In one aspect, closed form hydroxy derivatives of vitamin D₃ can be prepared as shown below.

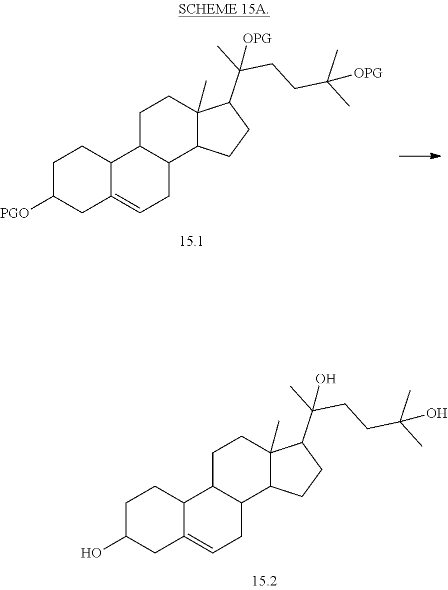

Compounds are represented in generic form, wherein X is a halide and with other substituents as noted in compound descriptions elsewhere herein. A more specific example is set forth below.

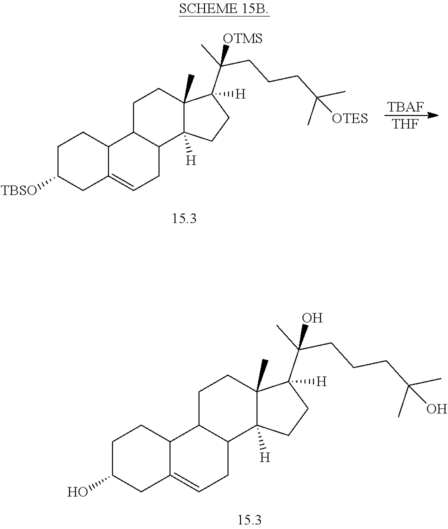

In one aspect, compounds of type 15.3, and similar compounds, can be prepared according to reaction Scheme 15B above. Thus, compounds of type 15.3 can be prepared by deprotection of an appropriate protected tertiary alcohol. The deprotection is carried out in the presence of an appropriate deprotecting agent, e.g., tetra-n-butylammonium (TBAF), in an appropriate temperature, e.g., tetrahydrofuran (THF). As can be appreciated by one skilled in the art, the above reaction provides an example of a generalized approach wherein compounds similar in structure to the specific reactants above (compounds similar to compounds of type 15.1), can be substituted in the reaction to provide substituted closed form hydroxy derivatives of vitamin D₃ similar to Formula 15.3.

### H. Compounds Useful as Intermediates Towards the Preparation of Hydroxy Derivatives of Vitamin D₃ (Closed Form-Route II)

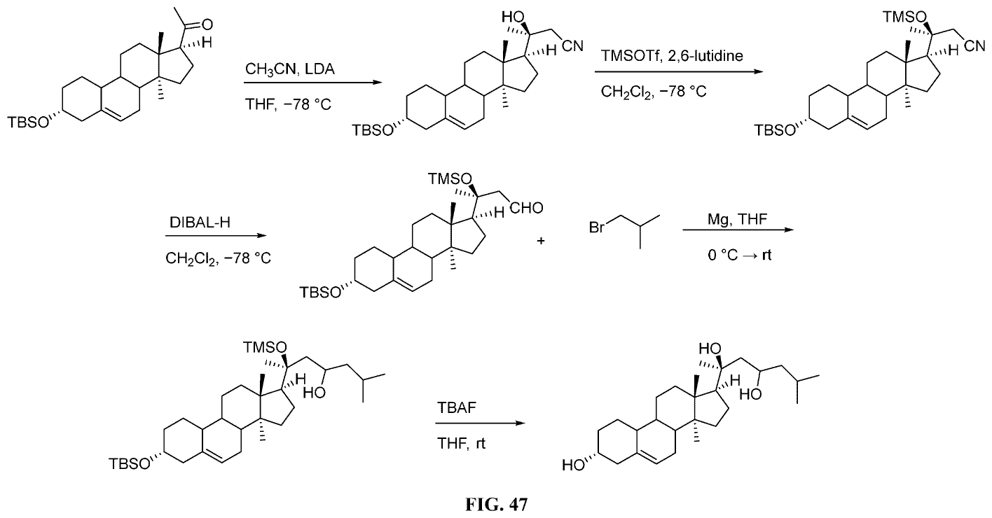

In one aspect, disclosed are compounds useful as intermediates towards the preparation of closed forms of chain hydroxy derivatives of vitamin D₃. See, e.g., FIG. 47. As detailed herein, hydroxy derivatives of vitamin D₃ have demonstrated utility as therapeutic agents for a variety of autoimmune disorders including, but not limited to, lupus erythematosus, rheumatoid arthritis, inflammatory bowel disease, psoriasis, and multiple sclerosis. Thus, in various aspects, disclosed are cyano derivatives, aldehydes, and protected tertiary alcohols useful as intermediates in the synthesis of closed form hydroxy derivatives of vitamin D₃.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Nitrile and Ester Derivatives

In one aspect, disclosed are nitrile and ester derivatives having a structure represented by a formula:

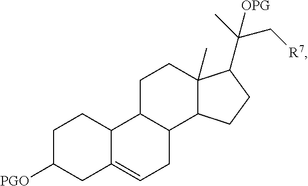

wherein R⁷ is selected from —CN and —CO₂R¹⁰; and wherein R¹⁰ is C1-C4 alkyl; and wherein each occurrence of PG is independently a silyl protecting group, or a pharmaceutically acceptable salt thereof.

In various aspects, each occurrence of PG is independently a silyl protecting group. In a further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, the silyl protecting group is selected from trimethylsilyl (TMS), triethylsilyl (TES), and tert-butyldimethylsilyl (TBS).

In various aspects, the compound has a structure represented by a formula:

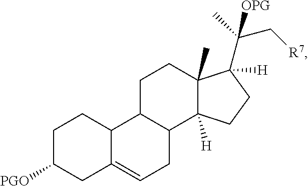

or a pharmaceutically acceptable salt thereof.

In various aspects, the compound has a structure represented by a formula:

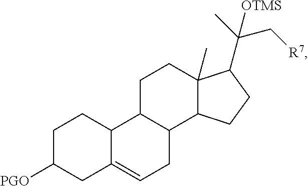

or a pharmaceutically acceptable salt thereof.

In various aspects, the compound has a structure represented by a formula:

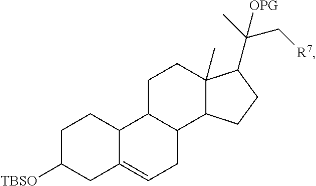

or a pharmaceutically acceptable salt thereof.

In various aspects, the compound has a structure represented by a formula:

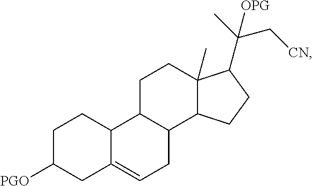

or a pharmaceutically acceptable salt thereof.

In various aspects, the compound has a structure represented by a formula:

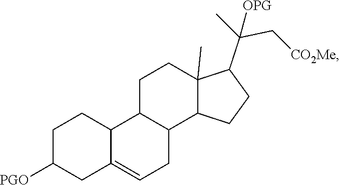

or a pharmaceutically acceptable salt thereof.

In various aspects, the compound has a structure:

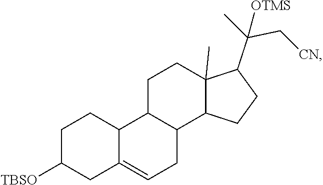

or a pharmaceutically acceptable salt thereof.

In various aspects, the compound has a structure:

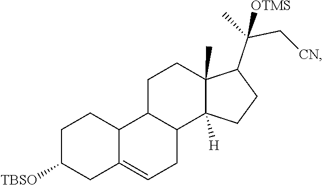

or a pharmaceutically acceptable salt thereof.

In various aspects, the compound has a structure:

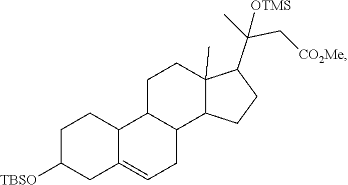

or a pharmaceutically acceptable salt thereof.

In various aspects, the compound has a structure:

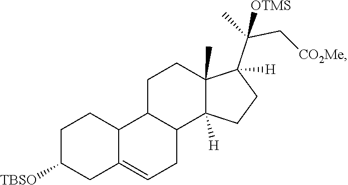

or a pharmaceutically acceptable salt thereof.

### A. R⁷ Groups

In one aspect, R⁷ is selected from —CN and —CO₂R¹⁰. In a further aspect, R⁷ is —CN. In a still further aspect, R⁷ is —CO₂R¹⁰. In an even still further aspect, R⁷ is —CO₂Me.

b. R¹⁰ Groups

In one aspect, R¹⁰ is a C1-C4 alkyl. In a further aspect, R¹⁰ is selected from methyl, ethyl, n-propyl, and i-propyl. In a still further aspect, R¹⁰ is selected from methyl and ethyl. In a yet further aspect, R¹⁰ is methyl. In an even further aspect, R¹⁰ is ethyl.

c. Prophetic Exemplary Cyano Derivatives

The following compound examples are prophetic, and can be prepared using the synthesis methods described herein and other general methods as needed as would be known to one skilled in the art. It is anticipated that the prophetic compounds would be useful in the synthesis of substituted vitamin D₃ derivatives.

In one aspect, a cyano derivative can be present as:

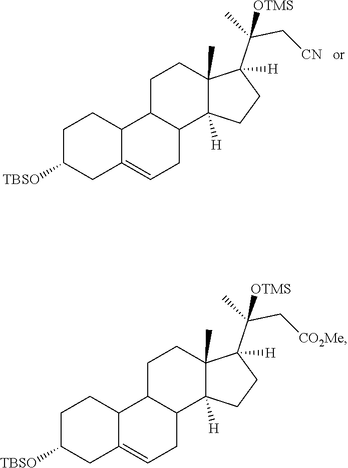

or a pharmaceutically acceptable salt thereof.

### 2. Aldehydes

In one aspect, disclosed are aldehydes having a structure represented by a formula:

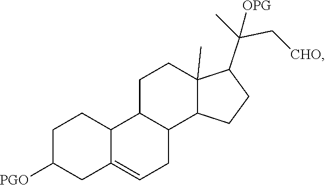

wherein each occurrence of PG is independently a silyl protecting group, or a pharmaceutically acceptable salt thereof.

In various aspects, each occurrence of PG is independently a silyl protecting group. In a further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, the silyl protecting group is selected from trimethylsilyl (TMS), triethylsilyl (TES), and tert-butyldimethylsilyl (TBS).

In various aspects, the compound has a structure represented by a formula:

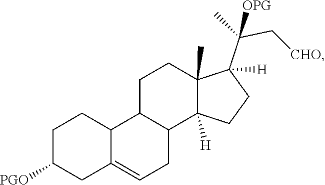

or a pharmaceutically acceptable salt thereof.

In various aspects, the compound has a structure represented by a formula:

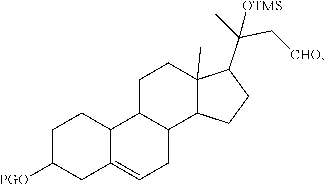

or a pharmaceutically acceptable salt thereof.

In various aspects, the compound has a structure represented by a formula:

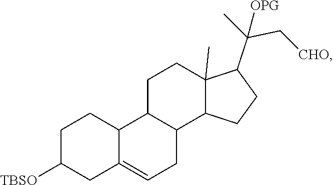

or a pharmaceutically acceptable salt thereof.

In various aspects, the compound has a structure:

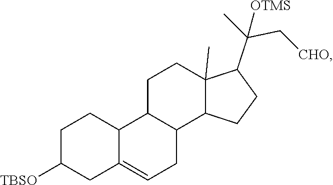

or a pharmaceutically acceptable salt thereof.

In various aspects, the compound has a structure:

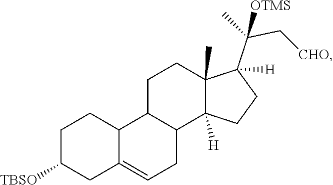

or a pharmaceutically acceptable salt thereof.

### A. Prophetic Exemplary Aldehydes

The following compound examples are prophetic, and can be prepared using the synthesis methods described herein and other general methods as needed as would be known to one skilled in the art. It is anticipated that the prophetic compounds would be useful in the synthesis of substituted vitamin D₃ derivatives.

In one aspect, an aldehyde can be:

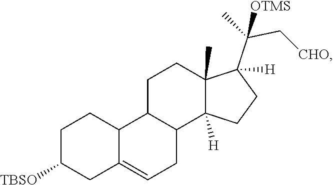

or a pharmaceutically acceptable salt thereof.

### 3. Protected Tertiary Alcohols

In one aspect, disclosed are protected tertiary alcohols having a structure represented by a formula:

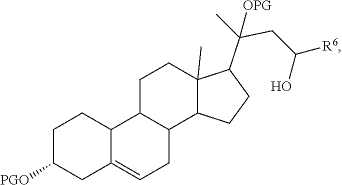

wherein R⁶ is a C1-C8 alkyl; and wherein each occurrence of PG is independently a silyl protecting group, or a pharmaceutically acceptable salt thereof.

In various aspects, each occurrence of PG is independently a silyl protecting group. In a further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a still further aspect, the silyl protecting group is selected from trimethylsilyl (TMS), triethylsilyl (TES), and tert-butyldimethylsilyl (TBS).

In various aspects, the compound has a structure represented by a formula:

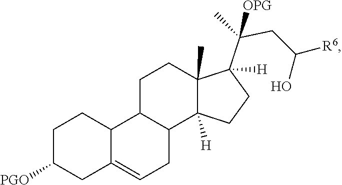

or a pharmaceutically acceptable salt thereof.

In various aspects, the compound has a structure represented by a formula:

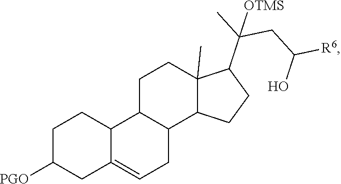

or a pharmaceutically acceptable salt thereof.

In various aspects, the compound has a structure represented by a formula:

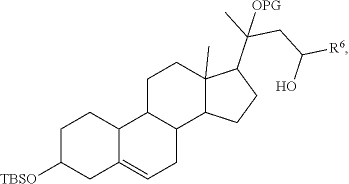

or a pharmaceutically acceptable salt thereof.

In various aspects, the compound has a structure represented by a formula:

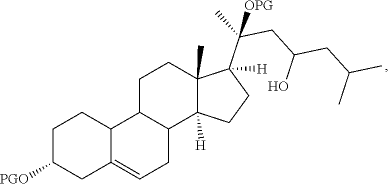

or a pharmaceutically acceptable salt thereof.

In various aspects, the compound has a structure:

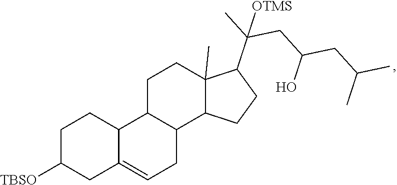

or a pharmaceutically acceptable salt thereof.

In various aspects, the compound has a structure:

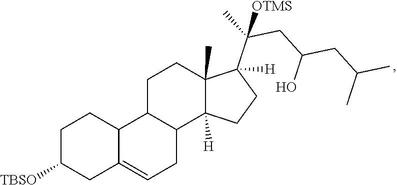

or a pharmaceutically acceptable salt thereof.

In various aspects, the compound has a structure:

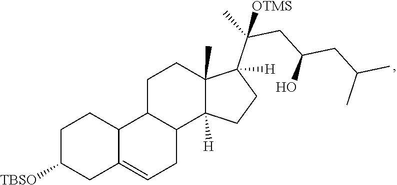

or a pharmaceutically acceptable salt thereof.

In various aspects, the compound has a structure:

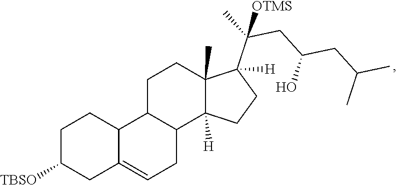

or a pharmaceutically acceptable salt thereof.

### A. R⁶ Groups

In one aspect, R⁶ is a C1-C8 alkyl. In a further aspect, R⁶ is a C1-C4 alkyl. In a still further aspect, R⁶ is selected from methyl, ethyl, n-propyl, i-propyl, n-butyl, isobutyl, secbutyl, and tert-butyl. In a yet further aspect, R⁶ is selected from methyl, ethyl, n-propyl, and i-propyl. In an even further aspect, R⁶ is selected from methyl and ethyl. In a still further aspect, R⁶ is ethyl. In yet a further aspect, R⁶ is methyl.

In various aspects, R⁶ is isobutyl.

b. Prophetic Protected Tertiary Alcohols

The following compound examples are prophetic, and can be prepared using the synthesis methods described herein and other general methods as needed as would be known to one skilled in the art. It is anticipated that the prophetic compounds would be useful in the synthesis of substituted vitamin D₃ derivatives.

In one aspect, a protected tertiary alcohol can be selected from:

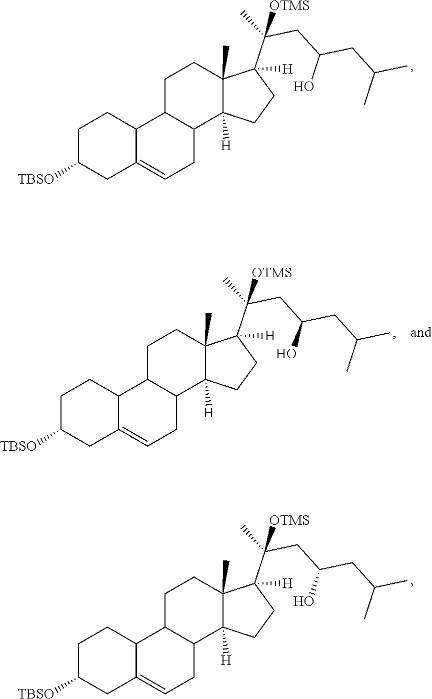

or a pharmaceutically acceptable salt thereof.

### 4. Closed Form Hydroxy Derivatives of Vitamin D₃

In one aspect, disclosed are closed form hydroxy derivatives of vitamin D₃ having a structure represented by a formula:

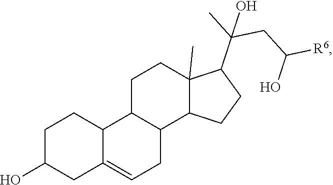

wherein R⁶ is a C1-C8 alkyl; or a pharmaceutically acceptable salt thereof.

In various aspects, the compound has a structure represented by a formula:

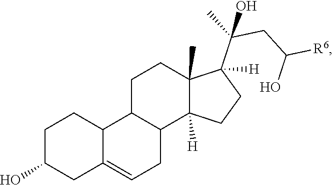

or a pharmaceutically acceptable salt thereof.

In various aspects, the compound has a structure represented by a formula:

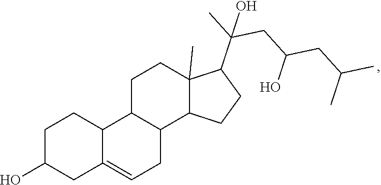 [SMILES: CC(C)CC(O)CC(C)(O)C1CCC2C3CC=C4CC(O)CCC4C3CCC21C]

or a pharmaceutically acceptable salt thereof.

In various aspects, the compound has a structure represented by a formula:

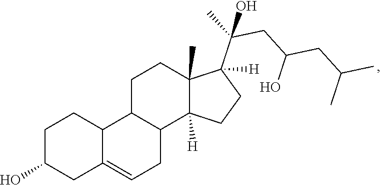 [SMILES: CC(C)CC(O)C[C@](C)(O)[C@H]1CC[C@H]2C3CC=C4C[C@H](O)CCC4C3CC[C@@]21C]

or a pharmaceutically acceptable salt thereof.

In various aspects, the compound has a structure represented by a formula:

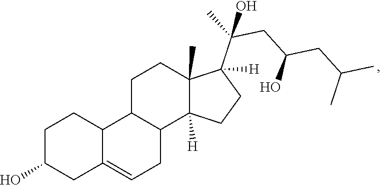 [SMILES: CC(C)C[C@@H](O)C[C@](C)(O)[C@H]1CC[C@H]2C3CC=C4C[C@H](O)CCC4C3CC[C@@]21C]

or a pharmaceutically acceptable salt thereof.

In various aspects, the compound has a structure represented by a formula:

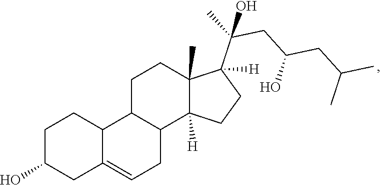 [SMILES: CC(C)C[C@H](O)C[C@](C)(O)[C@H]1CC[C@H]2C3CC=C4C[C@H](O)CCC4C3CC[C@@]21C]

or a pharmaceutically acceptable salt thereof.

### A. R⁶ Groups

In one aspect, R⁶ is a C1-C8 alkyl. In a further aspect, R⁶ is a C1-C4 alkyl. In a still further aspect, R⁶ is selected from methyl, ethyl, n-propyl, i-propyl, n-butyl, isobutyl, secbutyl, and tert-butyl. In a yet further aspect, R⁶ is selected from methyl, ethyl, n-propyl, and i-propyl. In an even further aspect, R⁶ is selected from methyl and ethyl. In a still further aspect, R⁶ is ethyl. In yet a further aspect, R⁶ is methyl.

In various aspects, R⁶ is isobutyl.

b. Prophetic Closed Form Hydroxy Derivatives of Vitamin D₃

The following compound examples are prophetic, and can be prepared using the synthesis methods described herein and other general methods as needed as would be known to one skilled in the art. Thus, in one aspect, a closed form hydroxy derivative of vitamin D₃ can be selected from:

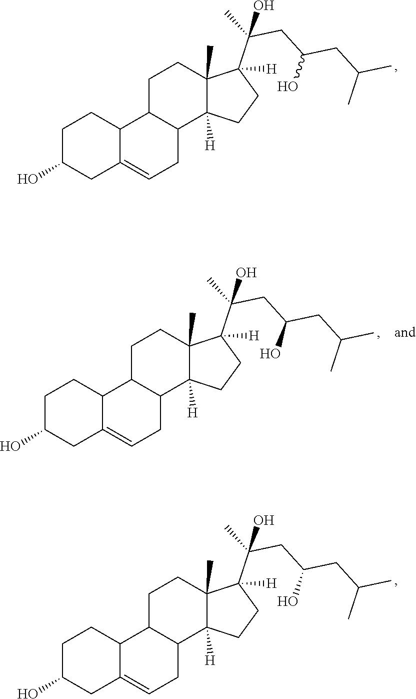 [SMILES: CC(C)CC(O)C[C@](C)(O)[C@H]1CC[C@H]2C3CC=C4C[C@H](O)CCC4C3CC[C@@]21C ; CC(C)C[C@@H](O)C[C@](C)(O)[C@H]1CC[C@H]2C3CC=C4C[C@H](O)CCC4C3CC[C@@]21C ; CC(C)C[C@H](O)C[C@](C)(O)[C@H]1CC[C@H]2C3CC=C4C[C@H](O)CCC4C3CC[C@@]21C]

or a pharmaceutically acceptable salt thereof.

### I. Methods of Making Hydroxy Derivatives of Vitamin D₃ (Closed Form-Route II)

In one aspect, disclosed are methods of making open chain hydroxy derivatives of vitamin D₃. See, e.g., FIG. 47. As detailed herein, hydroxy derivatives of vitamin D₃ have demonstrated utility as therapeutic agents for a variety of autoimmune disorders including, but not limited to, lupus erythematosus, rheumatoid arthritis, inflammatory bowel disease, psoriasis, and multiple sclerosis. Thus, in various aspects, hydroxy derivatives of vitamin D₃ can be prepared by adding an appropriate nucleophile to an appropriate ketone to provide the desired functionality (i.e., a nitrile or an ester derivative as detailed herein). The tertiary alcohol is then protected (i.e., to provide a protected tertiary alcohol as detailed herein) followed by selective reduction of a nitrile or an ester (i.e., to provide an aldehyde as described herein). Subsequently, the aldehyde can be reacted with an appropriate Grignard reagent (i.e., to provide a tertiary alcohol). At that time, the protecting groups can be removed, thereby providing an open chain hydroxy derivative of vitamin D₃.

### 1. Methods of Making Nitrile and Ester Derivatives

In one aspect, disclosed are methods of making a nitrile or an ester having a structure represented by a formula:

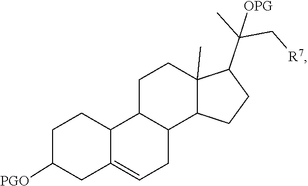

wherein R⁷ is selected from —CN and —CO₂R¹⁰; wherein R¹⁰ is C1-C4 alkyl; and wherein each occurrence of PG is independently a silyl protecting group, pharmaceutically acceptable salt thereof, the method reacting a compound having a structure represented by a formula:

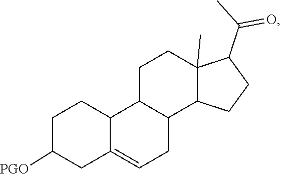

with a nucleophile having a structure represented by a formula:

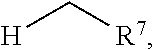

and then protecting the resultant tertiary alcohol.

In various aspects, reacting is in the presence of a base. In a further aspect, the base is selected from n-butyl lithium, 1,8-diazabicyclo[5.4.0]undec-7-ene (DBU), lithium diisopropylamide (LDA), sodium hydride, and potassium tert-butoxide. In a yet further aspect, the base is lithium diisopropylamide (LDA).

In various aspects, reacting is in an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, reacting is at a temperature of about −78° C.

In various aspects, the nitrile is selected from acetonitrile, propionitrile, and benzonitrile. In a yet further aspect, the nitrile is acetonitrile.

In various aspects, PG is selected from acetyl, benzoyl, benzyl, methoxyethoxymethyl ether (MEM), methoxymethyl ether (MOM), para-methoxybenzyl ether (PMB), para-methoxyphenyl ether (PMP), pivaloyl, tert-butyl ether, tetrahydropyranyl (THP), trityl, trimethylsilyl (TMS), triethylsilyl (TES), tert-butyldimethylsilyl (TBS), tert-butyldiphenylsilyl (TBDPS), and triisopropylsilyl (TIPS). In a still further aspect, the alcohol protecting group is a silyl protecting group. In a yet further aspect, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In an even further aspect, the silyl protecting group is triethylsilyl (TES).

In various aspects, R⁷ is —CN.

In various aspects, R⁷ is —CO₂R¹⁰. In a further aspect, R⁷ is —CO₂Me.

In various aspects, protecting is via addition of a silyl triflate. In a further aspect, the silyl triflate is trimethylsilyl triflate.

In various aspects, protecting is in the presence of a base. In a further aspect, the base is selected from pyridine, pyridinium chlorochromate, pyridinium dichromate, triethylamine, and N,N-diethylaniline. In a still further aspect, the base is pyridine.

In various aspects, protecting is at a temperature of about 0° C.

In various aspects, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a further aspect, the silyl protecting group is triethylsilyl (TES). In a still further aspect, the silyl protecting group is trimethylsilyl (TMS)

In various aspects, the compound has a structure represented by a formula:

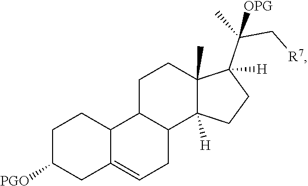

or a pharmaceutically acceptable salt thereof.

In various aspects, the compound has a structure represented by a formula:

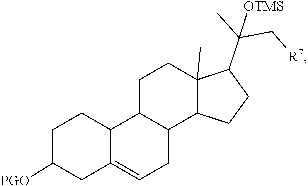

or a pharmaceutically acceptable salt thereof.

In various aspects, the compound has a structure represented by a formula:

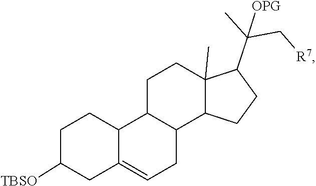

or a pharmaceutically acceptable salt thereof.

In various aspects, wherein the compound has a structure represented by a formula:

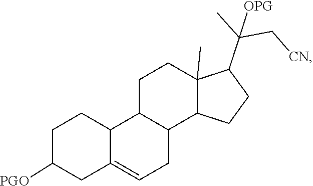

or a pharmaceutically acceptable salt thereof.

In various aspects, the compound has a structure represented by a formula:

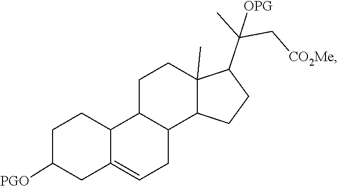

or a pharmaceutically acceptable salt thereof.

In various aspects, the compound has a structure:

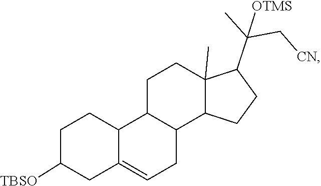

or a pharmaceutically acceptable salt thereof.

In various aspects, the compound has a structure:

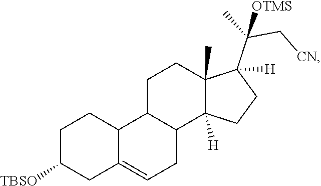

or a pharmaceutically acceptable salt thereof.

In various aspects, the compound has a structure:

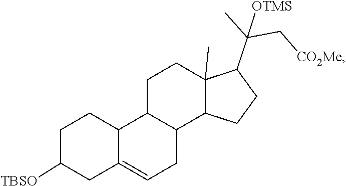

or a pharmaceutically acceptable salt thereof.

In various aspects, the compound has a structure:

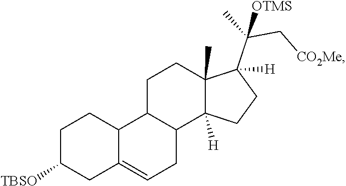

or a pharmaceutically acceptable salt thereof.

### A. Route I

In one aspect, substituted nitrile and ester analogs can be prepared as shown below.

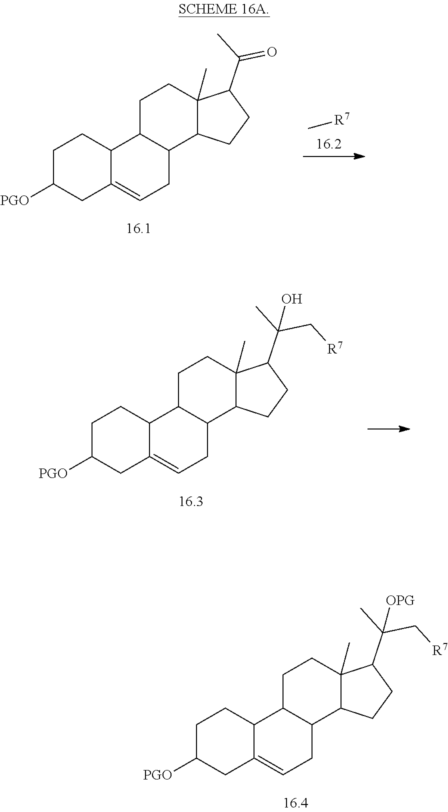

Compounds are represented in generic form, with substituents as noted in compound descriptions elsewhere herein. A more specific example is set forth below.

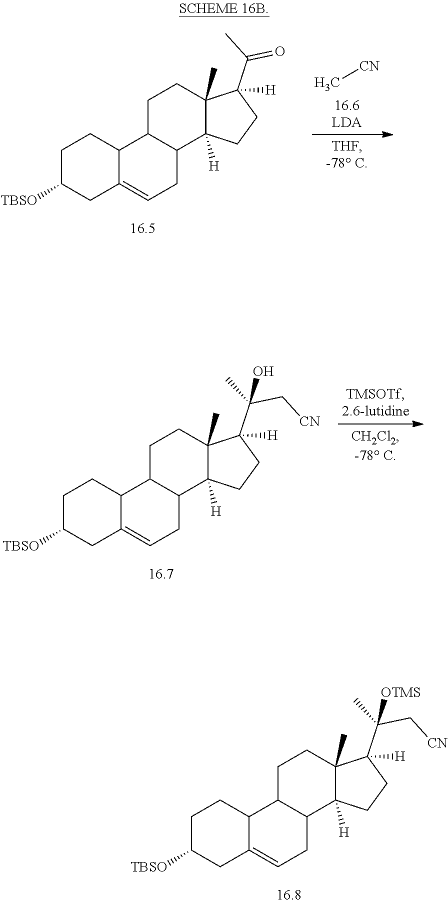

In one aspect, compounds of type 16.8, and similar compounds, can be prepared according to reaction Scheme 16B above. Thus, compounds of type 16.7 can be prepared by nucleophilic addition of an appropriate nitrile or ester, e.g., 16.6 as shown above, to an appropriate aldehyde, e.g., 16.5 as shown above. Appropriate nitriles, appropriate esters, and appropriate aldehydes are commercially available or prepared by methods known to one skilled in the art. The nucleophilic addition is carried out with an appropriate base, e.g., lithium diisopropylamine (LDA), in an appropriate solvent, e.g., tetrahydrofuran (THF), at an appropriate temperature, e.g., −78° C. Compounds of type 16.8 can be prepared by deprotection of an appropriate protected alcohol, e.g., 16.7 as shown above. The deprotection is carried out in the presence of an appropriate deprotecting agent, e.g., trimethylsilyl triflate, and an appropriate base, e.g., 2.6-lutidine, in an appropriate solvent, e.g., dichloromethane, at an appropriate temperature, e.g., −78° C. As can be appreciated by one skilled in the art, the above reaction provides an example of a generalized approach wherein compounds similar in structure to the specific reactants above (compounds similar to compounds of type 16.1, 16.2, and 16.3), can be substituted in the reaction to provide substituted nitrile and ester derivatives similar to Formula 16.4.

### 2. Methods of Making Aldehydes

In one aspect, disclosed are methods of making an aldehyde having a structure represented by a formula:

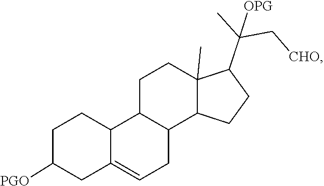

wherein each occurrence of PG is independently a silyl protecting group, or a pharmaceutically acceptable salt thereof, the method comprising reducing a compound having a structure represented by a formula:

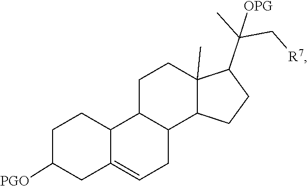

wherein R⁷ is selected from —CN and —CO₂R¹⁰; and wherein R¹⁰ is C1-C4 alkyl, or a pharmaceutically acceptable salt thereof.

In various aspects, reducing is in the presence of a reducing agent selected from sodium borohydride, lithium aluminum hydride (LAH), and diisobutylaluminum hydride (DIBAL-H). In a further aspect, reducing is in the presence of diisobutylaluminum hydride (DIBAL-H).

In various aspects, reducing is in the presence of an organic, nonpolar solvent.

In a further aspect, the organic, nonpolar solvent is selected from dichloromethane (DCM) and diethyl ether. In a still further aspect, the organic, nonpolar solvent is dichloromethane (DCM).

In various aspects, reducing is at a temperature of about −78° C.

In various aspects, the silyl protecting group is selected from trimethylsilyl (TMS), triethylsilyl (TES), tert-butyldimethylsilyl (TBS), tert-butyldiphenylsilyl (TBDPS), and triisopropylsilyl (TIPS). In a further aspect, the silyl protecting group is triethylsilyl (TES).

In various aspects, wherein R⁷ is —CN. In a further aspect, R⁷ is —CO₂R¹⁰. In a still further aspect, R⁷ is —CO₂Me.

In various aspects, the aldehyde has a structure represented by a formula:

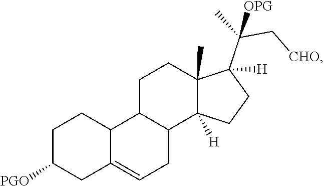

or a pharmaceutically acceptable salt thereof.

In various aspects, the aldehyde has a structure represented by a formula:

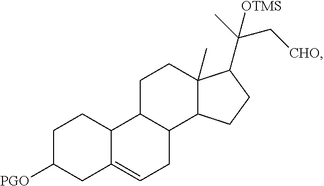

or a pharmaceutically acceptable salt thereof.

In various aspects, the aldehyde has a structure represented by a formula:

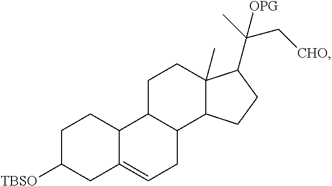

or a pharmaceutically acceptable salt thereof.

In various aspects, the aldehyde has a structure:

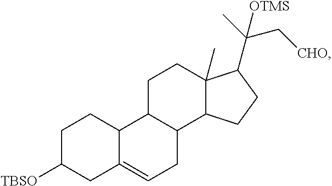

or a pharmaceutically acceptable salt thereof.

In various aspects, wherein the aldehyde has a structure:

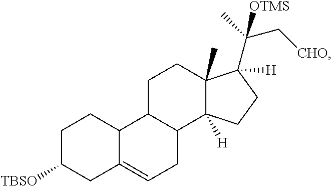

or a pharmaceutically acceptable salt thereof.
b. Route I

In one aspect, substituted aldehyde analogs can be prepared as shown below.

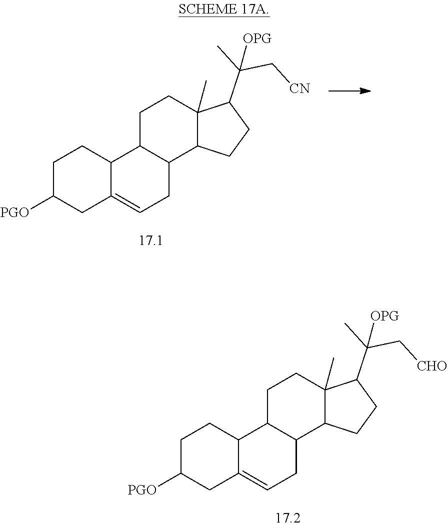

Compounds are represented in generic form, with substituents as noted in compound descriptions elsewhere herein. A more specific example is set forth below.

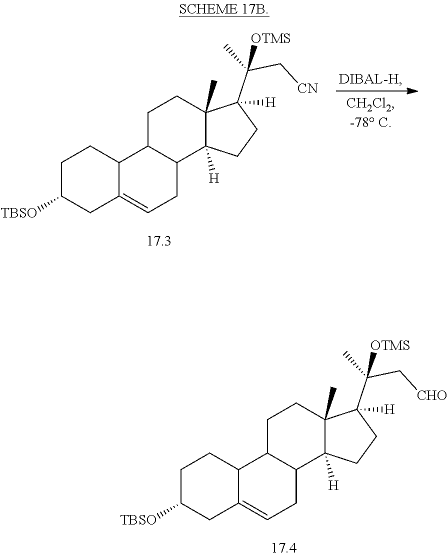

In one aspect, compounds of type 17.4, and similar compounds, can be prepared according to reaction Scheme 17B above. Thus, compounds of type 17.4 can be prepared by reduction of an appropriate nitrile or ester, e.g., 17.3 as shown above. Appropriate nitriles and appropriate esters are commercially available or prepared by methods known to one skilled in the art. The reduction is carried out with an appropriate reducing agent, e.g., diisobutylaluminum hydride, in an appropriate solvent, e.g., dichloromethane (DCM), at an appropriate temperature, e.g., −10° C., in the presence of an appropriate base, e.g., n-BuLi, for an appropriate amount of time, e.g., 1 h. As can be appreciated by one skilled in the art, the above reaction provides an example of a generalized approach wherein compounds similar in structure to the specific reactants above (compounds similar to compounds of type 17.1), can be substituted in the reaction to provide substituted aldehydes similar to Formula 17.2.

### 5. Methods of Making Protected Tertiary Alcohols

In one aspect, disclosed are methods of making a protected tertiary alcohol having a structure represented by a formula:

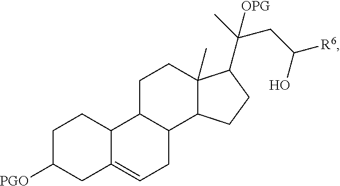

wherein R⁶ is a C1-C8 alkyl; and wherein each occurrence of PG is independently a silyl protecting group, or a pharmaceutically acceptable salt thereof, the method comprising reacting an aldehyde having a structure represented by a formula:

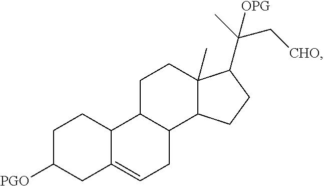

or a pharmaceutically acceptable salt thereof, and a Grignard reagent having a structure represented by a formula:

R⁶—Mg—X,

wherein X is a halide.

In various aspects, reacting is in the presence of an aprotic solvent. In a further aspect, the aprotic solvent is selected from diethyl ether (Et₂O), cyclopentyl methyl ether (CPME), and tetrahydrofuran (THF). In a still further aspect, the aprotic solvent is tetrahydrofuran (THF).

In various aspects, reacting is at a temperature of from about 0° C. to about 22° C.

In various aspects, R⁶ is a C1-C4 alkyl. In a further aspect, R⁶ is isobutyl.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a further aspect, each occurrence of PG is independently a silyl protecting group selected from trimethylsilyl (TMS) and tert-butyldimethylsilyl (TBS).

In various aspects, X is —Cl.

In various aspects, the aldehyde has a structure represented by a formula:

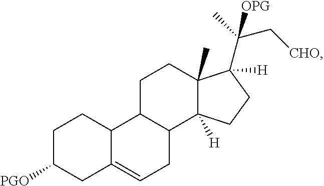

or a pharmaceutically acceptable salt thereof.

In various aspects, the aldehyde has a structure represented by a formula:

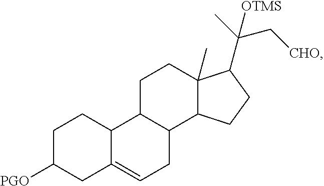

or a pharmaceutically acceptable salt thereof.

In various aspects, the aldehyde has a structure represented by a formula:

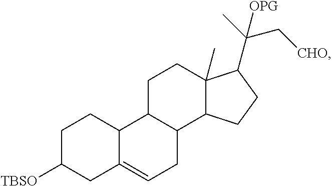

or a pharmaceutically acceptable salt thereof.

In various aspects, the aldehyde has a structure:

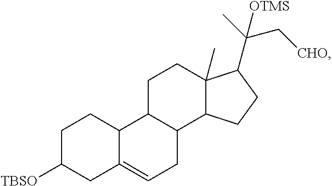

or a pharmaceutically acceptable salt thereof.

In various aspects, the aldehyde has a structure:

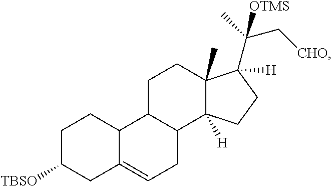

or a pharmaceutically acceptable salt thereof.

In various aspects, the Grignard reagent has a structure represented by a formula:

R⁶—MgCl,

In various aspects, the Grignard reagent has a structure:

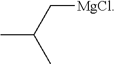

In various aspects, the compound has a structure represented by a formula:

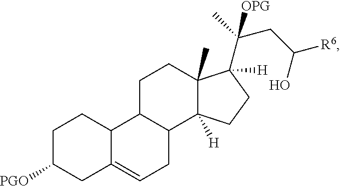

or a pharmaceutically acceptable salt thereof.

In various aspects, the compound has a structure represented by a formula:

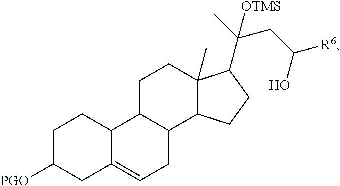

or a pharmaceutically acceptable salt thereof.

In various aspects, the compound has a structure represented by a formula:

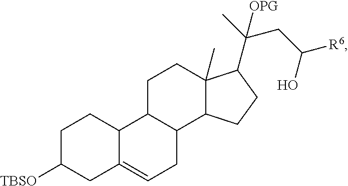

or a pharmaceutically acceptable salt thereof.

In various aspects, the compound has a structure represented by a formula:

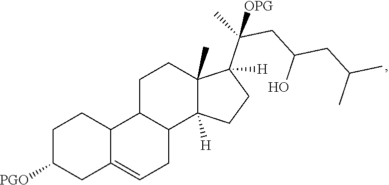

or a pharmaceutically acceptable salt thereof.

In various aspects, the compound has a structure represented by a formula:

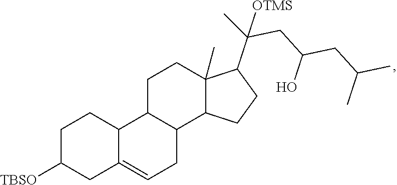

or a pharmaceutically acceptable salt thereof.

In various aspects, the compound has a structure represented by a formula:

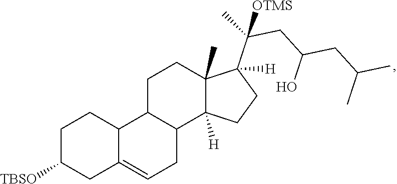

or a pharmaceutically acceptable salt thereof.

In various aspects, the compound has a structure represented by a formula:

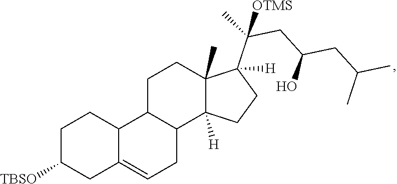

or a pharmaceutically acceptable salt thereof.

In various aspects, the compound has a structure represented by a formula:

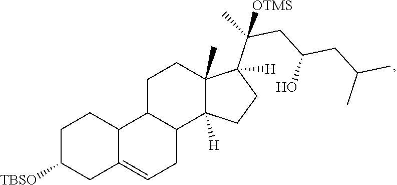

or a pharmaceutically acceptable salt thereof.

### A. Route I

In one aspect, substituted protected tertiary alcohols can be prepared as shown below.

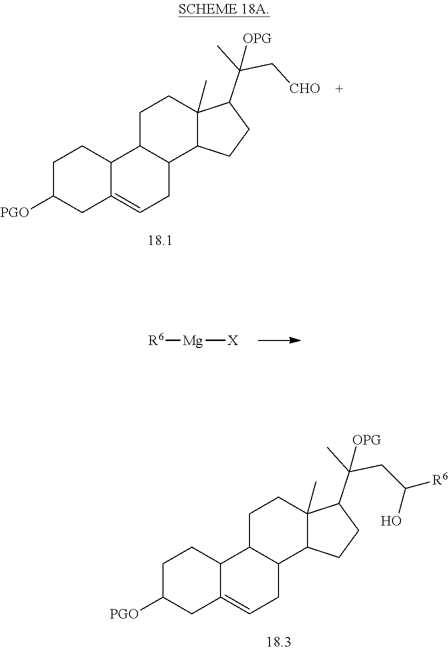

Compounds are represented in generic form, wherein X is a halide and with other substituents as noted in compound descriptions elsewhere herein. A more specific example is set forth below.

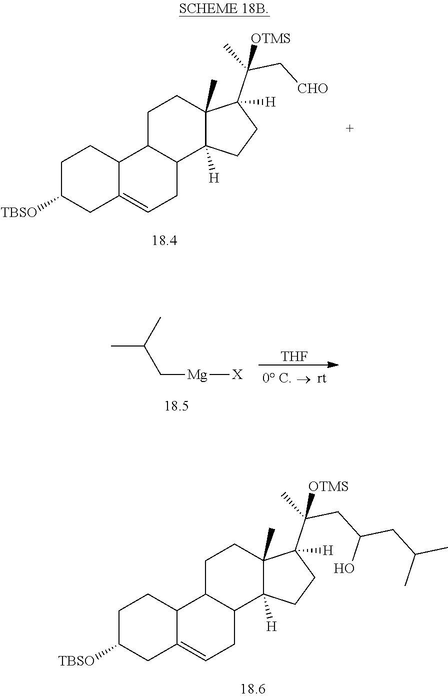

In one aspect, compounds of type 18.6, and similar compounds, can be prepared according to reaction Scheme 18B above. Thus, compounds of type 18.6 can be prepared using an appropriate ketone, e.g., 18.4 as shown above, and an appropriate Grignard reagent, e.g., 18.5 as shown above, where the Grignard reagent is commercially available or is prepared from an appropriate alkyl halide and magnesium metal. The Grignard reaction is carried out in the presence of an appropriate aprotic solvent, e.g., tetrahydrofuran (THF), at an appropriate temperature, e.g., 0° C. to room temperature. As can be appreciated by one skilled in the art, the above reaction provides an example of a generalized approach wherein compounds similar in structure to the specific reactants above (compounds similar to compounds of type 18.1 and 18.2), can be substituted in the reaction to provide substituted protected tertiary alcohol analogs similar to Formula 18.3.

### 6. Methods of Making Closed Form Hydroxy Derivatives of Vitamin D₃

In one aspect, disclosed are methods of making closed form hydroxy derivatives of vitamin D₃ having a structure represented by a formula:

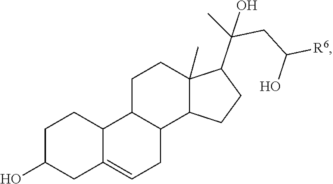

wherein R⁶ is a C1-C8 alkyl; and or a pharmaceutically acceptable salt thereof, the method comprising deprotecting an alcohol having a structure represented by a formula:

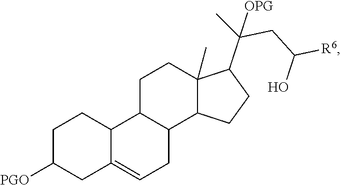

wherein each occurrence of PG is independently a silyl protecting group, or a pharmaceutically acceptable salt thereof.

In various aspects, deprotecting is in the presence of a deprotecting agent selected from potassium fluoride, cesium fluoride, tetrabutylammonium fluoride (TBAF), tris(dimethylamino)sulfur (trimethylsilyl)difluoride (TASF), tetrabutylammonium triphenyldifluorosilicate (TBAT), tetraphenylbismuth fluoride, cadmium fluoride, acidic aqueous tetrahydrofuran (THF), acidic methanol, and acetic acid. In a further aspect, deprotecting is in the presence of tetrabutylammonium fluoride (TBAF).

In various aspects, deprotecting is in an aprotic solvent.

In various aspects, the aprotic solvent is selected from diethyl ether (Et₂O), cyclopentyl methyl ether (CPME), and tetrahydrofuran (THF). In a further aspect, the aprotic solvent is tetrahydrofuran (THF).

In various aspects, wherein R⁶ is a C1-C4 alkyl. In a further aspect, R⁶ is isobutyl.

In various aspects, each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS). In a further aspect, each occurrence of PG is independently a silyl protecting group selected from trimethylsilyl (TMS), triethylsilyl (TES), and tert-butyldimethylsilyl (TBS).

In various aspects, the alcohol has a structure represented by a formula:

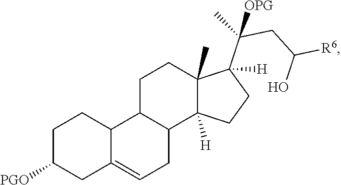

or a pharmaceutically acceptable salt thereof.

In various aspects, the alcohol has a structure represented by a formula:

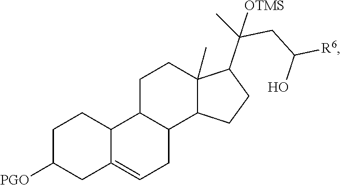

or a pharmaceutically acceptable salt thereof.

In various aspects, the alcohol has a structure represented by a formula:

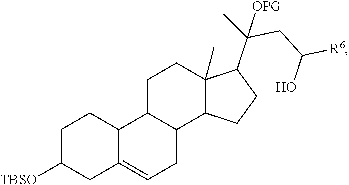

or a pharmaceutically acceptable salt thereof.

In various aspects, the alcohol has a structure represented by a formula:

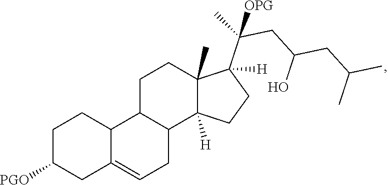

or a pharmaceutically acceptable salt thereof.

In various aspects, the alcohol has a structure represented by a formula:

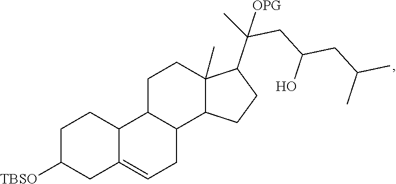

or a pharmaceutically acceptable salt thereof.

In various aspects, the alcohol has a structure represented by a formula:

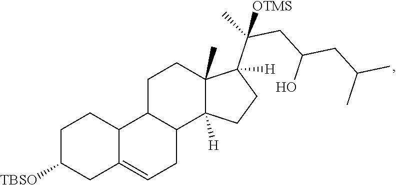

or a pharmaceutically acceptable salt thereof.

In various aspects, the alcohol has a structure represented by a formula:

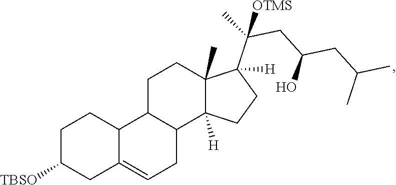

or a pharmaceutically acceptable salt thereof.

In various aspects, the alcohol has a structure represented by a formula:

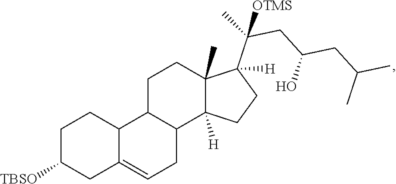

or a pharmaceutically acceptable salt thereof.

In various aspects, the compound has a structure represented by a formula:

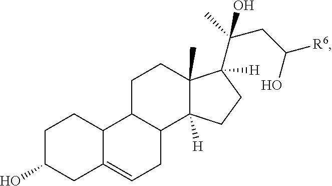

or a pharmaceutically acceptable salt thereof.

In various aspects, the compound has a structure represented by a formula:

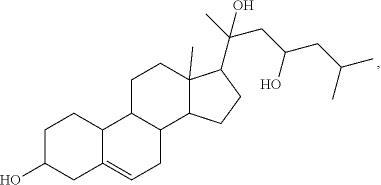 [SMILES: CC(C)CC(O)CC(C)(O)C1CCC2C3CC=C4CC(O)CCC4C3CCC21C]

or a pharmaceutically acceptable salt thereof.

In various aspects, the compound has a structure represented by a formula:

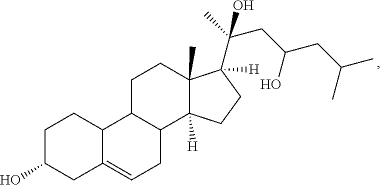 [SMILES: CC(C)CC(O)C[C@](C)(O)[C@H]1CC[C@H]2C3CC=C4C[C@H](O)CCC4C3CC[C@@]21C]

or a pharmaceutically acceptable salt thereof.

In various aspects, the compound has a structure represented by a formula:

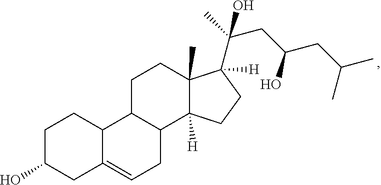 [SMILES: CC(C)C[C@@H](O)C[C@](C)(O)[C@H]1CC[C@H]2C3CC=C4C[C@H](O)CCC4C3CC[C@@]21C]

or a pharmaceutically acceptable salt thereof.

In various aspects, the compound has a structure represented by a formula:

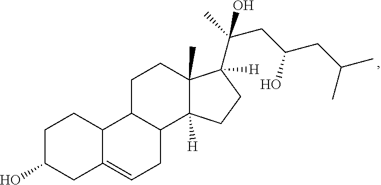 [SMILES: CC(C)C[C@H](O)C[C@](C)(O)[C@H]1CC[C@H]2C3CC=C4C[C@H](O)CCC4C3CC[C@@]21C]

or a pharmaceutically acceptable salt thereof.

### A. Route I

In one aspect, substituted alkyl alcohols can be prepared as shown below.

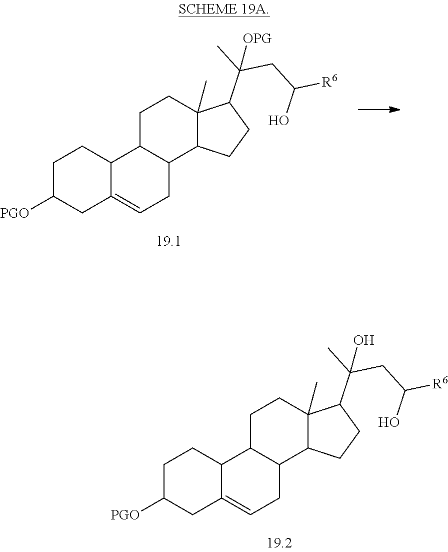

Compounds are represented in generic form, with substituents as noted in compound descriptions elsewhere herein. A more specific example is set forth below.

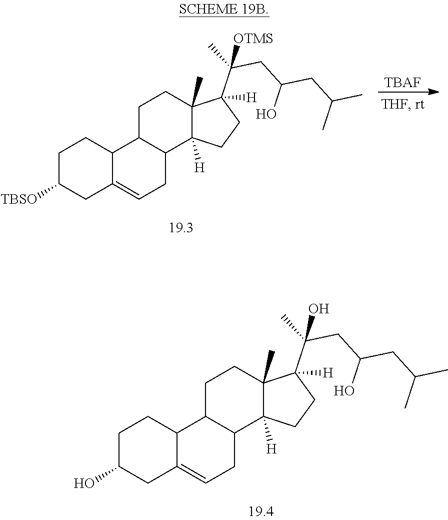

In one aspect, compounds of type 19.4, and similar compounds, can be prepared according to reaction Scheme 19B above. Thus, compounds of type 19.4 can be prepared by deprotection of an appropriate protected alcohol, e.g., 19.3, as shown above. The deprotection is carried out in an appropriate solvent, e.g., anhydrous THF, at an appropriate temperature, e.g., room temperature, in the presence of a deprotecting agent, e.g., tetra-n-butylammonium fluoride, for an appropriate amount of time, e.g., 18 h. As can be appreciated by one skilled in the art, the above reaction provides an example of a generalized approach wherein compounds similar in structure to the specific reactants above (compounds similar to compounds of type 19.1), can be substituted in the reaction to provide substituted closed form hydroxy derivatives of vitamin D₃ similar to Formula 19.2.

### J. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Experimental

### A. General Experimental Conditions

Optical rotations were measured in chloroform using a Perkin-Elmer model 343 polarimeter (Shelton, CT, USA). Ultraviolet (UV) absorption spectra were obtained on a Shimadzu UV-1800 spectrophotometer (Kyoto, Japan) in absolute ethanol. Nuclear magnetic resonance spectra were recorded in CDCl₃ solutions using Bruker AVANCE 300 MHz (Karlsruhe, Germany) and Bruker AVANCE 500 MHz instruments. Chemical shifts (δ) are reported in parts per million relative to (CH₃)₄Si (δ 0.00) or the solvent signal as an internal standard. Abbreviations used are singlet (s), doublet (d), triplet (t), quartet (q), multiplet (m), broad (br), narrow (narr). High-resolution mass spectra were recorded on Shimadzu LCMS-IT-TOF mass spectrometer using electrospray ionization (ESI) technique.

Reactions were carried out with magnetic stirring. All reactions involving moisture- or oxygen-sensitive compounds were carried out under dry argon atmosphere. Reaction temperatures refer to external bath temperatures. Tetrahydrofuran was distilled from Na/benzophenone; dichloromethane was distilled from P₂O₅. The organic extracts were dried over anhydrous MgSO₄, filtered and concentrated using a rotary evaporator with vacuum pump attached. Reactions were monitored by thin-layer chromatography (TLC) using aluminum-backed MERCK 60 silica gel plates (Darmstadt, Germany) (0.2 mm thickness). The chromatograms were visualized first with ultraviolet light (254 nm) and then by immersion in a cerium-molybdenum solution [10 g Ce(SO₄)₂×4 H₂O, 25 g phosphomolybdic acid, 60 mL H₂SO₄ and 940 mL H₂O] or p-anisaldehyde solution (5 mL H₂SO₄, 1.5 mL glacial AcOH, 3.7 mL p-anisaldehyde, 135 mL H₂O) followed by heating.

The purity of final compounds was determined by HPLC and they were judged at least 98% pure. For this purpose, straight-phase (SP) and reversed-phase (RP) high-performance liquid chromatography were performed on Shimadzu Prominence system equipped with SPD 20A UV detector (265 nm). For straight-phase a Zorbax RX-SIL semi-preparative 9.4×250 mm, 5 m column and for reverse-phase a Zorbax Eclipse XDB-C18 semi-preparative 9.4×250 mm, 5 m HPLC column were used. The purity and identity of the synthesized secosteroids were additionally confirmed by inspection of their ¹H NMR, ¹³C NMR and high-resolution mass spectra (HRMS).

### A. Synthesis of (5Z,22E)-(7R)-3B-[(Tert-Butyldimethylsilyl)Oxy]-9,10-Seco-8a-Ergosta-5,10(19),22-Triene-7,8-Diol (I).

A solution of potassium permanganate (4.80 g, 30.20 mmol) in water (140 mL) was added dropwise to a solution of vitamin D₂ (10.0 g, 25.20 mmol) in ethanol (1000 mL) at −20° C. The stirring was continued for 1.5 h at −20° C. and at room temperature overnight. Then a mixture was filtered through silica gel and concentrated. The residue was extracted with ethyl acetate (3×200 mL), the combined extracts were dried (MgSO₄) and concentrated. The crude triol was suspended in hexane and the precipitate was filtered off as a white solid (7.27 g, 67%). This product was dissolved in dry dimethylformamide (100 mL), the solution was cooled to 0° C. and dry imidazole (3.44 g, 50.52 mmol) was added under nitrogen. 4-Dimethylaminopyridine (0.411 g, 3.37 mmol) and tert-butyldimethylsilyl chloride (5.08 g, 33.68 mmol) were then added after 15 min and the mixture was stirred overnight at room temperature. The reaction was quenched by addition of brine and the mixture was extracted with diethyl ether (3×100 mL). The combined extracts were dried (MgSO₄) and concentrated. The residue was purified by column chromatography over silica using hexane/ethyl acetate (92:8) to afford monoprotected triol i as a white solid (8.44 g, 92%). m.p. 127-128° C.; [α]_{D}²⁴+75 (c 0.75, CHCl₃); ¹H NMR (300 MHz, CDCl₃) δ 0.056 and 0.061 (3H and 3H, each s, 2× SiCH₃), 0.81 (3H, s, 18-H₃), 0.82 and 0.83 (3H and 3H, each d, J=6.8 Hz, 26- and 27-H₃), 0.88 (9H, s, Si-t-Bu), 0.91 (3H, d, J=6.8 Hz, 24-CH₃), 0.98 (3H, d, J=6.6 Hz, 21-H₃), 2.20 (1H, ddd, J=13.1, 10.2, 1.7 Hz, 43-H), 2.40 (1H, dt, J=13.6, 4.2 Hz, 1β-H), 2.45 (1H, ddd, J=13.1, 4.2, 1.7 Hz, 4α-H), 3.71 (1H, tt, J=9.7, 4.2 Hz, 3α-H), 4.91 and 4.97 (1H and 1H, each s, 19-H₂), 4.92 (1H, d, J=9.8 Hz, 7-H), 5.14 (1H, dd, J=15.3, 8.3 Hz) and 5.20 (1H, dd, J=15.3, 7.8 Hz, 22- and 23-H), 5.52 (1H, dd, J=9.8, 1.2 Hz, 6-H); ¹³C NMR (75 MHz) δ −4.6, −4.5, 13.0, 17.7, 19.7, 20.0, 20.4, 20.7, 21.9, 25.9, 27.7, 33.1, 33.3, 36.6, 37.5, 40.0, 42.9, 44.0, 47.0, 57.4, 59.6, 70.8, 71.2, 74.9, 110.9, 124.6, 132.1, 135.5, 141.7, 145.8; HRMS (ESI) calcd. for C₃₄H₆₀O₃SiNa (M⁺+Na) 567.4209, measured 567.4214.

b. (2Z,5′S)-2-[5′-[(Tert-Butyldimethylsilyl)Oxy]-2′-Methylenecyclohexylidene]Ethanol (8) and (1R,3aR,4S,7aR)-7a-Methyl-1-[(1′R,2′E,4′R)-1′,4′,5′-Trimethyl-2′-Hexen-1′-yl]Octahydro-1H-Inden-4-ol (10).

Lead(IV) acetate (8.24 g, 18.56 mmol) was added in 4 portions to a solution of diol i (8.44 g, 15.48 mmol) in anhydrous CH₂Cl₂ (85 mL) and pyridine (3.7 mL) at 0° C. and the yellow suspension was vigorously stirred for 10 min. Then it was filtered off through Celite pad and concentrated. The residue was dissolved in a solution of cerium(III) chloride heptahydrate (11.53 g, 30.96 mmol) in methanol (110 mL) and the yellow suspension was treated with sodium borohydride (0.760 g, 20.12 mmol) at 0° C. After 15 min an additional portion of sodium borohydride was added (0.380 g, 10.06 mmol) and the stirring was continued for 2 h. Water (50 mL) and diethyl ether (100 mL) were the added and the layers were separated. Aqueous phase was extracted with diethyl ether (2×50 mL). The combined organic layers were washed with brine (20 mL), 5% solution of HCl (30 mL) and water (20 mL), dried (MgSO₄) and evaporated. The crude residue was purified by column chromatography. Elution with hexane/ethyl acetate (96:4) gave a mixture of Grundmann ketone and alcohol 10 (4.0 g). Further elution with hexane/ethyl acetate (92:8) gave a pure allylic alcohol 8 (2.37 g, 56%) as a colorless oil. The obtained mixture of Grundmann ketone and alcohol 10 (4.0 g) was dissolved in anhydrous THF (80 mL) and treated with a solution of lithium aluminum hydride (1 M in THF; 70 mL, 70 mmol) with stirring. After 1 h the reaction was quenched by slow addition of water and 5% solution of HCl. The residue was filtered through a Celite pad and the mixture was extracted with ethyl acetate (3×80 mL). The combined organic layers were washed with saturated solution of NaHCO₃ (30 mL), water (50 mL), dried (MgSO₄) and evaporated. The product was purified by column chromatography using hexane/ethyl acetate (96:4) to give pure alcohol 10 (3.54 g, 83%) as a colorless oil.

8: [α]_{D}²⁴+43 (c 0.95, CHCl₃); ¹H NMR (300 MHz, CDCl₃) δ 0.056 and 0.066 (3H and 3H, each s, 2× SiCH₃), 0.88 (9H, s, Si-t-Bu), 2.20 (1H, dd, J=12.2, 9.7 Hz, 6′β-H), 2.39 (1H, dt, J=13.6, 5.1 Hz, 3′β-H), 2.42 (1H, dd, J=12.9, 3.8 Hz, 6′α-H), 3.84 (1H, tt, J=8.7, 3.8 Hz, 5′α-H), 4.19 (1H, ddd, J=12.5, 6.2, 1.7 Hz, one of CH₂OH), 4.27 (1H, dd, J=12.5, 7.1 Hz, one of CH₂OH), 4.63 and 4.96 (1H and 1H, each s, C═CH₂), 5.44 (1H, dd, J=7.1, 6.2 Hz, 2-H); ¹³C NMR (75 MHz) δ −4.7, −4.6, 18.2, 25.9, 32.3, 36.0, 46.1, 59.9, 70.1, 111.9, 124.9, 140.6, 144.9; HRMS (ESI) calcd. for C₁₅H₃₉O₂SiNa (M⁺+Na) 291.1756, measured 291.1759.

10: [α]_{D}²⁴+11 (c 1.0, CHCl₃); ¹H NMR (300 MHz, CDCl₃) δ 0.82 and 0.83 (3H and 3H, each d, J=6.8 Hz, 5′-CH₃ and 6′-H₃), 0.91 (3H, d, J=6.8 Hz, 4′-CH₃), 0.94 (3H, s, 7a-CH₃), 0.98 (3H, d, J=6.6 Hz, 1′-CH₃), 4.07 (1H, narr m, 4α-H), 5.14 (1H, dd, J=15.6, 8.5 Hz) and 5.21 (1H, dd, J=15.6, 7.8 Hz, 2′- and 3′-H); ¹³C NMR (75 MHz) δ 13.7, 17.5, 17.6, 19.6, 19.9, 20.8, 22.5, 27.6, 33.1, 33.6, 39.8, 40.3, 41.8, 42.8, 52.7, 56.6, 69.4, 131.8, 135.7; HRMS (ESI) calcd. for C₁₉H₃₄ONa (M⁺+Na) 301.2507, measured 301.2499.

### c. (2Z,5′S)-2-{[5′-[(Tert-Butyldimethylsilyl)Oxy]-2′-Methylenecyclohexylidene]Ethyl}Diphenylphosphine Oxide (9)

n-BuLi (1.6 M solution in hexane; 255 μL, 0.409 mmol) was added to solution of allylic alcohol 8 (100 mg, 0.372 mmol) in anhydrous THF (1 mL) at 0° C. under nitrogen. A solution of dry tosyl chloride (78 mg, 0.409 mmol) in anhydrous THF (1 mL) was then added to the allylic alcohol-n-BuLi solution and the stirring was continued for 20 min at 0° C. In another dry flask, a solution of n-BuLi (465 μL, 0.774 mmol) was added to a solution of Ph₂PH (129 μL, 0.744 mmol) in anhydrous THF (2 mL) at 0° C. with stirring. The obtained deep red solution was siphoned under nitrogen pressure to the solution of tosylate until the deep-red color persisted (ca. ½ of the solution was added). The resulting mixture was stirred at 0° C. for additional 30 min and quenched by addition of H₂O (3 mL). The solvents were evaporated and the residue was dissolved in methylene chloride (2 mL) and stirred with 10% H₂O₂ (100 μL) at 0° C. for 1 h. The organic layer was separated, washed with cold aq. Na₂SO₃ and H₂O, dried (MgSO₄), and evaporated. The residue was purified by flash chromatography over silica, using hexane/ethyl acetate (8:2) to afford phosphine oxide 9 (127 mg, 75%) as a colorless powder. 9: m.p. 107-108° C.; [α]_{D}²⁴+43 (c 0.95, CHCl₃); ¹H NMR (300 MHz, CDCl₃) δ 0.021 and 0.026 (3H and 3H, each s, 2× SiCH₃), 0.86 (9H, s, Si-t-Bu), 1.51, 1.71, 2.13, 2.25 and 2.38 (each 1H, 5×m), 3.23 and 3.38 (1H and 1H, each m, 1-H₂), 3.57 (1H, tt, J=8.3, 3.6 Hz, 5′-H), 4.71 and 4.94 (1H and 1H, each s, C═CH₂), 5.37 (1H, m, 2-H), 7.73, 7.52 and 7.47 (4H, 2H and 4H, each m, Ar—H); ¹³C NMR (75 MHz) δ −4.7, −4.6, 14.2, 18.1, 21.0, 25.8, 25.9, 31.0, 31.3, 32.5, 36.3, 46.72, 46.73, 60.3, 70.45, 70.46, 111.5, 113.19, 113.23, 128.37, 128.42, 128.5, 128.6, 130.93, 130.98, 131.16, 131.20, 131.57, 131.58, 131.71, 131.72, 132.5, 132.93, 132.95, 133.4, 142.55, 142.61, 145.16, 145.17. HRMS (ESI) calcd. for C₂₇H₃₇O₂PSiNa (M⁺+Na) 475.2198, measured 475.2210.

### d. (1R,3aR,4S,7aR)-7a-Methyl-4-[(Triethylsilyl)Oxy]-1-[(1′R,2′E,4′R)-1′,4′,5′-Trimethyl-2′-Hexen-1′-yl]Octahydro-1H-Indene (II)

To a solution of the alcohol 10 (1.20 g, 4.30 mmol) in dry methylene chloride (20 mL) and 2,6-lutidine (0.748 mL, 6.46 mmol) was added triethylsilyl trifluoromethanesulfonate (1.2 mL, 5.16 mmol) at −78° C. and the mixture was stirred for 1 h. The reaction was quenched with saturated NaHCO₃ (10 mL) and extracted with methylene chloride (3×10 mL). The organic phase was dried (MgSO₄) and concentrated. The residue was purified by column chromatography on silica using hexane to give the protected compound ii (1.60 g, 95%) as a colorless oil. ii: [α]_{D}²⁴+8 (c 1.0, CHCl₃); ¹H NMR (300 MHz, CDCl₃) δ 0.56 (6H, q, J=7.9 Hz, 3× SiCH₂CH₃), 0.83 and 0.84 (3H and 3H, each d, J=6.8 Hz, 5′-CH₃ and 6′-H₃), 0.92 (3H, d, J=6.8 Hz, 4′-CH₃), 0.94 (3H, s, 7a-CH₃), 0.95 (9H, t, J=7.9 Hz, 3× SiCH₂CH₃), 0.99 (3H, d, J=6.6 Hz, 1′-CH₃), 4.03 (1H, narr m, 4α-H), 5.15 (1H, dd, J=15.3, 8.3 Hz) and 5.20 (1H, dd, J=15.3, 7.6 Hz, 2′- and 3′-H); ¹³C NMR (75 MHz) δ 4.9, 6.4, 6.8, 7.0, 13.8, 17.6, 17.8, 19.7, 20.0, 20.8, 23.1, 27.9, 33.2, 34.7, 39.9, 40.8, 42.1, 42.9, 53.3, 56.9, 69.5, 131.6, 135.9; HRMS (ESI) calcd. for C₂₅H₄₈OSiNa (M⁺+Na) 415.3372, measured 415.3380.

### e. (2S)-2-[(1′S,3A′R,4′S,7A′S)-7A′-Methyl-4′-[(Triethylsilyl)Oxy]Octahydro-1H-Inden-1′-yl)]Propanal (III)

To a solution of alkene ii (1.60 g, 4.07 mmol) in a mixture of acetone/water (6:1, 70 mL) was added 4-methylmorpholine N-oxide (NMO, 2.38 g, 20.35 mmol) followed by addition of the solution of osmium tetroxide in t-BuOH (2.5%, 2.6 mL). The mixture was heated at 60° C. for 24 h, then water and ethyl acetate were added and the layers were separated. Aqueous phase was extracted with ethyl acetate (2×30 mL). The combined organic phases were washed with brine (20 mL), dried (MgSO₄) and concentrated. The residue was purified by column chromatography on silica using hexane/ethyl acetate (9:1) to give the mixture of diastereomeric diols (1.21 g, 70%).

To a solution of these diols in THF (40 mL) was added saturated solution of NaIO₄ (1.82 g, 8.55 mmol) in water (10 mL) at 0° C. and the white suspension was stirred for 1 h. Then water was added and the aqueous phase was extracted with dichloromethane (3×50 mL). The combined organic solvents were washed with water (30 mL), dried (MgSO₄) and evaporated. The residue was purified by column chromatography on silica using hexane/ethyl acetate (99:1) to give an aldehyde iii (0.758 g, 82%) as a colorless oil. iii: [α]_{D}²⁴+42 (c 1.0, CHCl₃) 6 0.55 (6H, q, J=7.9 Hz, 3× SiCH₂CH₃), 0.94 (9H, t, J=7.9 Hz, 3×SiCH₂CH₃), 0.95 (3H, s, 7a′-CH₃), 1.08 (3H, d, J=6.9 Hz, 1-CH₃), 2.34 (1H, dqd, J=10.1, 6.8, 3.2 Hz, 2-H), 4.06 (1H, narr m, 4′α-H), 9.57 (1H, d, J=3.2 Hz, CHO); ¹³C NMR (75 MHz) δ 4.9, 6.9, 13.3, 13.9, 17.6, 19.7, 23.3, 26.2, 34.6, 40.5, 42.7, 49.2, 51.7, 52.4, 69.1, 205.3; HRMS (ESI) calcd. for C₁₉H₃₆O₂SiNa (M⁺+Na) 347.2382, measured 347.2386.

### f. [(1S,3aR,4S,7aS)-L-Acetyl-7a-Methyl-4-[(Triethylsilyl)Oxy]Octahydro-1H-Indene (11)

To a solution of aldehyde iii (1.30 g, 4.00 mmol) in toluene (15 mL) was added morpholine (0.488 mL, 5.20 mmol) and p-toluenesulfonic acid (38 mg, 0.20 mmol). The mixture was heated in the Dean-Stark apparatus at 80° C. overnight. The solvent was evaporated and the crude enamine was dissolved in acetone (30 mL). Alumina (0.489 g, 4.79 mmol) supported potassium permanganate (0.633 g, 4.00 mmol) was added in two portions. After 2 h additional portion of alumina (0.244 g, 2.40 mmol) supported potassium permanganate (0.316 g, 2.00 mmol) was added and the stirring was continued for 2 h. The reaction mixture was filtered through silica gel pad and the solvent was evaporated. The residue was purified by column chromatography on silica using hexane/ethyl acetate (99:1) to give ketone 11 (0.956 g, 77%) as a colorless oil. 11: [α]_{D}²⁴+98 (c 1.2, CHCl₃); ¹H NMR (300 MHz, CDCl₃) δ 0.55 (6H, q, J=7.8 Hz, 3× SiCH₂CH₃), 0.84 (3H, s, 7a-CH₃), 0.93 (9H, t, J=7.8 Hz, 3× SiCH₂CH₃), 2.08 (3H, s, CH₃CO), 2.46 (1H, t, J=8.6 Hz, 1α-H), 4.07 (1H, narr m, 4α-H); ¹³C NMR (75 MHz) δ 4.8, 6.9, 15.3, 17.7, 21.8, 23.2, 31.6, 34.4, 39.9, 43.8, 53.3, 64.5, 68.9, 209.6; HRMS (ESI) calcd. for C₁₈H₃₄O₂SiNa (M⁺+Na) 333.2226, measured 333.2230.

### g. [(1S,3aR,4S,7aS)-L-Acetyl-7a-Methyloctahydro-1H-Inden-4-ol (IV)

To a solution of the ketone 11 (0.376 g, 1.20 mmol) in THF (20 mL) was added tetra-n-butylammonium fluoride (1M in THF; 3.6 mL, 3.6 mmol) at room temperature and the mixture was stirred overnight. The reaction was quenched with brine (10 mL) and extracted with AcOEt (3×10 mL). The organic phases were washed with H₂O (10 mL), dried (MgSO₄) and concentrated. The residue was purified by column chromatography on silica using hexane/ethyl acetate (8:2) to give the compound iv (0.230 g, 97%) as a colorless oil. iv: [α]_{D}²⁴+136 (c 1.1, CHCl₃); ¹H NMR (300 MHz, CDCl₃) δ 0.87 (3H, s, 7a-CH₃), 2.10 (3H, s, CH₃CO), 2.49 (1H, t, J=9.1 Hz, 1α-H), 4.12 (1H, narr m, 4α-H); ¹³C NMR (75 MHz) δ 15.3, 17.4, 21.7, 22.7, 31.3, 33.5, 39.6, 43.4, 52.8, 64.3, 68.8, 209.3; HRMS (ESI) calcd. for C₁₂H₂₀O₂Na (M⁺+Na) 219.1361, measured 219.1370.

### h. (1S,4S,7aS)-1-Acetyl-7a-Methyloctahydro-1H-Inden-4-yl Acetate (12)

To a solution of ketone iv (0.220 g, 1.12 mmol) in dry methylene chloride (10 mL) was added triethylamine (0.47 mL, 3.36 mmol), 4-dimethylaminopyridine (DMAP; 0.034 g, 0.280 mmol) and acetic anhydride (0.16 mL, 1.68 mmol) at 0° C. The mixture was stirred at room temperature for 2 h, poured into water (5 mL) and extracted with methylene chloride (3×5 mL). The combined organic phases were washed with 5% solution of HCl (5 mL), saturated solution of NaHCO₃ (5 mL), water (5 mL), dried (MgSO₄) and concentrated. The residue was purified by column chromatography on silica using hexane/ethyl acetate (9:1) to give the compound 12 (0.261 g, 98%) as a colorless oil. 12: [α]_{D}²⁴+96 (c 1.2, CHCl₃); ¹H NMR (300 MHz, CDCl₃) δ 0.83 (3H, s, 7a-CH₃), 2.04 (3H, s, OCOCH₃), 2.12 (3H, s, CH₃CO), 2.49 (1H, t, J=9.2 Hz, 1α-H), 5.18 (1H, narr m, 4α-H); ¹³C NMR (75 MHz) δ 14.8, 17.9, 21.3, 21.7, 22.8, 30.3, 31.5, 39.1, 43.0, 51.5, 63.9, 70.6, 170.7, 209.0; HRMS (ESI) calcd. for C₁₄H₂₂O₃Na (M⁺+Na) 261.1467, measured 261.1472.

i. Synthesis of (1S,3aR,4S,7aS)-1-[(S)-2′-Hydroxy-6′-Methylheptan-2′-yl]-7a-Methyloctahydro-1H-Inden-4-ol (13)

To a suspension of activated magnesium turnings (105 mg, 4.40 mmol) in anhydrous THF (7 mL) was added a small crystal of iodine and 1-bromo-4-methylpentane (0.22 mL, 1.48 mmol). The mixture was heated to the boiling point and second portion of 1-bromo-4-methyl-pentane (0.22 mL, 1.48 mmol) was added. Stirring was continued for 1 h and then the mixture was cooled to 0° C. The generated Grignard reagent was then added to a solution of ketone 12 (70 mg, 0.295 mmol) in anhydrous THF (3 mL) at 0° C. and the stirring was continued for 2 h at this temperature and overnight at room temperature. The mixture was diluted with diethyl ether (10 mL) and saturated solution of NH₄Cl (10 mL) was carefully added at 0° C. The layers were separated and the aqueous was extracted with AcOEt (2×10 mL). The combined organic solvents were dried (MgSO₄) and concentrated. The residue was purified by column chromatography on silica using hexane/ethyl acetate (84:16) to give the compound 13 (70 mg, 85%) as a colorless oil. [α]24D+21 (c 0.6, CHCl₃); ¹H NMR (300 MHz, CDCl₃) δ 0.88 (6H, d, J=6.6 Hz, 6′-CH₃ and 7′-H₃), 1.15 (3H, s, 7a-CH₃), 1.27 (3H, s, 1′-H₃), 4.10 (1H, narr m, 4α-H); ¹³C NMR (75 MHz) δ 15.4, 17.4, 21.3, 21.9, 22.2, 22.5, 22.7, 26.3, 27.9, 33.4, 39.6, 40.8, 42.5, 44.1, 52.6, 57.9, 69.4, 75.1; HRMS (ESI) calcd. for C₁₈H₃₄O₂Na (M⁺+Na) 305.2457, measured 305.2462.

j. Synthesis of (1S,3aR,4S,7aS)-1-[(S)-2′-Hydroxy-6′-Methyl-6′-[(Triethylsilyl)Oxy]Heptan-2′-yl]-7a-Methyloctahydro-11H-Inden-4-ol (14)

The reaction of the ketone 12 (90 mg, 0.377 mmol) with the Grignard reagent, generated from 1-bromo-4-methyl-4-[(triethylsilyl)oxy]pentane, was performed as described above for 13. After the analogous procedure, the crude product was purified by column chromatography on silica using hexane/ethyl acetate (86:14) to give compound 14 (124 mg, 85%) as a colorless oil. [α]24D+16 (c 0.95, CHCl₃); ¹H NMR (300 MHz, CDCl₃) δ 0.56 (6H, q, J=7.5 Hz, 3× SiCH₂CH₃), 0.94 (9H, t, J=7.5 Hz, 3× SiCH₂CH₃), 1.14 (3H, s, 7a-CH₃), 1.19 (6H, s, 6′-CH₃ and 7′-H₃), 1.27 (3H, s, 1′-H₃), 4.09 (1H, narr m, 4α-H); ¹³C NMR (75 MHz) δ 6.8, 7.1, 15.4, 17.4, 19.0, 21.4, 22.2, 26.3, 29.8, 30.1, 33.4, 40.8, 42.6, 44.4, 45.6, 52.6, 57.9, 69.5, 73.4, 75.2; HRMS (ESI) calcd. for C₂₄H₄₈O₃SiNa (M⁺+Na) 435.3270, measured 435.3279.

k. Synthesis of (1S,3aR,7aR)-1-[(S)-2′-Hydroxy-6′-Methylheptan-2′-yl]-7a-Methyloctahydro-4H-Inden-4-One (15)

Molecular sieves 4 Å (70 mg) were added to a solution of the diol 13 (70 mg, 0.247 mmol) in anhydrous CH₂Cl₂ (2 mL) and the mixture was cooled to 0° C. 4-Methylmorpholine N-oxide (NMO, 44 mg, 0.371 mmol) and tetrapropylammonium perruthenate (TPAP, 44 mg, 0.124 mmol) were then added. The resulted dark mixture was stirred for 1.5 h and then filtered through Celite. The solvent was evaporated and the residue was purified by column chromatography on silica using hexane/ethyl acetate (86:14) to give the desired ketone (53 mg, 77%) as a colorless oil. [α]24D-21 (c 1.5, CHCl₃); ¹H NMR (300 MHz, CDCl₃) δ 0.83 (3H, s, 7a-CH₃), 0.89 (6H, d, J=6.6 Hz, 6′-CH₃ and 7′-H₃), 1.31 (3H, s, 1′-H₃), 2.45 (1H, dd, J=10.7, 7.0 Hz, 3α-H); ¹³C NMR (75 MHz) δ 14.4, 18.8, 21.8, 22.0, 22.6, 22.7, 23.9, 25.6, 27.9, 39.3, 39.5, 40.8, 44.1, 49.8, 58.2, 62.2, 74.6, 211.9; HRMS (ESI) calcd. for C₁₈H₃₂O₂Na (M⁺+Na) 303.2300, measured 303.2295.

### 1. Synthesis of (1S,3aR,7aR)-1-[(S)-2′-Hydroxy-6′-Methyl-6′-[(Triethylsilyl)Oxy]Heptan-2′-yl]-7a-Methyl Octahydro-4H-Inden-4-One (16)

The oxidation of the secondary hydroxyl group in 14 (145 mg, 0.353 mmol) was performed analogously as described above for 15. The crude product was purified by column chromatography over silica using hexane/ethyl acetate (88:12) to give the desired ketone (115 mg, 80%) as a colorless oil. [α]24D-13 (c 1.0, CHCl₃); ¹H NMR (300 MHz, CDCl₃) δ 0.56 (6H, q, J=7.5 Hz, 3× SiCH₂CH₃), 0.81 (3H, s, 7a-CH₃), 0.94 (9H, t, J=7.5 Hz, 3× SiCH₂CH₃), 1.20 (6H, s, 6′-CH₃ and 7′-H₃), 1.30 (3H, s, 1′-H₃), 2.42 (1H, dd, J=10.5, 6.9 Hz, 3α-H); ¹³C NMR (75 MHz) δ 6.8, 7.1, 14.4, 18.8, 19.0, 21.8, 24.0, 26.6, 29.8, 30.1, 39.3, 40.8, 44.4, 45.5, 49.8, 58.1, 62.2, 73.3, 74.7, 211.9; HRMS (ESI) calcd. for C₂₄H₄₆O₃SiNa (M⁺+Na) 433.3114, measured 433.3122.

m. Synthesis of (1S,3aR,7aR)-1-[(S)-2′-Hydroxy-6′-Methylheptan-2′-yl]-7a-Methyloctahydro-4H-Inden-4-One (17)

To a solution of hydroxy ketone 15 (76 mg, 0.271 mmol) in anhydrous CH₂Cl₂ (2 mL) was added 2,6-lutidine (63 μL, 0.542 mmol) and trimethylsilyl trifluoromethanesulfonate (74 μL, 0.406 mmol) at −78° C. and the mixture was stirred at this temperature for 1 h. Saturated NaHCO₃ (2 mL) was added and the mixture was extracted with dichloromethane (3×5 mL). The combined organic layers were washed with 5% HCl (5 mL), water (5 mL), dried (MgSO₄) and evaporated. The residue was purified by column chromatography on silica using hexane/ethyl acetate (99:1) to give desired protected ketone (80 mg, 84%) as a colorless oil. [α]24D-12 (c 1.4, CHCl₃); ¹H NMR (300 MHz, CDCl₃) δ 0.076 [9H, s, Si(CH₃)₃], 0.75 (3H, s, 7a-CH₃), 0.87 (6H, d, J=6.6 Hz, 6′-CH₃ and 7′-H₃), 1.32 (3H, s, 1′-H₃), 2.39 (1H, dd, J=10.8, 6.9 Hz, 3α-H); ¹³C NMR (75 MHz) δ 2.8, 14.4, 18.8, 21.8, 22.6, 22.7, 23.2, 24.0, 27.8, 27.9, 39.6, 39.8, 40.9, 45.0, 49.9, 57.4, 62.3, 78.0, 212.3; HRMS (ESI) calcd. for C₂₁H₄₀O₂SiNa (M⁺+Na) 375.2695, measured 375.2702.

n. Synthesis of (1S,3aR,7aR)-7a-Methyl-1-[(S)-6′-Methyl-6′-[(Triethylsilyl)Oxy]-2′-[(Trimethylsilyl)Oxy]Heptan-2′-yl]Octahydro-4H-Inden-4-One (18)

Protection of tertiary hydroxyl group in 16 (115 mg, 0.281 mmol) was performed analogously as described above for 17. The crude product was purified by column chromatography over silica using hexane/ethyl acetate (99:1) to give desired protected ketone (111 mg, 82%) as a colorless oil. [α]24D-9 (c 1.0, CHCl₃); ¹H NMR (300 MHz, CDCl₃) δ 0.078 [9H, s, Si(CH₃)₃], 0.57 (6H, q, J=7.5 Hz, 3× SiCH₂CH₃), 0.75 (3H, s, 7a-CH₃), 0.95 (9H, t, J=7.5 Hz, 3× SiCH₂CH₃), 1.20 (6H, s, 6′-CH₃ and 7′-H₃), 1.32 (3H, s, 1′-H₃), 2.38 (1H, dd, J=10.8, 6.8 Hz, 3α-H); ¹³C NMR (75 MHz) δ 2.8, 6.8, 7.1, 14.4, 18.8, 20.1, 21.9, 24.0, 27.8, 29.9, 30.1, 39.6, 40.9, 45.2, 45.8, 49.9, 57.5, 62.4, 73.2, 78.1, 212.4; HRMS (ESI) calcd. for C₂₇H₅₄O₃Si₂Na (M⁺+Na) 505.3509, measured 505.3515.

o. Synthesis of (20S)-3-[(Tert-Butyldimethylsilyl)Oxy]-20-[(Trimethylsilyl)Oxy]Vitamin D₃ (19).

To a solution of phosphine oxide 9 (395 mg, 0.860 mmol) in anhydrous THF (5 mL) was added n-BuLi (540 μL, 0.870 mmol) at −78° C. and the stirring of a deep red solution was continued for 30 min. A solution of the ketone 17 (154 mg, 0.430 mmol) in anhydrous THF (2 mL) was then added and the stirring was continued for 3 h. Saturated NH₄Cl (3 mL) was added and the mixture was extracted with diethyl ether (3×5 mL). The combined organic layers were washed with water (5 mL), dried (MgSO₄) and evaporated. The crude product was purified by column chromatography using hexane to give protected vitamin D compound 19 (204 mg, 81%) as a colorless oil. 19: [α]24D+61 (c 1.5, CHCl₃); ¹H NMR (300 MHz, CDCl₃) δ 0.070 [9H, s, Si(CH₃)₃], 0.67 (3H, s, 18-H₃), 0.88 (6H, d, J=6.6 Hz, 26- and 27-H₃), 0.90 (9H, s, Si-t-Bu), 1.29 (3H, s, 21-H₃), 2.12 (1H, m, 1α-H), 2.26 (1H, dd, J=13.2, 9.6 Hz, 4β-H), 2.36 (1H, dt, J=13.5, 4.6 Hz, 1β-H), 2.46 (1H, dd, J=13.2, 4.5 Hz, 4α-H), 2.82 (1H, br d, J˜13 Hz, 9β-H), 3.82 (1H, tt, J=9.6, 4.5 Hz, 3α-H), 4.80 and 5.01 (1H and 1H, each s, 19-H₂), 6.01 and 6.17 (1H and 1H, each d, J=11.2 Hz, 7- and 6-H); ¹³C NMR (75 MHz) δ −4.6, −4.5, 2.8, 13.9, 18.2, 21.9, 22.0, 22.6, 22.8, 23.3, 23.4, 25.9, 27.7, 27.9, 28.8, 32.8, 36.5, 39.8, 41.2, 45.2, 45.9, 46.9, 56.8, 57.5, 70.7, 78.5, 112.2, 118.1, 121.4, 136.2, 141.5, 145.4; HRMS (ESI) calcd. for C₃₆H₆₆O₂Si₂Na (M⁺+Na) 609.4499, measured 609.4508.

p. Synthesis of (20S)-3-[(Tert-Butyldimethylsilyl)Oxy]-25-[(Triethylsilyl)Oxy]-20-[(Trimethylsilil)Oxy]-Vitamin D₃ (20).

The Wittig-Horner reaction between ketone 18 (44 mg, 0.092 mmol) and phosphine oxide 9 (83 mg, 0.184 mmol) was performed analogously as described above for 17. The crude product was purified by column chromatography over silica using hexane/ethyl acetate (99:1) to give protected vitamin D compound 20 (60 mg, 91%) as a colorless oil. 20: [α]24D+54 (c 1.0, CHCl₃); ¹H NMR (300 MHz, CDCl₃) δ 0.069 [9H, s, Si(CH₃)₃], 0.57 (6H, q, J=7.5 Hz, 3× SiCH₂CH₃), 0.66 (3H, s, 18-H₃), 0.90 (9H, s, Si-t-Bu), 0.95 (9H, t, J=7.5 Hz, 3× SiCH₂CH₃), 1.20 (6H, s, 26- and 27-H₃), 1.30 (3H, s, 21-H₃), 2.12 (1H, m, 1α-H), 2.23 (1H, dd, J=13.2, 9.3 Hz, 4β-H), 2.36 (1H, dt, J=13.7, 4.8 Hz, 1β-H), 2.46 (1H, dd, J=13.2, 4.2 Hz, 4α-H), 2.82 (1H, br d, J˜12 Hz, 9β-H), 3.81 (1H, tt, J=9.3, 4.2 Hz, 3α-H), 4.80 and 5.00 (1H and 1H, each s, 19-H₂), 6.01 and 6.17 (1H and 1H, each d, J=11.2 Hz, 7- and 6-H); ¹³C NMR (75 MHz) δ −4.6, −4.5, 2.8, 6.8, 7.2, 13.9, 18.2, 20.2, 21.9, 22.0, 23.4, 25.9, 26.9, 27.7, 28.9, 29.8, 30.1, 32.8, 36.4, 41.1, 45.3, 45.8, 46.9, 56.8, 57.5, 70.7, 73.3, 78.6, 112.1, 118.1, 121.4, 136.2, 141.6, 145.4; HRMS (ESI) calcd. for C₄₂H₈₀O₃Si₃Na (M⁺+Na) 739.5313, measured 739.5322.

q. Synthesis of (20S)-20-Hydroxyvitamin D₃ (3).

Tetra-n-butylammonium fluoride (1M in THF; 4.3 mL, 4.3 mmol) was added to a solution of protected vitamin D compound 23 (84 mg, 0.143 mmol) in anhydrous THF (5 mL) and the mixture was stirred at room temperature overnight. Brine (3 mL) was added and the mixture was extracted with AcOEt (3×10 mL). The combined organic layers were washed with water (5 mL), dried (MgSO₄) and evaporated. The crude product was purified by HPLC (9.4 mm×25 cm Zorbax-Sil column, 4 mL/min) using hexane/2-propanol (95:5) solvent system; vitamin 3 (48 mg, 84%) was collected at R_{V}=23 mL. Analytical sample of the vitamin was obtained after reversed-phase HPLC (9.4 mm×25 cm Zorbax Eclipse XDB-C18 column, 4 mL/min) using methanol/water (9:1) solvent system (R_{V}=44 mL). [α]24D+21 (c 1.1, CHCl₃); ¹H NMR (300 MHz, CDCl₃) δ 0.72 (3H, s, 18-H₃), 0.87 (6H, d, J=6.6 Hz, 26- and 27-H₃), 1.27 (3H, s, 21-H₃), 2.17 (1H, ddd, J=13.5, 8.5, 4.8 Hz, 1α-H), 2.28 (1H, dd, J=13.2, 7.2 Hz, 4β-H), 2.39 (1H, ddd, J=13.5, 7.6, 4.8 Hz, 1β-H), 2.57 (1H, dd, J=13.2, 3.6 Hz, 4α-H), 2.82 (1H, br d, J˜13 Hz, 9β-H), 3.94 (1H, tt, J=7.3, 3.6 Hz, 3α-H), 4.82 and 5.04 (1H and 1H, each s, 19-H₂), 6.03 and 6.23 (1H and 1H, each d, J=11.2 Hz, 7- and 6-H); ¹³C NMR (75 MHz) δ 13.7, 21.9, 22.0, 22.1, 22.6, 22.7, 23.4, 26.6, 27.9, 28.9, 31.9, 35.2, 39.6, 40.8, 43.9, 45.9, 46.0, 56.5, 58.2, 69.2, 75.2, 112.5, 118.2, 122.3, 135.4, 141.5, 145.0; HRMS (ESI) calcd. for C₂₇H₄₅O₂(M⁺+Na) 401.3420, measured 401.3428.

r. Synthesis of (20S)-20,25-Dihydroxyvitamin D₃ (6).

The hydroxyl deprotection in the vitamin D compound 24 (60 mg, 0.083 mmol) was performed analogously as described above for 23. The crude product was purified by HPLC (9.4 mm×25 cm Zorbax-Sil column, 4 mL/min) using hexane/2-propanol (9:1) solvent system; vitamin 6 (26 mg, 75%) was collected at R_{V}=29 mL. Analytical sample of the vitamin was obtained after reversed-phase HPLC (9.4 mm×25 cm Zorbax Eclipse XDB-C18 column, 4 mL/min) using methanol/water (8:2) solvent system (R_{V}=37 mL). 6: [α]24D+20 (c 1.3, CHCl₃); ¹H NMR (300 MHz, CDCl₃) δ 0.72 (3H, s, 18-H₃), 1.22 (6H, s, 26- and 27-H₃), 1.28 (3H, s, 21-H₃), 2.17 (1H, ddd, J=13.4, 8.5, 4.6 Hz, 1α-H), 2.28 (1H, dd, J=13.2, 7.3 Hz, 4β-H), 2.40 (1H, ddd, J=13.4, 7.5, 4.6 Hz, 1β-H), 2.57 (1H, dd, J=13.2, 3.5 Hz, 4α-H), 2.81 (1H, br d, J˜13 Hz, 9β-H), 3.94 (1H, tt, J=7.3, 3.5 Hz, 3α-H), 4.81 and 5.05 (1H and 1H, each s, 19-H₂), 6.03 and 6.22 (1H and 1H, each d, J=11.2 Hz, 7- and 6-H); ¹³C NMR (75 MHz) δ 13.8, 18.9, 21.9, 22.0, 23.3, 26.5, 28.8, 29.2, 29.5, 31.9, 35.2, 40.8, 43.9, 44.4, 45.8, 46.0, 56.5, 58.3, 69.2, 71.0, 75.2, 112.5, 118.2, 122.3, 135.5, 141.4, 145.1; HRMS (ESI) calcd. for C₂₇H₄₅O₃(M⁺+H) 417.3369, measured 417.3372.

s. Synthesis of (3S)-3-[(1′S,3A′R,4′S,7A′S)-7A′-Methyl-4′-[(Triethylsilyl)Oxy]-Octahydro-1H-Inden-1′-yl)]-3-Hydroxybutanenitrile (21).

To a solution of acetonitrile (0.45 mL, 8.69 mmol) in anhydrous THF (20 mL) was added LDA (2M in THF/heptane/ethylbenzene; 4.3 mL, 8.60 mmol) at −78° C. The yellow mixture was stirred at this temperature for 30 min. A solution of ketone 11 (0.60 g, 1.93 mmol) in anhydrous THF (10 mL) was added and stirring was continued for 2 h. Saturated NH₄Cl (10 mL) was slowly added and the mixture was extracted with ethyl acetate (3×15 mL). The combined organic layers were washed with water (10 mL), dried (MgSO₄) and evaporated. The residue was purified by column chromatography on silica using hexane/ethyl acetate (85:15) to give hydroxy nitrile 21 (0.624 g, 92%) as a colorless oil. 21: [α]24D+29 (c 1.0, CHCl₃); ¹H NMR (300 MHz, CDCl₃) δ 0.54 (6H, q, J=7.8 Hz, SiCH₂CH₃), 0.94 (9H, t, J=7.8 Hz, SiCH₂CH₃), 1.11 (3H, s, 7a′-CH₃), 1.49 (3H, s, 4-H₃), 2.43 and 2.49 (1H and 1H, each d, J=16.5 Hz, 2-H₂), 4.05 (1H, narr m, 4′α-H); ¹³C NMR (75 MHz) δ 4.8, 6.9, 15.4, 17.5, 21.6, 22.6, 27.1, 32.4, 34.3, 40.8, 42.9, 52.9, 57.8, 69.2, 73.6, 117.9; HRMS (ESI) calcd. for C₂₀H₃₇NO₂SiNa (M⁺+Na) 374.2491, measured 374.2495.

t. Synthesis of (3S)-3-[(1′S,3A′R,4′S,7A′S)-4′-Hydroxy-7A′-Methyl-Octahydro-11H-Inden-1′-yl)]-3-Hydroxybutanenitrile (22).

To a solution of nitrile 21 (0.458 g, 1.30 mmol) in CH₂Cl₂ (20 mL) was added a solution of hydrogen chloride in dioxane (4 M; 0.98 mL, 3.92 mmol) at 0° C. and the mixture was stirred for 30 min. Then, water (5 mL) and NaHCO₃ (10 mL) were added and the layers were separated. The aqueous phase was extracted with dichloromethane (2×10 mL) and the combined organic layers were washed with water (10 mL), dried (MgSO₄) and evaporated. The residue was purified by column chromatography on silica using hexane/ethyl acetate (7:3) to give diol 22 (0.302 g, 98%) as a colorless oil. 22: [α]24D+14 (c 1.0, CHCl₃); ¹H NMR (300 MHz, CDCl₃) δ 1.13 (3H, s, 7a′-CH₃), 1.51 (3H, s, 4-H₃), 2.44 and 2.50 (1H and 1H, each d, J=16.6 Hz, 2-H₂), 4.10 (1H, narr m, 4′-H); ¹³C NMR (75 MHz) δ 15.3, 17.3, 21.5, 22.1, 27.1, 32.6, 33.4, 40.5, 42.8, 52.4, 57.6, 69.1, 73.5, 117.8; HRMS (ESI) calcd. for C₁₄H₂₃NO₂Na (M⁺+Na) 260.1626, measured 260.1620.

u. Synthesis of (3S)-3-Hydroxy-3-[(1′S,3A′R,7A′R)-7A′-Methyl-4′-Oxo-Octahydro-1H-Inden-1′-yl)Butanenitrile (23).

The oxidation of the secondary hydroxyl group in 22 (73 mg, 0.307 mmol) was performed analogously as described above for 13. The crude product was purified by column chromatography over silica using hexane/ethyl acetate (7:3) to give the ketone 23 (68 mg, 95%) as a colorless oil. 23: [α]24D-19 (c 0.97, CHCl₃); ¹H NMR (300 MHz, CDCl₃) δ 0.80 (3H, s, 7a′-CH₃), 1.55 (3H, s, 4-H₃), 2.48 and 2.55 (1H and 1H, each d, J=16.5 Hz, 2-H₂); ¹³C NMR (75 MHz) δ 14.2, 18.8, 21.9, 23.7, 27.5, 32.5, 38.9, 40.7, 49.7, 57.7, 61.8, 73.0, 117.6, 211.1; HRMS (ESI) calcd. for C₁₄H₂₁NO₂Na (M⁺+Na) 258.1470, measured 258.1465.

v. (3S)-3-[(1′S,3A′R,7A′R)-7A′-Methyl-4′-Oxo-Octahydro-1H-Inden-1′-yl)]-3-[(Trimethylsilyl)Oxy]Butanenitrile (24).

To a solution of hydroxy nitrile 23 (89 mg, 0.378 mmol) in anhydrous CH₂Cl₂ (4 mL) was added pyridine (300 μL, 3.78 mmol) and trimethylsilyl trifluoromethanesulfonate (206 μL, 1.14 mmol) at 0° C. and the mixture was stirred at this temperature for 1 h. Saturated NaHCO₃ (3 mL) was added and the mixture was extracted with dichloromethane (3×5 mL). The combined organic layers were washed with 5% HCl (5 mL), water (5 mL), dried (MgSO₄) and evaporated. The residue was purified by column chromatography on silica using hexane/ethyl acetate (9:1) to give ketone 24 (101 mg, 86%) as a colorless oil. 24: [α]24D-15 (c 0.97, CHCl₃); ¹H NMR (300 MHz, CDCl₃) δ 0.14 [9H, s, Si(CH₃)₃], 0.75 (3H, s, 7a′-CH₃), 1.57 (3H, s, 4-H₃), 2.41 and 2.49 (1H and 1H, each d, J=16.4 Hz, 2-H₂); ¹³C NMR (75 MHz) δ 2.4, 14.2, 18.8, 22.0, 23.8, 27.9, 32.5, 39.2, 40.7, 49.7, 58.5, 61.9, 75.9, 117.8, 211.4; HRMS (ESI) calcd. for C₁₇H₂₉NO₂SiNa (M⁺+Na) 330.1865, measured 330.1870.

### w. (20S)-3-[(Tert-Butyldimethylsilyl)Oxy]-22-Cyano-20-[(Trimethylsilyl)Oxy]-23,24,25,26,27-Pentanorvitamin D₃ (25).

The Wittig-Homer reaction between ketone 24 (100 mg, 0.325 mmol) and phosphine oxide 11 (294 mg, 0.650 mmol) was performed analogously as described above for 17. The crude product was purified by column chromatography over silica using hexane/ethyl acetate (96:4) to give protected vitamin D compound 25 (152 mg, 87%) as a colorless oil. 25: [α]24D+52.6 (c 1.0, CHCl₃); ¹H NMR (300 MHz, CDCl₃) δ 0.064 and 0.070 (3H and 3H, each s, 2× SiCH₃), 0.14 [9H, s, Si(CH₃)₃], 0.66 (3H, s, 18-H₃), 0.88 (9H, s, Si-t-Bu), 1.54 (3H, s, 21-H₃), 2.23 (1H, dd, J=13.0, 9.1 Hz, 4β-H), 2.36 (1H, m, 1β-H), 2.40 and 2.55 (1H and 1H, each d, J=16.3 Hz, 22-H₂), 2.44 (1H, dd, J=13.0, 3.1 Hz, 4α-H), 2.82 (1H, br d, J 12 Hz, 9β-H), 3.82 (1H, tt, J=9.1, 4.3 Hz, 3α-H), 4.78 and 5.01 (1H and 1H, each s, 19-H₂), 6.01 and 6.15 (1H, and 1H, each d, J=11.2 Hz, 6- and 7-H); ¹³C NMR (75 MHz) δ −4.6, −4.5, 2.5, 13.7, 18.2, 21.9, 22.3, 23.2, 25.9, 27.8, 28.7, 32.5, 32.8, 36.4, 40.8, 45.9, 46.9, 56.4, 58.9, 70.6, 76.5, 112.2, 118.2, 118.6, 121.2, 136.8, 140.5, 145.5; HRMS (ESI) calcd. for C₃₂H₅₅NO₂Si₂Na (M⁺+Na) 564.3669, measured 564.3675.

### x. (20S)-3-[(Tert-Butyldimethylsilyl)Oxy]-22-Formyl-20-[(Trimethylsilyl)Oxy]-23,24,25,26,27-Pentanorvitamin D₃ (26).

To a solution of nitrile 25 (105 mg, 0.193 mmol) in anhydrous CH₂Cl₂ (5 mL) was added diisobutylaluminum hydride (1.20 mL, 1.20 mmol) at −10° C. Potassium sodium tartrate solution (2 mL) was added after 1 h and the mixture was extracted with CH₂Cl₂ (3×5 mL). The combined organic layers were washed with water, dried (MgSO₄) and evaporated. The crude product was purified by column chromatography using hexane/ethyl acetate (98:2) to give aldehyde 26 (65 mg, 62%) as a colorless oil. 26: [α]24D+50.5 (c 1.4, CHCl₃); ¹H NMR (300 MHz, CDCl₃) δ 0.08 and 0.09 (3H and 3H, each s, 2× SiCH₃), 0.14 [9H, s, Si(CH₃)₃], 0.70 (3H, s, 18-H₃), 0.91 (9H, s, Si-t-Bu), 1.53 (3H, s, 21-H₃), 2.25 (1H, m, 4β-H), 2.39 (1H, dt, J=13.6, 4.8 Hz, 1β-H), 2.42 (1H, dd, J=13.2, 4.5 Hz, 4α-H), 2.50 and 2.65 (1H and 1H, each dd, J=14.0, 3.4 Hz, 22-H₂), 2.83 (1H, br d, J˜11.5 Hz, 9β-H), 3.84 (1H, tt, J=9.0, 4.0 Hz, 3α-H), 4.80 and 5.03 (1H and 1H, each m, 19-H₂), 6.03 and 6.17 (1H and 1H, each d, J=11.2 Hz, 7- and 6-H), 9.79 (1H, t, J=3.4 Hz, CHO; ¹³C NMR (75 MHz) δ −4.61, −4.60, 2.6, 14.0, 18.2, 21.9, 22.6, 23.3, 25.9, 28.7, 28.8, 32.8, 36.4, 41.0, 46.1, 46.9, 56.5, 57.5, 60.4, 70.6, 76.9, 112.2, 118.5, 121.2, 136.6, 140.7, 145.4, 202.1; HRMS (ESI) calcd. for C₃₂H₅₆O₃Si₂Na (M⁺+Na) 567.3666, measured 567.3678.

### y. (20S,23R)- and (20S,23S)-3-[(Tert-Butyldimethylsilyl)Oxy]-23-Hydroxy-20-[(Trimethylsilyl)Oxy]-Vitamin D₃ (27a, b).

To a solution of aldehyde 26 (37 mg, 0.068 mmol) in anhydrous THF (2 mL) was added isobutylmagnesium chloride (2 M in THF; 68 μL, 0.136 mmol) at 0° C. and the mixture was stirred at rt overnight. Then, saturated NH₄Cl (1 mL) was added and the mixture was extracted with AcOEt (3×3 mL). The combined organic layers were washed with water (2 mL), dried (MgSO₄) and evaporated. The crude product was purified by column chromatography using hexane/ethyl acetate (95:5) to give mixture of alcohols 27a, b (29 mg, 70%) as a colorless oil.

### z. (20S,23R)- and (20S,23S)-20,23-Dihydroxyvitamin D₃ (4a, B).

The hydroxyl deprotection in the vitamin D compounds 31a, b (54 mg, 0.090 mmol) was performed analogously as described above for 23. The products were separated by RP-HPLC (9.4 mm×25 cm Zorbax Eclipse XDB-C18 column, 4 mL/min) using methanol/water (85:15) solvent system. Pure vitamin 4b (16 mg) was collected at R_{V}=28 mL and 4a (11 mg) at R_{V}=48 mL (total yield 74%).

4a: [α]24D+25.1 (c 0.9, CHCl₃); 1H NMR (300 MHz, CDCl₃) δ 0.63 (3H, s, 18-H₃), 0.92 (6H, d, J=6.6 Hz, 26- and 27-H₃), 1.30 (3H, s, 21-H₃), 2.17 (1H, ddd, J=13.1, 8.6, 4.7 Hz, 1α-H), 2.27 (1H, dd, J=13.1, 7.6 Hz, 4β-H), 2.39 (1H, m, 1β-H), 2.56 (1H, dd, J=13.1, 3.8 Hz, 4α-H), 2.82 (1H, dd, J=11.8, 3.9 Hz, 9β-H), 3.93 (1H, m, 3α-H), 4.13 (1H, m, 23-H), 4.80 and 5.04 (1H and 1H, each m, 19-H₂), 6.03 and 6.21 (1H and 1H, each d, J=11.2 Hz, 7- and 6-H); 13C NMR (75 MHz) δ 14.1, 21.9, 22.1, 22.5, 23.3, 23.6, 24.2, 28.2, 28.8, 31.9, 35.2, 40.7, 45.6, 45.9, 47.9, 49.2, 56.5, 67.2, 69.2, 76.2, 112.5, 118.4, 122.2, 135.7, 141.2, 145.1; HRMS (ESI) calcd. for C₂₇H₄₅O₃ (M⁺+H) 417.3369, measured 417.3374.

4b: [α]24D+17.6 (c 0.9, CHCl₃); 1H NMR (300 MHz, CDCl₃) δ 0.71 (3H, s, 18-H₃), 0.90 and 0.92 (6H, each d, J=6.6 Hz, 26- and 27-H₃), 1.39 (3H, s, 21-H₃), 2.19 (1H, ddd, J=13.1, 8.7, 4.8 Hz, 1α-H), 2.27 (1H, dd, J=13.1, 7.5 Hz, 4β-H), 2.39 (1H, m, 10-H), 2.56 (1H, dd, J=13.1, 3.8 Hz, 4α-H), 2.81 (1H, br d, J˜12 Hz, 9β-H), 3.93 (1H, m, 3α-H), 4.09 (1H, m, 23-H), 4.80 and 5.04 (1H and 1H, each m, 19-H₂), 6.03 and 6.21 (1H and 1H, each d, J=11.2 Hz, 7- and 6-H); 13C NMR (75 MHz) δ 13.8, 21.9, 22.2, 22.4, 23.2, 23.3, 24.3, 26.0, 28.8, 31.9, 35.2, 40.9, 45.9, 46.0, 46.9, 47.4, 56.5, 61.2, 66.8, 69.2, 76.8, 112.5, 118.3, 122.2, 135.6, 141.2, 145.1; HRMS (ESI) calcd. for C₂₇H₄₅O₃ (M⁺+H) 417.3369, measured 417.3375.

Aa. Exemplary Hydroxy Derivatives of Vitamin D

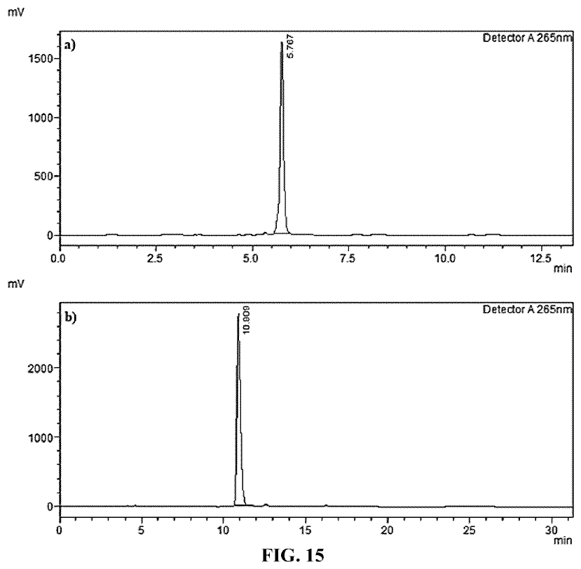

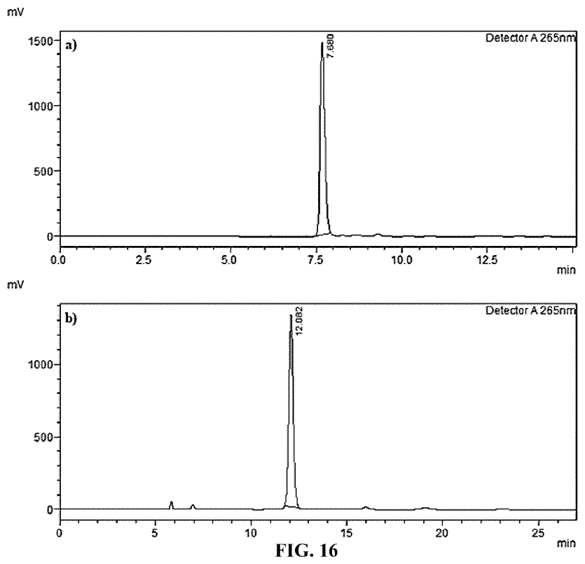

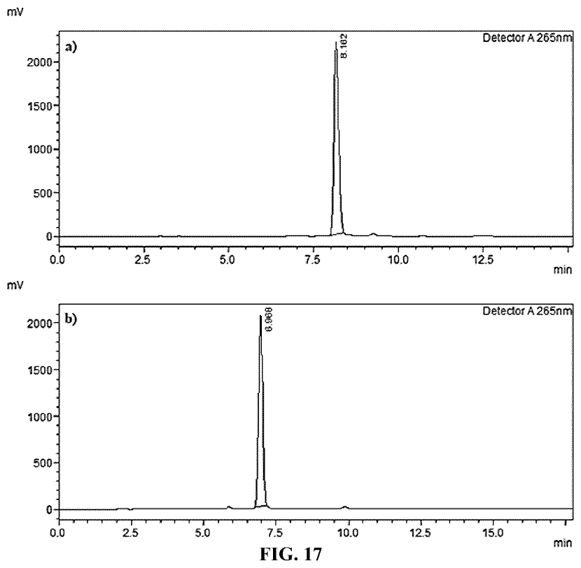

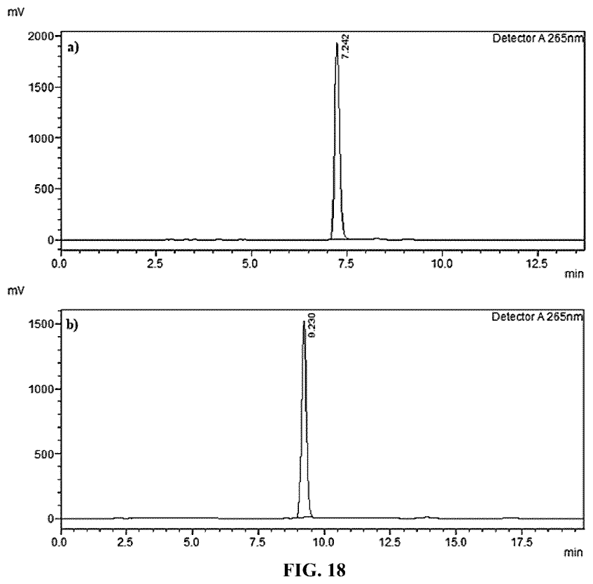

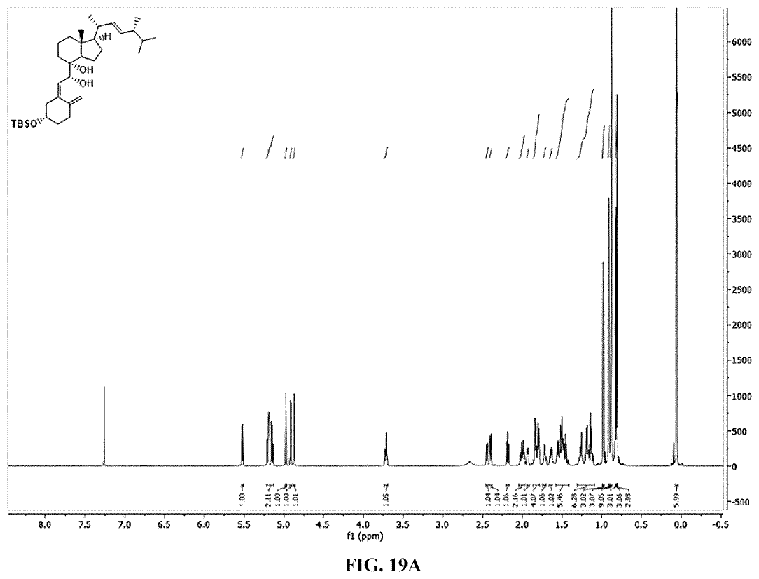

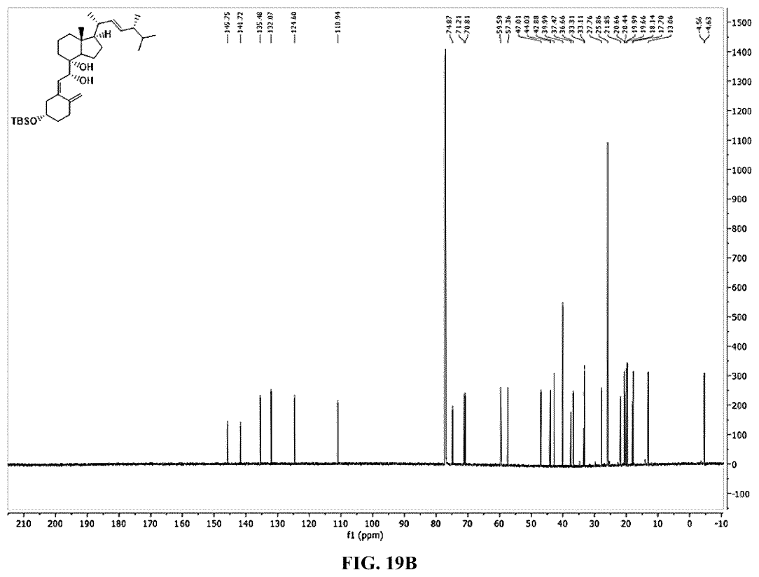

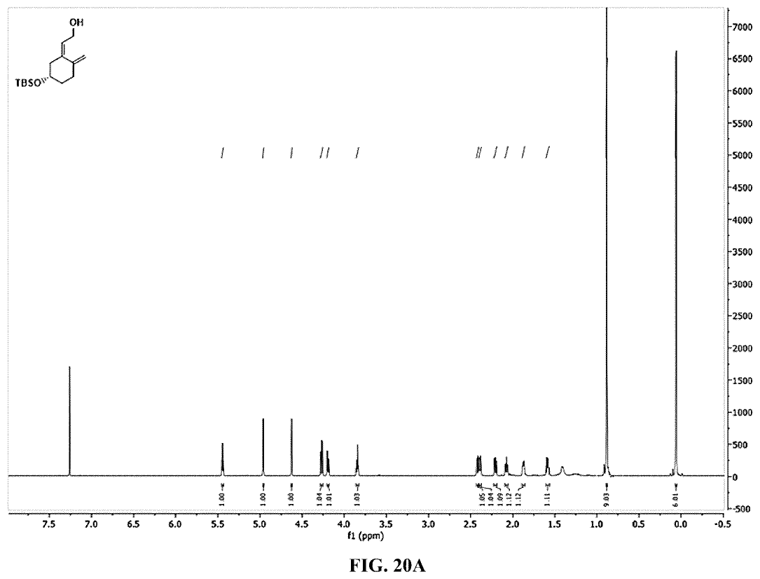

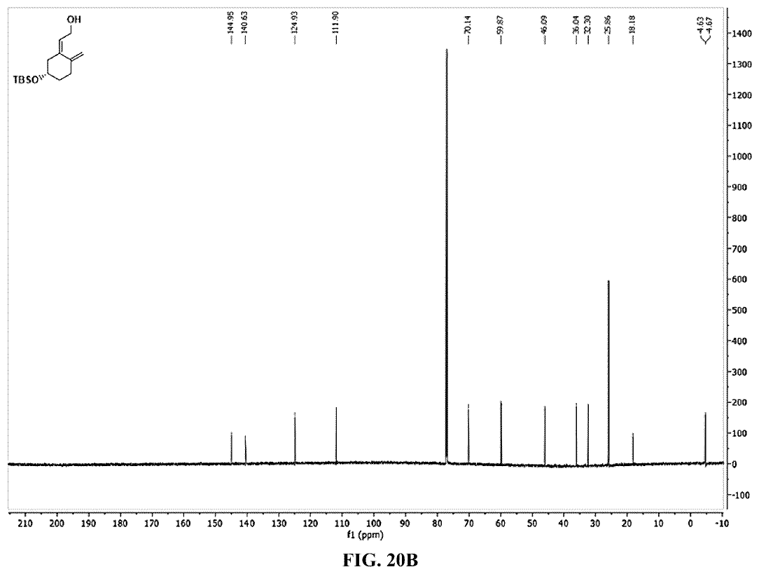

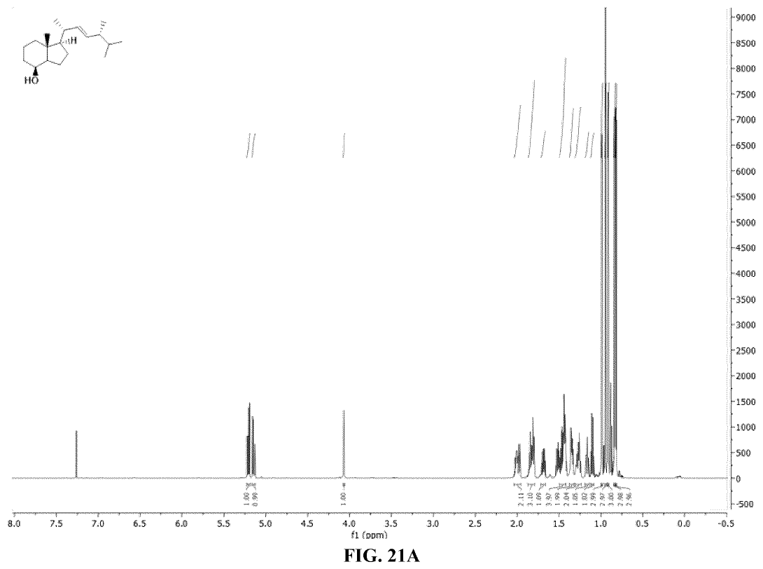

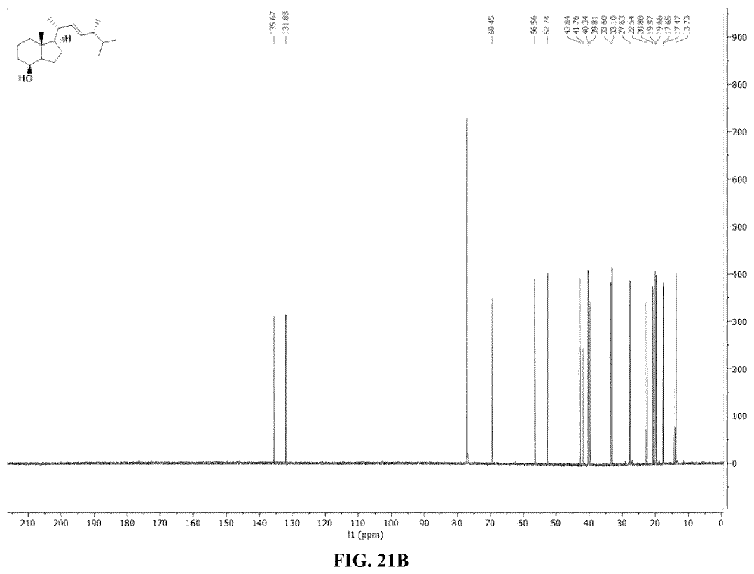

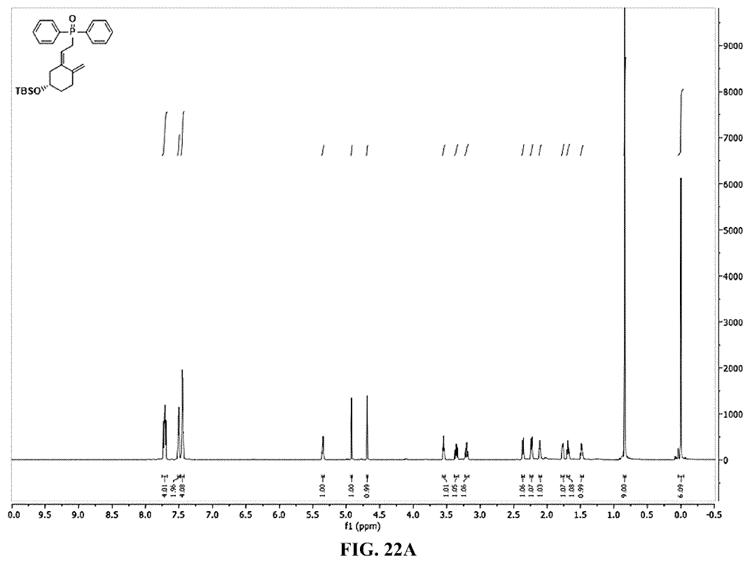

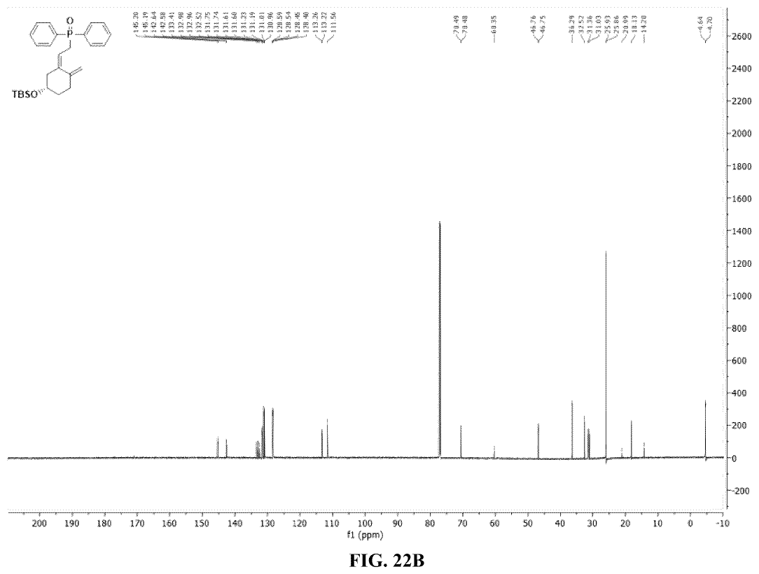

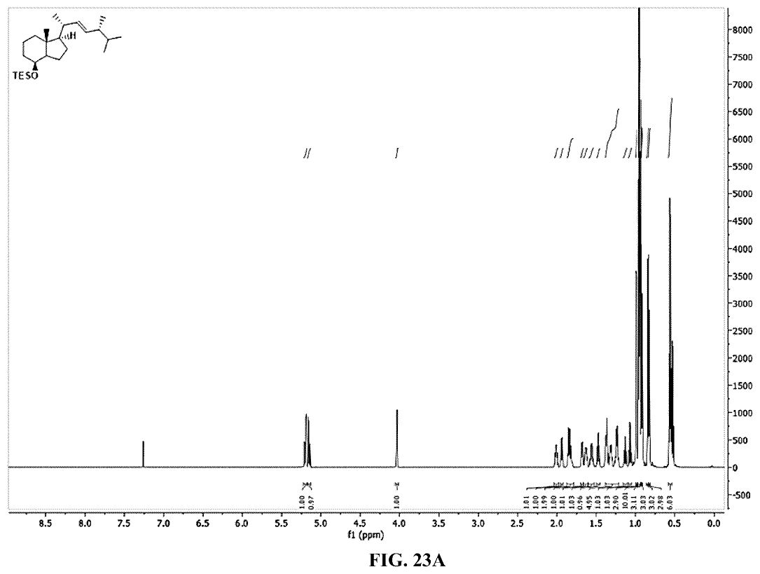

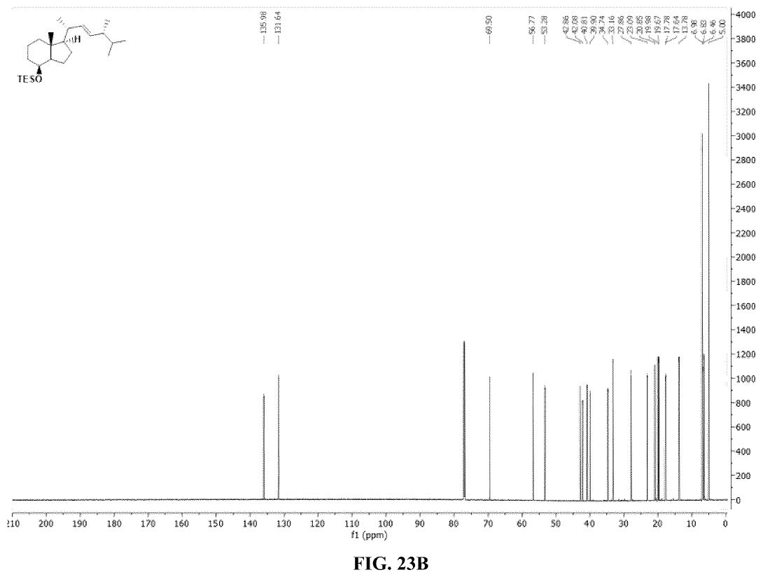

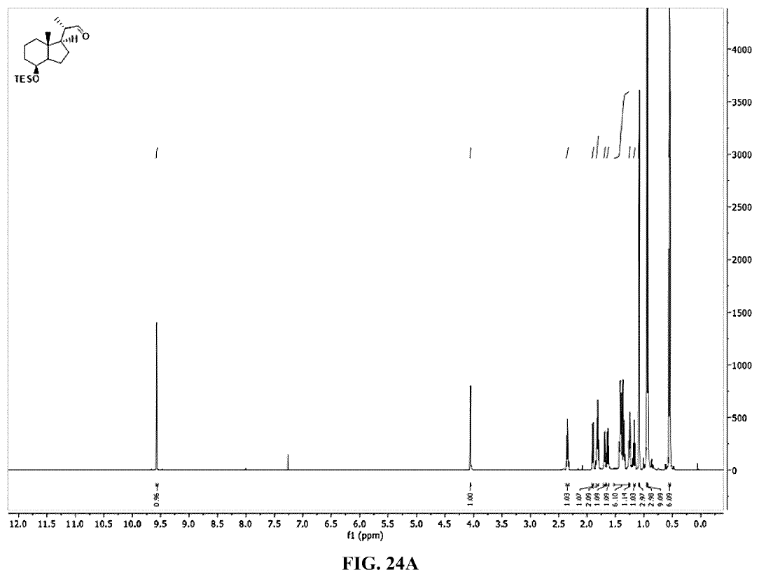

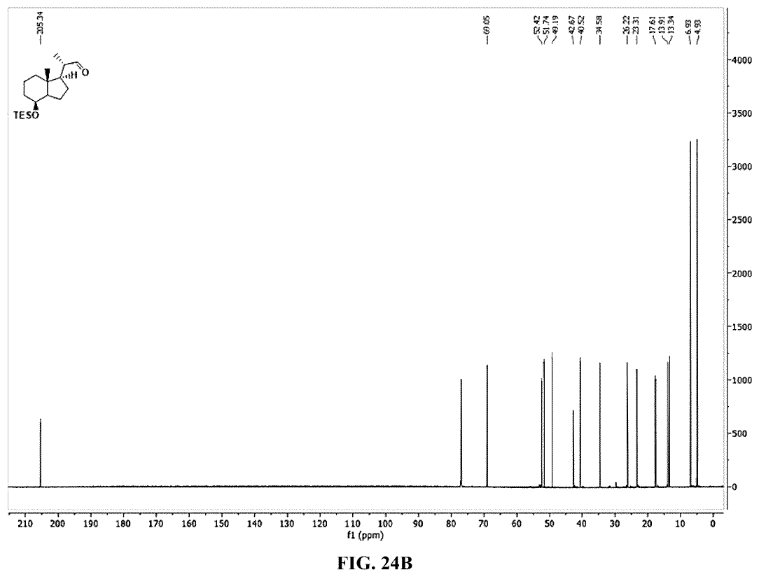

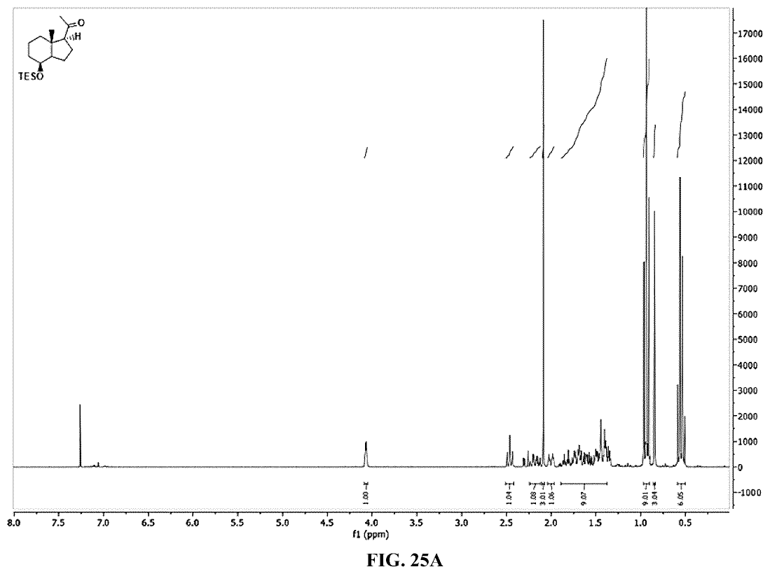

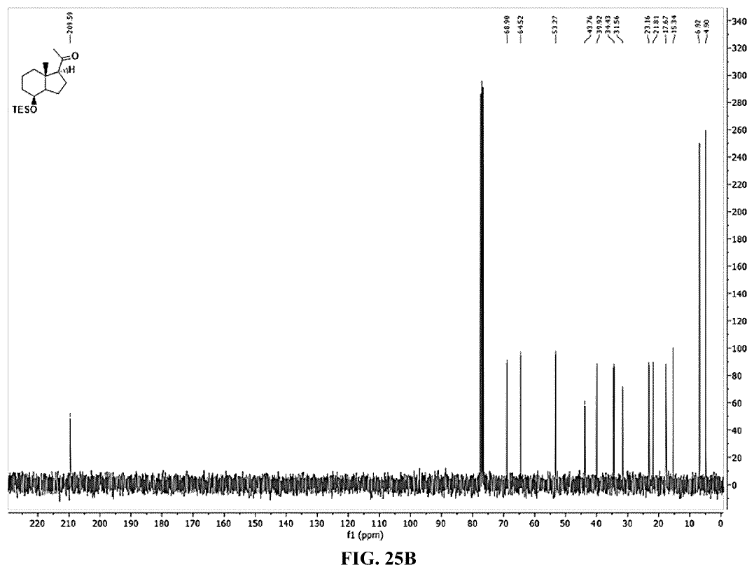

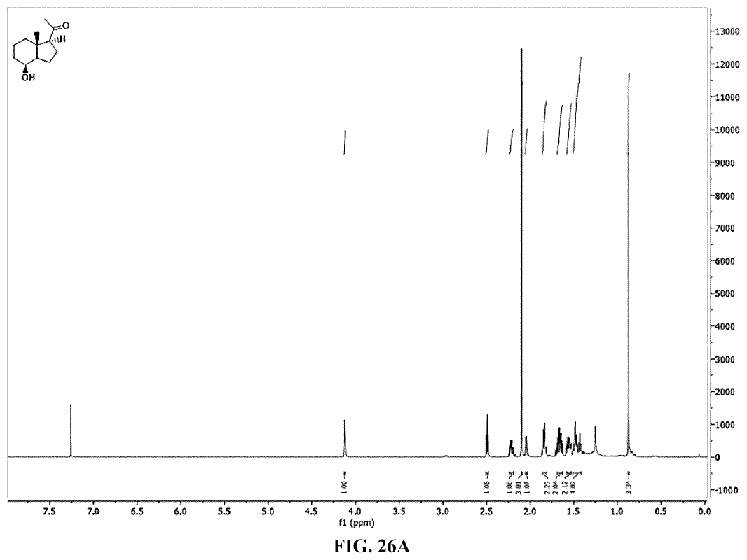

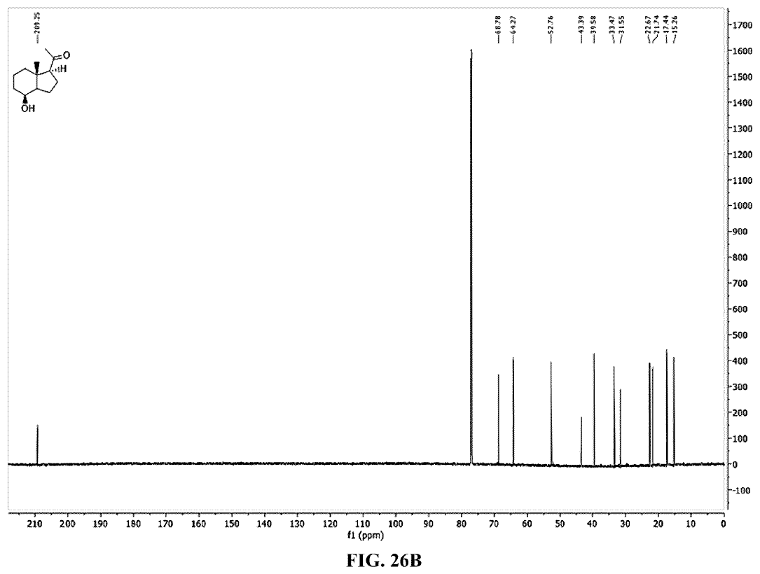

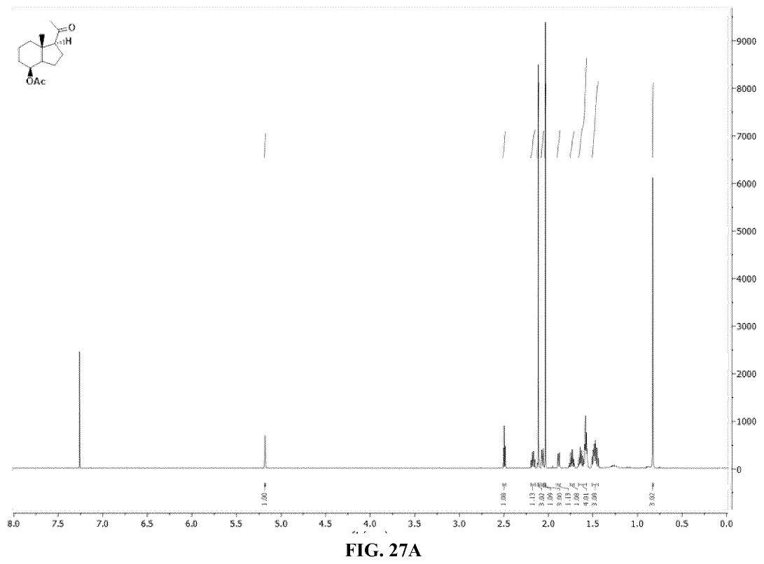

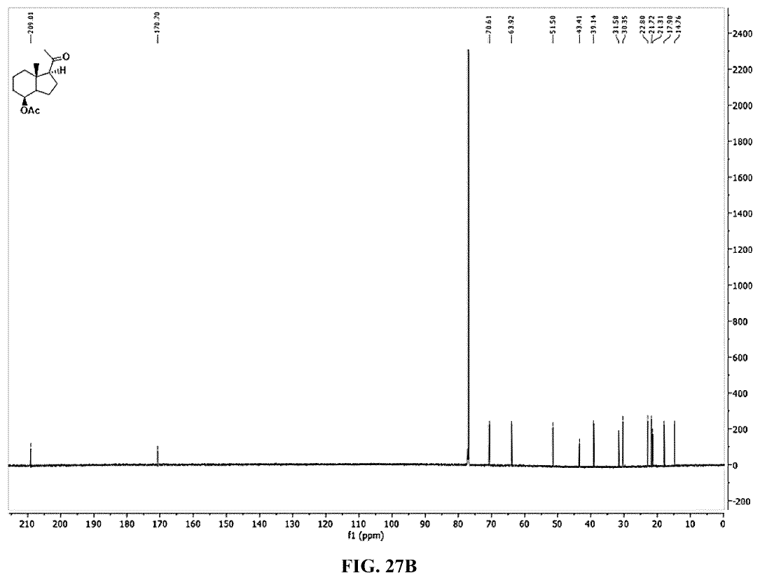

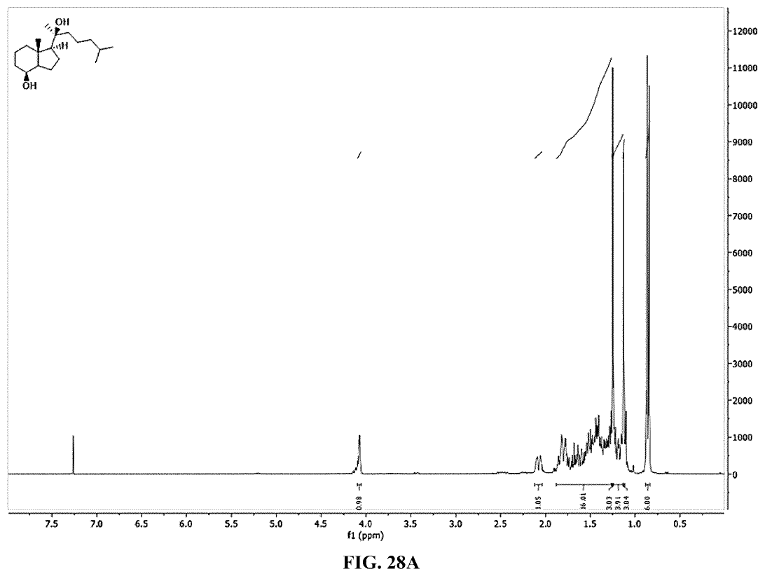

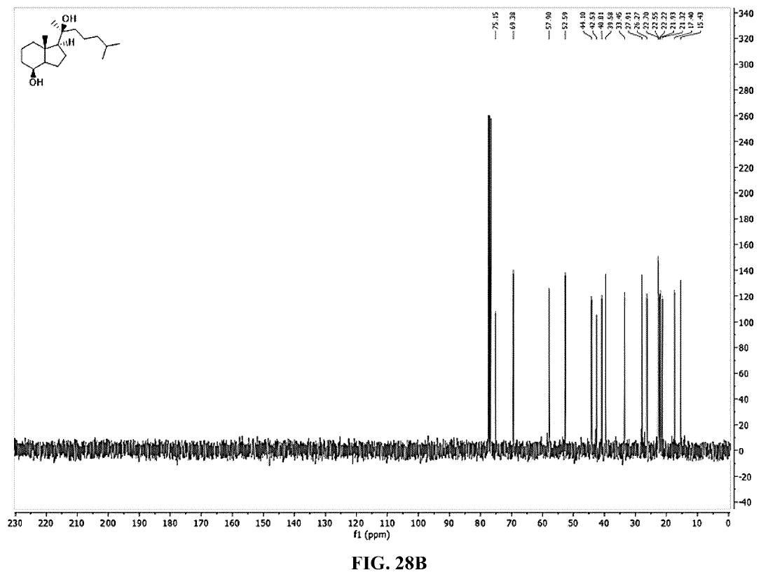

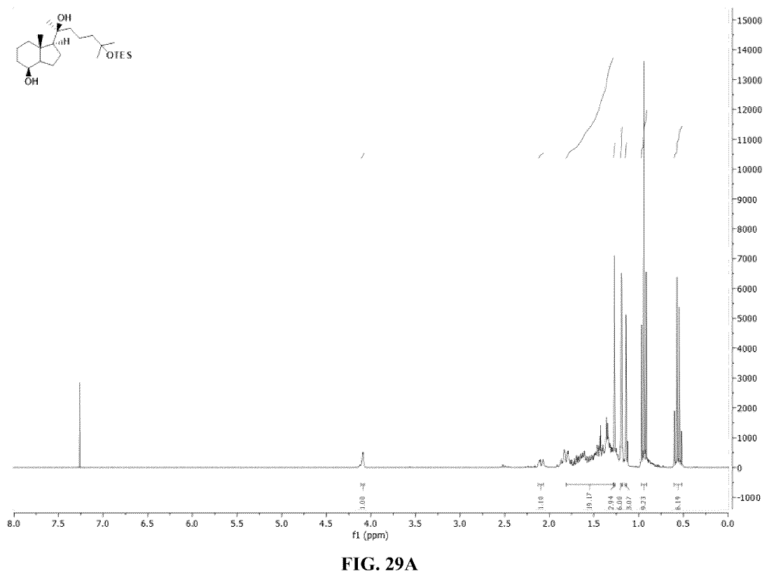

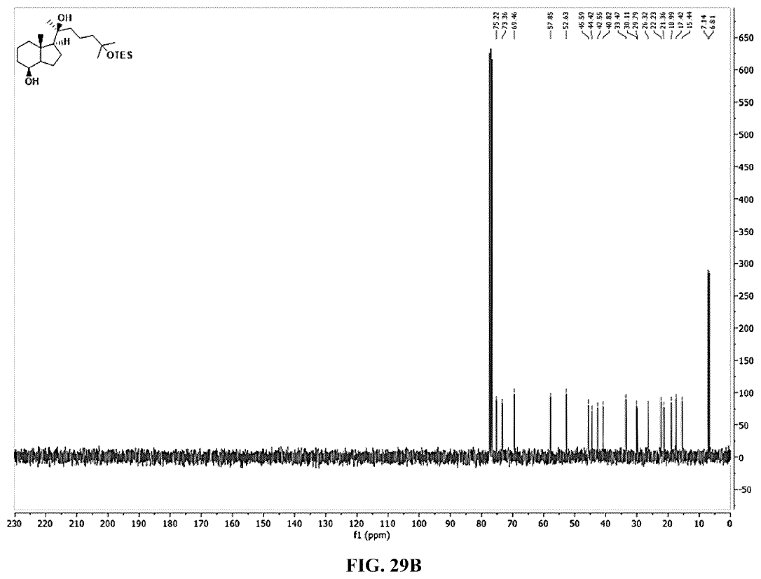

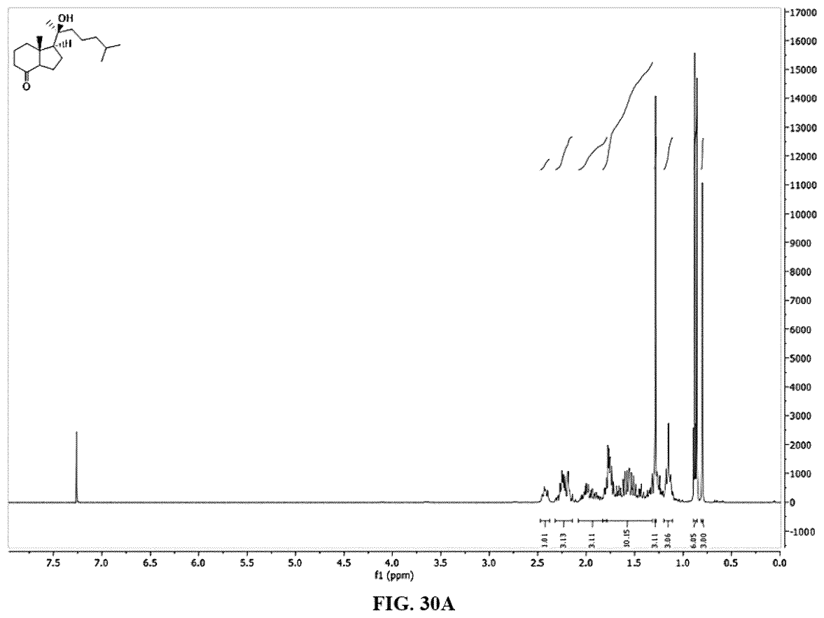

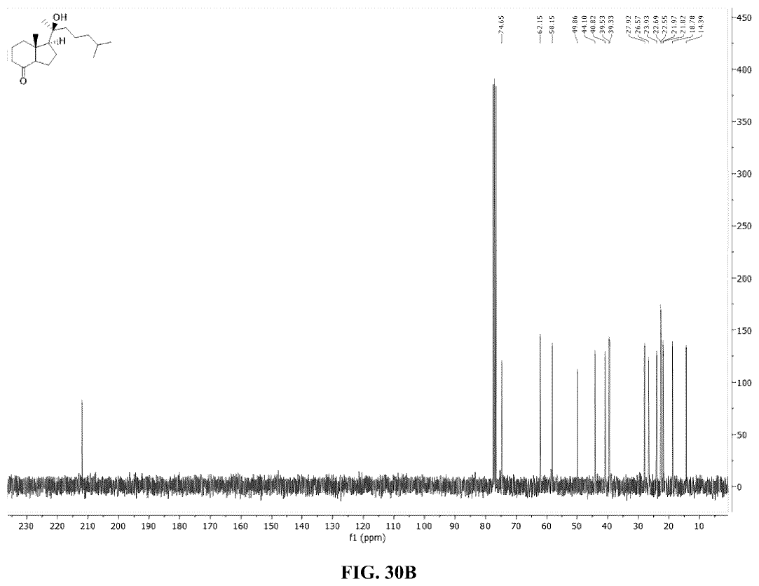

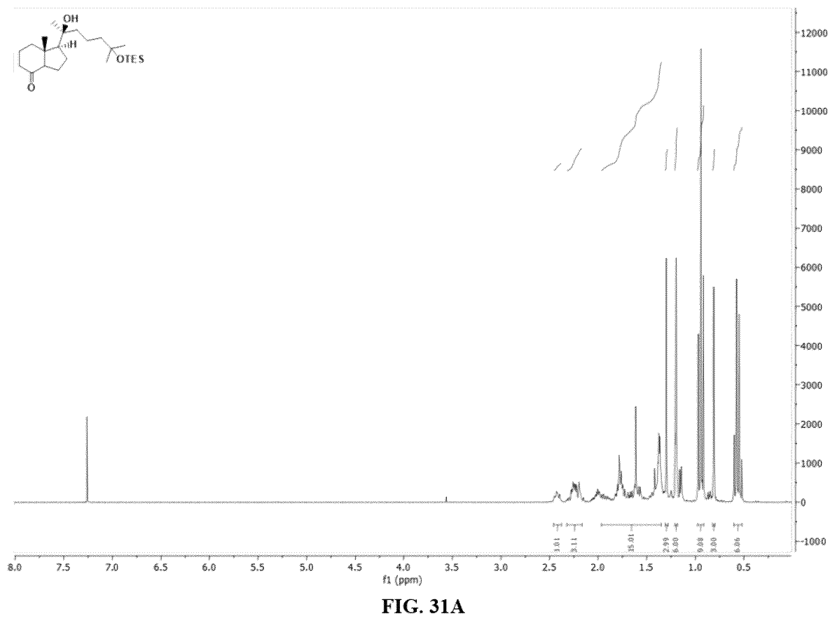

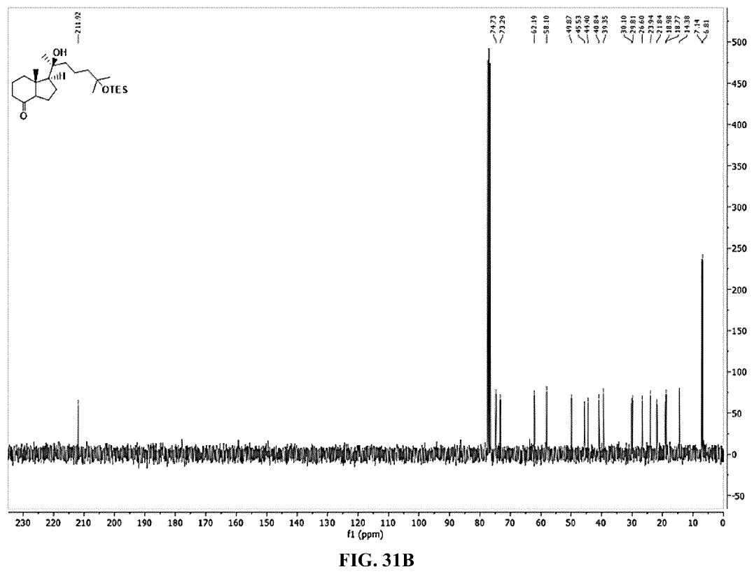

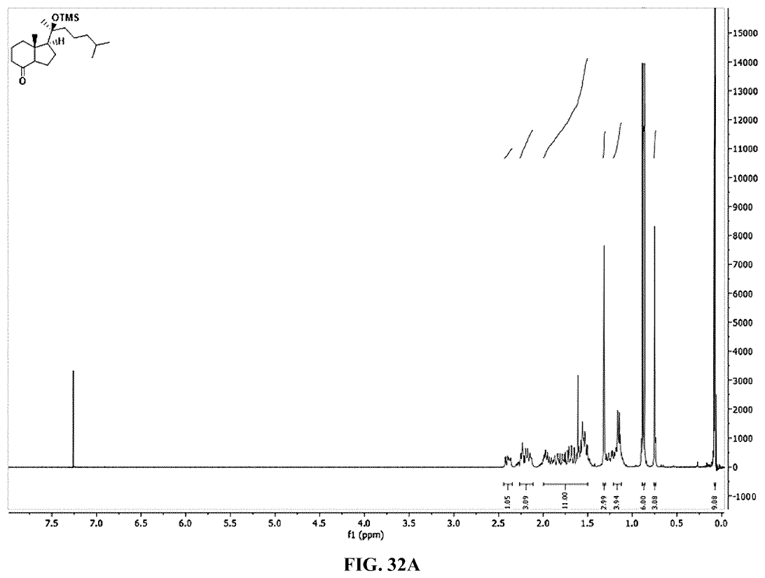

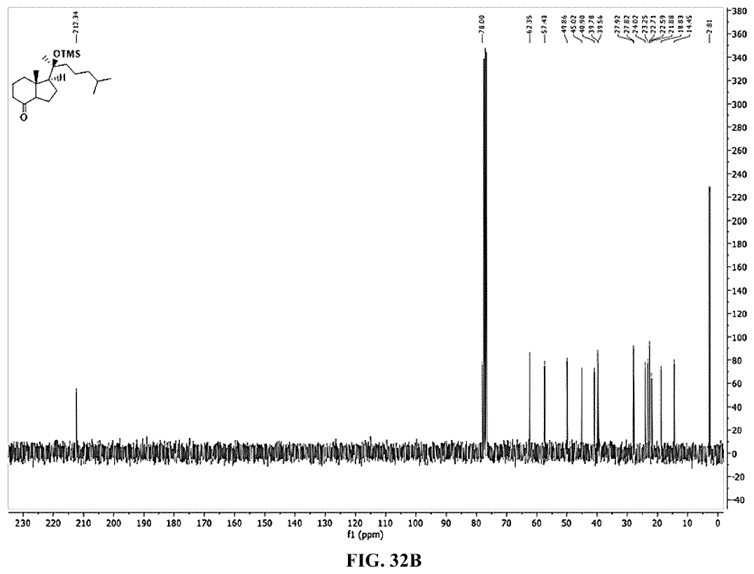

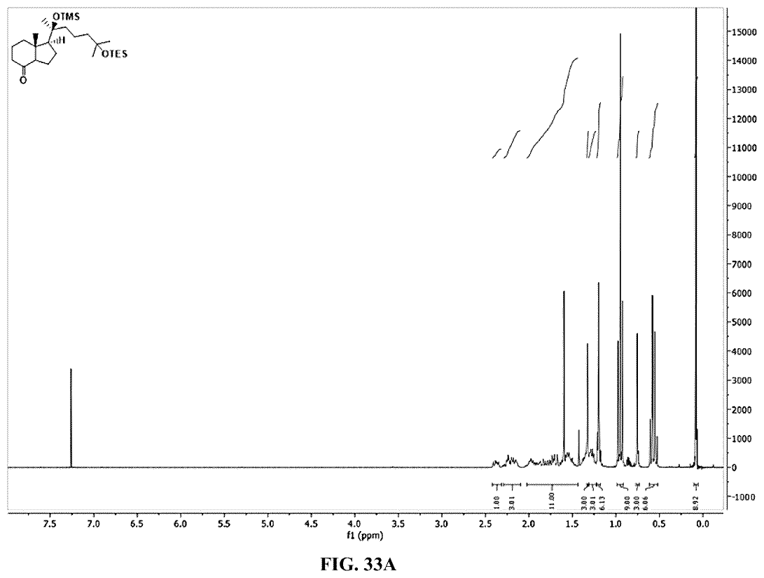

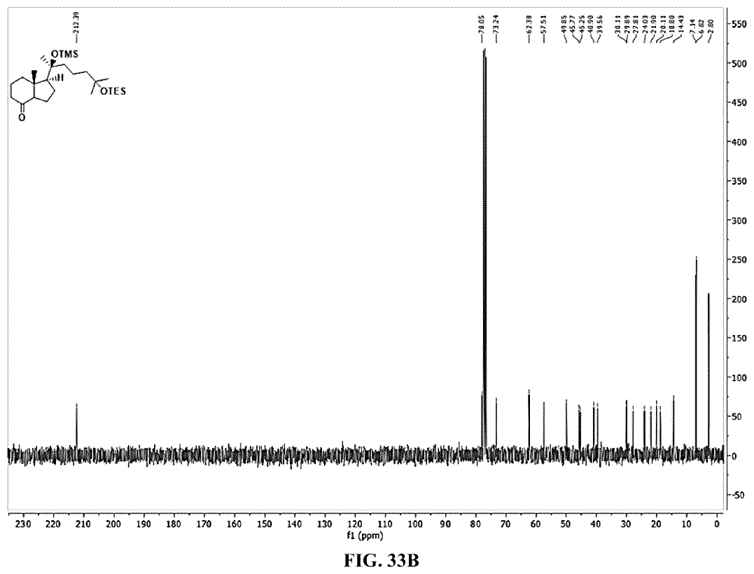

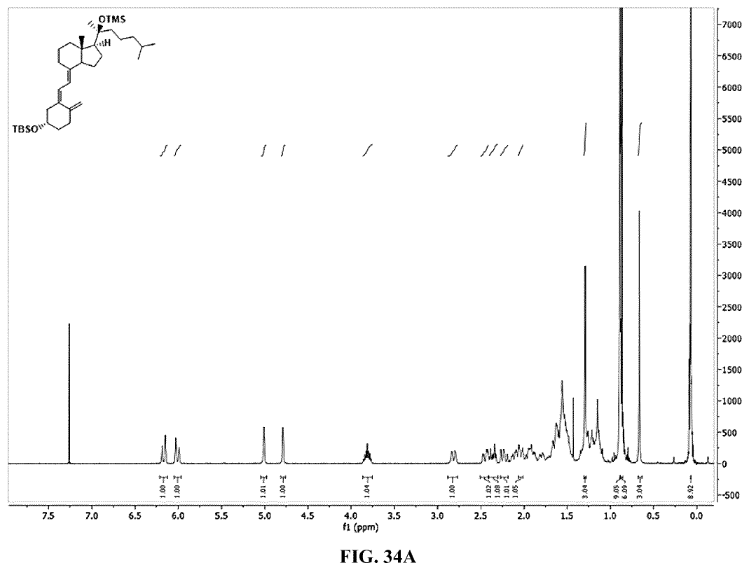

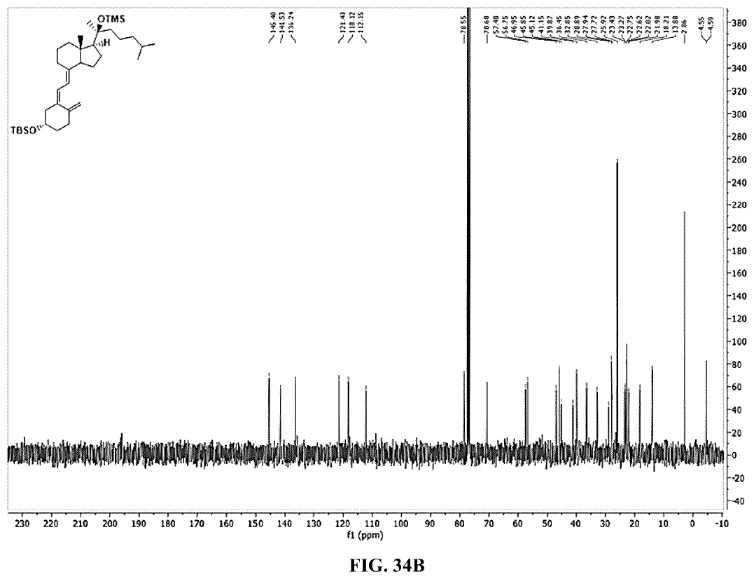

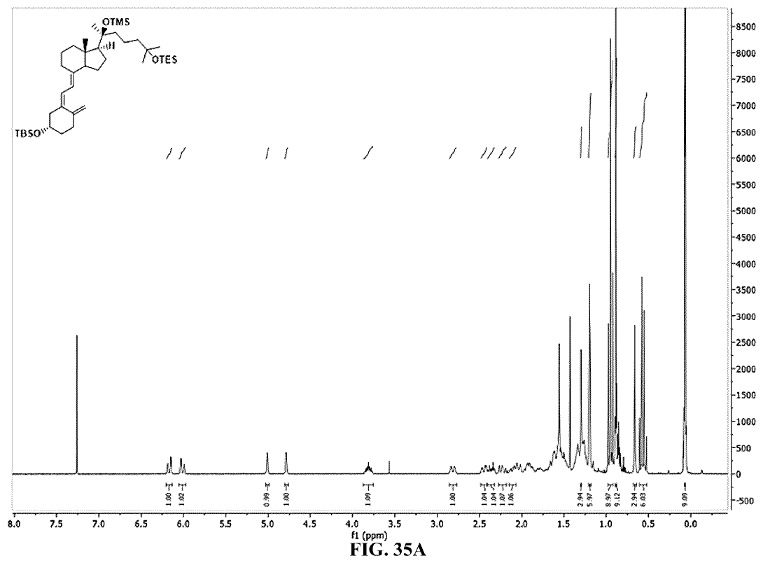

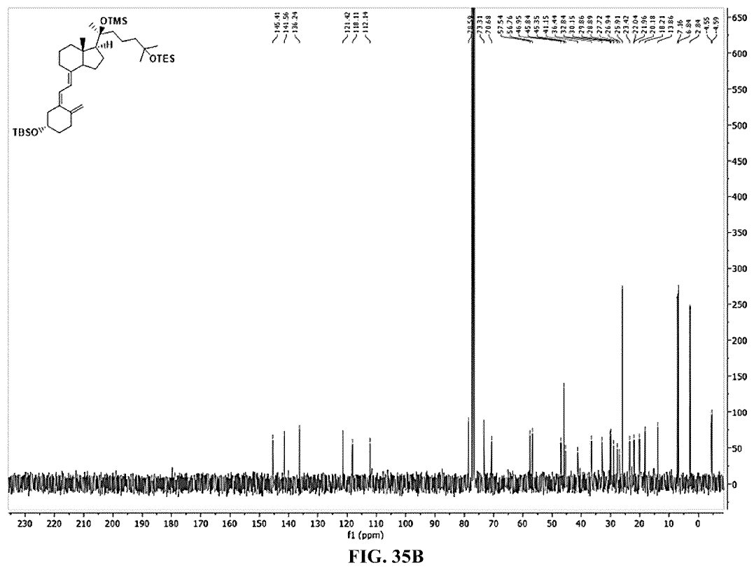

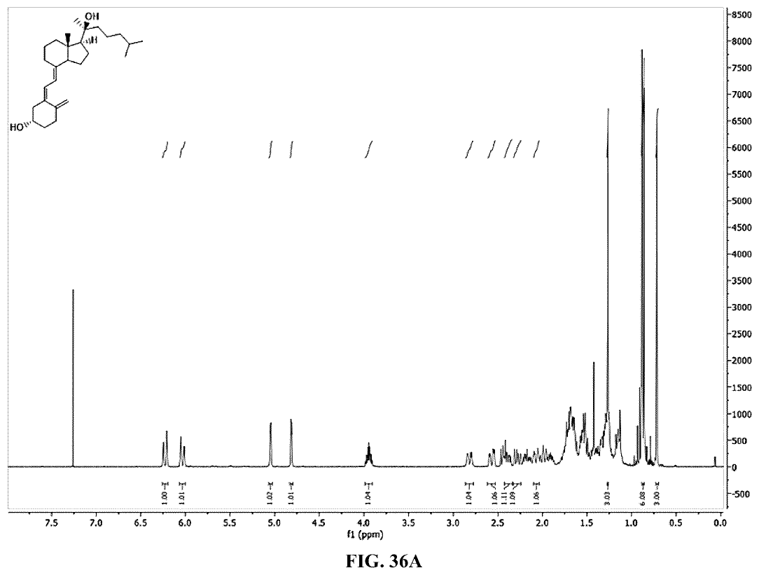

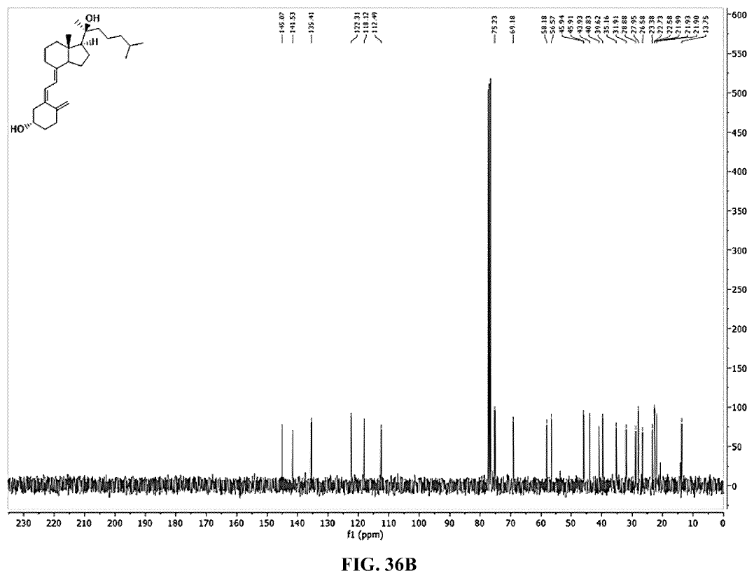

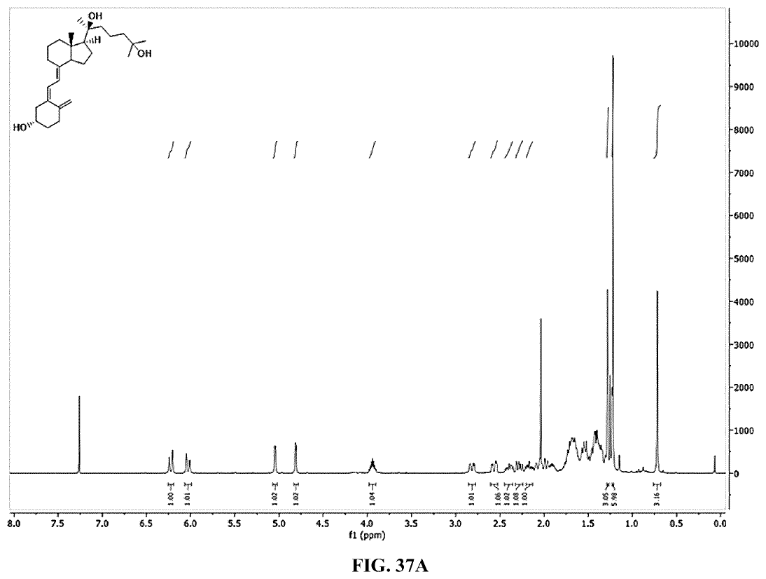

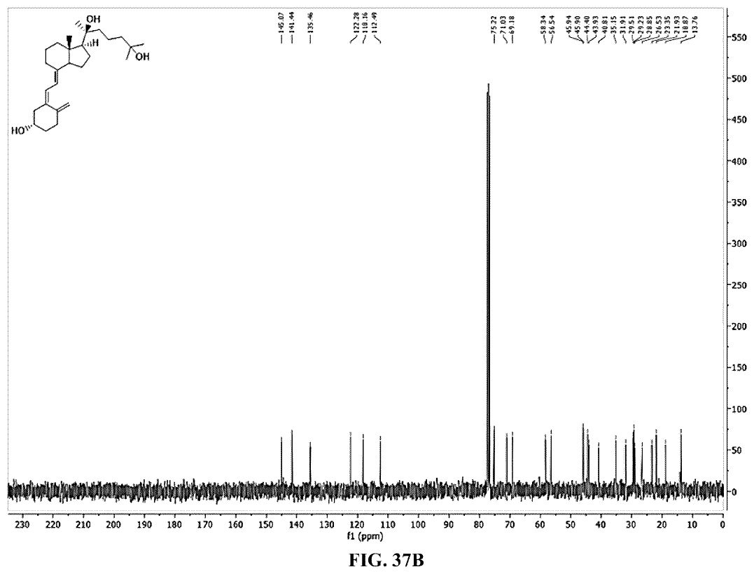

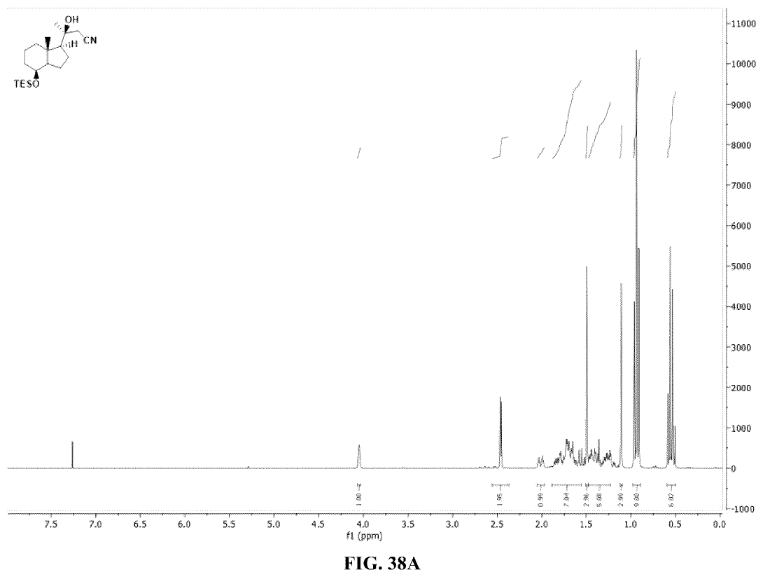

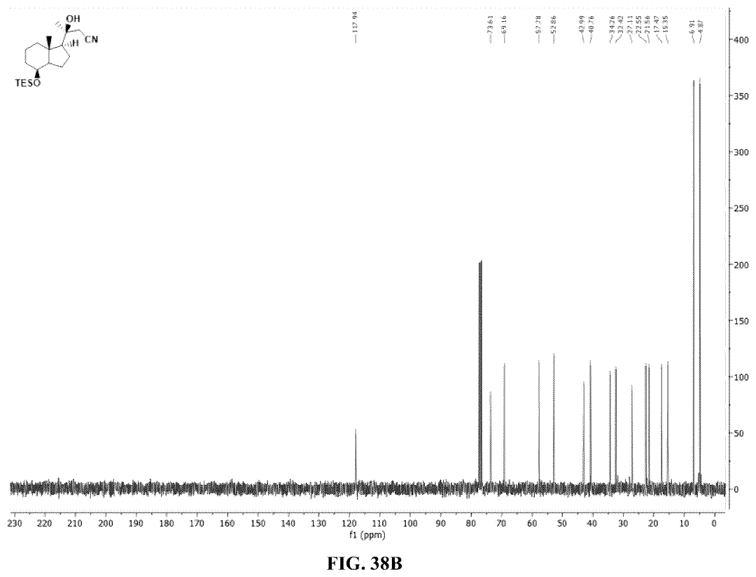

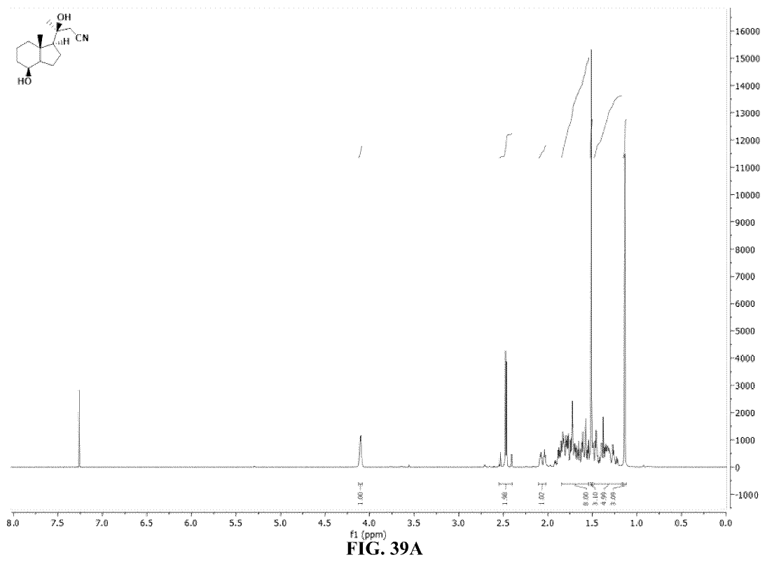

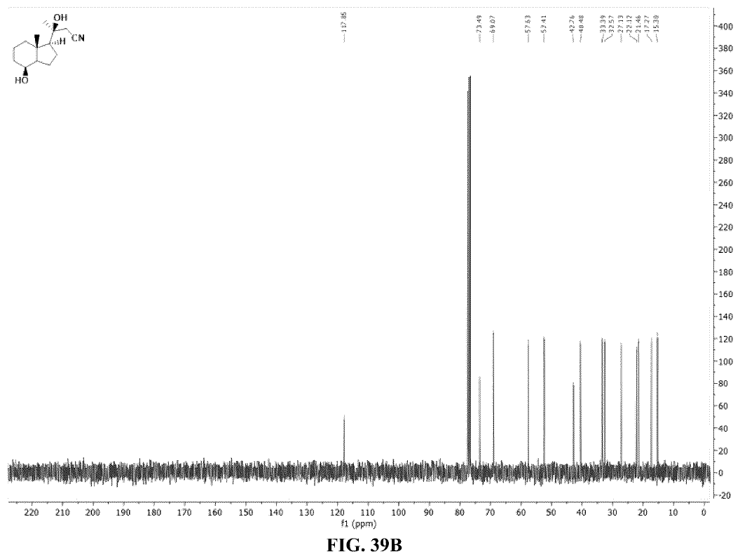

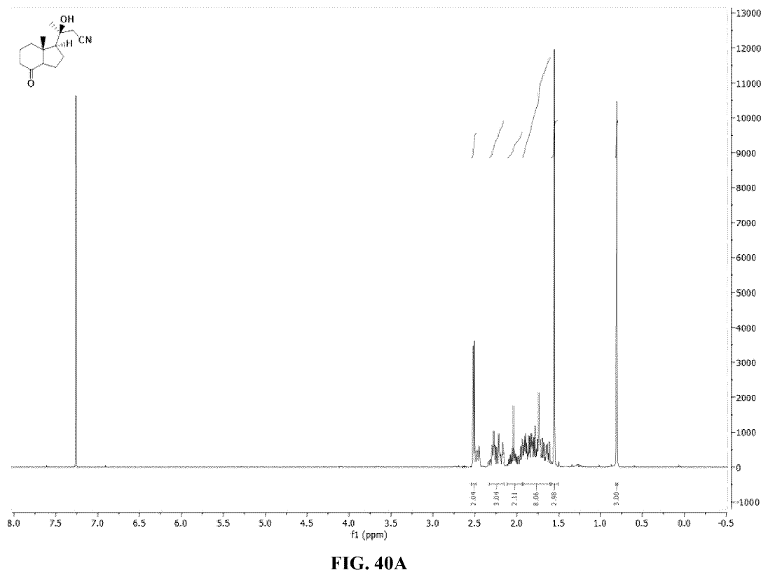

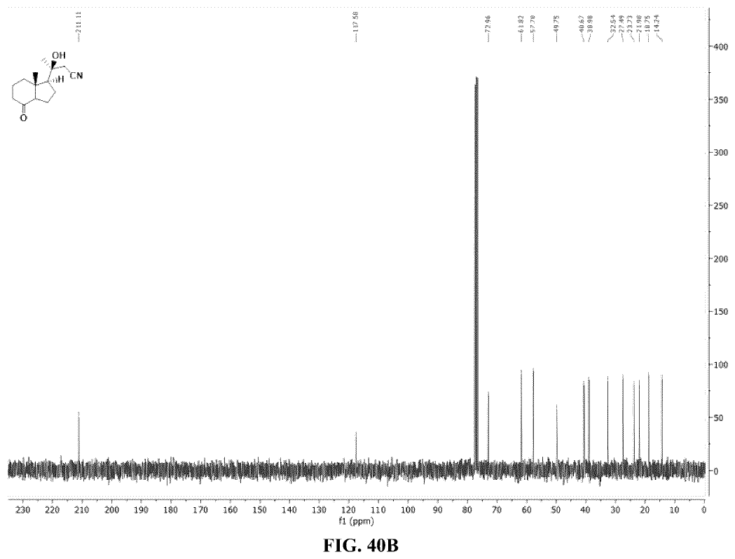

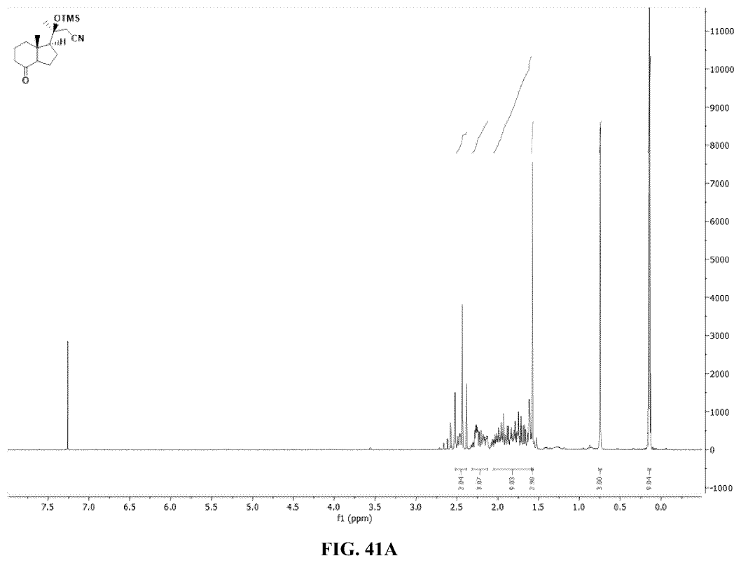

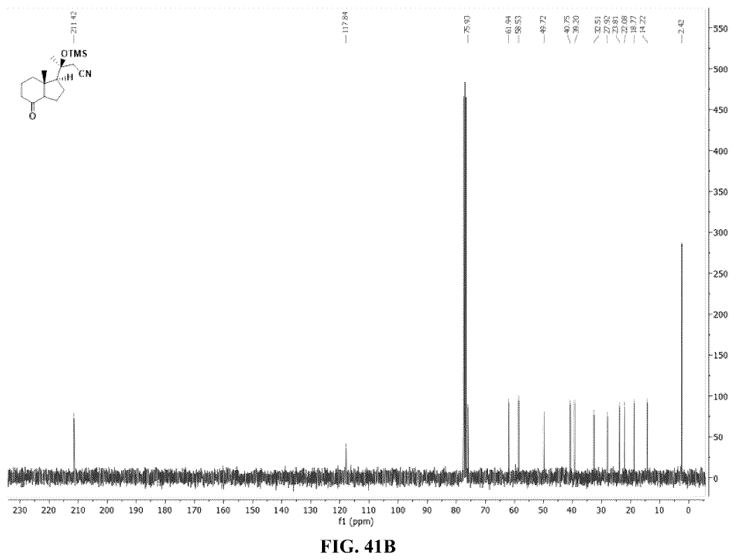

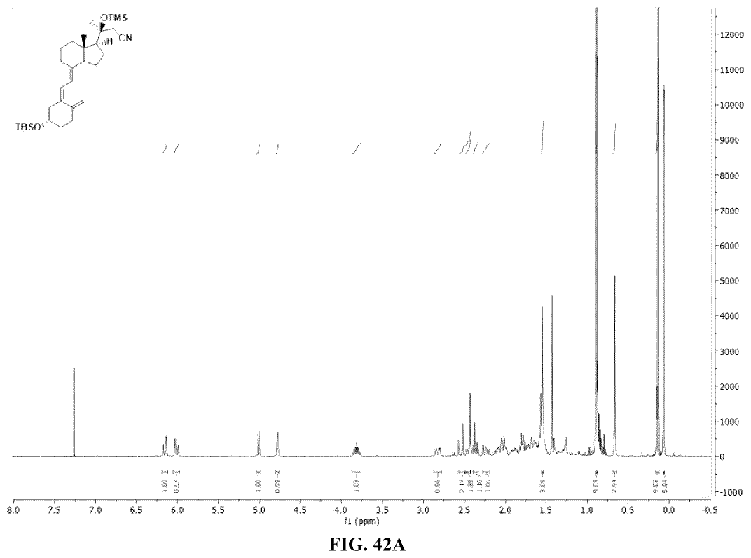

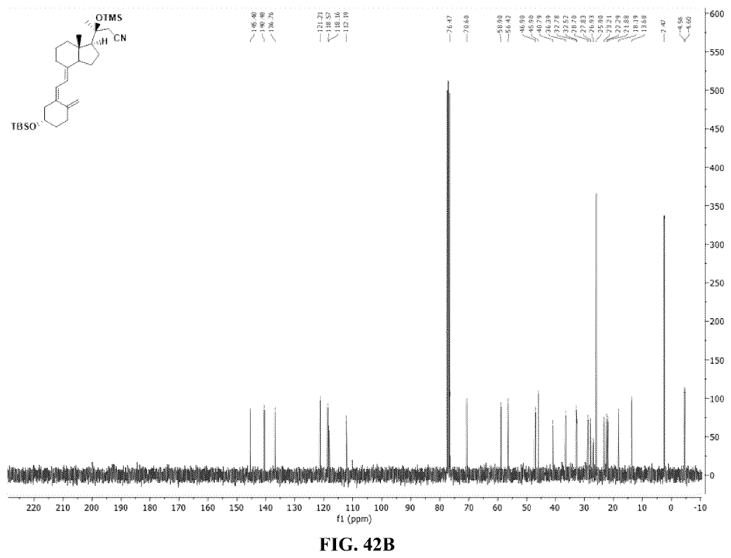

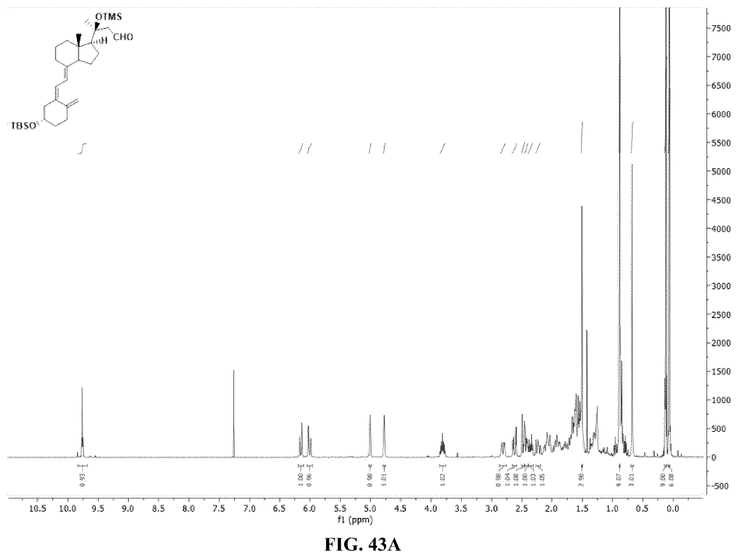

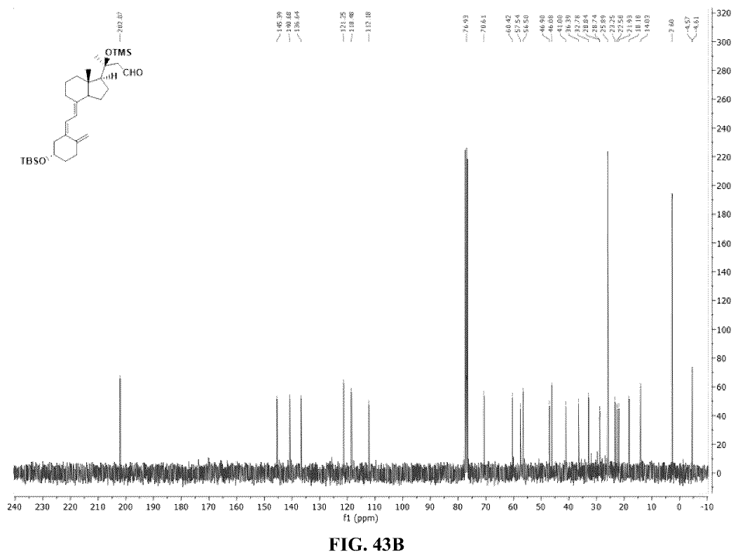

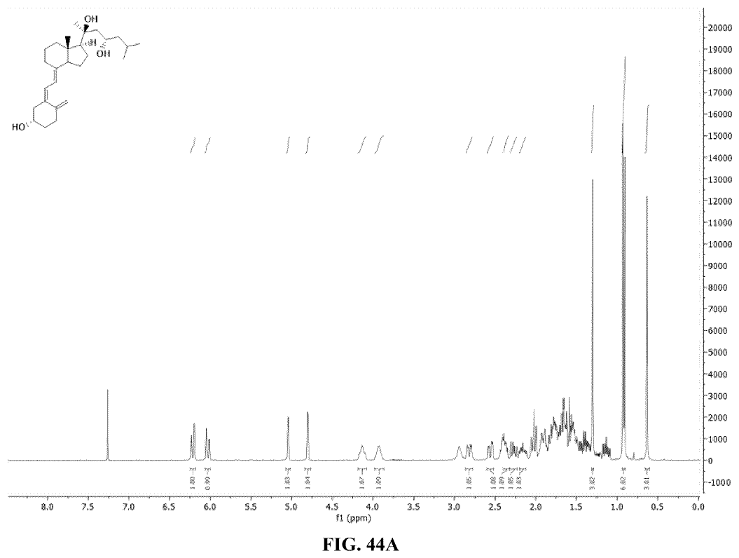

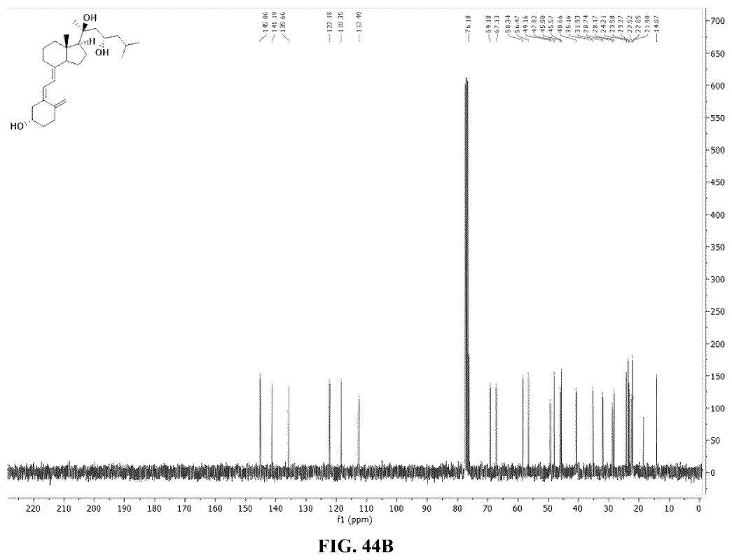

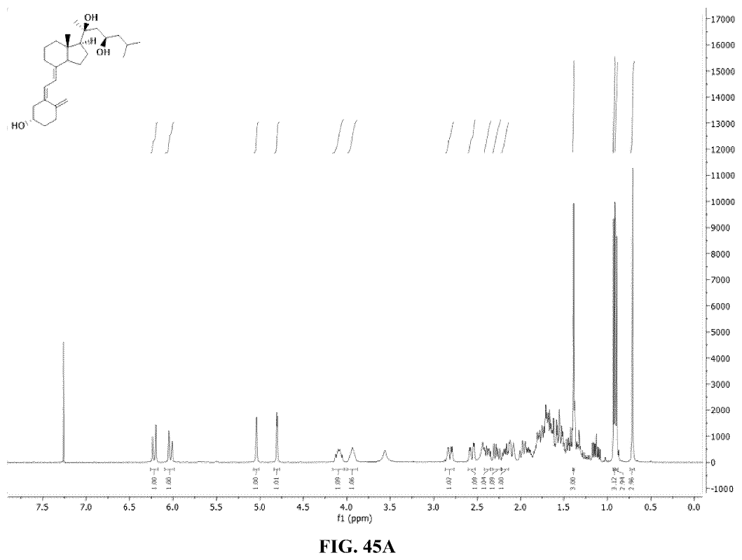

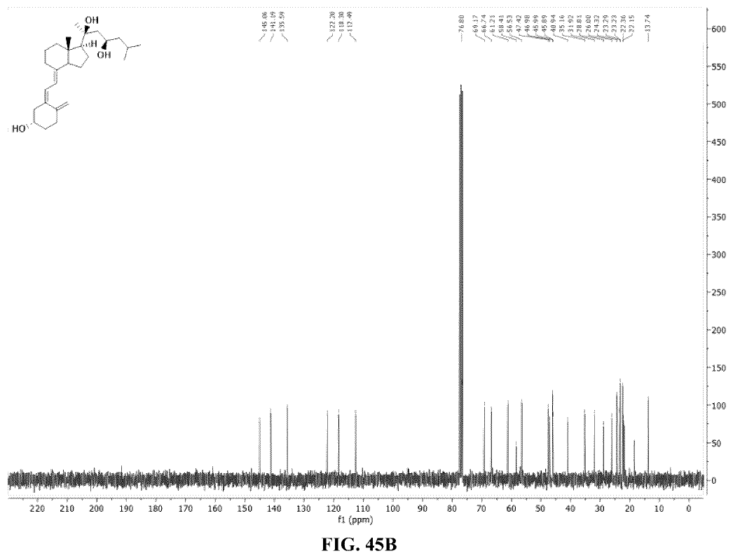

All vitamin D hydroxy derivatives synthesized gave single sharp peaks on HPLC and were judged at least 98% pure (Table 1). Two HPLC systems (straight- and reversed-phase) with isocratic elution were employed (FIG. 15-FIG. 18). FIG. 15 shows straight-phase (a) and reversed-phase (b) HPLC chromatograms of 20S(OH)D₃ (3). FIG. 16 shows straight-phase (a) and reversed-phase (b) HPLC chromatograms of 20S,23S(OH)₂D₃ (4a). FIG. 17 shows straight-phase (a) and reversed-phase (b) HPLC chromatograms of 20S,23R(OH)₂D₃ (4b). FIG. 18 shows straight-phase (a) and reversed-phase (b) HPLC chromatograms of 20S,25(OH)₂D₃ (6). The purity and identity of the synthesized vitamins were additionally confirmed by inspection of their ¹H and ¹³C NMR and high-resolution mass spectra.

TABLE 1

| | | HPLC Retention Volumes | |
|---|---|---|---|
| | Compound | Straight-phase^{a} | Reversed-phase^{b} |
| Compound | No. | (hexane/2-propanol) | (methanol/water) |
| 20S(OH)D₃ | 3 | h/p (95:5) | m/w (9:1) |
| | | Rᵥ = 23 mL | Rᵥ = 44 mL |
| 20S,23S(OH)₂D₃ | 4a | h/p (95:5) | m/w (85:15) |
| | | Rᵥ = 31 mL | Rᵥ = 48 mL |
| 20S,23R(OH)₂D₃ | 4b | h/p (95:5) | m/w (85:15) |
| | | Rᵥ = 33 mL | Rᵥ = 28 mL |
| 20S,25(OH)₂D₃ | 6 | h/p (9:1) | m/w (8:2) |
| | | Rᵥ = 29 mL | Rᵥ = 37 mL |

^{a}Zorbax RX-Sil; 9.4 × 250 mm column;
^{b}Zorbax Eclipse XDB-C18; 9.4 × 250 mm column.

Additional exemplary compounds were prepared using the method detailed above.

### 2. Measurement of CYP27B1 Activity on Secosteroids

Mouse CYP27B1 was expressed in E. coli and purified (Tang E K, et. al., (2010) Drug Metabolism and Disposition, 38(9) 1553-9). CYP27B1 activity was measured on substrate incorporated into the membrane of phospholipid vesicles as described previously. Briefly, phospholipid vesicles were prepared by sonication of dioleoyl phosphatidylcholine, cardiolipin and secosteroid. Vesicles (510 μM phospholipid) were then incubated at 37° C. with CYP27B1 (0.2 μM) in buffer comprising 20 mM Hepes, pH 7.4, 100 mM NaCl, 0.1 mM EDTA, 0.1 mM and dithiothreitol, adrenodoxin (15 μM), adrenodoxin reductase (0.4 μM), glucose-6-phosphate (2 mM), glucose-6-phosphate dehydrogenase (2 U/mL) and NAPDH (50 μM) in a volume of 0.5 ml. After reaction (5 or 20 min), the incubation mixture was extracted 3-times with 2.5 ml dichloromethane, extracts combined, dried under N2 gas and dissolved in 64% methanol. Samples were analysed by reverse phase HPLC on a Grace Alltima column (25×0.46 cm) using a gradient of 64 to 100% methanol for 15 min followed by 100% methanol for 30 min, at a flow rate of 0.5 ml/min. Metabolites were detected with a UV detector at 265 nm.

### 3. Nuclear Receptor Analyses

### A. VDR Translocation Assay

To study the ligand-induced VDR translocation from cytoplasm to the nucleus, SKMEL-188 melanoma cells transduced with pLenti-CMV-VDR-EGFP-pgk-puro were used, which express VDR-GFP fusion protein, and followed the protocols described previously (Kim T K, et. al., (2012) Mol Cell Endocrinol 361(1-2) 143-52; Slominski A T, et. al., (2011) Am J Physiol Cell Physiol 300(3) C526-41; Slominski A T, et. al., (2017) Sci Rep 7(1) 11434). Briefly, cells were grown on a 96 well plate using Ham's F10 media containing 5% charcoal-treated FBS until 70% confluence. The cells were treated with the secosteroids for 1.5 h in a 96 well plate followed by treatment of NucBlue™ Live ReadyProbes™ Reagent (Invitrogen, Waltham, MA, USA) using the manufacturer's protocol. The ratio of cell number with a fluorescent nucleus (GFP) to the total cell number (DAPI) was determined using Cytation 5 (Biotek, Winooski, VT, USA).

b. AhR Binding Assay

The binding of chemically synthesized secosteroids to AhR was tested using the Human AhR Reporter Assay System (Indigo Biosciences, State College, PA, USA) following the manufacturer's protocol and as detailed previously (Slominski A T, et. al., (2018) Int J Mol Sci 19(10)). Briefly, the cells including the luciferase reporter gene linked to an AhR-responsive promoter were treated with the compounds for 23 h and the luminescence signal was measured using Cytation 5 (Biotek, Winooski, VT, USA) after treatment with the detection substrate provided in the kit system.

c. LXR Binding Assay

The bindings were performed using the LanthaScreen TR-FRET LXRa/0 Coactivator kit (Thermo Fisger Scientific, Inc., Waltham, MA) as described previously (Slominski A T, et. al., (2021) Sci Rep 11(1) 8002). Briefly, TR-FRET ratios were calculated by dividing the emission at 520 nm by that at 495 nm using Synergy neo2 (BioTek, Winooski, VT).

d. Molecular Modeling

To predict binding poses of vitamin D3 derivatives: 20S(OH)D₃, 20S,23S(OH)2D₃, 20S,23R(OH)2D3 and 20S,25(OH)2D3 towards various receptors including VDR, RORα and RORγ, liver X receptors (LXRα and LXRβ) and aryl hydrocarbon receptor (AhR), a receptor-based approach (molecular docking) was used. Crystal structures for VDR (PDBID: 1DB1) (Rochel N, et. al., (2000) Molecular cell 5(1) 173-179), RORα (PDBID: 1S0X) (Kallen J, et. al., (2004) Journal of Biological Chemistry 279(14) 14033-14038), RORγ (PDBID: 3L0J) (Jin L, et. al., (2010) Molecular Endocrinology 24(5) 923-929), LXRα (PDBID: 5AVI) (Matsui Y, et. al., (2015) Bioorganic & medicinal chemistry letters 25(18) 3914-3920) and LXRβ (PDBID: 5HJP) (Stachel S J, et. al., (2016) Journal of Medicinal Chemistry 59(7) 3489-3498), and a human AhR homology model from a previous study (Slominski A T, et. al., (2018) Int J Mol Sci 19(10)) were used for the docking studies. Openbabel (O'Boyle N M, et. al., (2011) Journal of Cheminformatics 3(1) 33) software was used to convert the ligands of vitamin D₃ derivatives in PDB format to PDBQT format. Receptors were loaded into AutoDock Tools (Trott O, et. al., (2010) Journal of Computational Chemistry 31(2) 455-461) software for preparation: polar hydrogen atoms were added; water molecules, ion molecules, and bound ATP were removed; and lastly, Kollman Charges were added. Prepared receptors were output in PDBQT format for molecular docking in Autodock Vina (Trott O, et. al., (2010) Journal of Computational Chemistry 31(2) 455-461). To evaluate the receptor-ligand interaction, 3D and 2D interactions between vitamin D₃ derivatives with each studied receptor were performed using PyMol (Schrodinger, LLC, The PyMOL Molecular Graphics System, Version 1.8, 2015) software for 3D interactions and Maestro (Maestro, Schrödinger, LLC, New York, NY, 2020) for 2D interaction map.

### 4. Cell Culture Studies

### A. Proliferation

Human dermal fibroblasts isolated from the skin of black neonates (HDFn) and human epidermal (HaCaT) keratinocytes cultured as described previously (Slominski A, et. al., (2013) The Journal of Clinical Endocrinology and Metabolism 98(2) E298-303; Slominski A T, et. al., (2011) Am J Physiol Cell Physiol 300(3) C526-41) were used for the experiments. Briefly, HDFn were plated onto 96-well plates at a confluence of about 70% in their seventh passage. Both HDFn and HaCaT keratinocytes were treated with 20S(OH)D₃, 20S,25(OH)2D₃, 20S,23S(OH)2D₃, 20S,23R(OH)2D₃ or 1α,25(OH)2D₃ (Sigma-Aldrich, St. Louis, MO, USA) dissolved in ethanol and diluted in DMEM containing charcoal-treated serum, as previously described (Janjetovic Z, et. al., (2021) Endocrinology 162(1); Chaiprasongsuk A, et. al., (2020) Free Radic Biol Med 155 87-98; Slominski A T, et. al., (2011) Am J Physiol Cell Physiol 300(3) (2011) C526-41). The vehicle control comprised 0.1% ethanol. Proliferation was estimated using MTS (Promega, Madison, WI) assay. The number of viable cells was measured in 6 replicates.

b. Gene Expression

RNA isolation and reverse transcription were performed as described previously (Slominski A T, et. al., (2021) Sci Rep 11(1) 8002). Quantitative RT-PCR was carried out using Sybr green Master Mix (Thermo Scientific, Waltham, MA), in triplicates as previously described (Slominski A T, et. al., (2021) Sci Rep 11(1) 8002; Janjetovic Z, et. al., (2014) J Pineal Res 57(1) 90-102). Human epidermal keratinocytes (HEKn) isolated from foreskin of white neonates were cultured until the third passage before treatment as described in the literature (Chaiprasongsuk A, et. al., (2019) Redox Biol 24 101206; Janjetovic Z, et. al., (2009) PloS one 4(6) e5988). Cells were treated with vitamin D₃ derivatives for 8 h or 24 h and harvested for RNA isolation. Approximately 300 ng of RNA was used for cDNA synthesis and an equal amount of cDNA was used for PCR reactions. Cyclophilin B or GAPDH was used as an internal control. Primers sequences are listed in (Chaiprasongsuk A, et. al., (2020) Free Radic Biol Med 155 87-9845, Slominski A T, et. al., (2017) Sci Rep 7(1) 11434). Reactions were done in triplicate.

c. Protein Expression

HEKn were cultured until the third passage before treatment. Keratinocytes were plated in a 96-well plate and at 80% confluence, 10-7 M secosteroids (or 0.1% ethanol control) were added and cells were incubated for 48 h. Cells were fixed and stained with anti-involucrin (1:20, GTX-14504; Genetex, Irvine, CA, USA) anti-CK10 (1:300, Santa Cruz Biotechnology), anti-catalase (1:200, Santa Cruz Biotechnology) or anti-CK14 antibody with Alexa Fluor® 488 (1:500, Santa Cruz Biotechnology) and imaged at 10× magnification in Cytation 5 reader as described before (Chaiprasongsuk A, et. al., (2020) Free Radic Biol Med 155 87-9845; Slominski A T, et. al., (2017) Sci Rep 7(1) 11434; Chaiprasongsuk A, et. al., (2020) Int J Mol Sci 21(24)). Nuclei were stained with blue DAPI. Fluorescence intensity was measured using the Cytation 5 reader and ImageJ, and data were analyzed using Graph Pad Prizm.

d. Human and T Cell Activation and Analysis

Human peripheral blood mononuclear cells (PBMC) from two healthy individuals were obtained from a pre-existing respository. Their use was approved by the University of Alabama at Birmingham Institutional Review Board (Human Subject Assurance Number FWA00005960) as an exempt protocol #4 (Dr C. Raman, P.I.). The protocol was classified for exempt status under 45CFR46.102 (f) in that is does not involve “human subjects” as defined therein. Informed consent is waived in accord with 45CFR46.116 (d). In 48 well plates, 0.5×106 PBMC/well were cultured in RPMI1640 containing, 5% charcoal adsorbed FBS and antibiotics and T cells were activated using 0.5 g/ml anti-CD3 (clone 145-2C11) and 1 μg/ml anti-CD28 (clone 37.51) for 24 h (Janjetovic Z, et. al., (2021) Endocrinology 162(1), Chaiprasongsuk A, et. al., (2020) Free Radic Biol Med 155 87-98, Slominski A T, et. al., (2011) Am J Physiol Cell Physiol 300(3) C526-41, McGuire D J, et. al., (2014) Eur J Immunol 44(4) 1137-42, Sestero C M, et. al., (2012) J Immunol 189(6) 2918-30). The culture was treated with 10-7 M 20S(OH)D₃, 20S,25(OH)2D₃, 20S,23S(OH)2D₃, 20S,23R(OH)2D₃ or 1α,25(OH)2D₃ (Sigma-Aldrich, St. Louis, MO, USA) dissolved in ethanol and diluted in culture medium at the same time of addition of anti-CD3 and anti-CD28. The vehicle control comprised of 0.1% ethanol. At the end of assay the cells were stained with antibodies to CD4 (clone RPA-T4, BV-421), CD19 (clone HB19, BV-510), CD14 (clone HCD14, Alexa 700), CD137 (clone 4B4-1, BV605), CD8a (clone RPA-T8, FITC), ICOS (clone C398.4A, PE/Dazzle), PD1 (clone NAT105, PE), CD80 (clone 2D10, PerCP/Cy5.5), OX40 (clone ACT35, PE/Cy7), CD69 (clone FN50, APC), HLA-DR (clone L243, APC/Fire 750). All antibodies were from Biolegend except anti-CD8 was from Thermo Fisher. The stained cells were analyzed using a CytoFLEX (Beckman Coulter) flow cytometer.

For unbiased analysis of the multidimensional data, acomputational algorithm-based approaches was utilized (Quintelier K, et. al., (2021) Nature Protocols 16(8) 3775-3801). The flow cytometry standard (FCS) data files from each individual and treatment (two individuals, six treatments including ethanol, a total of 12 data files) were processed through the following workflow—compensation, gating on live cells, gating for single cells and subtraction of CD19+(B cells) and CD14+(monocytes). B cells and monocytes were removed from the analysis because only T cells (CD4+ and CD8+) are activated by anti-CD3 and anti-CD28. After these initial gating steps, a key word was added to each of the 12 FCS files, down sampled to 50,000 events/sample and concatenated. Down sampling ascertains that each sample is equally weighted, and concatenation ascertains that statistical analysis is applied equally. The concatenated files were first subjected to TriMAP analysis, a computational algorithm that clusters populations based on triplet relatedness followed by FlowSOM algorithm-based clustering (Quintelier K, et. al., (2021) Nature Protocols 16(8) 3775-3801). To perform these analyses, plugins within FlowJo (BD, CA, USA) were used.

### 5. Results

### A. Chemical Synthesis and Confirmation of the Structures

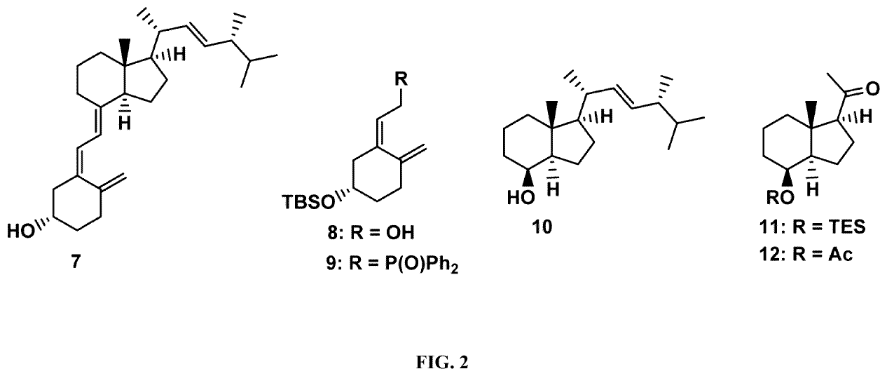

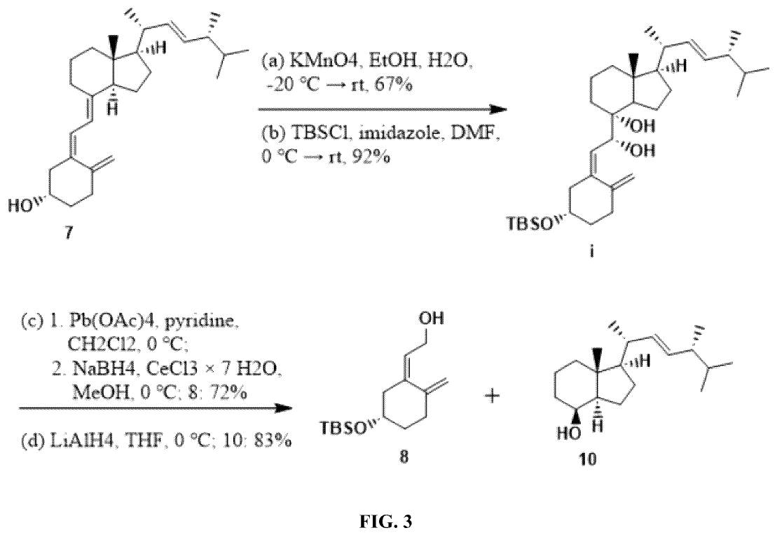

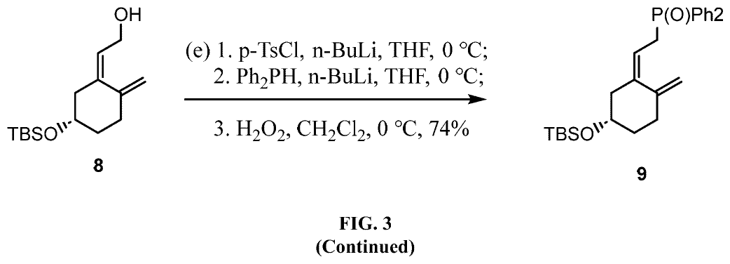

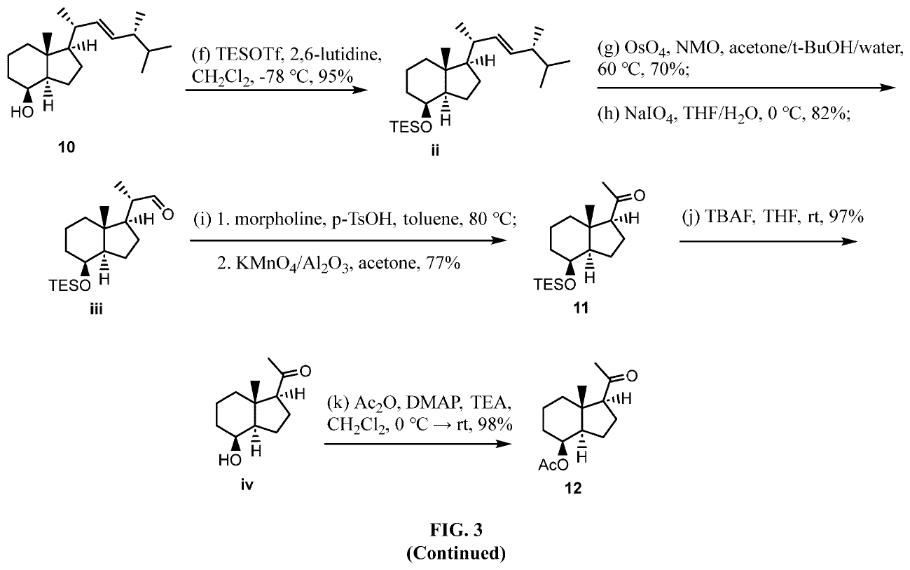

An efficient synthetic route for vitamin D₃ derivatives 20S(OH)D₃ and its 23- as well as 25-hydroxylated analogs 20S,23S(OH)₂D₃, 20S,23R(OH)₂D₃, 20S,25(OH)₂D₃ were designed. Convergent synthesis was used for the preparation of these target compounds where the key stage was a Wittig-Horner coupling of the corresponding Grundmann ketone (CD-ring fragment) with A-ring phosphine oxide. Starting from vitamin D₂ (7) (FIG. 2), the required building blocks 8-12 were prepared using a slight modification of literature procedures; the synthetic steps (FIG. 3).

The ketone 12 was treated with two different Grignard reagents (Li W, et. al., (2010) Steroids 75(12) 926-35, Sibilska-Kaminski I K, et. al., (2020) J Med Chem 63(13) 7355-7368) providing diastereomerically pure 20S-diols 13 and 14 (FIG. 4). Secondary hydroxyl groups in 13 and 14 were efficiently oxidized to the corresponding ketones 15 and 16 with tetrapropylammonium perruthenate in the presence of N-methylmorpholine-N-oxide. Tertiary hydroxyls were then protected as TMS ethers providing CD-ring building blocks 17 and 18. These hydrindanones were then coupled with the known A-ring phosphine oxide 9 (Wang Q, et. al., (2015) Steroids 104 153-62) to furnish protected vitamin D₃ derivatives 19 and 20. Their deprotection followed by RP-HPLC purification provided the target 20-hydroxy compounds 3 and 6 in good yields and high purity.

Methyl ketone 11 was then used for the preparation of desired 20S,23S(OH)₂D₃ as well as its epimer 20S,23R(OH)₂D₃. As a consequence of the steric effect of both 7a′-methyl group and 4′-OTES substituents, only the 3S-epimer 21 was isolated when the ketone 11 was treated with the nitrile anion, generated from acetonitrile and LDA. The analogous, highly stereoselective addition of Grignard reagents has been thoroughly studied by us previously (Li W, et. al., (2010) Steroids 75(12) 926-35, Sibilska-Kaminski I K, et. al., (2020) J Med Chem 63(13) 7355-7368). The secondary hydroxyl group in 21 was deprotected and the resulting hydrindanol 22 was subjected to the oxidation with TPAP/NMO leading to the ketone 23. Protection of the tertiary hydroxyl group in this compound by its treatment with trimethylsilyl triflate provided the desired building block 24. Wittig-Horner olefination was used as a coupling reaction of the hydrindanone 24 and phosphine oxide 9. The formed nitrile 25, possessing the vitamin D skeleton, was reduced with diisobutylaluminum hydride to the corresponding aldehyde 26. In this compound elongation of the side chain was effected by its Grignard reaction with i-butyl magnesium chloride providing a mixture of diastereomeric alcohols 27a, b. Finally, removal of silyl protecting groups and efficient separation by high-performance liquid chromatography (HPLC) provided the target epimeric (20S,23S)- and (20S,23R)-dihydroxylated vitamin D₃ compounds (4a, b).

Without wishing to be bound by theory, it is believed that the above scheme represents a significant improvement to the synthetic route for the preparation of 20S(OH)D₃ compared to the method described by Li et al (Wang Q, et. al., (2015) Steroids 104 153-62). The advantage of the synthesis reported here is the application of a different protecting group of the tertiary hydroxyl at C-20. Thus, the use of trimethylsilyl ether (OTMS), being significantly more labile than EOM ether, allowed for the final vitamin D compound to be obtained with significantly increased (ca. fourfold) overall yield. Moreover, using analogous conditions, 20S,25(OH)₂D₃ has also been successfully prepared, representing another active metabolite of vitamin D₃, that so far has only been obtained enzymatically (Tang E K, et. al., (2013) Drug metabolism and disposition: the biological fate of chemicals 41(5) 1112-24).

The synthesis of 23-hydroxylated derivatives presented above has also been significantly improved in comparison with the previously reported synthetic path (Lin Z, et. al., (2016) J Med Chem 59(10) 5102-8) involving a series of linear transformations. The epimeric 20,23-dihydroxyvitamin D₃ metabolites were obtained in high yield, easily separated by HPLC and individual compounds were biologically evaluated. The synthetic strategy applied gave a marked (ca. 5 times) increase in the overall yield of the target secosteroids, avoiding the irradiation step used in past routes which limits the scale of the conversion of the 7-dehydrocholesterol analog to the corresponding secosteroid. Moreover, almost all steps are executed on the optically pure compounds; formation of C-23 stereoisomers takes place at the final stage of the synthesis.

b. Hydroxylation at C1A

All four chemically synthesized secosteroids were converted to their expected 1α-hydroxy derivatives by CYP27B1, and identified on the basis of previous studies on the enzymatically and/or chemically derived substrates (Li W, et. al., (2010) Steroids 75(12) 926-35, Tang E K, et. al., (2013) Drug metabolism and disposition: the biological fate of chemicals 41(5) 1112-24, Lin Z, et. al., (2016) J Med Chem 59(10) 5102-8, Tang E K, et. al., (2010) Drug metabolism and disposition: the biological fate of chemicals 38(9) 1553-9). The metabolism of chemically synthesized secosteroids by CYP27B1 are shown in Table 2. Secosteroids were incorporated into phospholipid vesicles at a ratio of 0.025 mol secosteroid/mol phospholipid and incubated with mouse CYP27B1 (0.2 μM) for 20 min for all substrates except 20S,25(OH)₂D₃ which was incubated for 5 min. Extracted samples were analyzed by reverse-phase HPLC. Data are mean±SD, n=3. The rates varied dramatically with a high rate of 1α-hydroxylation being observed for 20S,25(OH)₂D₃, consistent with previous reports for this substrate produced enzymatically (Tang E K, et. al., (2013) Drug metabolism and disposition: the biological fate of chemicals 41(5) 1112-24). The naturally occurring diastereomer of 20,23(OH)₂D₃, the 20S,23S-isomer, was metabolized approximately twice as fast as the non-natural 20S,23R-isomer, as noted in a previous study (Lin Z, et. al., (2016) J Med Chem 59(10) 5102-8). 20S(OH)D₃ was a relatively poorer substrate for CYP27B1 compared to 20S,25(OH)₂D₃, also consistent with a previous report (Tang E K, et. al., (2013) Drug metabolism and disposition: the biological fate of chemicals 41(5) 1112-24). In summary, the efficiency of metabolism of D₃ derivatives by CYP27B1 is as follows: 20S,25(OH)₂D₃>>20S,23S(OH)₂D₃>20S(OH)D₃=20S,23R(OH)₂D₃.

TABLE 2

| | 1α-hydroxylase activity |
|---|---|
| Secosteroid | (mol product/min/mol CYP27B1) |
| 20S(OH)D3 | 0.135 ± 0.005 |
| 20S,23R(OH)2D3 | 0.117 ± 0.027 |
| 20S,23S(OH)2D3 | 0.258 ± 0.020 |
| 20S,25(OH)2D3 | 7.98 ± 0.24 |

c. Regulation of Cell Proliferation and Differentiation Program

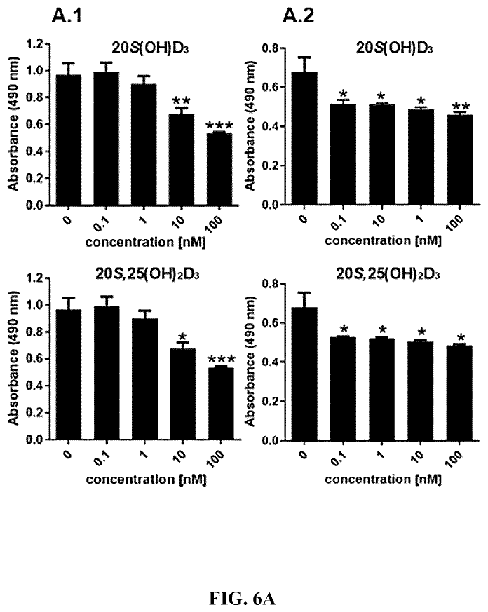

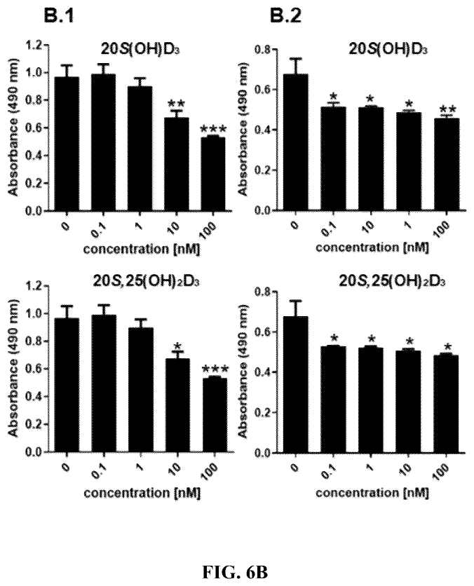

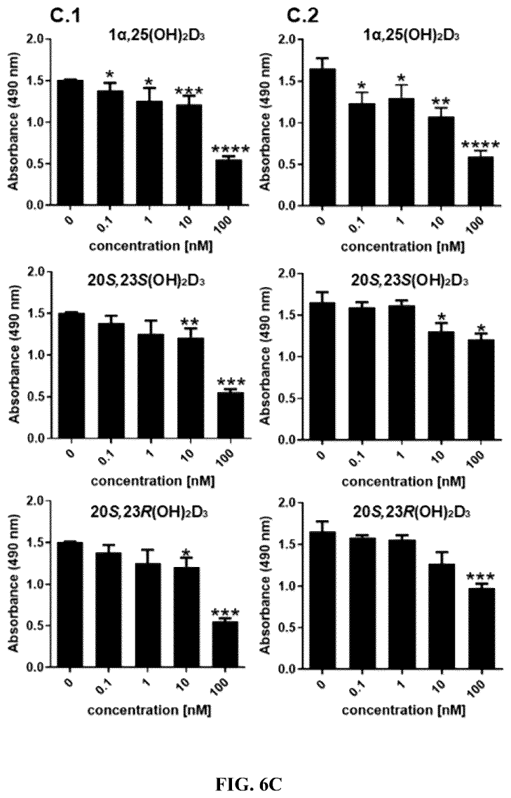

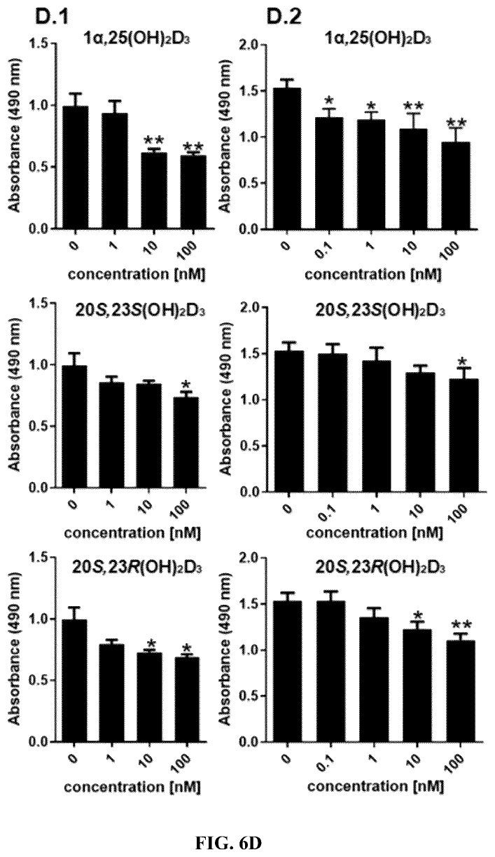

The anti-proliferative properties of 20S(OH)D₃ and 20S,23(OH)₂D₃ (anjetovic Z, et. al., (2021) Endocrinology 162(1), Slominski A T, et. al., (2014) J Steroid Biochem Mol Biol 144PA 28-39, Janjetovic Z, et. al., (2010) Journal of cellular physiology 223(1) 36-48, Slominski A, et. al., (2013) The Journal of clinical endocrinology and metabolism 98(2) E298-303, Podgorska E, et. al., (2021) Cancers (Basel) 13(13), Skobowiat C, et. al., (2017) Oncotarget 8(6) 9823-9834, Chen J, et. al., (2014) Anticancer Res 34(5) 2153-63, Zbytek B, et. al., (2008) J Invest Dermatol 128(9) 2271-80, Janjetovic Z, et. al., (2009) PloS one 4(6) e5988, Slominski A T, et. al., (2012) Anticancer Res 32(9) 3733-42, Brozyna A A, et. al., (2020) Nutrients 12(11)) and to some degree of 20S,25(OH)₂D₃(Tieu E W, et. al., (2012) Biochem Pharmacol 84(12) 1696-704, Slominski A T, et. al., (2017) J Steroid Biochem Mol Biol 173 42-56) are well established. The capabilities of the newly synthesized secosteroids to inhibit proliferation were tested and found that 20S,25(OH)₂D₃ inhibits fibroblast and keratinocyte proliferation in similar manner to its precursor, 20S(OH)D₃ (FIG. 6A-D). Similar effects were also observed for both 20S,23(OH)₂D₃ isomers (FIG. 6A-D), which is consistent with previously reported data (Lin Z, et. al., (2016) J Med Chem 59(10) 5102-8).

Referring to FIG. 6A-D, inhibition of cell proliferation by 20S(OH)D₃, 20S,25(OH)₂D₃, 20S,23S(OH)₂D₃ and 20S,23R(OH)₂D₃ in comparison to 1α,25(OH)₂D₃ is represented. Human epidermal (HaCaT) keratinocytes (FIG. 6A and FIG. 6C) or dermal fibroblasts (FIG. 6B and FIG. 6D) were treated for 24 h (1) or 48 h (2) with graded concentrations of the compounds: 0.1, 1, 10, or 100 nM. Ethanol (0.1%) served as a negative control. Data are means±SD (n=6) and were analyzed using Student's t-test with p<0.05 (*), p<0.01 (**) or p<0.001 (***).

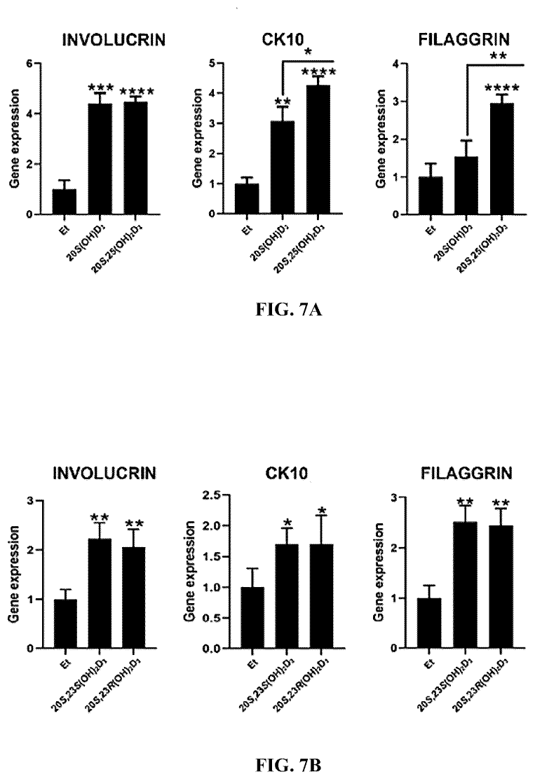

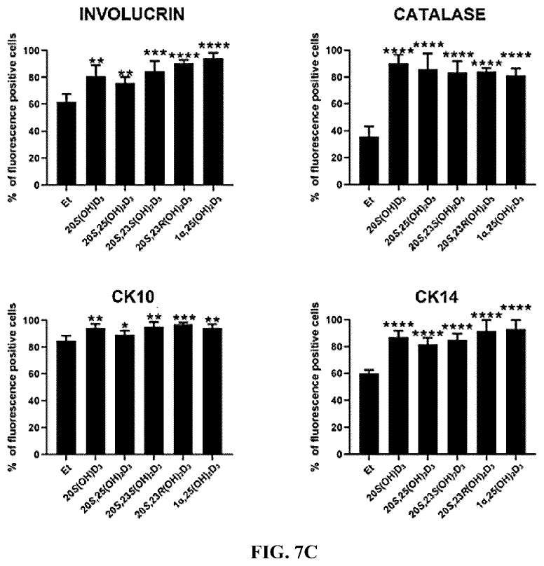

Again, analysis of human keratinocytes showed that 20S,25(OH)₂D₃ was slightly better at inducing gene expression of involucrin, CK10, and filaggrin, indicative of keratinocyte differentiation, compared to 20S(OH)D₃ (FIG. 7A) with 20S,23R(OH)₂D₃ and 20S,25(OH)₂D₃ showing similar effects on the expression of these genes (FIG. 7B). At the protein level, all compounds including 20S(OH)D₃, 20S,23S(OH)₂D₃, 20S,23R(OH)₂D₃ and 20S,25(OH)₂D₃ showed similar net biological effects in increasing the levels of involucrin, CK10, CK14, and catalase (FIG. 7C). These results are consistent with previously published pro-differentiation properties of 20S(OH)D₃ and 20S,23S(OH)₂D₃(Slominski A T, et. al., (2010) PloS one 5(3) e9907, Chaiprasongsuk A, et. al., (2020) Free Radic Biol Med 155 87-98, Slominski A T, et. al., (2014) J Steroid Biochem Mol Biol 144PA 28-39, Janjetovic Z, et. al., (2010) Journal of cellular physiology 223(1) 36-48, Zbytek B, et. al., (2008) J Invest Dermatol 128(9) 2271-80, Janjetovic Z, et. al., (2009) PloS one 4(6) e5988, Slominski A T, et. al., (2015) J Steroid Biochem Mol Biol 148 52-63, which now extend to include 20S,25(OH)₂D₃ and 20S,23R(OH)₂D₃.

Referring to FIG. 7A-D, stimulation of keratinocyte differentiation by 20S(OH)D₃, 20S,25(OH)₂D₃, 20S,23S(OH)₂D₃ and 20S,23R(OH)₂D₃ in comparison to 1α,25(OH)₂D₃. Human neonatal epidermal keratinocytes were treated for 8 (FIG. 7A), 24 h (FIG. 7B) or 48 h (FIG. 7C and FIG. 7D) with 0.1, 1, 10, or 100 nM secosteroid or 0.1% ethanol (negative control). FIG. 7A and FIG. 7B represent relative gene expression studies with cyclophilin B used as internal control and presented as fold change vs vehicle control.

FIG. 7C and FIG. 7D represent protein expression studies. Values are presented as percentage of fluorescence positive cells (FIG. 7C) or as mean fluorescence per cell (% of all cells (FIG. 7D). The values in FIG. 7D were calculated as mean green fluorescence vs DAPI blue fluorescence measured in ImageJ. Data are means±SD (n=3) and were analyzed using Student's t-test with p<0.05 (*), p<0.01 (**) or p<0.001 (***).

d. Nuclear Receptor Signaling

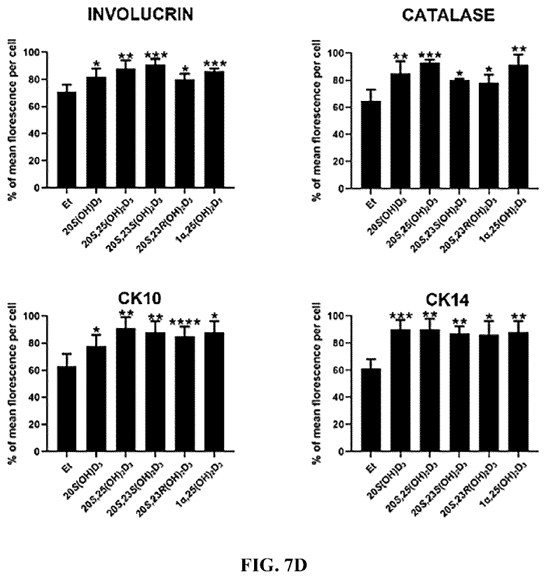

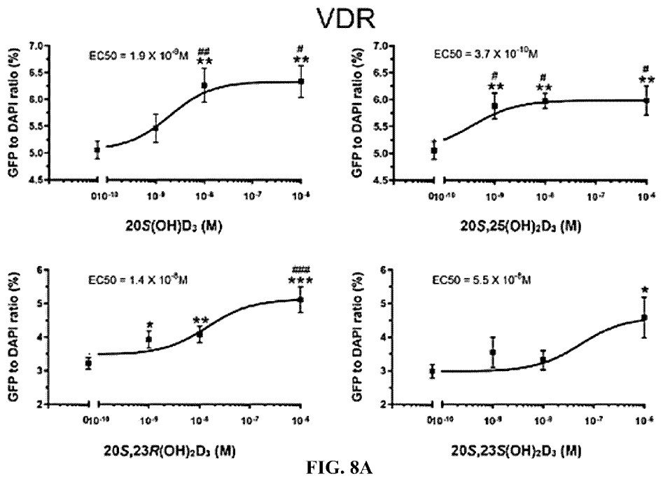

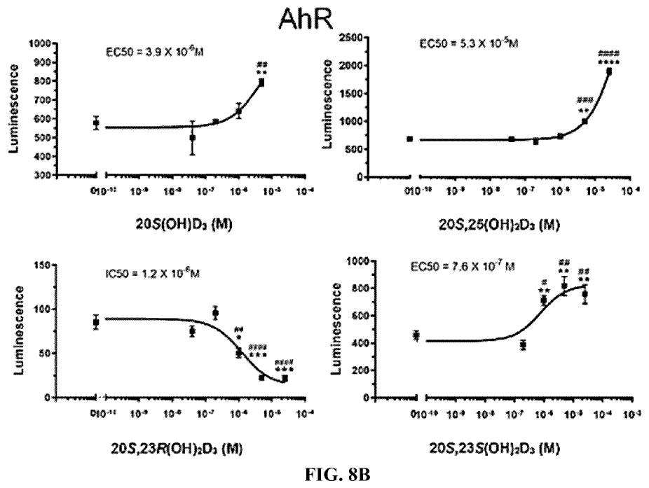

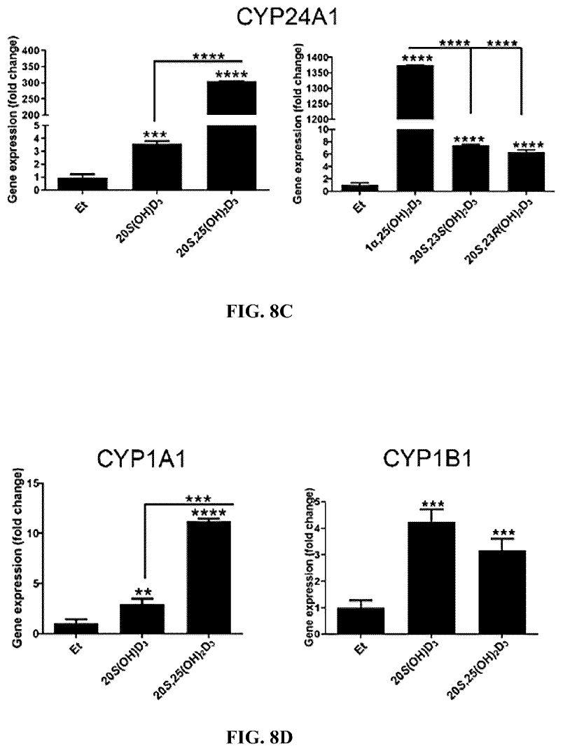

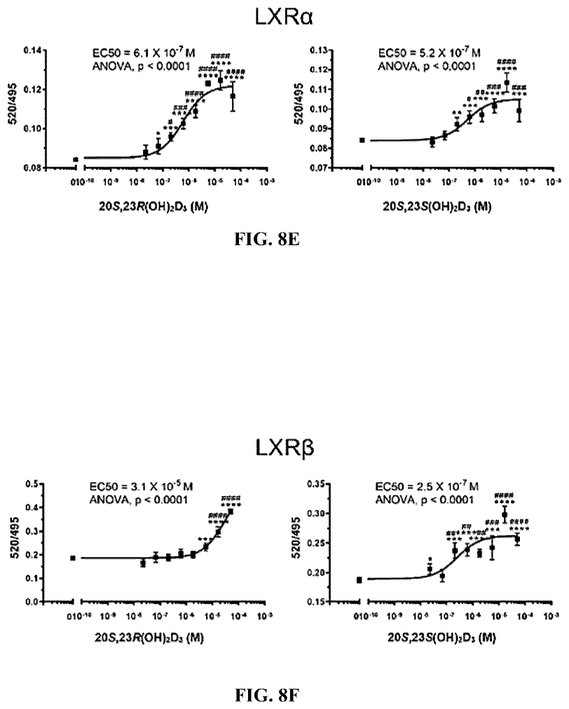

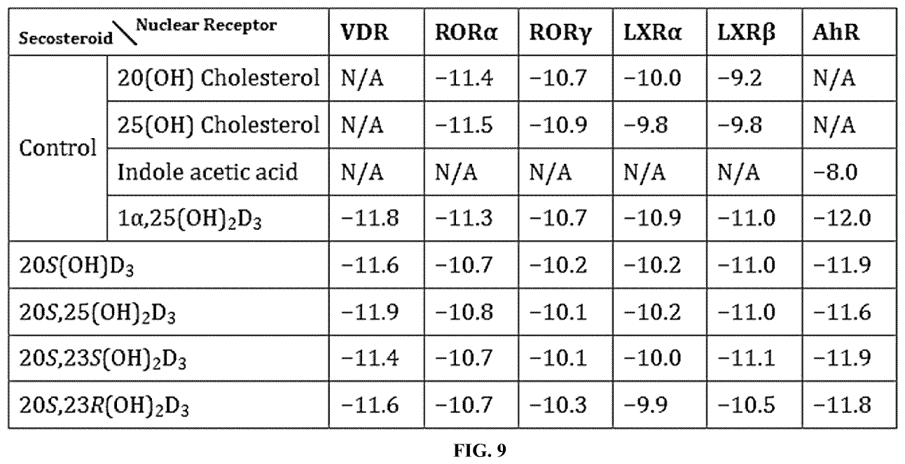

To better define the properties of the newly synthesized secosteroids, their activities on the VDR, AhR and LXR were tested by performing functional assays which were further validated by qPCR of downstream enzymes (FIGS. 8A-F) and molecular modeling (FIG. 9-13). Binding affinities of top-scored docking complexes of vitamin D₃ hydroxy derivatives with VDR, AhR, LXRs (LXRα, LXRβ) and RORs (RORα, RORγ) from molecular docking are presented in FIG. 9, known ligands for each receptor were used as controls. 20S(OH)D₃, 20S,25(OH)₂D₃, 20S,23S(OH)₂D₃ and 20S,23R(OH)₂D₃ have favorable energies for binding to the ligand binding domain of VDR, AhR, LXRs (LXRα, LXRβ) and RORs (RORα, RORγ), and are comparable to the binding energies of known ligands for each receptor, including 1α,25(OH)₂D₃.

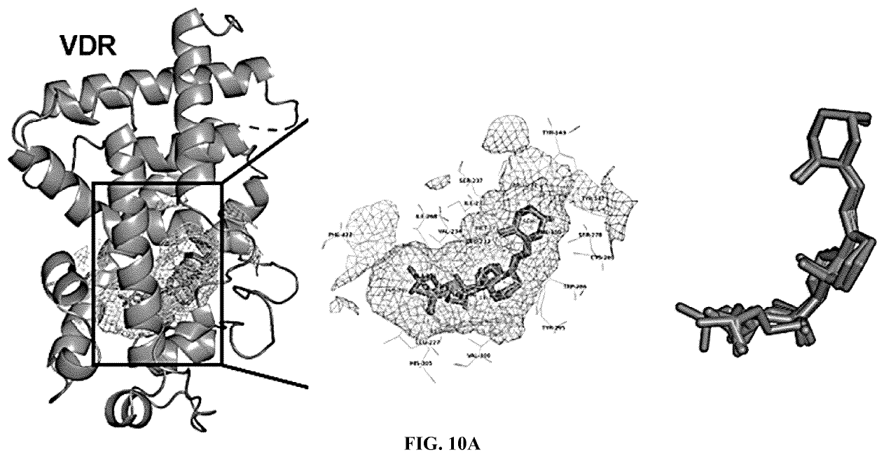

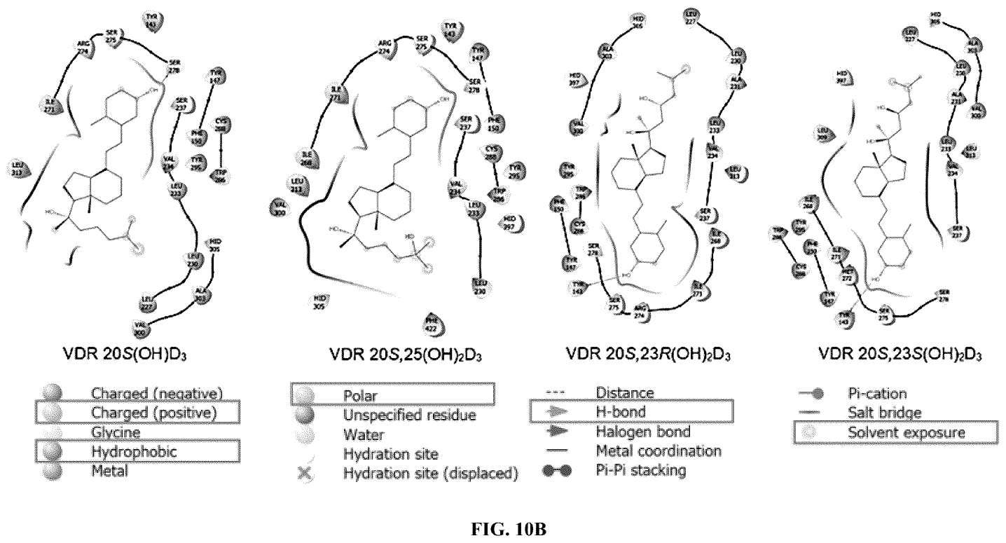

All of the newly synthesized compounds induced VDR translocation to the nucleus (FIG. 8A) in a manner similar to that reported previously for some of these secosteroids (Kim T K, et. al., (2012) Mol Cell Endocrinol 361(1-2) 143-52, Slominski A T, et. al., (2018) J Steroid Biochem Mol Biol 177 159-170). CYP24A1 is a downstream target of the activated VDR and it was observed that 20S,25(OH)₂D₃ caused much greater stimulation of CYP24A1 expression than 20S(OH)D₃. There was no significant difference in the expression of CYP24A1 caused by 20S,23S(OH)₂D₃ and 20S,23R(OH)₂D₃ but both of these caused significantly less expression than 1α,25(OH)₂D₃(FIG. 8C). The results of these assays are supported by molecular docking results of these secosteroids with the VDR (FIG. 9 and FIG. 10). 20S(OH)D₃, 20S,25(OH)₂D₃, 20S,23S(OH)₂D₃ and 20S,23R(OH)₂D₃ all have favorable to binding energies for interaction with VDR (FIG. 9) and bind to the same LBD of VDR (FIG. 10).

Referring to FIG. 10A and FIG. 10B, shows the binding pattern of selected vitamin D₃ derivatives with VDR. FIG. 10A shows 3D binding modes for 20S(OH)D₃, 20S,25(OH)₂D₃, 20S,23S(OH)₂D₃ and 20S,23R(OH)₂D₃ in ligand binding domain of VDR (cartoon in gray) include the zoomed view. The binding pocket shown as light blue meshing area. FIG. 10B shows the 2D interaction map of 20S(OH)D₃, 20S,25(OH)₂D₃, 20S,23S(OH)₂D₃ and 20S,23R(OH)₂D₃ with VDR (image generated with Maestro (v12.4)).

In summary, these data and analyses are consistent with previous studies on the VDR, which showed that 20S(OH)D₃ and 20S,23(OH)₂D₃ act on the genomic site of the VDR with lower efficiency than their 1α-hydroxy derivatives, as a consequence of their different interactions with amino acids of the LBD (Slominski A T, et. al., (2014) J Steroid Biochem Mol Biol 144PA 28-39, Kim T K, et. al., (2012) Mol Cell Endocrinol 361(1-2) 143-52, Lin Z, et. al., (2018) Sci Rep 8(1) 1478, Lin Z, et. al., (2016) J Med Chem 59(10) 5102-8, Slominski A T, et. al., (2018) J Steroid Biochem Mol Biol 177 159-170). The better efficiency of 20S,25(OH)₂D₃ in stimulating the expression of CYP24A1 in comparison to other secosteroids could be secondary to its very efficient hydroxylation by CYP27B1 (Table 2) (Tang E K, et. al., (2013) Drug metabolism and disposition: the biological fate of chemicals 41(5) 1112-24).

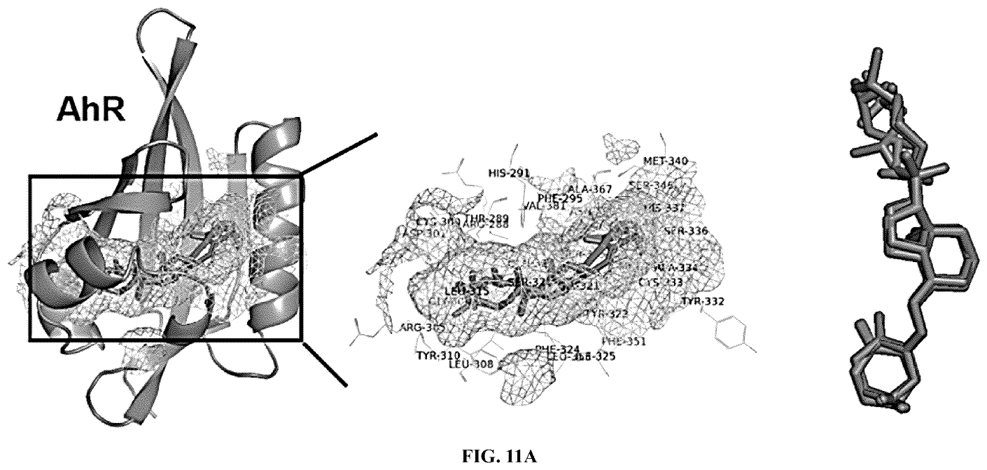

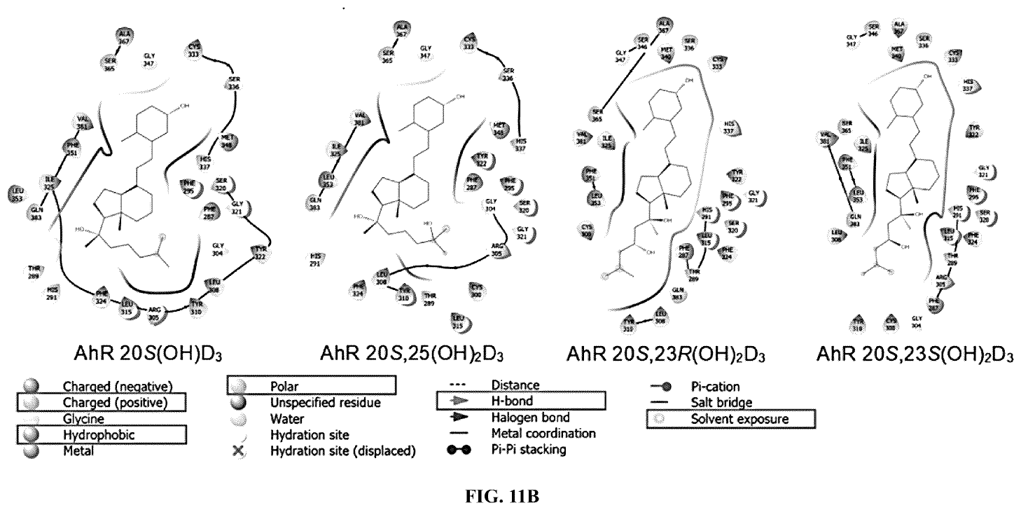

Newly-synthesized secosteroids were found to interact with the AhR (FIG. 9, FIG. 10B, and FIG. 10D), similar to a previous study (Slominski A T, et. al., (2018) Int J Mol Sci 19(10)) except that 20S,23R(OH)₂D₃ was found to act as a reverse agonist. Molecular modeling predicts that 20S(OH)D₃, 20S,25(OH)₂D₃, 20S,23S(OH)₂D₃ and 20S,23R(OH)₂D₃ share the same binding region in AhR. 20S(OH)D₃ and 20S,25(OH)₂D₃ have the similar binding postures with AhR, while the binding postures of 20S,23R(OH)₂D₃ and 20S,23S(OH)₂D₃ with AhR are very similar (FIG. 11A and FIG. 11B).

The binding between AhR and D₃ derivatives is mainly through hydrophobic interactions. Hydrophobic residues Cys333, Phe295, Phe287, Tyr322, Leu308, Tyr310, Leu315, Leu353, Ile325, Val381 and Ala367 in AhR are directly involved in the binding with all four molecules of 20S(OH)D₃, 20S,25(OH)₂D₃, 20S,23S(OH)₂D₃ and 20S,23R(OH)₂D₃(FIG. 11B).

Referring to FIG. 11A and FIG. 11B, the binding pattern of selected vitamin D₃ derivatives with AhR is shown. FIG. 11A shows 3D binding modes for 20S(OH)D₃, 20S,25(OH)₂D₃, 20S,23S(OH)₂D₃ and 20S,23R(OH)₂D₃ in ligand binding domain of VDR (cartoon in gray) include the zoomed view. The binding pocket shown as light blue meshing area. FIG. 11B shows the 2D interaction map of 20S(OH)D₃, 20S,25(OH)₂D₃, 20S,23S(OH)₂D₃ and 20S,23R(OH)₂D₃ with VDR (image generated with Maestro (v12.4)).

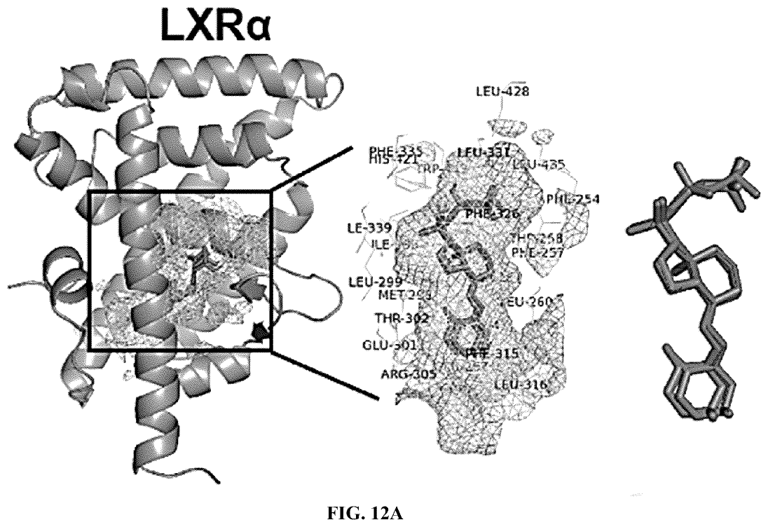

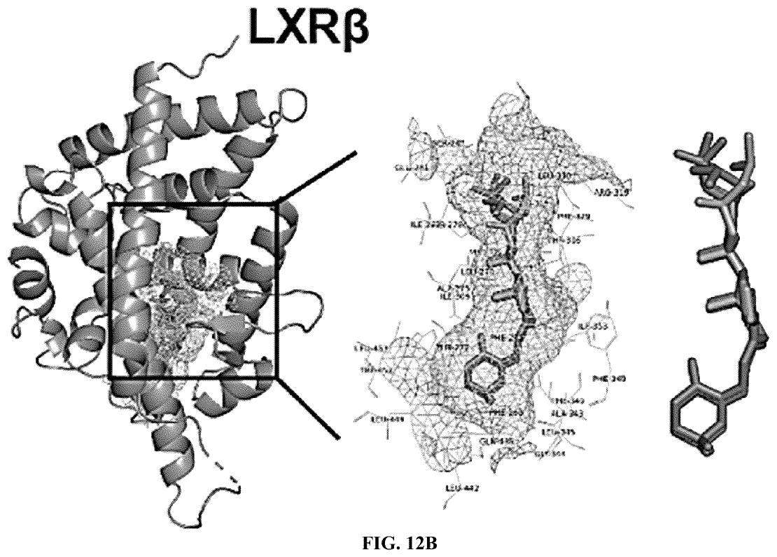

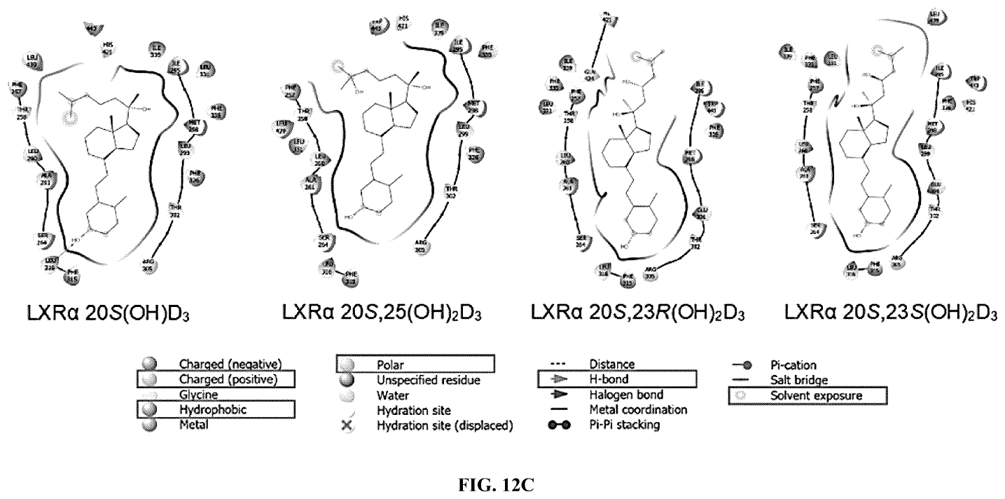

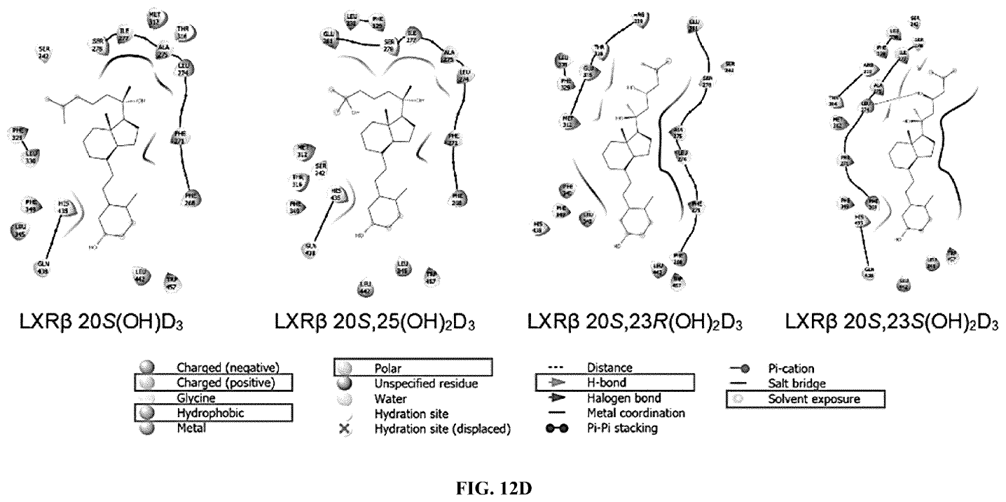

Recently it was reported that 20S(OH)D₃, 20S,23(OH)₂D₃, 20S,25(OH)₂D₃ and other D₃-hydroxy derivatives can act as ligands on LXR (Slominski A T, et. al., (2021) Sci Rep 11(1) 8002). Therefore, interactions of 20S,23S(OH)₂D₃ versus 20S,23R(OH)₂D₃ with the LBD of LXRα and β (FIG. 8E and FIG. 8F) were compared using a functional assay and molecular modeling (FIG. 9 and FIG. 12). The functional assays using LanthaScreen TR-FRET LXRα and β coactivator assays has confirmed that 20S,23(OH)₂D₃ isomers are agonists on the LXRα and β (Slominski A T, et. al., (2021) Sci Rep 11(1) 8002), with some differences (FIG. 8E and FIG. 8F). Vitamin D₃ derivatives had better affinity on LXRβ than on LXRα. When binding with LXRs, 20S(OH)D₃, 20S,25(OH)₂D₃, 20S,23S(OH)₂D₃ and 20S,23R(OH)₂D₃ all bind to the same binding pocket of either LXRα or LXRβ (FIG. 12A and FIG. 12B). For LXRα, 20S(OH)D₃ and 20S,25(OH)₂D₃ have the similar binding postures, whereas 20S,23S(OH)₂D₃ and 20S,23R(OH)₂D₃ have the similar binding posture but twisted from C20 compared to that of 20S(OH)D₃ and 20S,25(OH)₂D₃(FIG. 12A and FIG. 12C). For LXRβ, similar observation based on 2D and 3D interaction maps (FIG. 12B and FIG. 12D). 20S,23S(OH)₂D₃ has a better affinity with LXRβ compared to that of 20S,23R(OH)₂D₃(FIG. 9), which is also observed in experimental data (FIG. 8E and FIG. 8F). 20S(OH)D₃, 20S,25(OH)₂D₃, 20S,23S(OH)₂D₃ and 20S,23R(OH)₂D₃ binding postures to LXRα (FIG. 12C) are very similar to their binding postures to LXRβ (FIG. 12D).

The binding between LXRα and D₃ derivatives is mainly through hydrophobic interactions. Hydrophobic residues Ile295, Phe335, Met298, Leu299, Phe315, Leu316, Ala261, Leu260, Phe257 and Le439 in LXRα are directly involved in the binding with all four molecules of 20S(OH)D₃, 20S,25(OH)₂D₃, 20S,23S(OH)₂D₃ and 20S,23R(OH)₂D₃(FIG. 12C). The binding of 20S(OH)D₃ to LXRα involved the formation of a hydrogen bond between the hydroxyl group on C3 of 20S(OH)D₃ with Leu316, while the binding of 20S,23S(OH)₂D₃ with LXRβ involved formation of a hydrogen bond between the 23-hydroxyl group of 20S,23S(OH)₂D₃ and Leu274 (FIG. 12D).

Referring to FIG. 12A-D, a representative data of the binding pattern of selected vitamin D₃ derivatives with LXRs is shown. FIG. 12A shows 3D binding modes for 20S(OH)D₃, 20S,25(OH)₂D₃, 20S,23S(OH)₂D₃ and 20S,23R(OH)₂D₃ in ligand binding domain of LXRα (cartoon in gray). FIG. 12B shows 3D binding modes for 20S(OH)D₃, 20S,25(OH)₂D₃, 20S,23S(OH)₂D₃ and 20S,23R(OH)₂D₃ in ligand binding domain of LXRβ (cartoon in gray) including a zoomed view. The binding pocket is shown as a light blue meshing area. FIG. 12C shows a 2D interaction map of 20S(OH)D₃, 20S,25(OH)₂D₃, 20S,23S(OH)₂D₃ and 20S,23R(OH)₂D₃ with LXRα. FIG. 12D shows a 2D interaction map of 20S(OH)D₃, 20S,25(OH)₂D₃, 20S,23S(OH)₂D₃ and 20S,23R(OH)₂D₃ with LXRβ (image generated with Maestro (v12.4)).

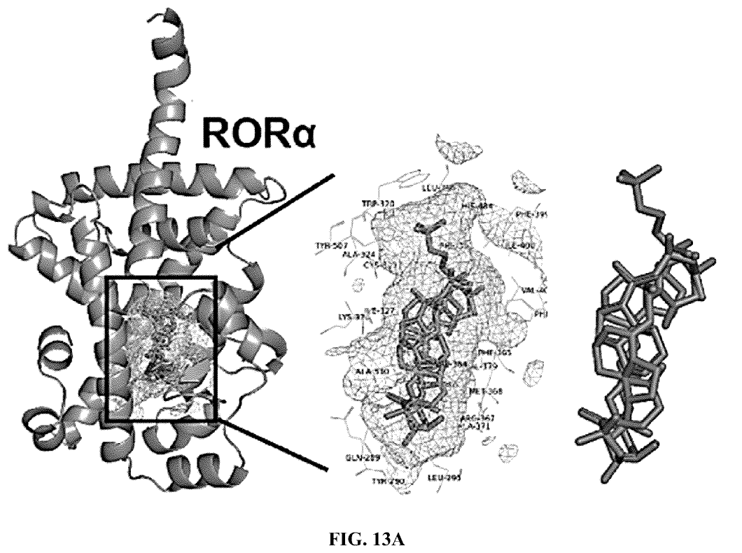

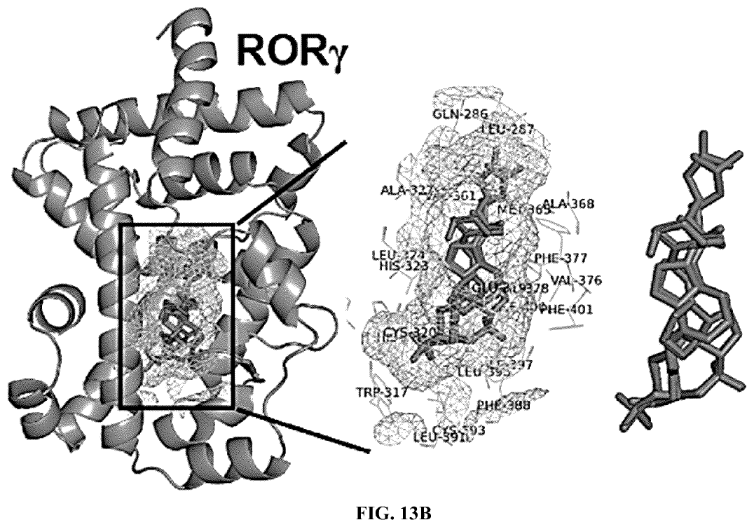

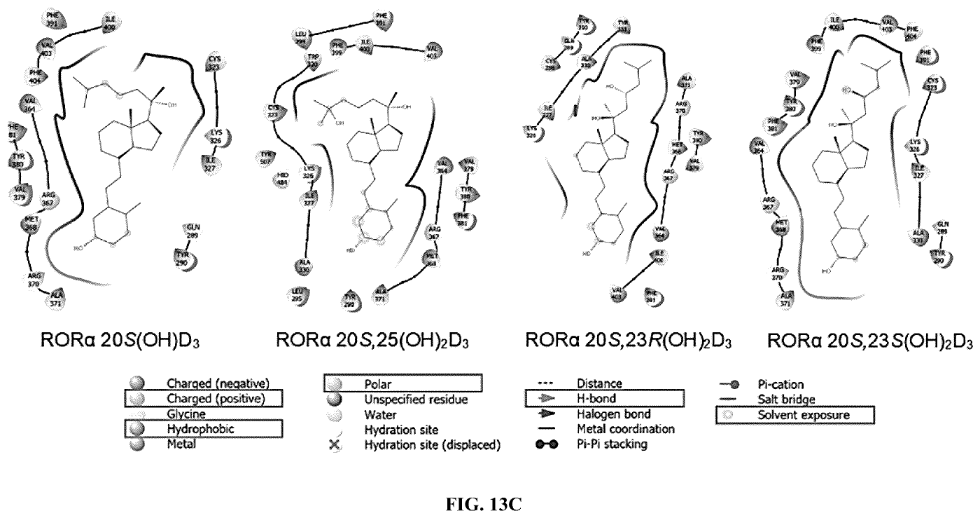

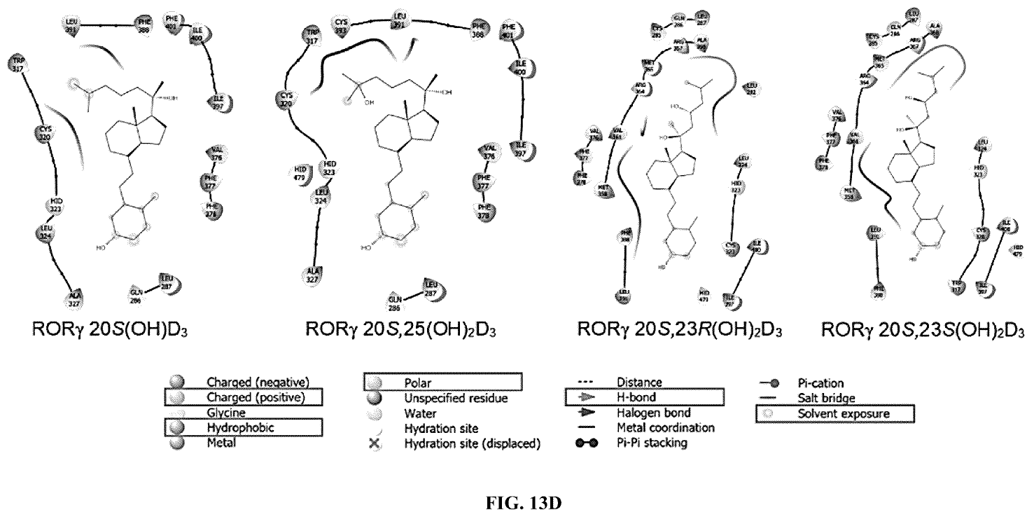

Lastly, molecular modeling of 20S(OH)D₃, 20S,25(OH)₂D₃, 20S,23S(OH)₂D₃ and 20S,23R(OH)₂D₃ with LBD of the RORs (FIG. 9 and FIG. 13) was performed. 20S(OH)D₃, 20S,25(OH)₂D₃, 20S,23S(OH)₂D₃ and 20S,23R(OH)₂D₃ all bind to the same binding pocket of both RORα and RORγ (FIG. 13A and FIG. 13B). For RORα, 20S(OH)D₃ and 20S,25(OH)₂D₃ share a similar binding posture to each other, distinct from 20S,23S(OH)₂D₃ and 20S,23R(OH)₂D₃ which are also similar to each other (FIG. 13C). For RORγ, again 20S(OH)D₃ and 20S,25(OH)₂D₃ show similar binding postures to each other, as do 20S,23R(OH)₂D₃ and 20S,23S(OH)₂D₃(FIG. 13D). The bindings between RORs and D₃ derivatives are mainly mediated through hydrophobic interactions. Hydrophobic residues Ile327, Tyr290, Met368, Val379, Tyr380, Val364, Val403, Phe391 and Ile400 in RORα are directly involved in the binding with all four D₃ derivatives (FIG. 13C). Hydrophobic residues Leu287, Phe378, Phe377, Val376, Ile397, Ile400, Phe388, Leu391, Cys320 and Leu324 in RORγ are directly involved in the binding with all four molecules of 20S(OH)D₃, 20S,25(OH)₂D₃, 20S,23S(OH)₂D₃ and 20S,23R(OH)₂D₃(FIG. 13D). These modeling data not only support previous studies identifying novel hydroxy derivatives of D₃ as inverse agonists on RORs (Slominski A T, et. al., (2017) J Steroid Biochem Mol Biol 173 42-56, Slominski A T, et. al., (2014) FASEB J 28(7) 2775-89), but also extend such observations to include 20S,23R(OH)₂D₃ and 20S,25(OH)₂D₃.

Referring to FIG. 13A-D, representative data illustrating the binding pattern of selected vitamin D₃ derivatives with RORs is shown. FIG. 13A shows 3D binding modes for 20S(OH)D₃, 20S,25(OH)₂D₃, 20S,23S(OH)₂D₃ and 20S,23R(OH)₂D₃ in ligand binding domain of RORα (cartoon in gray). FIG. 13B shows 3D binding modes for 20S(OH)D₃, 20S,25(OH)₂D₃, 20S,23S(OH)₂D₃ and 20S,23R(OH)₂D₃ in ligand binding domain of RORγ (cartoon in gray) including a zoomed view. The binding pocket is shown as a light blue meshing area. FIG. 13C shows a 2D interaction map of 20S(OH)D₃, 20S,25(OH)₂D₃, 20S,23S(OH)₂D₃, and 20S,23R(OH)₂D₃ with RORα. FIG. 13D shows a 2D interaction map of 20S(OH)D₃, 20S,25(OH)₂D₃, 20S,23S(OH)₂D₃, and 20S,23R(OH)₂D₃ with RORγ (image generated with Maestro (v12.4)).

In summary, chemically synthesized 20S(OH)D₃, 20S,23S(OH)₂D₃, 20S,23R(OH)₂D₃, and 20S,25(OH)₂D₃ showed the ability to act on the VDR, AhR, LXR and ROR, consistent with previous reports (Kim T K, et. al., (2012) Mol Cell Endocrinol 361(1-2) 143-52, Slominski A T, et. al., (2017) J Steroid Biochem Mol Biol 173 42-56, Lin Z, et. al., (2018) Sci Rep 8(1) 1478, Slominski A T, et. al., (2014) FASEB J 28(7) 2775-89, Slominski A T, et. al., (2021) Sci Rep 11(1) 8002, Slominski A T, et. al., (2018) Int J Mol Sci 19(10), and Lin Z, et. al., (2017) Sci Rep 7(1) 10193), with some differences as indicated above. These studies provide a background for future exciting studies on the exact nature of these interactions between the secosteroids and their receptors, and the differences between different analogs.

e. Different Vitamin D Compounds have Distinct and Overlapping Effects on Activation of T Cells in Human Peripheral Blood

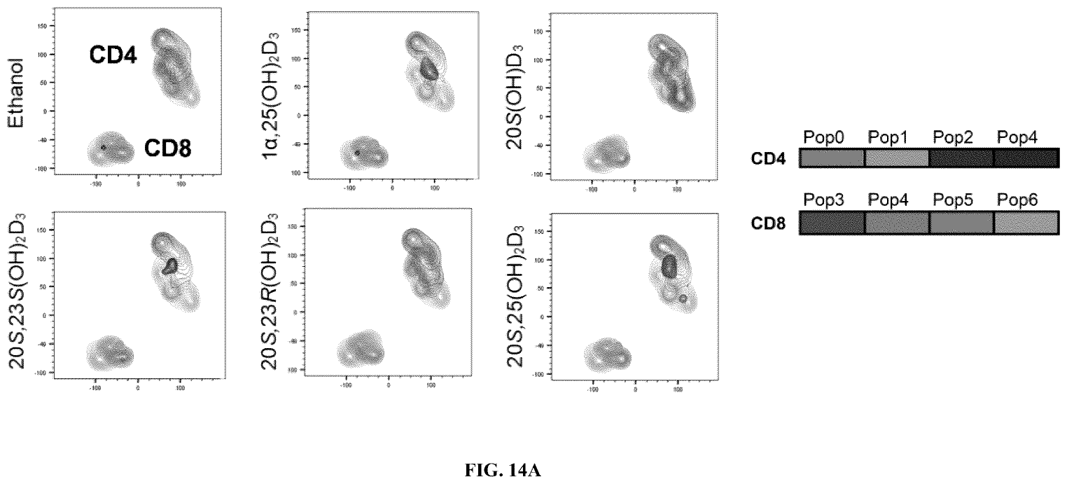

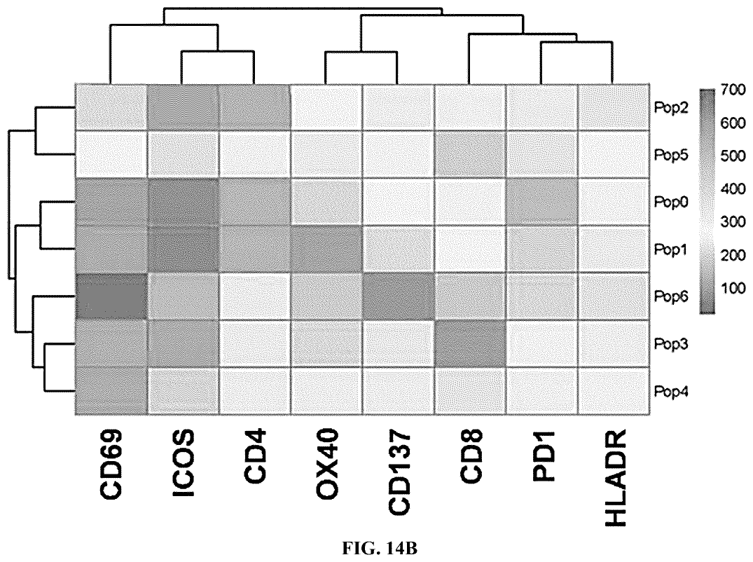

To determine if different vitamin D₃ compounds variably alter activation human CD4 and CD8 T cell, PBMC from healthy individuals were stimulated with anti-CD3 and anti-CD28 to activate T cells in the absence (ethanol) or presence of 20S(OH)D₃, 20S,23S(OH)₂D₃, 20S,23R(OH)₂D₃, 20S,25(OH)₂D₃, or 1α,25(OH)₂D₃ for 24 h. The cells were stained for the expression of lineage markers (CD4, CD8, CD14, CD19) and activation induced markers (AIM; CD69, ICOS, OX40, CD137, PD1 and HLADR) (Boppana S, et. al., (2021) PLoS Pathog 17(7) e1009761, Grifoni A, et. al., (2021) Cell Host Microbe 29(7) 1076-1092). The data was analysed using an unsupervised algorithm-based approach combining TriMAP followed by self-organizing map (FlowSOM) cluster overlay as described in above (FIG. 14A and FIG. 14B). Unsupervised algorithm-based analyses of multi-dimensional data enables unbiased identification of discovery as well as changes in cell populations (Anchang B, et. al., (2016) Nat Protoc 11(7) 1264-79, Palit S, et. al., (2019) Front Immunol 10 1515). Based on the expression levels of CD4, CD8 and the AIM, four populations each of CD4 and CD8 T cells were identified. Populations 2 and 4 within CD4 T cells were differentially altered by the vitamin D₃ compounds (FIG. 14A). For example, compared to ethanol treated cells, 1α,25(OH)₂D₃, 20S,23S(OH)₂D₃, and 20S,25(OH)₂D₃ treated cells contained fewer cells that down modulated CD4 and expressed increased levels of CD69 and ICOS (population 4, FIG. 14B). In contrast, 20S(OH)D₃ treatment increased the proportion of cells with population 4 phenotype and 20S,23(OH)₂D₃ had no effect on this population. Within CD8 T cells population 6 (cells that express ICOS) were differentially modulated by the vitamin D₃ compounds compared to ethanol treated cells (FIG. 14A and FIG. 14B). The unbiased algorithm-based computational analysis, which used statistical “R code” reveals with accuracy that the different vitamin D compounds have overlapping and distinct effects in their ability to modulate T cell activation.

Referring to FIG. 14A and FIG. 14B, shows differential and overlapping effects of secosteroids on activation of CD4 and CD8 T cells from human peripheral blood. FIG. 14A shows FlowSOM analysis shows that the different vitamin D₃ secosteroids variably alter CD4 and CD8 T cell activation. FIG. 14B shows heat map of FlowSOM clusters representing the level of expression of activation induced markers (AIM) on CD4 and CD8 T cell population shown in FIG. 14A. T cells in peripheral blood from two healthy individuals were activated with anti-CD3 and anti-CD28 in the absence (ethanol) or presence of 10⁻⁷ M of each of the vitamin D₃ secosteroids for 24 h. The cells were stained for lineage markers (CD4, CD8, CD19, CD14) and a panel of activation induced markers (CD69, ICOS, OX40, CD137, PD1, HLADR) and expression data were collected using a flow cytometer. The gating strategy was live, single cell followed by CD4 and CD8 T cell populations. The data file output following CD4 and CD8 T cell gating from each individual were concatenated into one file and subjected first to the dimensionality reduction algorithm TriMAP, which clusters cell populations based on relatedness using three points. The data shows that CD4 and CD8 T cell populations cluster independently as expected. Following TriMAP analysis, clustering was performed with Self-Organizing Maps (FlowSOM) and the identified clusters were overlayed on to TriMAP clusters. Pop0 to Pop6 are subpopulations of cells based on expression levels of all markers shown in the heat map. Unbiased analyses using TriMap dimensionality reduction clustering followed by FlowSOM “R” algorithm-based clustering defined these subpopulations based on the expression levels of the markers. The algorithm-based clustering analysis takes into consideration the expression levels CD4, CD8 and all AIMs. The expression levels for each marker in specific FlowSOM populations is output as a heat map B. The heat map reflects the levels of expression of each marker within each subpopulation (Pop0-Pop6) and is not by itself reflective of response to a vitamin D₃ analogue.

### 6. Discussion

Efficient chemical synthetic routes for 20S(OH)D₃, 20S,23S(OH)₂D₃, 20S,23R(OH)₂D₃, and 20S,25(OH)₂D₃ were established. Convergent synthesis was used for the preparation of these target compounds where the key stage was a Wittig-Horner coupling of the corresponding Grundmann ketone (CD-ring fragment) with A-ring phosphine oxide. The resulting secosteroids were substrates for 1α-hydroxylation by CYP27B1 with the following selectivity: 20S,25(OH)₂D₃>>20S,23S(OH)₂D₃>20S(OH)D₃=20S,23R(OH)₂D₃. Functional assays and molecular modeling have shown that they act on the VDR, AhR, LXR and RORα and γ with some notable differences. 20S,25(OH)₂D₃ showed stronger stimulation of CYP24A1, a gene downstream of the VDR, than the other secosteroids which may be explained by its more efficient hydroxylation by CYP27B1. On the other hand, there was an interesting change from agonistic activity of 20S,23S(OH)₂D₃ on the AhR to inverse agonism by its 23R-isomer (20S,23R(OH)₂D₃). In addition, 20S,23R(OH)₂D₃ was less efficient in activation of LXRβ than 20S,23S(OH)₂D₃. This work provides the background for future studies on the different modes of nuclear receptor signaling by hydroxy-D₃ derivatives under study. Interestingly, the major phenotypic effects caused by the synthesized secosteroids were very similar and included inhibition of cell proliferation in skin cells and induction of keratinocyte differentiation. Finally, these secosteroids showed immunomodulatory effects on human PBMC stimulated by coactivating peptides with net effects of downregulation of proinflammatory responses. Interestingly, the pattern of expression of lymphocytic markers was both overlapping and differential indicating a difference in mechanism of action depending on the position of the OH group and its configuration in the D₃ side chain. These secosteroids showed differential effects in comparison to classical 1α,25(OH)₂D₃. This work underpins new possibilities for exciting studies on the anti-inflammatory actions of the D₃ hydroxy derivatives in order to establish efficient methods to treat inflammatory and autoimmune diseases.

In summary, new efficient routes of chemical synthesis for 20S(OH)D₃, 20S,23S(OH)₂D₃, 20S,23R(OH)₂D₃, and 20S,25(OH)₂D₃ were designed allowing their production for in vivo testing and further clarification of different mechanisms of action as well as future preclinical studies on autoimmune disorders including lupus erythematosus, rheumatoid arthritis, inflammatory bowel disease, psoriasis and multiple sclerosis.

### K. REFERENCES

Holick M F, MacLaughlin J A, Doppelt S H, Regulation of cutaneous previtamin D₃ photosynthesis in man: skin pigment is not an essential regulator, Science 211(4482) (1981) 590-3.
Holick M F, Tian X Q, Allen M, Evolutionary importance for the membrane enhancement of the production of vitamin D₃ in the skin of poikilothermic animals, Proc Natl Acad Sci USA 92(8) (1995) 3124-6.
Bikle D D, Vitamin D: an ancient hormone, Exp Dermatol 20(1) (2011) 7-13.
Bikle D D, Vitamin D: newly discovered actions require reconsideration of physiologic requirements, Trends Endocrinol Metab 21(6) (2010) 375-84.
Holick M F, Vitamin D: A millenium perspective, J Cell Biochem 88(2) (2003) 296-307.
Jenkinson C, The vitamin D metabolome: An update on analysis and function, Cell Biochem Funct (2019).
Tuckey R C, Cheng C Y S, Slominski A T, The serum vitamin D metabolome: What we know and what is still to discover, J Steroid Biochem Mol Biol 186 (2019) 4-21.
Bikle D D, Vitamin D receptor, UVR, and skin cancer: a potential protective mechanism, J Invest Dermatol 128(10) (2008) 2357-61.
Bikle D D, Vitamin D metabolism and function in the skin, Mol Cell Endocrinol 347(1-2) (2011) 80-9.
Holick M F, Vitamin D deficiency, N Engl J Med 357(3) (2007) 266-81.
Plum L A, DeLuca H F, Vitamin D, disease and therapeutic opportunities, Nature reviews. Drug discovery 9(12) (2010) 941-55.
Bikle D D, Vitamin D: Newer Concepts of Its Metabolism and Function at the Basic and Clinical Level, J Endocr Soc 4(2) (2020) bvz038.
Carlberg C, Vitamin D Genomics: From In Vitro to In Vivo, Front Endocrinol (Lausanne) 9 (2018) 250.
Zmijewski M A, Carlberg C, Vitamin D receptor(s): In the nucleus but also at membranes?, Exp Dermatol (2020).
Bouillon R, Marcocci C, Carmeliet G, Bikle D, White J H, Dawson-Hughes B, Lips P, Munns C F, Lazaretti-Castro M, Giustina A, Bilezikian J, Skeletal and Extraskeletal Actions of Vitamin D: Current Evidence and Outstanding Questions, Endocrine Reviews 40(4) (2018) 1109-1151.
Slominski A T, Brozyna A A, Zmijewski M A, Jozwicki W, Jetten A M, Mason R S, Tuckey R C, Elmets C A, Vitamin D signaling and melanoma: role of vitamin D and its receptors in melanoma progression and management, Lab Invest 97(6) (2017) 706-724.
Hewison M, An update on vitamin D and human immunity, Clinical Endocrinology 76(3) (2012) 315-325.
Suaini N H A, Zhang Y, Vuillermin P J, Allen K J, Harrison L C, Immune Modulation by Vitamin D and Its Relevance to Food Allergy, Nutrients 7(8) (2015) 6088-6108.
Bikle D D, Vitamin D and the skin: Physiology and pathophysiology, Rev Endocr Metab Disord 13(1) (2012) 3-19.
Reichrath J, Saternus R, Vogt T, Endocrine actions of vitamin D in skin: Relevance for photocarcinogenesis of non-melanoma skin cancer, and beyond, Mol Cell Endocrinol 453 (2017) 96-102.
Grant W B, Roles of Solar UVB and Vitamin D in Reducing Cancer Risk and Increasing Survival, Anticancer research 36(3) (2016) 1357-70.
Chang S H, Chung Y, Dong C, Vitamin D Suppresses Th17 Cytokine Production by Inducing C/EBP Homologous Protein (CHOP) Expression, Journal of Biological Chemistry 285(50) (2010) 38751-38755.
Szodoray P, Nakken B, Gaal J, Jonsson R, Szegedi A, Zold E, Szegedi G, Brun J G, Gesztelyi R, Zeher M, Bodolay E, The complex role of vitamin D in autoimmune diseases, Scandinavian journal of immunology 68(3) (2008) 261-9.
Daniel C, Sartory N A, Zahn N, Radeke H H, Stein J M, Immune Modulatory Treatment of Trinitrobenzene Sulfonic Acid Colitis with Calcitriol Is Associated with a Change of a T Helper (Th) 1/Th17 to a Th2 and Regulatory T Cell Profile, Journal of Pharmacology and Experimental Therapeutics 324(1) (2008) 23-33
Wöbke T K, Sorg B L, Steinhilber D, Vitamin D in inflammatory diseases, Frontiers in Physiology 5 (2014) 244.
Chun R F, Liu P T, Modlin R L, Adams J S, Hewison M, Impact of vitamin D on immune function: lessons learned from genome-wide analysis, Frontiers in Physiology 5 (2014) 151.
Rossini M, Maddali Bongi S, La Montagna G, Minisola G, Malavolta N, Bernini L, Cacace E, Sinigaglia L, Di Munno O, Adami S, Vitamin D deficiency in rheumatoid arthritis: prevalence, determinants and associations with disease activity and disability, Arthritis research & therapy 12(6) (2010) R216.
Palm N W, Rosenstein R K, Medzhitov R, Allergic host defences, Nature 484(7395) (2012) 465-472.
Ikeda U, Wakita D, Ohkuri T, Chamoto K, Kitamura H, Iwakura Y, Nishimura T, 1α,25-Dihydroxyvitamin D₃ and all-trans retinoic acid synergistically inhibit the differentiation and expansion of Th17 cells, Immunology Letters 134(1) (2010) 7-16.
Mayne C G, Spanier J A, Relland L M, Williams C B, Hayes C E, 1,25-Dihydroxyvitamin D₃ acts directly on the T lymphocyte vitamin D receptor to inhibit experimental autoimmune encephalomyelitis, European Journal of Immunology 41(3) (2011) 822-832.
Hartmann B, Riedel R, Jors K, Loddenkemper C, Steinmeyer A, Zugel U, Babina M, Radbruch A, Worm M, Vitamin D Receptor Activation Improves Allergen-Triggered Eczema in Mice, J Invest Dermatol 132(2) (2012) 330-336.
Slominski A T, Janjetovic Z, Fuller B E, Zmijewski M A, Tuckey R C, Nguyen M N, Sweatman T, Li W, Zjawiony J, Miller D, Chen T C, Lozanski G, Holick M F, Products of vitamin D₃ or 7-dehydrocholesterol metabolism by cytochrome P450scc show anti-leukemia effects, having low or absent calcemic activity, PloS one 5(3) (2010) e9907.
Wang J, Slominski A, Tuckey R C, Janjetovic Z, Kulkarni A, Chen J, Postlethwaite A E, Miller D, Li W, 20-hydroxyvitamin D₃ inhibits proliferation of cancer cells with high efficacy while being non-toxic, Anticancer research 32(3) (2012) 739-46.
Biankin A V, Waddell N, Kassahn K S, Gingras M-C, Muthuswamy L B, Johns A L, Miller D K, Wilson P J, Patch A-M, Wu J, Chang D K, Cowley M J, Gardiner B B, Song S, Harliwong I, Idrisoglu S, Nourse C, Nourbakhsh E, Manning S, Wani S, Gongora M, Pajic M, Scarlett C J, Gill A J, Pinho A V, Rooman I, Anderson M, Holmes O, Leonard C, Taylor D, Wood S, Xu Q, Nones K, Lynn Fink J, Christ A, Bruxner T, Cloonan N, Kolle G, Newell F, Pinese M, Scott Mead R, Humphris J L, Kaplan W, Jones M D, Colvin E K, Nagrial A M, Humphrey E S, Chou A, Chin V T, Chantrill L A, Mawson A, Samra J S, Kench J G, Lovell J A, Daly R J, Merrett N D, Toon C, Epari K, Nguyen N Q, Barbour A, Zeps N, Kakkar N, Zhao F, Qing Wu Y, Wang M, Muzny D M, Fisher W E, Charles Brunicardi F, Hodges S E, Reid J G, Drummond J, Chang K, Han Y, Lewis L R, Dinh H, Buhay C J, Beck T, Timms L, Sam M, Begley K, Brown A, Pai D, Panchal A, Buchner N, De Borja R, Denroche R E, Yung C K, Serra S, Onetto N, Mukhopadhyay D, Tsao M-S, Shaw P A, Petersen G M, Gallinger S, Hruban R H, Maitra A, Iacobuzio-Donahue C A, Schulick R D, Wolfgang C L, Morgan R A, Lawlor R T, Capelli P, Corbo V, Scardoni M, Tortora G, Tempero M A, Mann K M, Jenkins N A, Perez-Mancera P A, Adams D J, Largaespada D A, Wessels L F A, Rust A G, Stein L D, Tuveson D A, Copeland N G, Musgrove E A, Scarpa A, Eshleman J R, Hudson T J, Sutherland R L, Wheeler D A, Pearson J V, McPherson J D, Gibbs R A, Grimmond S M, Pancreatic cancer genomes reveal aberrations in axon guidance pathway genes, Nature 491(7424) (2012) 399-405.
Slominski A T, Li W, Kim T K, Semak I, Wang J, Zjawiony J K, Tuckey R C, Novel activities of CYP11A1 and their potential physiological significance, J Steroid Biochem Mol Biol 151 (2015) 25-37.
Tuckey R C, Li W, Zjawiony J K, Zmijewski M A, Nguyen M N, Sweatman T, Miller D, Slominski A, Pathways and products for the metabolism of vitamin D₃ by cytochrome P450scc, The FEBS journal 275(10) (2008) 2585-96.
Slominski R M, Raman C, Elmets C, Jetten A M, Slominski A, Tuckey R C, The significance of CYP11A¹ expression in skin physiology and pathology, Mol Cell Endocrinol Available online 12 Mar. 2021, 111238 (2021) 111238.
Slominski A T, Kim T K, Shehabi H Z, Semak I, Tang E K, Nguyen M N, Benson H A, Korik E, Janjetovic Z, Chen J, Yates C R, Postlethwaite A, Li W, Tuckey R C, In vivo evidence for a novel pathway of vitamin D₃ metabolism initiated by P450scc and modified by CYP27B1, FASEB J 26(9) (2012) 3901-15.
Slominski A T, Kim T K, Li W, Tuckey R C, Classical and non-classical metabolic transformation of vitamin D in dermal fibroblasts, Exp Dermatol 25(3) (2016) 231-2.
Slominski A T, Kim T K, Li W, Postlethwaite A, Tieu E W, Tang E K, Tuckey R C, Detection of novel CYP11A1-derived secosteroids in the human epidermis and serum and pig adrenal gland, Sci Rep 5 (2015) 14875.
Jenkinson C, Desai R, Slominski A T, Tuckey R C, Hewison M, Handelsman D J, Simultaneous measurement of 13 circulating vitamin D₃ and D₂ mono and dihydroxy metabolites using liquid chromatography mass spectrometry, Clin Chem Lab Med (2021).
Kim T K, Atigadda V, Brzeminski P, Fabisiak A, Tang E K Y, Tuckey R C, Slominski A T, Detection of 7-Dehydrocholesterol and Vitamin D₃ Derivatives in Honey, Molecules 25(11) (2020).
Slominski A T, Chaiprasongsuk A, Janjetovic Z, Kim T K, Stefan J, Slominski R M, Hanumanthu V S, Raman C, Qayyum S, Song Y, Song Y, Panich U, Crossman D K, Athar M, Holick M F, Jetten A M, Zmijewski M A, Zmijewski J, Tuckey R C, Photoprotective Properties of Vitamin D and Lumisterol Hydroxyderivatives, Cell Biochem Biophys 78(2) (2020) 165-180.
Janjetovic Z, Postlethwaite A, Kang H S, Kim T K, Tuckey R C, Crossman D K, Qayyum S, Jetten A M, Slominski A T, Antifibrogenic Activities of CYP11A1-derived Vitamin D₃-hydroxyderivatives Are Dependent on RORgamma, Endocrinology 162(1) (2021).
Chaiprasongsuk A, Janjetovic Z, Kim T K, Tuckey R C, Li W, Raman C, Panich U, Slominski A T, CYP11A1-derived vitamin D₃ products protect against UVB-induced inflammation and promote keratinocytes differentiation, Free Radic Biol Med 155 (2020) 87-98.
Chaiprasongsuk A, Janjetovic Z, Kim T K, Jarrett S G, D'Orazio J A, Holick M F, Tang E K Y, Tuckey R C, Panich U, Li W, Slominski A T, Protective effects of novel derivatives of vitamin D₃ and lumisterol against UVB-induced damage in human keratinocytes involve activation of Nrf2 and p53 defense mechanisms, Redox Biol 24 (2019) 101206.
Slominski A T, Brozyna A A, Zmijewski M A, Janjetovic Z, Kim T K, Slominski R M, Tuckey R C, Mason R S, Jetten A M, Guroji P, Reichrath J, Elmets C, Athar M, The role of classical and novel forms of vitamin D in the pathogenesis and progression of non-melanoma skin cancers, Advances in Experimental Medicine and Biology in press (2020).
Slominski A T, Kim T K, Li W, Yi A K, Postlethwaite A, Tuckey R C, The role of CYP11A1 in the production of vitamin D metabolites and their role in the regulation of epidermal functions, J Steroid Biochem Mol Biol 144PA (2014) 28-39.
Janjetovic Z, Tuckey R C, Nguyen M N, Thorpe E M Jr., Slominski A T, 20,23-dihydroxyvitamin D₃, novel P450scc product, stimulates differentiation and inhibits proliferation and NF-kappaB activity in human keratinocytes, Journal of cellular physiology 223(1) (2010) 36-48.
Slominski A, Janjetovic Z, Tuckey R C, Nguyen M N, Bhattacharya K G, Wang J, Li W, Jiao Y, Gu W, Brown M, Postlethwaite A E, 20S-hydroxyvitamin D₃, noncalcemic product of CYP11A1 action on vitamin D₃, exhibits potent antifibrogenic activity in vivo, The Journal of clinical endocrinology and metabolism 98(2) (2013) E298-303.
Slominski A T, Kim T K, Li W, Yi A K, Postlethwaite A, Tuckey R C, The role of CYP11A1 in the production of vitamin D metabolites and their role in the regulation of epidermal functions, J Steroid Biochem Mol Biol 144 Pt A (2014) 28-39.
Podgorska E, Kim T K, Janjetovic Z, Urbanska K, Tuckey R C, Bae S, Slominski A T, Knocking out the Vitamin D Receptor Enhances Malignancy and Decreases Responsiveness to Vitamin D₃ Hydroxyderivatives in Human Melanoma Cells, Cancers (Basel) 13(13) (2021).
Tieu E W, Tang E K, Chen J, Li W, Nguyen M N, Janjetovic Z, Slominski A, Tuckey R C, Rat CYP24A1 acts on 20-hydroxyvitamin D₃ producing hydroxylated products with increased biological activity, Biochem Pharmacol 84(12) (2012) 1696-704.
Tongkao-On W, Carter S, Reeve V E, Dixon K M, Gordon-Thomson C, Halliday G M, Tuckey R C, Mason R S, CYP11A1 in skin: an alternative route to photoprotection by vitamin D compounds, J Steroid Biochem Mol Biol 148 (2015) 72-8.
Skobowiat C, Oak A S, Kim T K, Yang C H, Pfeffer L M, Tuckey R C, Slominski A T, Noncalcemic 20-hydroxyvitamin D₃ inhibits human melanoma growth in in vitro and in vivo models, Oncotarget 8(6) (2017) 9823-9834.
Chen J, Wang J, Kim T K, Tieu E W, Tang E K, Lin Z, Kovacic D, Miller D D, Postlethwaite A, Tuckey R C, Slominski A T, Li W, Novel vitamin D analogs as potential therapeutics: metabolism, toxicity profiling, and antiproliferative activity, Anticancer research 34(5) (2014) 2153-63.
Kim T K, Wang J, Janjetovic Z, Chen J, Tuckey R C, Nguyen M N, Tang E K, Miller D, Li W, Slominski A T, Correlation between secosteroid-induced vitamin D receptor activity in melanoma cells and computer-modeled receptor binding strength, Mol Cell Endocrinol 361(1-2) (2012) 143-52.
Slominski A T, Kim T K, Hobrath J V, Oak A S W, Tang E K Y, Tieu E W, Li W, Tuckey R C, Jetten A M, Endogenously produced nonclassical vitamin D hydroxy-metabolites act as “biased” agonists on VDR and inverse agonists on RORalpha and RORgamma, J Steroid Biochem Mol Biol 173 (2017) 42-56.
Lin Z, Marepally S R, Goh E S Y, Cheng C Y S, Janjetovic Z, Kim T K, Miller D D, Postlethwaite A E, Slominski A T, Tuckey R C, Peluso-Iltis C, Rochel N, Li W, Investigation of 20S-hydroxyvitamin D₃ analogs and their 1alpha-OH derivatives as potent vitamin D receptor agonists with anti-inflammatory activities, Sci Rep 8(1) (2018) 1478.
Slominski A T, Kim T K, Takeda Y, Janjetovic Z, Brozyna A A, Skobowiat C, Wang J, Postlethwaite A, Li W, Tuckey R C, Jetten A M, RORalpha and ROR gamma are expressed in human skin and serve as receptors for endogenously produced noncalcemic 20-hydroxy- and 20,23-dihydroxyvitamin D, FASEB J 28(7) (2014) 2775-89.
Slominski A T, Kim T K, Qayyum S, Song Y, Janjetovic Z, Oak A S W, Slominski R M, Raman C, Stefan J, Mier-Aguilar C A, Atigadda V, Crossman D K, Golub A, Bilokin Y, Tang E K Y, Chen J Y, Tuckey R C, Jetten A M, Song Y, Vitamin D and lumisterol derivatives can act on liver X receptors (LXRs), Sci Rep 11(1) (2021) 8002.
Slominski A T, Kim T K, Janjetovic Z, Brozyna A A, Zmijewski M A, Xu H, Sutter T R, Tuckey R C, Jetten A M, Crossman D K, Differential and Overlapping Effects of 20,23(OH)₂D₃ and 1,25(OH)₂D₃ on Gene Expression in Human Epidermal Keratinocytes: Identification of AhR as an Alternative Receptor for 20,23(OH)₂D₃, Int J Mol Sci 19(10) (2018).
Li W, Chen J, Janjetovic Z, Kim T K, Sweatman T, Lu Y, Zjawiony J, Tuckey R C, Miller D, Slominski A, Chemical synthesis of 20S-hydroxyvitamin D₃, which shows antiproliferative activity, Steroids 75(12) (2010) 926-35.
Sibilska-Kaminski I K, Sicinski R R, Plum L A, DeLuca H F, Synthesis and Biological Activity of 2,22-Dimethylene Analogues of 19-Norcalcitriol and Related Compounds, J Med Chem 63(13) (2020) 7355-7368.
Wang Q, Lin Z, Kim T K, Slominski A T, Miller D D, Li W, Total synthesis of biologically active 20S-hydroxyvitamin D₃, Steroids 104 (2015) 153-62.
Tang E K, Chen J, Janjetovic Z, Tieu E W, Slominski A T, Li W, Tuckey R C, Hydroxylation of CYP11A1-derived products of vitamin D₃ metabolism by human and mouse CYP27B1, Drug metabolism and disposition: the biological fate of chemicals 41(5) (2013) 1112-24.
Lin Z, Marepally S R, Ma D, Kim T K, Oak A S, Myers L K, Tuckey R C, Slominski A T, Miller D D, Li W, Synthesis and Biological Evaluation of Vitamin D₃ Metabolite 20S,23S-Dihydroxyvitamin D₃ and Its 23R Epimer, J Med Chem 59(10) (2016) 5102-8.
Tang E K, Li W, Janjetovic Z, Nguyen M N, Wang Z, Slominski A, Tuckey R C, Purified mouse CYP27B1 can hydroxylate 20,23-dihydroxyvitamin D₃, producing 1alpha,20,23-trihydroxyvitamin D₃, which has altered biological activity, Drug metabolism and disposition: the biological fate of chemicals 38(9) (2010) 1553-9.
Zbytek B, Janjetovic Z, Tuckey R C, Zmijewski M A, Sweatman T W, Jones E, Nguyen M N, Slominski A T, 20-Hydroxyvitamin D₃, a product of vitamin D₃ hydroxylation by cytochrome P450scc, stimulates keratinocyte differentiation, J Invest Dermatol 128(9) (2008) 2271-80.
Janjetovic Z, Zmijewski M A, Tuckey R C, DeLeon D A, Nguyen M N, Pfeffer L M, Slominski A T, 20-Hydroxycholecalciferol, product of vitamin D₃ hydroxylation by P450scc, decreases N F-kappaB activity by increasing IkappaB alpha levels in human keratinocytes, PloS one 4(6) (2009) e5988.
Slominski A T, Janjetovic Z, Kim T K, Wright A C, Grese L N, Riney S J, Nguyen M N, Tuckey R C, Novel vitamin D hydroxyderivatives inhibit melanoma growth and show differential effects on normal melanocytes, Anticancer research 32(9) (2012) 3733-42.
Brozyna A A, Kim T K, Zablocka M, Jozwicki W, Yue J, Tuckey R C, Jetten A M, Slominski A T, Association among Vitamin D, Retinoic Acid-Related Orphan Receptors, and Vitamin D Hydroxyderivatives in Ovarian Cancer, Nutrients 12(11) (2020).
Slominski A T, Janjetovic Z, Kim T K, Wasilewski P, Rosas S, Hanna S, Sayre R M, Dowdy J C, Li W, Tuckey R C, Novel non-calcemic secosteroids that are produced by human epidermal keratinocytes protect against solar radiation, J Steroid Biochem Mol Biol 148 (2015) 52-63.
Slominski A T, Brozyna A A, Skobowiat C, Zmijewski M A, Kim T K, Janjetovic Z, Oak A S, Jozwicki W, Jetten A M, Mason R S, Elmets C, Li W, Hoffman R M, Tuckey R C, On the role of classical and novel forms of vitamin D in melanoma progression and management, J Steroid Biochem Mol Biol 177 (2018) 159-170.
Lin Z, Chen H, Belorusova A Y, Bollinger J C, Tang E K Y, Janjetovic Z, Kim T K, Wu Z, Miller D D, Slominski A T, Postlethwaite A E, Tuckey R C, Rochel N, Li W, 1alpha,20S-Dihydroxyvitamin D₃ Interacts with Vitamin D Receptor: Crystal Structure and Route of Chemical Synthesis, Sci Rep 7(1) (2017) 10193.
Boppana S, Qin K, Files J K, Russell R M, Stoltz R, Bibollet-Ruche F, Bansal A, Erdmann N, Hahn B H, Goepfert P A, SARS-CoV-2-specific circulating T follicular helper cells correlate with neutralizing antibodies and increase during early convalescence, PLoS Pathog 17(7) (2021) e1009761.
Grifoni A, Sidney J, Vita R, Peters B, Crotty S, Weiskopf D, Sette A, SARS-CoV-2 human T cell epitopes: Adaptive immune response against COVID-19, Cell Host Microbe 29(7) (2021) 1076-1092.
Anchang B, Hart T D, Bendall S C, Qiu P, Bjornson Z, Linderman M, Nolan G P, Plevritis S K, Visualization and cellular hierarchy inference of single-cell data using SPADE, Nat Protoc 11(7) (2016) 1264-79.
Palit S, Heuser C, de Almeida G P, Theis F J, Zielinski C E, Meeting the Challenges of High-Dimensional Single-Cell Data Analysis in Immunology, Front Immunol 10 (2019) 1515.
Slominski A T, Kim T K, Janjetovic Z, Tuckey R C, Bieniek R, Yue J, Li W, Chen J, Nguyen M N, Tang E K, Miller D, Chen T C, Holick M, 20-Hydroxyvitamin D₂ is a noncalcemic analog of vitamin D with potent antiproliferative and prodifferentiation activities in normal and malignant cells, Am J Physiol Cell Physiol 300(3) (2011) C526-41.
Slominski A T, Kim T K, Hobrath J V, Janjetovic Z, Oak A S W, Postlethwaite A, Lin Z, Li W, Takeda Y, Jetten A M, Tuckey R C, Characterization of a new pathway that activates lumisterol in vivo to biologically active hydroxylumisterols, Sci Rep 7(1) (2017) 11434.
Rochel N, Wurtz J, Mitschler A, Klaholz B, Moras D, The crystal structure of the nuclear receptor for vitamin D bound to its natural ligand, Molecular cell 5(1) (2000) 173-179.
Kallen J, Schlaeppi J-M, Bitsch F, Delhon I, Fournier B, Crystal structure of the human RORα ligand binding domain in complex with cholesterol sulfate at 2.2 Å, Journal of Biological Chemistry 279(14) (2004) 14033-14038
Jin L, Martynowski D, Zheng S, Wada T, Xie W, Li Y, Structural basis for hydroxycholesterols as natural ligands of orphan nuclear receptor RORγ, Molecular endocrinology 24(5) (2010) 923-929.
Matsui Y, Yamaguchi T, Yamazaki T, Yoshida M, Arai M, Terasaka N, Honzumi S, Wakabayashi K, Hayashi S, Nakai D, Discovery and structure-guided optimization of tert-butyl 6-(phenoxymethyl)-3-(trifluoromethyl) benzoates as liver X receptor agonists, Bioorganic & medicinal chemistry letters 25(18) (2015) 3914-3920.
Stachel S J, Zerbinatti C, Rudd M T, Cosden M, Suon S, Nanda K K, Wessner K, DiMuzio J, Maxwell J, Wu Z, Identification and in vivo evaluation of liver X receptor (3-selective agonists for the potential treatment of Alzheimer's disease, Journal of medicinal chemistry 59(7) (2016) 3489-3498.
O'Boyle N M, Banck M, James C A, Morley C, Vandermeersch T, Hutchison G R, Open Babel: An open chemical toolbox, Journal of Cheminformatics 3(1) (2011) 33.
Morris G M, Huey R, Lindstrom W, Sanner M F, Belew R K, Goodsell D S, Olson A J, AutoDock4 and AutoDockTools4: Automated docking with selective receptor flexibility, Journal of computational chemistry 30(16) (2009) 2785-91.
Trott O, Olson A J, AutoDock Vina: Improving the speed and accuracy of docking with a new scoring function, efficient optimization, and multithreading, Journal of Computational Chemistry 31(2) (2010) 455-461
Schrodinger, LLC, The PyMOL Molecular Graphics System, Version 1.8, 2015.
Maestro, Schrödinger, LLC, New York, N Y, 2020.
Janjetovic Z, Nahmias Z P, Hanna S, Jarrett S G, Kim T K, Reiter R J, Slominski A T, Melatonin and its metabolites ameliorate ultraviolet B-induced damage in human epidermal keratinocytes, J Pineal Res 57(1) (2014) 90-102.
Chaiprasongsuk A, Janjetovic Z, Kim T K, Schwartz C J, Tuckey R C, Tang E K Y, Raman C, Panich U, Slominski A T, Hydroxylumisterols, Photoproducts of Pre-Vitamin D₃, Protect Human Keratinocytes against UVB-Induced Damage, Int J Mol Sci 21(24) (2020).
McGuire D J, Rowse A L, Li H, Peng B J, Sestero C M, Cashman K S, De Sarno P, Raman C, CD5 enhances Th17-cell differentiation by regulating IFN-gamma response and RORgammat localization, Eur J Immunol 44(4) (2014) 1137-42.
Sestero C M, McGuire D J, De Sarno P, Brantley E C, Soldevila G, Axtell R C, Raman C, CD5-dependent CK2 activation pathway regulates threshold for T cell anergy, J Immunol 189(6) (2012) 2918-30.
Quintelier K, Couckuyt A, Emmaneel A, Aerts J, Saeys Y, Van Gassen S, Analyzing high-dimensional cytometry data using FlowSOM, Nature Protocols 16(8) (2021) 3775-3801.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 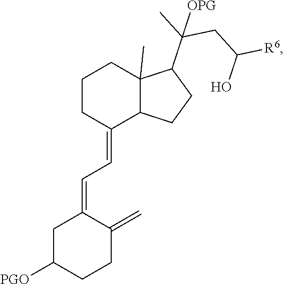
wherein R⁶ is a C1-C8 alkyl; and
wherein each occurrence of PG is independently a silyl protecting group,
or a pharmaceutically acceptable salt thereof.

1. (canceled)

2. The compound of claim 1, wherein R⁶ is a C1-C4 alkyl.

2. (canceled)

3. The compound of claim 1, wherein each occurrence of PG is independently a silyl protecting group selected from trimethylsilyl (TMS), triethylsilyl (TES), tert-butyldimethylsilyl (TBS), tert-butyldiphenylsilyl (TBDPS), and triisopropylsilyl (TIPS).

3. (canceled)

4. The compound of claim 1, wherein each occurrence of PG is independently a silyl protecting group selected from trimethylsilyl (TMS) and tert-butyldimethylsilyl (TBS).

4. (canceled)

5. The compound of claim 1, wherein the compound has a structure represented by a formula: 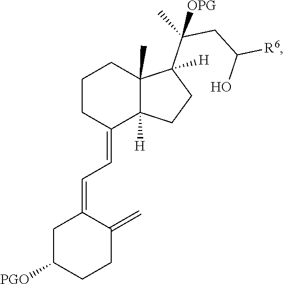
or a pharmaceutically acceptable salt thereof.

5. (canceled)

6. The compound of claim 1, wherein the compound has a structure represented by a formula: 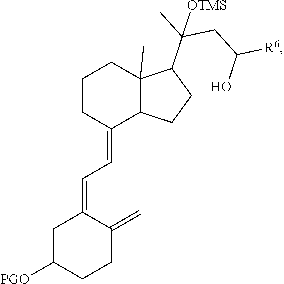
or a pharmaceutically acceptable salt thereof.

6. (canceled)

7. The compound of claim 1, wherein the compound has a structure represented by a formula: 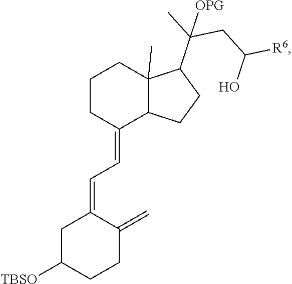
or a pharmaceutically acceptable salt thereof.

7. (canceled)

8. The compound of claim 1, wherein the compound has a structure represented by a formula: 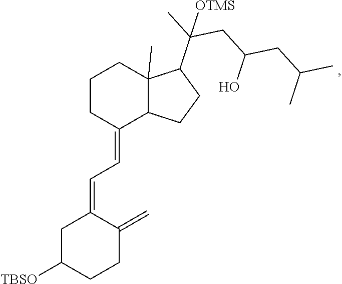
or a pharmaceutically acceptable salt thereof.

8. (canceled)

9. The compound of claim 1, wherein the compound has a structure represented by a formula: 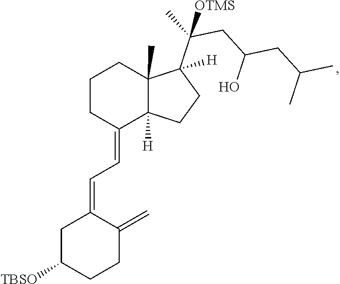
or a pharmaceutically acceptable salt thereof.

9. (canceled)

10. A method of making a compound having a structure represented by a formula: 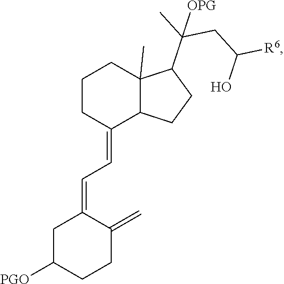
wherein R⁶ is a C1-C8 alkyl; and
wherein each occurrence of PG is independently a silyl protecting group,
or a pharmaceutically acceptable salt thereof, the method comprising reacting an aldehyde having a structure represented by a formula: 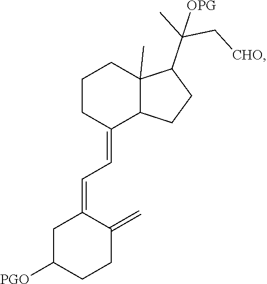
or a pharmaceutically acceptable salt thereof, and a Grignard reagent having a structure represented by a formula:
R⁶—Mg—X,
wherein X is a halide.

10. (canceled)

11. The method of claim 10, wherein reacting is in the presence of an aprotic solvent.

11. (canceled)

12. The method of claim 11, wherein the aprotic solvent is selected from diethyl ether (Et₂O), cyclopentyl methyl ether (CPME), and tetrahydrofuran (THF).

12. (canceled)

13. The method of claim 11, wherein the aprotic solvent is tetrahydrofuran (THF).

13. (canceled)

14. The method of claim 10, wherein reacting is at a temperature of from about 0° C. to about 22° C.

14. (canceled)

15. A method of making a compound having a structure represented by a formula: 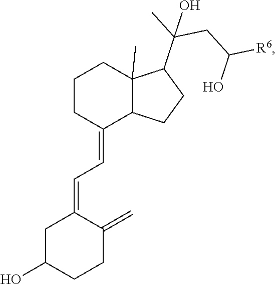
wherein R⁶ is a C1-C8 alkyl,
or a pharmaceutically acceptable salt thereof, the method comprising deprotecting an alcohol having a structure represented by a formula: 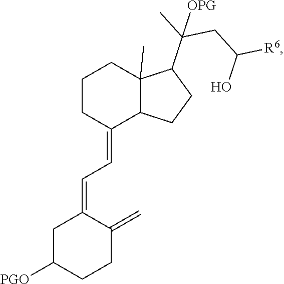
wherein each occurrence of PG is independently a silyl protecting group,
or a pharmaceutically acceptable salt thereof.

15. (canceled)

16. The method of claim 15, wherein deprotecting is in the presence of a deprotecting agent selected from potassium fluoride, cesium fluoride, tetrabutylammonium fluoride (TBAF), tris(dimethylamino)sulfur (trimethylsilyl)difluoride (TASF), tetrabutylammonium triphenyldifluorosilicate (TBAT), tetraphenylbismuth fluoride, cadmium fluoride, acidic aqueous tetrahydrofuran (THF), acidic methanol, and acetic acid.

16. (canceled)

17. The method of claim 15, wherein deprotecting is in the presence of tetrabutylammonium fluoride (TBAF).

17. (canceled)

18. The method of claim 15, wherein deprotecting is in an aprotic solvent.

18. (canceled)

19. The method of claim 18, wherein the aprotic solvent is selected from diethyl ether (Et₂O), cyclopentyl methyl ether (CPME), and tetrahydrofuran (THF).

19. (canceled)

20. The method of claim 18, wherein the aprotic solvent is tetrahydrofuran (THF).

20. (canceled)
